(19) United States
(12) Patent Application Publication
(10) Pub. No.: US 2021/0285965 A1
(43) Pub. Date: Sep. 16, 2021

(54) PROTEOMIC SCREENING FOR DISEASES

(71) Applicant: Seattle Children's Hospital d/b/a Seattle Children's Research Institute, Seattle, WA (US)

(72) Inventors: Sihoun Hahn, Clyde Hill, WA (US); Christopher Collins, Seattle, WA (US); Remwilyn Dayuha, Lynnwood, WA (US); Fan Yi, Shoreline, WA (US)

(73) Assignee: Seattle Children's Hospital d/b/a Seattle Children's Research Institute, Seattle, WA (US)

(21) Appl. No.: 17/191,256

(22) Filed: Mar. 3, 2021

Related U.S. Application Data
(60) Provisional application No. 62/984,744, filed on Mar. 3, 2020.

(51) Int. Cl. G01N 33/68

(52) U.S. Cl. CPC G01N 33/6893; G01N 2800/24; G01N 2570/00

(57) ABSTRACT

Clinical diagnosis and newborn screening for primary immunodeficiencies (PIDDs) is described. The PIDDs include X-linked chronic granulomatous disease (X-CGD), X-linked lymphoproliferative syndrome (XLP1; SH2D1A deficiency), familial hemophagocytic lymphohistiocytosis 2 (FHL2), ataxia telangiectasia (AT), common variable immunodeficiency (CVID; B-cell dysfunctions), severe combined immunodeficiency (SCID), adenosine deaminase (ADA) deficiency, and dedicator of cytokinesis 8 (DOCKS) deficiency. Additionally, cell specific markers for platelets (CD42), natural killer (NK) cells (CD56), and T cells (CD3epsilon and CD3delta) can be used as secondary markers to provide support for diagnosis of disease.

### CROSS-REFERENCE TO RELATED APPLICATION

This application claims priority to U.S. Provisional Patent Application No. 62/984,744 filed Mar. 3, 2020, which is incorporated herein by reference in its entirety as if fully set forth herein.

### STATEMENT REGARDING FEDERALLY SPONSORED RESEARCH OR DEVELOPMENT

This invention was made with government support under HD098180 and AI123135 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 2G85042_ST25.txt. The text file is 201 KB, was created on Mar. 2, 2021, and is being submitted electronically via EFS-Web.

### FIELD OF THE DISCLOSURE

The current disclosure provides clinical diagnosis and newborn screening for primary immunodeficiency disorders (PIDDs). The PIDDs include X-linked chronic granulomatous disease (X-CGD), X-linked lymphoproliferative syndrome (XLP1; SH2D1A deficiency), familial hemophagocytic lymphohistiocytosis 2 (FHL2), ataxia telangiectasia (AT), common variable immunodeficiency (CVID; B-cell dysfunctions), severe combined immunodeficiency (SCID), adenosine deaminase (ADA) deficiency, and dedicator of cytokinesis 8 (DOCKS) deficiency. The disclosed assays can detect these disorders from dried blood spots already routinely collected at the time of birth, among other sources and time points. Additionally, cell specific markers for platelets (CD42), natural killer (NK) cells (CD56), and T cells (CD3epsilon and CD3delta) can be used as secondary markers to provide support for diagnosis of disease. Early detection of these disorders will greatly improve patient outcome as each of them can be fatal once symptoms emerge.

### BACKGROUND OF THE DISCLOSURE

There are a number of diseases with effective treatments available. However, for a number of these diseases, once symptoms emerge, the disease is already fatal or has led to irreversible damage. Examples of such diseases include primary immunodeficiency disorders (PIDDs).

Primary Immunodeficiency Disorders (PIDD), also referred to as inborn errors of immunity (1E1), are a group of more than 416 rare genetic disorders in which components of the immune system are missing or improperly functioning. Examples of PIDD X-linked chronic granulomatous disease (X-CGD), X-linked lymphoproliferative syndrome (XLP1; SH2D1A deficiency), familial hemophagocytic lymphohistiocytosis 2 (FHL2), ataxia telangiectasia, common variable immunodeficiency (CVID; B-cell dysfunctions), severe combined immunodeficiency (SCID), adenosine deaminase (ADA) deficiency, dedicator of cytokinesis 8 (DOCKS) deficiency. Early detection of PIDDs is extremely important in controlling and preventing potentially life-threatening infections and chronic sequelae.

The treatment of PIDDs would be significantly enhanced if diagnosis could be made before clinical symptoms emerge. Newborn screening (NBS) is a standard public preventive mandatory screening test for the 4 million babies born every year in the U.S. NBS usually involves a blood test performed 24 to 48 hours after birth. The screening uses a few drops of blood from a newborn's heel deposited on filter paper. The paper containing dried blood spots (DBS) can be stored until the tests are conducted.

To conduct NBS assessments, punches of dried blood are taken from the DBS and laboratory tests are performed to detect the presence or absence of specific substances within the blood (called markers or biomarkers) that are indicative of disorders not apparent at birth but that cause serious health problems later in life. Though the disorders screened vary from state to state, most states screen for phenylketonuria, primary congenital hypothyroidism, cystic fibrosis, and sickle cell disease. NBS has proven to be highly effective at improving patient outcomes and avoiding long-term disability in affected individuals, while at the same time reducing healthcare costs.

SCID has recently been added to NBS panels. Unfortunately, for most of the other life-threatening but treatable “non-SCID” immunodeficiencies, there are currently no broad-based, cost-effective screening methods available.

One reason that many disorders are not screened even though it would be beneficial is that laboratory tests have been unable to reliably measure markers associated with the disorders. One reason for this inability is that the disorders are associated with markers that are either extremely low in concentrations or not specific to the target disorders. In this scenario, it can be difficult to reliably establish the correlation between the level of the markers and the status of the patients.

Peptide immunoaffinity enrichment coupled to selected reaction monitoring mass spectrometry (immuno-SRM) is a method that enables precise quantification of low abundance markers. Utilization of immuno-SRM generally involves the following steps: (i) selection of target proteins that are indicative of the presence or absence of a disorder; (ii) treatment of a biological sample that would include the target protein, if present, with enzymes to digest all proteins in the biological sample into smaller fragments called peptides; (iii) enrichment for selected peptide markers derived from the target protein and (iv) analysis and quantification of the enriched peptides of interest in a mass spectrometer.

Recently, Jung et al., (J. Proteome Res. 2017; 16: 862-871) described use of immuno-SRM to quantify a protein marker, ATP7B, associated with Wilson Disease from DBS. This was the first demonstration that immuno-SRM could be used to reliably detect a low-abundant marker from stored DBS, opening the possibility of using the approach to screen for a wider panel of disorders.

Signature peptide markers and antibodies that bind to them have been developed to diagnose SCID, WAS, XLA, cystinosis, and WD by immuno-SRM (PCT/US2019/054856).

However, many aspects of such an assay depend on the disorder being diagnosed, the biomarkers available for each disorder, the ability to develop molecular entities that can enrich for peptides of interest, and the behavior of each peptide of interest in the mass spectrometer. All of these aspects and more require careful consideration and experimentation to achieve an assay that can reliably detect disorders. In particular embodiments, diagnosis of disorders is conducted with an NBS panel using DBS before clinical symptoms emerge.

### SUMMARY OF THE DISCLOSURE

The current disclosure describes development of multiplexed assays that can be used to screen subjects (e.g., newborns) for X-linked chronic granulomatous disease (X-CGD), X-linked lymphoproliferative syndrome (XLP1; SH2D1A deficiency), familial hemophagocytic lymphohistiocytosis 2 (FHL2), ataxia telangiectasia (AT), common variable immunodeficiency (CVID; B-cell dysfunctions), severe combined immunodeficiency (SCID), adenosine deaminase (ADA) deficiency, and dedicator of cytokinesis 8 (DOCKS) deficiency. In particular embodiments, the methods include identifying individuals with X-linked or IL2RG deficient SCID. The assays can significantly improve the outcome for affected individuals by reliably diagnosing these disorders before devastating and often fatal clinical symptoms emerge. In particular embodiments, cell specific markers for platelets (CD42), natural killer (NK) cells (CD56), and T cells (CD3epsilon (CD3ε) and CD3delta (CD3δ)) can be used as secondary markers to provide support for diagnosis of disease. In particular embodiments, CD3ε and CD3δ can be used as primary markers to diagnose SCID. The assays can detect the presence or absence of markers associated with these disorders using dried blood spots (DBS) already routinely collected as part of existing newborn screening (NBS) procedures. In particular embodiments, buccal swab samples, peripheral mononuclear blood cell (PBMC) samples, or white blood cell (WBCs) samples obtained from a subject can be used in the assays.

The current disclosure describes peptides associated with each of the disorders that can be reliably detected and quantified using peptide immunoaffinity enrichment coupled to selected reaction monitoring mass spectrometry (immuno-SRM). The current disclosure also provides high affinity antibodies that can be used to enrich for the peptides.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. Protein targets and peptide sequences used for peptide immunoaffinity enrichment coupled to selected reaction monitoring mass spectrometry (immuno-SRM-MS) for X-linked chronic granulomatous disease (X-CGD), X-linked lymphoproliferative syndrome (XLP1; SH2D1A deficiency), familial hemophagocytic lymphohistiocytosis 2 (FHL2), ataxia telangiectasia (AT), common variable immunodeficiency (CVID; B-cell dysfunctions), severe combined immunodeficiency (SCID), adenosine deaminase (ADA) deficiency, dedicator of cytokinesis 8 (DOCK8) deficiency, and secondary markers for platelets (CD42), natural killer (NK) cells (CD56), and T cells (CD3epsilon (CD3ε) and CD3delta (CD3δ)). In particular embodiments, CD3ε and CD3δ can be used as primary markers to diagnose SCID. Total mass, parent ion mass, daughter y-ion masses, and daughter b-ion masses are also shown.

FIG. 2. Schematic illustrating the process of immuno-SRM-MS.

FIG. 3. Fragment spectra for each target peptide with selected transitions.

FIG. 4. Endogenous multiple reaction monitoring (MRM) traces for signature peptides.

FIGS. 5A, 5B. Linearity curves for each signature peptide. (FIG. 5A) High fmol range plots represent the full analyzed concentration range. (FIG. 5B) Low fmol range plots represent enlarged low concentration regions from FIG. 5A plots.

FIG. 6. Patient information including measured concentrations of all signature peptides, diagnoses, genetic information and present treatments. N/A=Not Available.

FIG. 7A, 7B. Immuno-SRM analysis of platelet markers CD42 128 and CD42 154 show corresponding changes in platelet levels. ****p<0.001.

FIGS. 8A-8C. Immuno-SRM analysis of primary signature peptides CYBB 509, DOCK8 1272, and ADA 93 in X-CGD, DOCK8 deficiency, and ADA deficiency patients. *p<0.05.

FIG. 9. Levels of NK Cell marker CD56 122 found in DBS of PIDD patients.

FIGS. 10A-10C. Comparison of PIDD patient identification using HT and standard Immuno-SRM. ***=p<0.005.

FIG. 11. Exemplary sequences of the disclosure including:

| SEQ ID | |
|---|---|
| NO: | Description |
| 22 | anti-CYBB 509 heavy chain coding sequence (clone EB0611C-1E12-H2) with |
| | leader sequence |
| 23 | anti-CYBB 509 heavy chain amino acid sequence with leader sequence |
| 24 | anti-CYBB 509 heavy chain amino acid sequence without leader sequence |
| 25 | anti-CYBB 509 variable heavy domain coding sequence with leader sequence |
| 26 | anti-CYBB 509 variable heavy domain amino acid sequence with leader sequence |
| 28 | anti-CYBB 509 anti-CYBB 509 light chain coding sequence (clone EB0611C- |
| | 1E12-K2) with leader sequence |
| 29 | anti-CYBB 509 light chain amino acid sequence with leader sequence |
| 30 | anti-CYBB 509 light chain amino acid sequence without leader sequence |
| 31 | anti-CYBB 509 variable light domain coding sequence with leader sequence |
| 32 | anti-CYBB 509 variable light domain amino acid sequence with leader sequence |
| 40 | anti-PRF 215 heavy chain coding sequence (clone EB0610D-9E12-H3) with |
| | leader sequence |
| 41 | anti-PRF 215 heavy chain amino acid sequence with leader sequence |
| 42 | anti-PRF 215 heavy chain amino acid sequence without leader sequence |
| 43 | anti-PRF 215 variable heavy domain coding sequence with leader sequence |
| 44 | anti-PRF 215 variable heavy domain amino acid sequence with leader sequence |
| 46 | anti-PRF 215 anti-CYBB 509 light chain coding sequence (clone EB0610D-9E12- |
| | K1) with leader sequence |
| 47 | anti-PRF 215 light chain amino acid sequence with leader sequence |
| 48 | anti-PRF 215 light chain amino acid sequence without leader sequence |
| 49 | anti-PRF 215 variable light domain coding sequence with leader sequence |
| 50 | anti-PRF 215 variable light domain amino acid sequence with leader sequence |
| 58 | anti-ATM 1561 heavy chain coding sequence (clone EB0608D-4A10-H4) with |
| | leader sequence |
| 59 | anti-ATM 1561 heavy chain amino acid sequence with leader sequence |
| 60 | anti-ATM 1561 heavy chain amino acid sequence without leader sequence |
| 61 | anti-ATM 1561 variable heavy domain coding sequence with leader sequence |
| 62 | anti-ATM 1561 variable heavy domain amino acid sequence with leader sequence |
| 64 | anti-ATM 1561 light chain coding sequence (clone EB608D-4A10-K1) with leader |
| | sequence |
| 65 | anti-ATM 1561 light chain amino acid sequence with leader sequence |
| 66 | anti-ATM 1561 light chain amino acid sequence without leader sequence |
| 67 | anti-ATM 1561 variable light domain coding sequence with leader sequence |
| 68 | anti-ATM 1561 variable light domain amino acid sequence with leader sequence |
| 76 | anti-CD19 41 variable heavy domain coding sequence with leader sequence |
| 77 | anti-CD19 41 variable heavy domain amino acid sequence with leader sequence |
| 79 | anti-CD19 41 variable light domain coding sequence with leader sequence |
| 80 | anti-CD19 41 variable light domain amino acid sequence with leader sequence |
| 88 | anti-ADA 93 heavy chain coding sequence (clone EB0608A-3C12-H2) with leader |
| | sequence |
| 89 | anti-ADA 93 heavy chain amino acid sequence with leader sequence |
| 90 | anti-ADA 93 heavy chain amino acid sequence without leader sequence |
| 91 | anti-ADA 93 variable heavy domain coding sequence with leader sequence |
| 92 | anti-ADA 93 variable heavy domain amino acid sequence with leader sequence |
| 94 | anti-ADA 93 light chain coding sequence (clone EB0608A-3C12-K2) with leader |
| | sequence |
| 95 | anti-ADA 93 light chain amino acid sequence with leader sequence |
| 96 | anti-ADA 93 light chain amino acid sequence without leader sequence |
| 97 | anti-ADA 93 variable light domain coding sequence with leader sequence |
| 98 | anti-ADA 93 variable light domain amino acid sequence with leader sequence |
| 106 | anti-DOCK8 1272 variable heavy domain coding sequence (Hybridoma SJSH5- |
| | 1C2-2E) with leader sequence |
| 107 | anti-DOCK8 1272 variable heavy domain amino acid sequence with leader |
| | sequence |
| 109 | anti-DOCK8 1272 variable light domain coding sequence (Hybridoma SJSH5-1C2- |
| | 2E) with leader sequence |
| 110 | anti-DOCK8 1272 variable light domain amino acid sequence with leader |
| | sequence |
| 118 | anti-CD42 128 variable heavy domain coding sequence (Hybridoma SJSH4 3B8- |
| | 1b) with leader sequence |
| 119 | anti-CD42 128 variable heavy domain amino acid sequence with leader sequence |
| 121 | anti-CD42 128 variable light domain coding sequence (Hybridoma SJSH4 3B8-1b) |
| | with leader sequence |
| 122 | anti-CD42 128 variable light domain amino acid sequence with leader sequence |
| 130 | anti-CD42 154 variable heavy domain coding sequence (Hybridoma SJSH4- |
| | 1H11c) with leader sequence |
| 131 | anti-CD42 154 variable heavy domain amino acid sequence with leader sequence |
| 133 | anti-CD42 154 variable light domain coding sequence (Hybridoma SJSH4-1H11c) |
| | with leader sequence |
| 134 | anti-CD42 154 variable light domain amino acid sequence with leader sequence |
| 142 | anti-CD56 122 heavy chain coding sequence (clone EB0608C-3H4-H2) with |
| | leader sequence |
| 143 | anti-CD56 122 heavy chain amino acid sequence with leader sequence |
| 144 | anti-CD56 122 heavy chain amino acid sequence without leader sequence |
| 145 | anti-CD56 122 variable heavy domain coding sequence with leader sequence |
| 146 | anti-CD56 122 variable heavy domain amino acid sequence with leader sequence |
| 148 | anti-CD56 122 light chain coding sequence (clone EB0608C-3H4-K2) with leader |
| | sequence |
| 149 | anti-CD56 122 light chain amino acid sequence with leader sequence |
| 150 | anti-CD56 122 light chain amino acid sequence without leader sequence |
| 151 | anti-CD56 122 variable light domain coding sequence with leader sequence |
| 152 | anti-CD56 122 variable light domain amino acid sequence with leader sequence |
| 166 | anti-SH2D1A (SAP) 19 heavy chain coding sequence (clone EB0612B-2F4-H1) |
| | with leader sequence |
| 167 | anti-SH2D1A (SAP) 19 heavy chain coding sequence (clone EB0612B-2F4-H1) |
| | without leader sequence |
| 168 | anti-SH2D1A (SAP) 19 heavy chain amino acid sequence with leader sequence |
| 169 | anti-SH2D1A (SAP) 19 heavy chain amino acid sequence without leader |
| | sequence |
| 170 | anti-SH2D1A (SAP) 19 variable heavy domain coding sequence with leader |
| | sequence |
| 171 | anti-SH2D1A (SAP) 19 variable heavy domain coding sequence without leader |
| | sequence |
| 172 | anti-SH2D1A (SAP) 19 variable heavy domain amino acid sequence with leader |
| | sequence |
| 174 | anti-SH2D1A (SAP) 19 light chain coding sequence (clone EB0612B-2F4-K1) with |
| | leader sequence |
| 175 | anti-SH2D1A (SAP) 19 light chain coding sequence (clone EB0612B-2F4-K1) |
| | without leader sequence |
| 176 | anti-SH2D1A (SAP) 19 light chain amino acid sequence with leader sequence |
| 177 | anti-SH2D1A (SAP) 19 light chain amino acid sequence without leader sequence |
| 178 | anti-SH2D1A (SAP) 19 variable light domain coding sequence with leader |
| | sequence |
| 179 | anti-SH2D1A (SAP) 19 variable light domain coding sequence without leader |
| | sequence |
| 180 | anti-SH2D1A (SAP) 19 variable light domain amino acid sequence with leader |
| | sequence |
| 182 | anti-CYBB 509 heavy chain coding sequence (clone EB0611C-1E12-H2) without |
| | leader sequence |
| 183 | anti-CYBB 509 variable heavy domain coding sequence without leader sequence |
| 184 | anti-CYBB 509 light chain coding sequence (clone EB0611C-1E12-K2) without |
| | leader sequence |
| 185 | anti-CYBB 509 variable light domain coding sequence without leader sequence |
| 186 | anti-PRF 215 heavy chain coding sequence (clone EB0610D-9E12-H3) without |
| | leader sequence |
| 187 | anti-PRF 215 variable heavy domain coding sequence without leader sequence |
| 188 | anti-PRF 215 light chain coding sequence (clone EB0610D-9E12-K1) without |
| | leader sequence |
| 189 | anti-PRF 215 variable light domain coding sequence without leader sequence |
| 190 | anti-ATM 1561 heavy chain coding sequence (clone EB0608D-4A10-H4) without |
| | leader sequence |
| 191 | anti-ATM 1561 variable heavy domain coding sequence without leader sequence |
| 192 | anti-ATM 1561 light chain coding sequence (clone EB0608D-4A10-K1) without |
| | leader sequence |
| 193 | anti-ATM 1561 variable light domain coding sequence without leader sequence |
| 194 | anti-CD19 41 variable heavy domain coding sequence without leader sequence |
| 195 | anti-CD19 41 variable light domain coding sequence without leader sequence |
| 196 | anti-ADA 93 heavy chain coding sequence (clone EB0608A-3C12-H2) without |
| | leader sequence |
| 197 | anti-ADA 93 variable heavy domain coding sequence without leader sequence |
| 198 | anti-ADA 93 light chain coding sequence (clone EB0608A-3C12-K2) without |
| | leader sequence |
| 199 | anti-ADA 93 variable light domain coding sequence without leader sequence |
| 200 | anti-DOCK8 1272 variable heavy domain coding sequence (Hybridoma SJSH5- |
| | 1C2-2E) without leader sequence |
| 201 | anti-DOCK8 1272 variable light domain coding sequence (Hybridoma SJSH5-1C2- |
| | 2E) without leader sequence |
| 202 | anti-CD42 128 variable heavy domain coding sequence (Hybridoma SJSH4 3B8- |
| | 1b) without leader sequence |
| 203 | anti-CD42 128 variable light domain coding sequence (Hybridoma SJSH4 3B8-1b) |
| | without leader sequence |
| 204 | anti-CD42 154 variable heavy domain coding sequence (Hybridoma SJSH4- |
| | 1H11c) without leader sequence |
| 205 | anti-CD42 154 variable light domain coding sequence (Hybridoma SJSH4-1H11c) |
| | without leader sequence |
| 206 | anti-CD56 122 heavy chain coding sequence (clone EB0608C-3H4-H2) without |
| | leader sequence |
| 207 | anti-CD56 122 variable heavy domain coding sequence without leader sequence |
| 208 | anti-CD56 122 light chain coding sequence (clone EB0608C-3H4-K2) without |
| | leader sequence |
| 209 | anti-CD56 122 variable light domain coding sequence without leader sequence |

### DETAILED DESCRIPTION

There are a number of diseases with effective treatments available. However, for a number of these diseases, once symptoms emerge, the disease is already fatal or has led to irreversible damage. Primary immunodeficiency disorders (PIDDs) is a good example of this.

Primary Immunodeficiency Disorders (PIDDs), also referred to as inborn errors of immunity (1E1), are a group of more than 416 rare genetic disorders in which components of the immune system are missing or improperly functioning. Although individually rare, the combined incidence of PIDDs is estimated to be about 1 in 1200 (Tangye et al. Journal of Clinical Immunology, 2020. In Press; McCusker, C., J. Upton, and R. Warrington, Primary immunodeficiency. Allergy, asthma, and clinical immunology: official journal of the Canadian Society of Allergy and Clinical Immunology, 2018. 14(Suppl 2): p. 61-61; Kobrynski et al. J Clin Immunol, 2014. 34(8): p. 954-61). Once diagnosed and treated appropriately, patients can often lead relatively normal lives (Kaveri et al. Clin Exp Immunol, 2011. 164 Suppl 2: p. 2-5; Raje, N. and C. Dinakar, Immunology and allergy clinics of North America, 2015. 35(4): p. 599-623). Curative therapies are also possible, depending on the disorder, with hematopoietic stem cell transplantation (HSCT), enzyme replacement therapy (ERT), or gene therapy (Raje, N. and C. Dinakar, Immunology and allergy clinics of North America, 2015. 35(4): p. 599-623; Aydin et al. J Clin Immunol, 2015. 35(2): p. 189-98; Gaspar et al. Blood, 2009. 114(17): p. 3524-32; Moratto et al., Blood, 2011. 118(6): p. 1675-84; Parta et al., Journal of Clinical Immunology, 2017. 37(6): p. 548-558; Staal et al. Frontiers in Pediatrics, 2019. 7(443); Ferrua et al., Lancet Haematol, 2019. 6(5): p. e239-e253). Almost ubiquitously, early detection of PIDDs is extremely important in controlling and preventing potentially life-threatening infections and chronic sequelae (Grunebaum et al., JAMA, 2006. 295(5): p. 508-18; Kanariou et al. Curr Opin Hematol, 2018. 25(1): p. 7-12).

Early intervention is limited by the difficulty in diagnosing PIDDs clinically and the lack of straightforward population screening tools. Laboratory evaluations are typically elicited by evidence of recurrent and/or chronic infections. After clinical evaluation, laboratory tests that are required for a diagnostic confirmation often involve technically demanding analyses, including immune cell subset analysis, protein expression, and/or enzymatic activity in patient's white blood cells (Bonilla et al., Journal of Allergy and Clinical Immunology, 2015. 136(5): p. 1186-1205.e78). Currently it is not possible to perform these clinical diagnostics from dried blood spots (DBS) as all the tests require either whole blood or isolated peripheral blood mononuclear cell (PBMC) samples. Genetic sequencing is most often used as a final confirmation (Bonilla et al., Journal of Allergy and Clinical Immunology, 2015. 136(5): p. 1186-1205.e78).

An operationally simple mass spectrometry assay using a less invasively collected sample by heel stick would allow for rapid screening of suspected PIDDs. The sensitivity and specificity of tandem mass spectrometry (MS/MS) based proteomic assay can be utilized to reliably measure extremely low abundance peptides in DBS extracts and thus quantify the proteins they represent (Collins et al., Frontiers in Immunology, 2018. 9(2756); Collins et al. Frontiers in Immunology, 2020. 11; deWilde et al., Clin Chem, 2008. 54(12): 1961-8). Furthermore, an assay capable of detecting PIDD patients using DBS would be applicable in newborn screening (NBS) and allow for patient identification before the onset of potentially fatal infections. Newborn screening for T-Cell receptor excision circles (TRECs) and Kappa-deleting element recombination circles (KRECs) from DBS does exist for severe combined immunodeficiency (SCID) and some forms of agammaglobulinemia. However, TREC/KREC analysis frequently misses several SCID subtypes (e.g. ADA, ZAP70, and MHC deficiency) and NBS for other PIDDs does not currently exist due to the lack of screening methods (Kwan et al., JAMA, 2014. 312(7): p. 729-38; Chan and Puck, J Allergy Clin Immunol, 2005. 115(2): p. 391-8; Baker et al., J Allergy Clin Immunol, 2009. 124(3): p. 522-7; Chase et al. Curr Opin Allergy Clin Immunol, 2010. 10(6): p. 521-5; la Marca et al., J Allergy Clin Immunol, 2013. 131(6): p. 1604-10). Nevertheless, recent advances have demonstrated the ability to screen metabolites related to Adenosine Deaminase (ADA) deficient SCID and purine nucleoside phosphorylase deficiency, and epigenetic markers for immune cell profiles (la Marca et al., Journal of Pharmaceutical and Biomedical Analysis, 2014. 88: p. 201-206; Baron et al., Sci Transl Med, 2018. 10(452); la Marca et al., Journal of Allergy and Clinical Immunology, 2014. 134(1): p. 155-159.e3).

SCID is a group of rare disorders caused by mutations in different genes involved in the development and function of infection-fighting immune cells such as T cells and B cells. More than a dozen genes have been implicated in SCID. Most often SCID is inherited in an autosomal recessive pattern, in which both copies of a particular gene, one inherited from the mother and one from the father, contain defects. X-linked or IL2RG deficient SCID (X-SCID) primarily affects males and is caused by mutations in the gene encoding an interleukin (IL) receptor subunit common gamma chain (IL2RG), located on the X chromosome. This receptor subunit is shared by at least six different interleukin receptor complexes, including those of IL2, IL4, IL7, IL9, IL15, and IL21. Mutations in the IL2RG gene lead to severe defects in the immune system through the blockade of multiple cytokine pathways important for lymphocyte development and function. Males with this type of SCID have white blood cells that grow and develop abnormally. As a consequence, they have low numbers of T cells and natural killer cells, and their B cells do not function. SCID patients are usually affected by severe bacterial, viral, or fungal infections early in life and often are afflicted with scarring of the lungs, chronic diarrhea, and failure to thrive. The condition is fatal, usually within the first year or two of life, unless infants receive immune-restoring treatments, such as transplants of blood-forming stem cells, gene therapy, or enzyme therapy.

The best-known form of autosomal recessive SCID is caused by adenosine deaminase (ADA) deficiency, in which infants lack the ADA enzyme necessary for T-cell survival. People with SCID lack virtually all immune protection from bacteria, viruses, and fungi, and therefore, are prone to repeated and persistent infections that can be life-threatening. Most individuals with ADA deficiency are diagnosed with SCID in the first 6 months of life. Over time, affected individuals may develop chronic lung damage, malnutrition, and other health problems. ADA is an enzyme that is necessary for the breakdown of purines. Current treatment options include enzyme replacement therapy, allogeneic haematopoietic stem cell transplant (HSCT), and autologous gene therapy.

X-linked chronic granulomatous disease (X-CGD) is the most common form of CGD, an inherited PIDD that increases the body's susceptibility to infections caused by certain bacteria and fungi. Patients with CGD have defective neutrophils that cannot fight infection because these cells cannot produce hydrogen peroxide. Masses of immune cells called granulomas form at sites of infection or inflammation. These severe infections can include skin or bone infections and abscesses in internal organs. Children with CGD are often healthy at birth but develop severe infections in infancy or early childhood. X-CGD is caused by mutations in the CYBB gene, which encodes a protein called cytochrome b-245, beta chain (also known as p91-phox). Cytochrome b-245, along with other subunits, forms an enzyme complex called NADPH oxidase, which plays an essential role in the immune system. Therapeutic options for CGD include prophylactic antibiotics and antifungal medications, interferon-gamma injections, and aggressive management of acute infections. Bone marrow transplantation can cure CGD; however, this therapy is complex and transplant candidates and donors must be carefully selected.

X-linked lymphoproliferative syndrome (XLP1; SH2D1A deficiency) is a rare, inherited PIDD characterized by a defective immune system response to infection with the Epstein-Barr virus (EBV) and may result in severe, life-threatening hepatitis, abnormally low levels of antibodies or immunoglobulins (hypogammaglobulinemia) in the blood and body secretions, and/or malignancies of certain types of lymphoid tissue (B-cell lymphomas). XLP1 is caused by mutations in the SH2D1A gene, which encodes a protein that plays a major role in the bidirectional stimulation of T and B cells. If affected individuals are identified before EBV exposure, infusion with immunoglobulins (intravenous gammaglobulin) with EBV antibodies may be recommended to help prevent life-threatening infectious mononucleosis and the onset of other symptoms. In affected individuals who are diagnosed with XLP subsequent to EBV exposure, treatment may include antibiotic medications to help prevent opportunistic infections and/or intravenous gammaglobulin therapy. Affected individuals who develop B-cell lymphoma may be treated with surgery, radiation, and/or chemotherapy.

Familial hemophagocytic lymphohistiocytosis 2 (FHL2) is a disorder in which the immune system produces too many activated immune cells (lymphocytes) including T cells, natural killer cells, B cells, and macrophages (histiocytes). Excessive amounts of immune system proteins called cytokines are also produced. This overactivation of the immune system causes fever and damages the liver and spleen, resulting in enlargement of these organs. Blood-producing cells in the bone marrow are also destroyed in a process called hemophagocytosis. As a result, affected individuals have anemia and a reduction in the number of platelets. A reduction in platelets may cause easy bruising and abnormal bleeding. FHL2 is caused by mutations in the PRF1 gene, which encodes a perforin protein found in T cells and natural killer (NK) cells that is involved in cell destruction and regulation of the immune system. Allogeneic hematopoietic cell transplantation can be a cure for FHL2. Emapalumab is FDA-approved as a treatment of adult and pediatric patients with primary hemophagocytic lymphohistiocytosis.

Ataxia telangiectasia (AT) is a rare inherited disorder that affects the nervous system, immune system, and other body systems. This disorder is characterized by progressive difficulty with coordinating movements (ataxia) beginning in early childhood and by small clusters of enlarged blood vessels called telangiectases, which occur in the eyes and on the surface of the skin. Affected individuals have a weakened immune system, are prone to lung infections, and have an increased risk of developing cancer. Affected individuals tend to have high amounts of a protein called alpha-fetoprotein (AFP) in their blood. Ataxia telangiectasia is caused by mutations in the ATM gene, which encodes an enzyme that plays a role in regulating cell division following DNA damage. Treatment for ataxia telangiectasia is directed toward control of symptoms and include: therapy with an antibiotic drug, postural drainage of the bronchial tubes and lungs, and gammaglobulin injections for respiratory infections; avoidance of undue exposure to sunlight to control spread and severity of telangiectasias; Vitamin E therapy; administration of the drug diazepam (Valium) to help slurred speech and involuntary muscle contractions; and physical therapy.

Common variable immunodeficiency (CVID; B-cell dysfunctions) is one of the most common forms of PIDD. It is characterized by an antibody deficiency that leaves the immune system unable to defend against bacteria and viruses, resulting in recurrent and severe infections primarily affecting the ears, sinuses, and respiratory tract. Approximately 25 percent of people with CVID have an autoimmune disorder that can affect blood cells, causing low numbers of white cells or platelets, anemia, arthritis and other conditions such as endocrine disorders. In some forms of CVID, patients develop granulomas in the lungs, lymph nodes, liver, skin or other organs. CVID has many genetic origins but typically features dysfunctional, reduced, or absent B-cells. Treatment for CVID includes immunoglobulin replacement, preventative antibiotics, and management of autoimmune and granulomatous disease.

Dedicator of cytokinesis 8 (DOCK8) deficiency is a genetic disorder characterized by elevated immunoglobulin E levels, increased eosinophil (type of disease fighting white blood cell) levels, and recurrent staphylococcus and viral infections. DOCK8 is a protein involved in regulating the actin skeleton of the cell and may also be a tumor suppressor, since DOCK8 is lost in many cancers and people with DOCK8 deficiency are prone to developing malignancies. Treatment for DOCK8 deficiency focuses on preventing and treating infections and include: broad-spectrum antibiotics; topical antibiotics; systemic antibiotics including trimethoprim/sulfamethoxazole, penicillins, and cephalosporins and antifungals; sodium cromoglycate to improve white blood cell function; isotretinoin for skin conditions; intravenous immunoglobulin; interferon gamma; omalizumab; Cyclosporine A; HSCT; bone marrow transplantation; and surgical treatment.

CD42, also known as glycoprotein Ib (GPIb), is a component of the GPIb-V-IX complex on platelets. The GPIb-V-IX complex binds von Willebrand factor (VWF) to allow platelet adhesion and platelet plug formation at sites of vascular injury.

CD56, also known as neural cell adhesion molecule (NCAM), is a homophilic binding glycoprotein expressed on the surface of neurons, glia, and skeletal muscle. In particular, the expression of CD56 is associated with NK cells. In addition to being a marker of neural lineage commitment, CD56 expression is also found in the hematopoietic system. CD56 has been detected on other lymphoid cells, including gamma delta (γδ) T cells and activated CD8+ T cells, as well as on dendritic cells. NCAM has been implicated as having a role in cell-cell adhesion, neurite outgrowth, synaptic plasticity, and learning and memory.

CD3epsilon (CD3ε) is a polypeptide that forms part of the T cell receptor (TCR)-CD3 complex. The CD3ε chain, along with CD3δ, CD3γ, and CD3 ζ chains, are responsible for transmitting TCR-mediated signals across the cell membrane to activate downstream signaling pathways important for adaptive immune responses. In addition to its role in signal transduction during T-cell activation, CD3ε functions in correct T cell development. CD3ε initiates the TCR-CD3 complex assembly by forming the two heterodimers CD3δ/CD3ε and CD3γ/CD3ε. CD3ε also functions in internalization and cell surface down-regulation of TCR-CD3 complexes via endocytosis sequences present in the CD3ε cytoplasmic region.

CD3delta (CD3δ) is a polypeptide that forms part of the T cell receptor (TCR)-CD3 complex. The CD3δ chain, along with CD3ε, CD3γ, and CD3ζ chains, are responsible for transmitting TCR-mediated signals across the cell membrane to activate downstream signaling pathways important for adaptive immune responses. In addition to its role in signal transduction during T-cell activation, CD3δ functions in correct intracellular TCR-CD3 complex assembly and surface expression. CD3δ plays an important role in thymocyte (lymphocyte within the thymus gland) differentiation. In the absence of a functional TCR-CD3 complex, thymocytes are unable to differentiate properly. CD3δ establishes a functional link between the TCR and co-receptors CD4 and CD8 by interacting with CD4 and CD8, an interaction that allows activation and positive selection of CD4 or CD8 T cells.

X-CGD, XLP1, FHL2, AT, CVID, SCID, ADA deficiency, and DOCK8 deficiency would be strong candidates for newborn screening (NBS) if robust screening methods could identify patients from dried blood spots (DBS) during the neonatal period. For example, as the majority of congenital PIDDs result in the reduction or absence of specific proteins, direct quantification of these target proteins represents an attractive potential screening tool.

To conduct NBS assessments, punches of dried blood are taken from the DBS and laboratory tests are performed to detect the presence or absence of specific substances within the blood (called markers or biomarkers) that are indicative of disorders not apparent at birth but that cause serious health problems later in life. Though the disorders screened vary from state to state, most states screen for phenylketonuria, primary congenital hypothyroidism, cystic fibrosis, and sickle cell disease. NBS has proven to be highly effective at improving patient outcomes and avoiding long-term disability in affected individuals, while at the same time reducing healthcare costs. Unfortunately, detection is often limited by the extremely low protein concentrations in blood cells and limited blood volume present in DBS.

Tandem mass spectrometry (MS/MS) was first applied to NBS in the 1990s, paving the way for rapid screening of multiple metabolites and thus several diseases from DBS samples collected at birth (Chace J Mass Spectrom. Wiley-Blackwell; 2009; 44: 163-170; Millington et al. J. Inherit. Metab. Dis. 1990; 13: 321-324; Sweetman et al. Pediatrics. 2006; 117: S308-S314; Almannai et al. Curr. Opin. Pediatr. 2016; 28: 694-699; Watson et al. Genet. Med. Nature Publishing Group; 2006. pp. 1S-252S; Chace et al. Clin. Chem. 1993; 39: 66-71). Selected reaction monitoring mass spectrometry (SRM-MS) performed on triple quadrupole mass spectrometers further enabled the precise, high-throughput, and analytically-robust quantification of specific biomarkers; as such, it is now the standard of care at clinical NBS laboratories across the world (Chace DH J Mass Spectrom. Wiley-Blackwell; 2009; 44: 163-170; Chace & Kalas. Clinical Biochemistry. 2005; 38: 296-309. Dott et al. American Journal of Medical Genetics Part A. Wiley Subscription Services, Inc., A Wiley Company; 2006; 140: 837-842).

MS/MS relies on the measurement of concentrated upstream metabolites for detection of various inborn errors of metabolism with specific enzyme deficiencies. This excludes its application to diseases, such as PIDD, where no accumulated metabolites are present or currently verified. For this reason, protein-based assays such as flow cytometry or western blotting have been used as first-line investigative methods for diseases such as Wiskott-Aldrich Syndrome (WAS) and its milder phenotype, X-linked thrombocytopenia (XLT), where most mutations lead to absent or decreased protein products (Qasim et al. Br. J. Haematol. 2001; 113: 861-865; Jin et al. Blood. American Society of Hematology; 2004; 104: 4010-4019). These approaches require that intact blood samples or cells from patients be available, making population-based screening or testing of patients from resource-poor areas impossible.

It was previously shown that an MS-based approach for the quantification of signature peptides for BTK, WASP, and a T-Cell marker CD3ε from tryptic digests of PBMCs can be used to screen X-linked agammaglobulinemia (XLA), WAS, and SCID, respectively (Kerfoot et al., Proteomics Clin Appl, 2012. 6(7-8): p. 394-402). CD3ε was chosen as a general representation of T-Cell number as all SCID patients share T-Cell lymphopenia despite genetic heterogeneity. Each patient in the blinded study was deficient in the signature peptide specific for their respective disease (i.e., XLA patient lacking Bruton's Tyrosine Kinase (BTK) and WAS patient missing WAS protein (WASP), etc.).

SRM-MS utilizes proteolytically-generated signature peptides as stoichiometric surrogates of a protein of interest. This may, in turn, be used to estimate the number of a particular cell-type expressing that protein in a sample (i.e. quantification of CD3ε for an indication of the amount of CD3+ T-cells in blood). The high specificity of MS for each signature peptide is conferred by three physiochemical properties—its mass, retention times upon high-performance liquid chromatography (HPLC) separation, and resultant target-specific fragmentation patterns (Kennedy et al. Nat. Methods. 2014; 11: 149-155). Despite these advances, with a typical limit of quantification ranging from 100 to 1000 ng protein/mL, the use of complex matrices such as blood or plasma often precludes accurate quantification of low-abundance targets by SRM-MS based assays. This limits applicability to many PIDD that result in absent or decreased levels of target proteins expressed only intracellularly (de Saint Basile et al. J. Clin. Invest. American Society for Clinical Investigation; 2004; 114: 1512-1517).

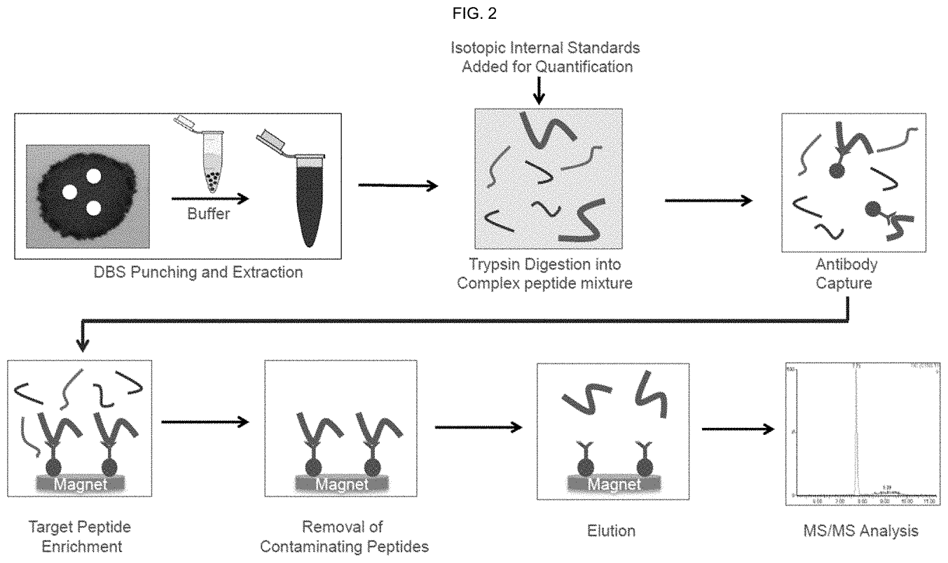

Peptide immunoaffinity enrichment coupled to SRM (immuno-SRM), also referred to as Stable Isotope Standards and Capture by Anti-Peptide Antibodies (SISCAPA), increases the sensitivity of SRM-MS assays by utilizing anti-peptide antibodies to purify and enrich peptides of interest from a complex biologic sample prior to SRM-MS analysis (Zhao et al. J Vis Exp. 2011; 53: 2812; Whiteaker et al. Mol. Cell Proteomics. American Society for Biochemistry and Molecular Biology; 2010; 9: 184-196; Whiteaker et al. Mol. Cell Proteomics. American Society for Biochemistry and Molecular Biology; 2012; 11: M111.015347; Kuhn et al. Clin. Chem. 2009; 55: 1108-1117; Anderson et al. J Proteome Res. 2004; 3(2): 235-244; Collins et al., Frontiers in Immunology, 2018. 9(2756); Collins et al. Frontiers in Immunology, 2020. 11; Jung et al., J Proteome Res, 2017. 16(2): p. 862-871). A representative immuno-SRM process is illustrated in FIG. 2.

Immuno-affinity enrichment of signature peptide biomarkers using anti-peptide antibodies isolates peptides of interest from complex biological matrices. This simplifies the sample matrix, reduces background, and concentrates analytes to enhance the sensitivity of the liquid chromatography-tandem mass spectrometry (LC-MS/MS) assay (Anderson et al., J Proteome Res, 2004. 3(2): p. 235-44; Anderson and Hunter, Mol Cell Proteomics, 2006. 5(4): p. 573-88). Immuno-SRM allows for quantification of proteins present at low picomolar concentrations in blood with high reproducibility (Whiteaker et al., Mol Cell Proteomics, 2010. 9(1): p. 184-96; Whiteaker et al., J Proteome Res, 2014. 13(4): p. 2187-96; Hoofnagle et al., Clin Chem, 2008. 54(11): p. 1796-804; Hoofnagle et al., Clin Chem, 2016. 62(1): p. 48-69; Kuhn et al., Mol Cell Proteomics, 2012. 11(6): p. M111.013854). Using this methodology, in a blinded screen of 40 patient samples, all were significantly reduced in their respective peptides and diagnostic cutoffs allowed for the positive identification of every molecularly confirmed case of XLA (n=26), WAS (n=11) and 2 of 3 cases of SCID (Collins et al., Frontiers in Immunology, 2018. 9(2756)).

Many aspects of an immuno-SRM assay depend on the disorder being diagnosed, the biomarkers available for each disorder, the ability to develop molecular entities that can enrich for peptides of interest, and the behavior of each peptide of interest in the mass spectrometer. All of these aspects and more require careful consideration and experimentation to achieve a reliable assay that can reliably detect disorders. In particular embodiments, diagnosis of disorders is conducted with an NBS panel using DBS before clinical symptoms emerge.

The present disclosure provides a multiplexed immuno-SRM method to reliably diagnose X-CGD, XLP1, FHL2, AT, CVID, SCID, ADA deficiency, and DOCK8 deficiency. Additionally, cell specific markers, CD42, CD56, CD3ε, and CD3δ can be used as secondary markers to provide supporting information for diagnosis by quantifying platelets (CD42), natural killer (NK) cells (CD56), and T cells (CD3ε and CD3δ). In particular embodiments, CD3ε and CD3δ can be used as primary markers to diagnose SCID. The multiplexed immuno-SRM assay disclosed herein can utilize anti-peptide antibodies generated against peptides of proteins reduced or absent in X-CGD, XLP1, FHL2, AT, CVID, SCID, ADA deficiency, and DOCK8 deficiency.

The following aspects of the disclosure are now described in more detail: (I) Collection and Processing of Biological Samples; (II) Peptide Markers for X-CGD, XLP1, FHL2, AT, CVID, SCID, ADA deficiency, and DOCK8 deficiency, and secondary peptide markers, CD42, CD56, CD3ε, and CD3δ; (Ill) Enzymatic Digestion of Proteins in a Biological Sample; (IV) Antibodies to Enrich for the Peptide Markers; (V) Variants; (VI) Enrichment Strategies for Peptides; (VII) Liquid Chromatography (LC); (VIII) Mass Spectrometry (MS); (IX) Methods of Use; (X) Kits; (XI) Exemplary Embodiments; (XII) Experimental Examples; and (XIII) Closing Paragraphs. These headings are provided for organization purposes only and do not limit the scope or interpretation of the disclosure.

### (I) COLLECTION AND PROCESSING OF BIOLOGICAL SAMPLES

In particular embodiments, biological samples that can be used in the methods of the present disclosure include samples derived from blood or cells. In particular embodiments, samples used in the methods of the present disclosure are DBS. In particular embodiments, whole blood from a subject can be prepared by placing blood onto a filter paper card and allowing the blood to dry.

In particular embodiments, whole blood from a subject can be collected in any anticoagulant. In particular embodiments, whole blood from a subject can be collected in heparin. DBS can be prepared by pipetting 50-100 μL (e.g., 70 μL) blood/spot onto filter paper card (e.g., Protein Saver™ 903® Card, Whatman Inc, Piscataway, N.J.), and allowed to dry at room temperature. In particular embodiments, blood is allowed to dry on filter paper card overnight. DBS can be stored, for example, in sealed plastic bags at −80° C. until use. In particular embodiments, the whole DBS can be used in the immuno-SRM assays of the disclosure. In particular embodiments, one or more 3-mm punches from the DBS can be used in the immuno-SRM assays of the disclosure. In particular embodiments, DBS can be solubilized with 0.1% Triton X-100 in 50 mM ammonium bicarbonate.

In particular embodiments, samples used in the methods of the present disclosure include cells obtained from buccal swabs or mucosal samples. In particular embodiments, mucosal samples include oral, nasal, genital, and rectal samples (Espinosa-de Aquino et al. (2017) Methods in Ecology and Evolution 8:370-378). In particular embodiments, buccal swab samples include cells from the cheek or mouth. In particular embodiments, buccal swab samples can be obtained from a subject following a protocol described in the following: CHLA. (2016, Apr. 4). Buccal Swab Collection Procedure. CHLA-Clinical Pathology; (2016, Jul. 27). Buccal DNA Collection Instructions. Pathway Genomics; (2017, Dec. 14). Instruction for Buccal Swab Sample Collection. Otogenetics; PDXL PDXL. (2017, Nov. 28). Buccal Swab collection procedure—PersonalizedDx Labs [Video]. YouTube. On World Wide Web at youtu.be/3ftvHkfM71o?t=146; and Centers of Disease Control and Prevention (CDC). (2020, Jul. 8). Interim Guidelines for collecting, handling, and testing clinical specimens for Covid-19. On World Wide Web at cdc.gov/coronavirus/2019-ncov/lab/guidelines-clinical-specimens.html.

In particular embodiments, buccal swab samples can be obtained from a subject with the following protocol. Prior to sample collection, the patient does not smoke, eat, drink, chew gum or brush their teeth for at least 30 minutes. A swab is carefully removed from the package, making sure the tip does not touch any objects or surfaces. The swab is inserted into the buccal cavity, which is located to one side of the mouth between the cheek, teeth and upper gum. The tip of the swab is pressed inside of one cheek and rubbed back and forth, up and down, in a circular motion. The handle is rotated during the rub to cover the entire tip with cells from the cheek. The tip is not allowed to touch the teeth, gums and lips during the collection process. The swab is not allowed to be over saturated with saliva. After collection, the swab is removed from the mouth without touching the teeth, gums or lips. The swab is allowed to air dry at room temperature for at least 30 minutes. The swab, with the handle removed, may be stored in a cryogenic vial. The steps may be repeated with a second swab on the opposite cheek. Buccal swab samples may be stored at 2-8° C. for up to 72 hours after collection or in the freezer at −80° C. or below if longer than 72 hours. In particular embodiments, the collection of cells with the buccal swab may be for at least 30 seconds. In particular embodiments, the collection of cells with the buccal swab may be collected from maximum mucosal surfaces. In particular embodiments, one to five buccal swab samples may be collected per subject. In particular embodiments, the buccal swab sample may be air dried on a sterile surface for at least five min, at least 10 min, at least 15 min, at least 20 min, at least 25 min, at least 30 min, or longer. In particular embodiments, the subject may rinse their mouth with clean water prior to sample collection. In particular embodiments, the area of sample collection may be moistened with saline using a separate swab. In particular embodiments, buccal swab samples may be stored at 25° C., 20° C., 15° C., 10° C., 5° C., 0° C., −5° C., −10° C., −15° C., −20° C., or below. In particular embodiments, buccal swab samples may be stored at −20° C. for one to two weeks. In particular embodiments, buccal samples may be collected from a water and/or mouthwash rinse instead of a swab (Michalczyk et al. (2004) BioTechniques 37(2):262-269).

In particular embodiments, cells from a buccal swab sample can be solubilized with 0.1% Triton X-100 in 50 mM ammonium bicarbonate. In particular embodiments, proteins may be isolated from buccal swab samples following the protocol described in Espinosa-de Aquino et al. (2017). In particular embodiments, cells from buccal swab samples may be extracted with an appropriate buffer such as TRIzol (Thermo Fisher Scientific, Waltham, Mass.) and the supernatant after nucleic acid precipitation may be used for protein extraction. In particular embodiments, proteins may be precipitated with acetone, the protein pellet may be resuspended in an appropriate buffer (e.g., guanidine hydrochloride in 95% ethanol supplemented with 2.5% glycerol), the pellet may be dispersed by sonication, the pellet may be centrifuged and washed, the pellet may be dried, and the pellet may be solubilized in an appropriate buffer (e.g., PBS and sodium dodecyl sulfate). In particular embodiments, the solubilized pellet may be heated at 100° C. and then centrifuged to obtain a supernatant for use.

In particular embodiments, samples used in the methods of the present disclosure include peripheral blood mononuclear cells (PBMCs). PBMCs come from peripheral blood and originate from hematopoietic stem cells (HSCs) that reside in the bone marrow. A PBMC is a blood cell with a round nucleus and can include many types of cells including monocytes, lymphocytes (including T cells, B cells, and NK cells), dendritic cells, and stem cells. PBMCs can be isolated by density gradient centrifugation (e.g., Ficoll-Paque) and/or by leukapheresis (Kerfoot et al., Proteomics Clin Appl, 2012. 6(7-8):394-402; Grievink et al. (2016) Biopresery Biobank 14(5):410-415; Corkum et al. (2015) BMC Immunol. 16:48; Jia et al. (2018) Biopresery Biobank 16(2):82-91). Density gradient centrifugation separates cells by cell density. In particular embodiments, whole blood or buffy coat layer may be layered over or under a density medium without mixing of the two layers followed by centrifugation. In particular embodiments, the PBMC appears as a thin white layer at the interface between the plasma and the density gradient medium. In particular embodiments, Vacutainer® blood draw tubes containing Ficoll-Hypaque and a gel plug that separates the Ficoll solution from the blood to be drawn can be used (cell preparation tubes (CPT™, BD Biosciences, San Jose, Calif.); Puleo et al. (2017) Bio-protocol 7(2): e2103). In particular embodiments, SepMate™ tubes (STEMCELL™ Technologies, Vancouver, Calif.) designed with an insert to keep the density gradient medium and the sample from mixing prior to centrifugation can be used. A leukapheresis machine is an automated device that takes whole blood from a donor and separates out the target PBMC fraction using high-speed centrifugation while returning the remaining portion of the blood, including plasma, red blood cells, and granulocytes, back to the donor. In particular embodiments, isolated PBMCs can be solubilized with 0.1% Triton X-100 in 50 mM ammonium bicarbonate.

In particular embodiments, samples used in the methods of the present disclosure include white blood cells (WBCs). WBCs, also known as leukocytes, are part of the immune system and protect the body from infections and foreign invaders. In particular embodiments, WBCs include granulocytes (polymorphonuclear cells), lymphocytes (mononuclear cells), and monocytes (mononuclear cells). In particular embodiments, enrichment for mononuclear cells or polymorphonuclear cells can use positive selection or negative selection of particular cell types with antibody-conjugated magnetic beads (Zhou et al. (2012) Clinical and Vaccine Immunology 19(7):1065-1074). In particular embodiments, WBCs include lymphocytes and monocytes but not granulocytes. In particular embodiments, WBCs can be isolated by a process that lyses erythrocytes using a lysing reagent such as ammonium chloride. In particular embodiments, mononuclear WBCs can be isolated by density gradient centrifugation as described above. In particular embodiments, isolated WBCs can be solubilized with 0.1% Triton X-100 in 50 mM ammonium bicarbonate.

### (II) PEPTIDE MARKERS FOR X-CGD, XLP1, FHL2, AT, CVID, SCID, ADA DEFICIENCY, AND DOCK8 DEFICIENCY, AND SECONDARY PEPTIDE MARKERS CD42, CD56, CD3ε, AND CD3δ

There are many theoretical proteolytic peptides from target proteins. Those can be potential candidates for monoclonal antibody production. Nonetheless, the best potential candidate peptides were chosen after screening their characteristics by MS/MS. Those signature peptides with the highest sensitivity and specificity were selected to develop corresponding monoclonal antibodies and validated using clinical samples. In particular embodiments, multiple peptides and antibodies can be included in a multiplex analysis to increase the throughput of an immuno-SRM assay and reduce the cost and time required by the assay.

Typically, one or two signature proteotypic peptides which are unique to the protein of interest and that are consistently observed in MS experiments are selected to stoichiometrically represent the protein of interest (Mallick et al. Nat Biotechnol 2007; 25: 125-131). Signature peptides can be selected by detection in previous MS experiments, use of computational tools to predict the peptides most likely observable by MS, or a combination of both. In particular embodiments, tryptic peptides 5-22 amino acids in length with moderate hydrophobicity can be selected. Very hydrophilic and very hydrophobic peptides can be less stable due to retention time variation in HPLC and loss to surfaces. In particular embodiments, Methionine residues (oxidation), N-terminal glutamine (cyclization), asparagine followed by glycine or proline (prone to deamidation), and dibasic termini (e.g. neighboring lysine or arginine residues such as KK, KR, RR, RK have the potential for variable digestion efficiency) can be undesirable (Whiteaker and Paulovich Clin Lab Med. 2011; 31(3): 385-396). Shorter peptides and those containing proline residues can be better targets for SRM (Lange et al. Molecular Systems Biology 2008; 4: 222).

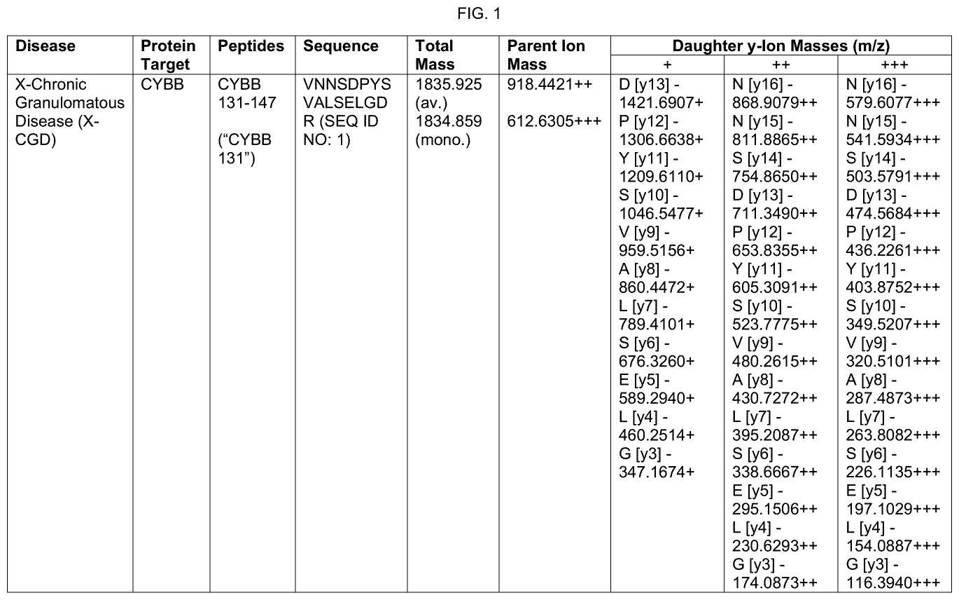

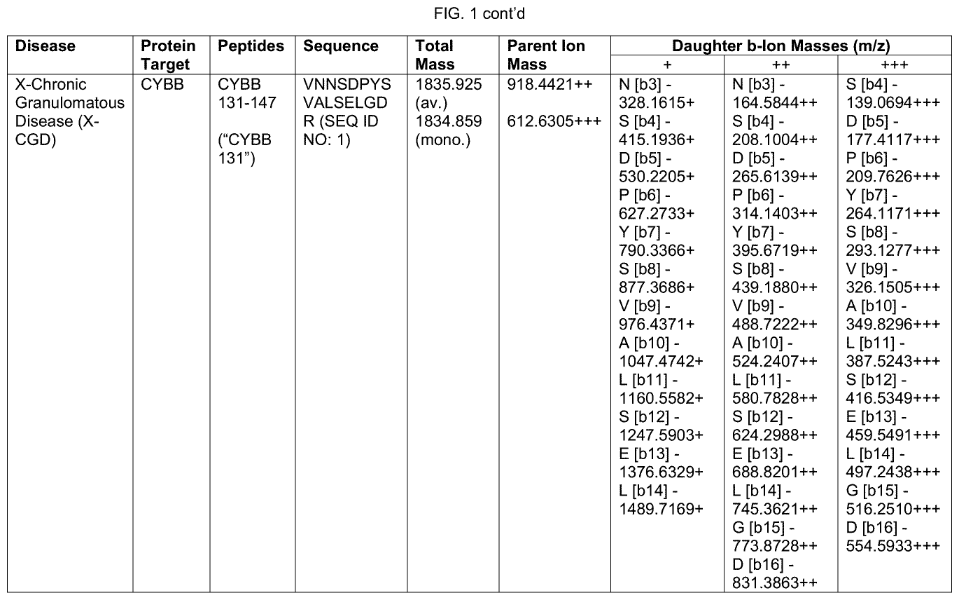

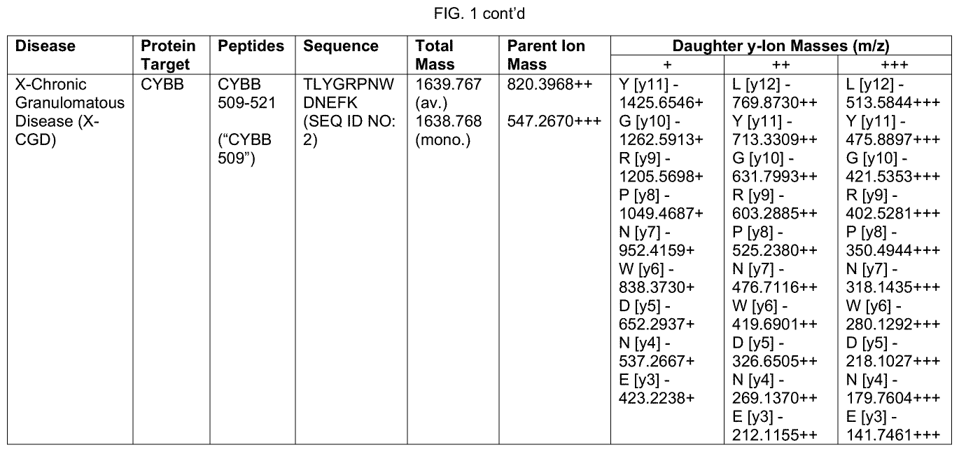

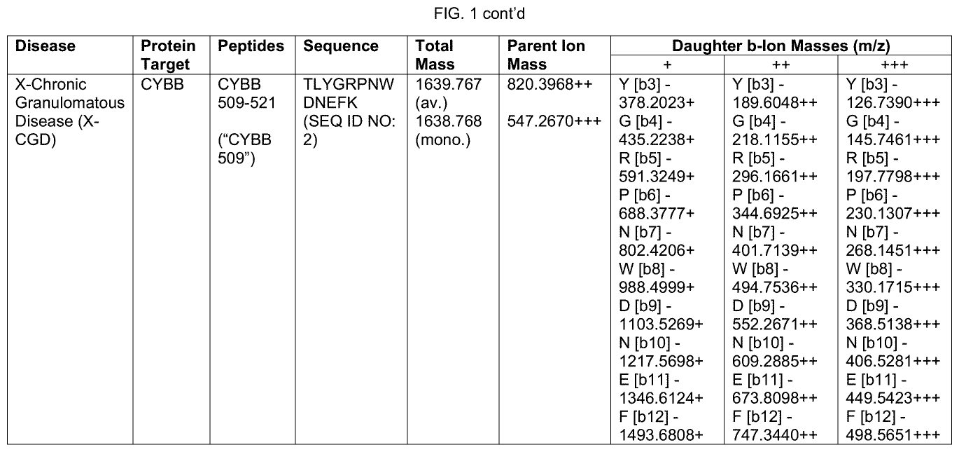

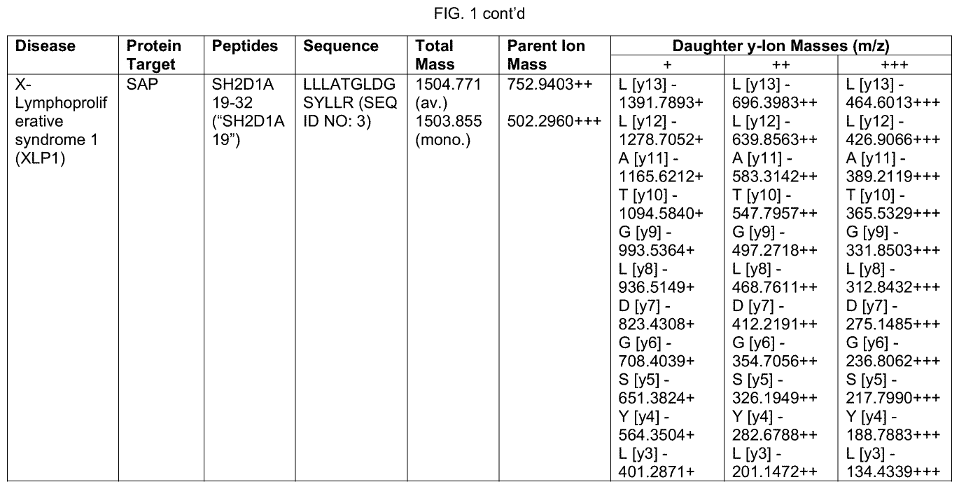

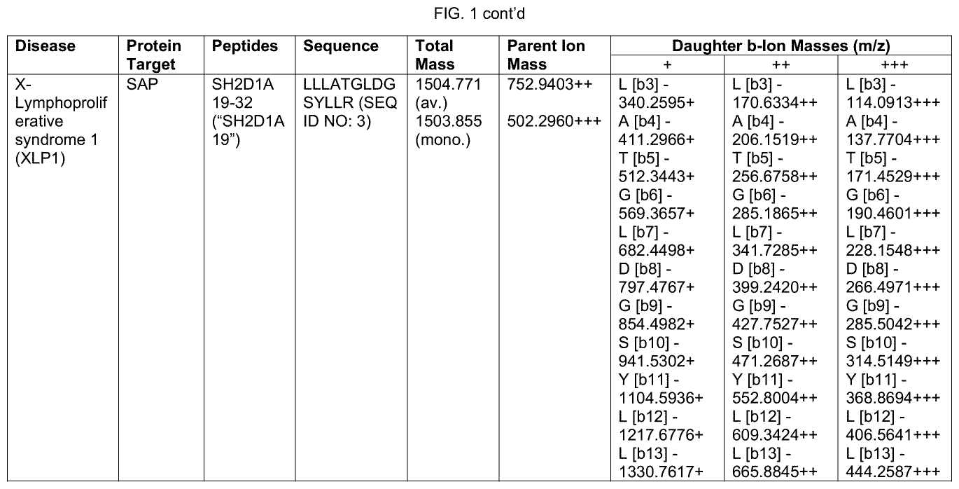

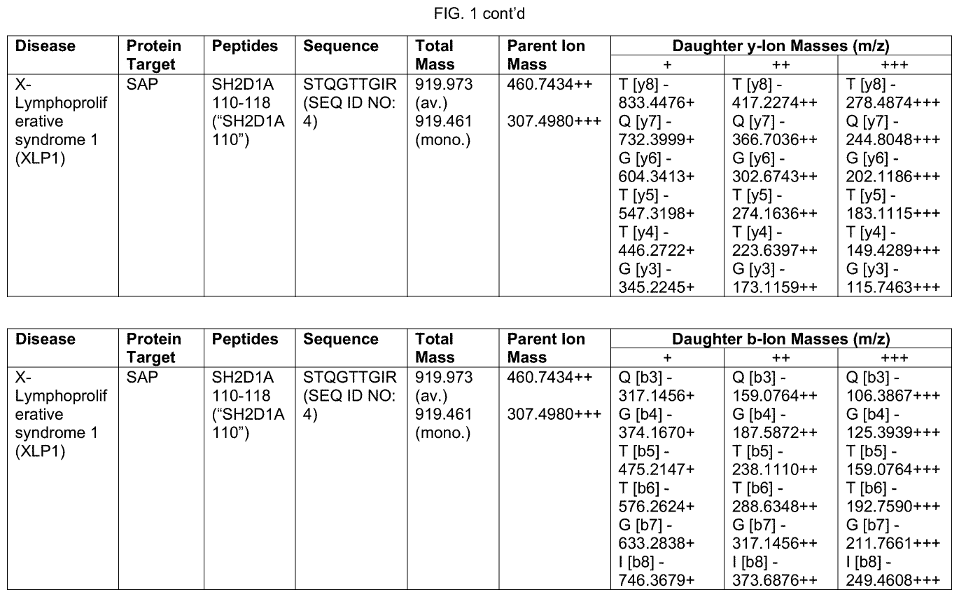

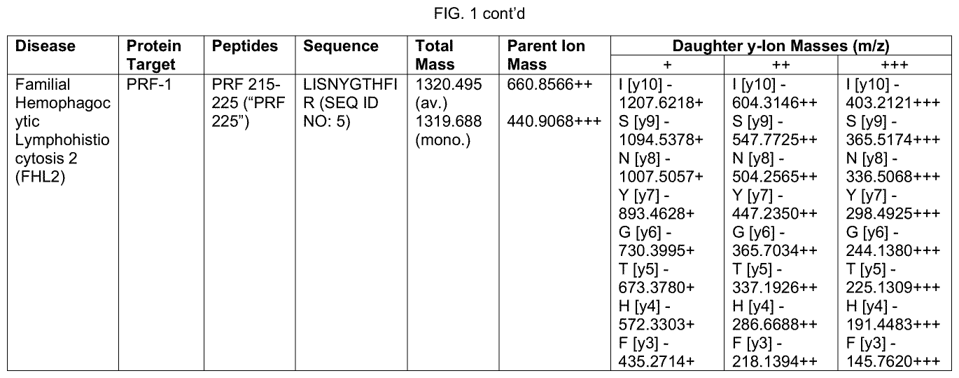

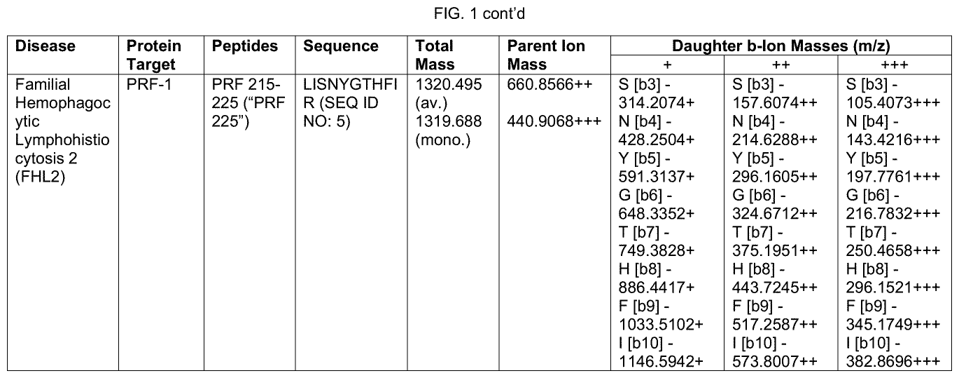

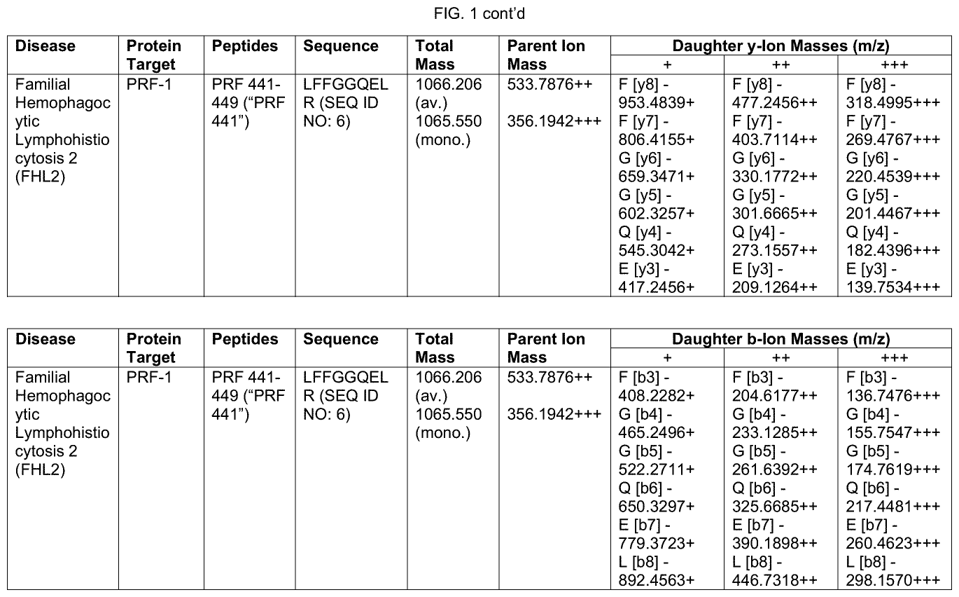

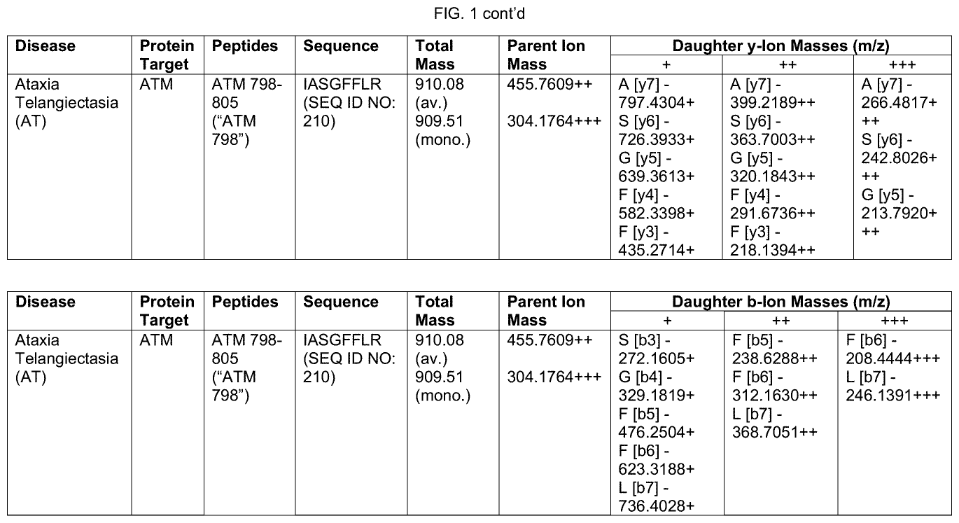

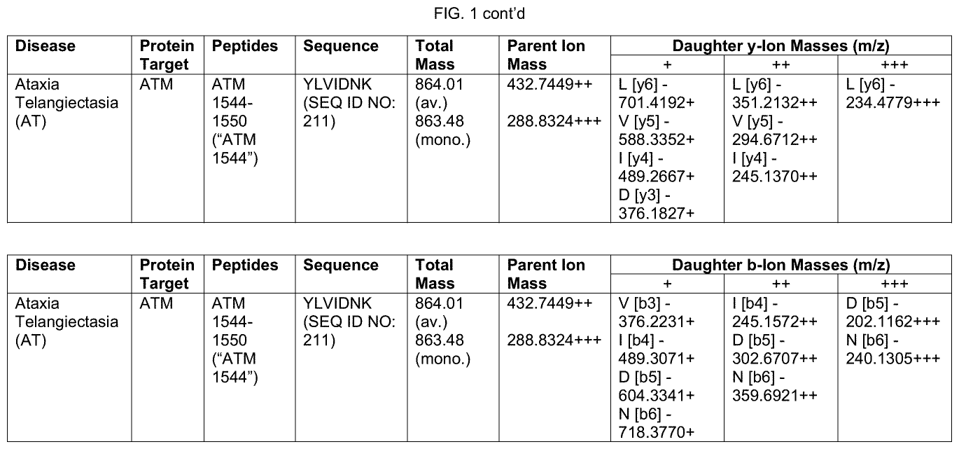

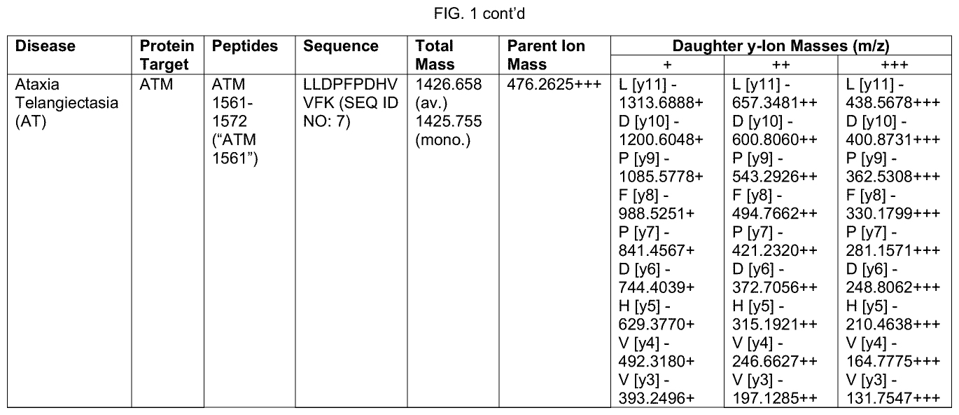

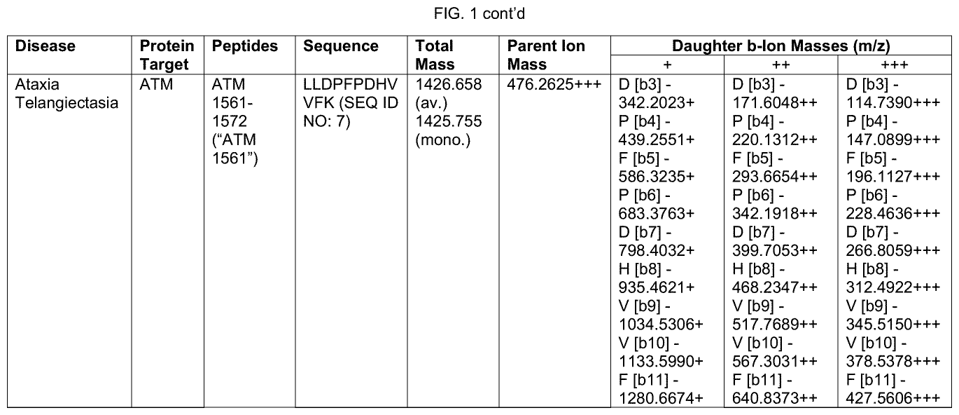

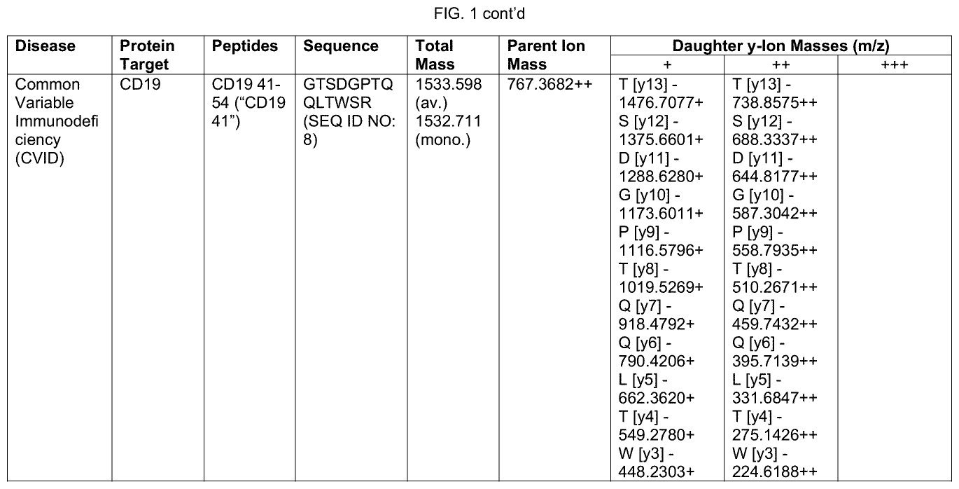

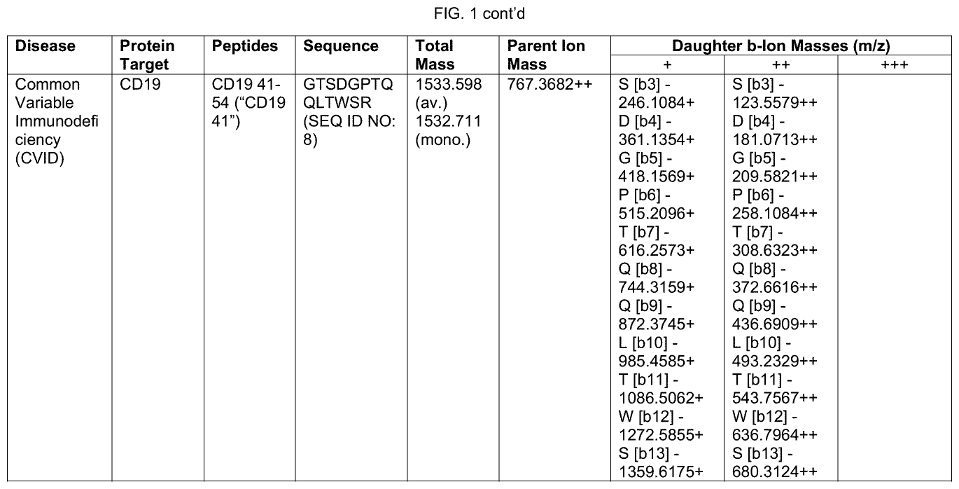

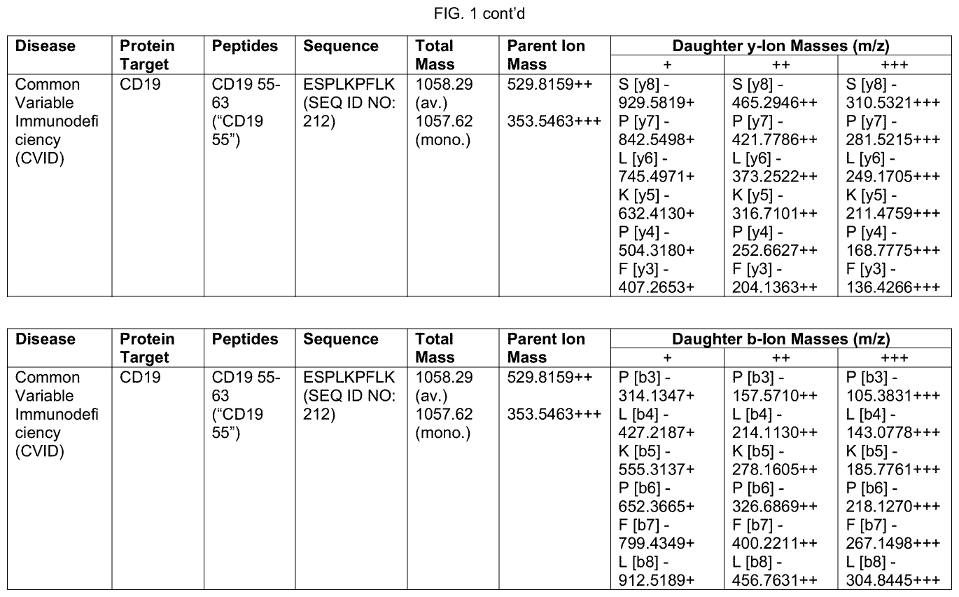

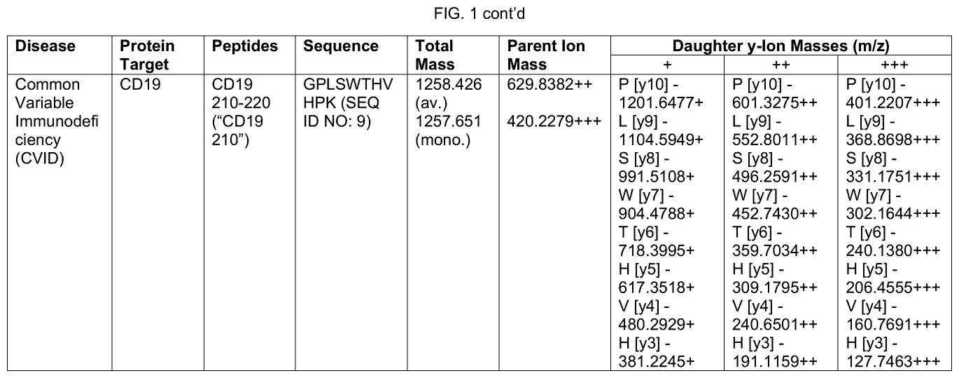

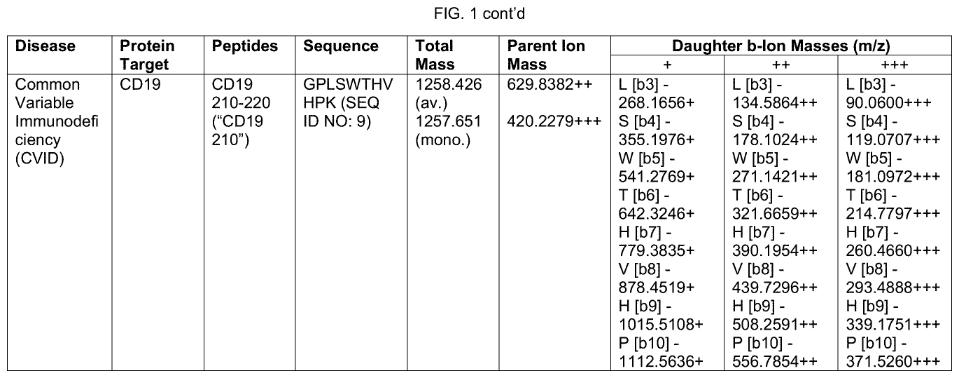

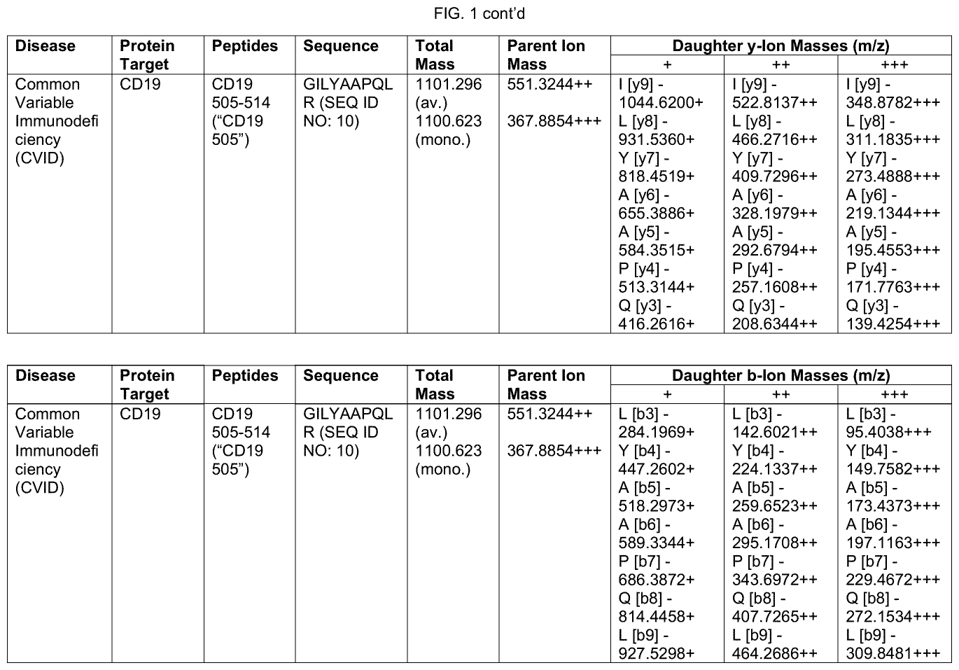

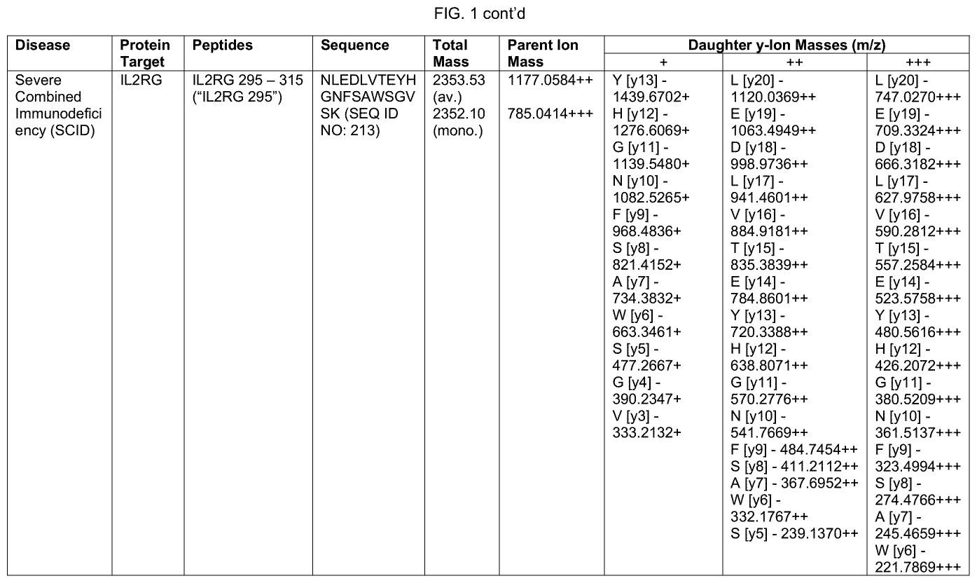

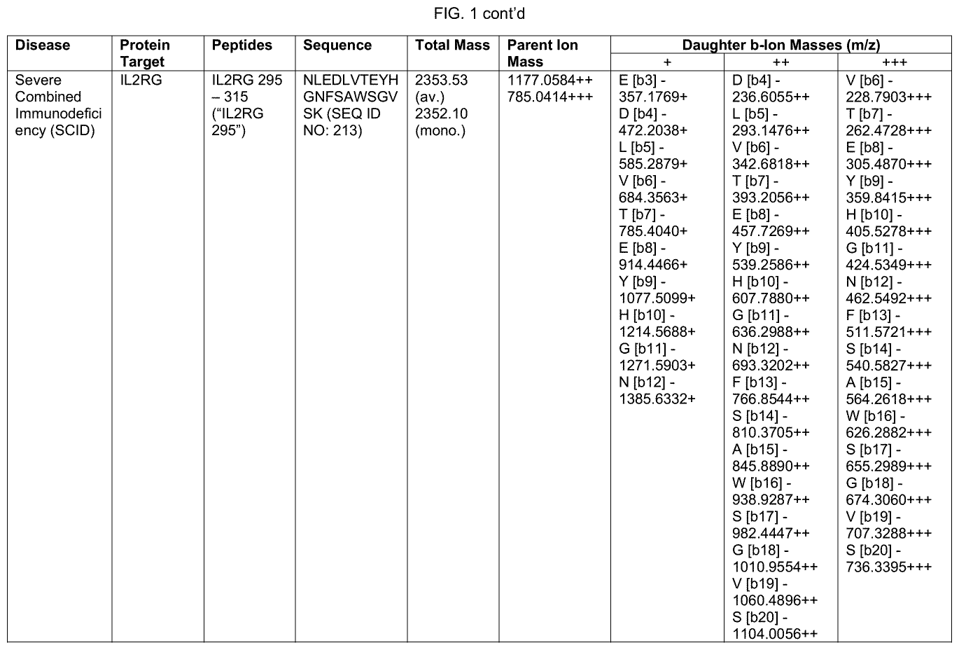

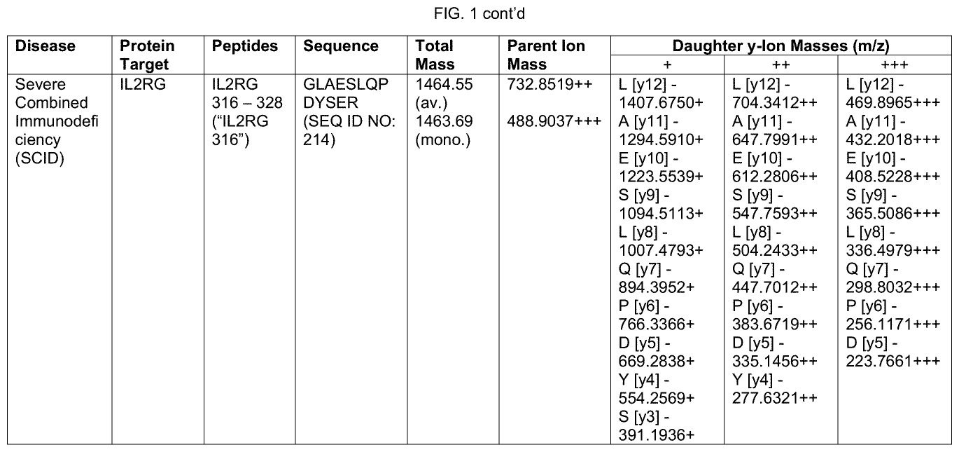

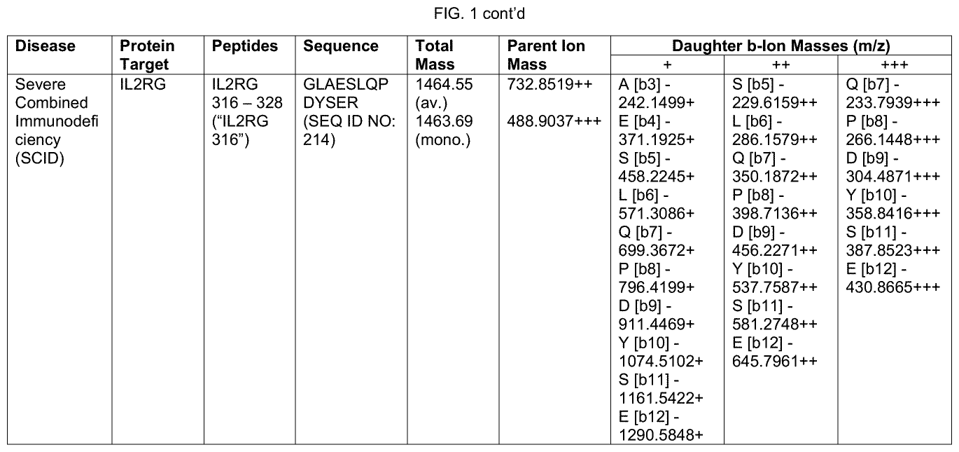

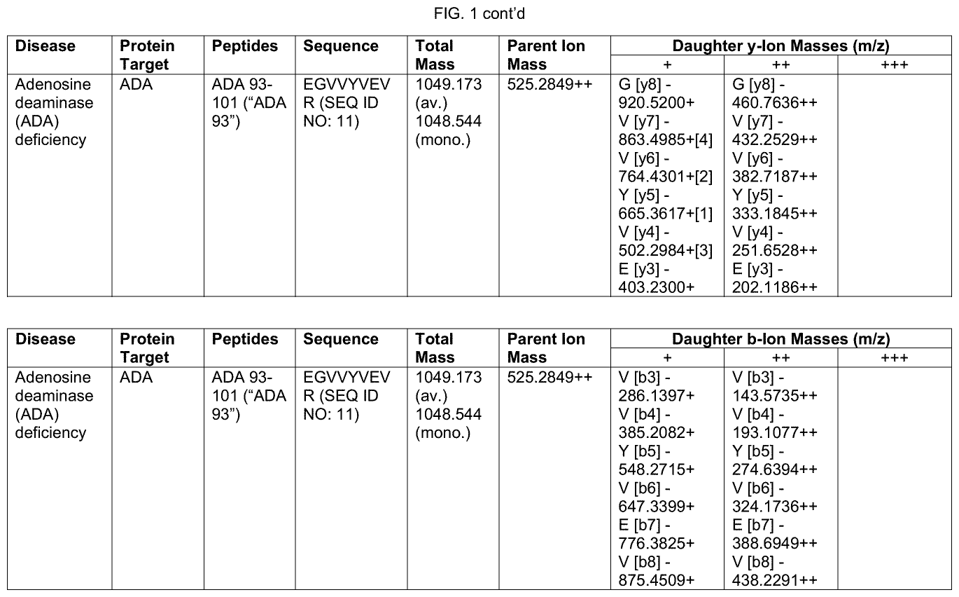

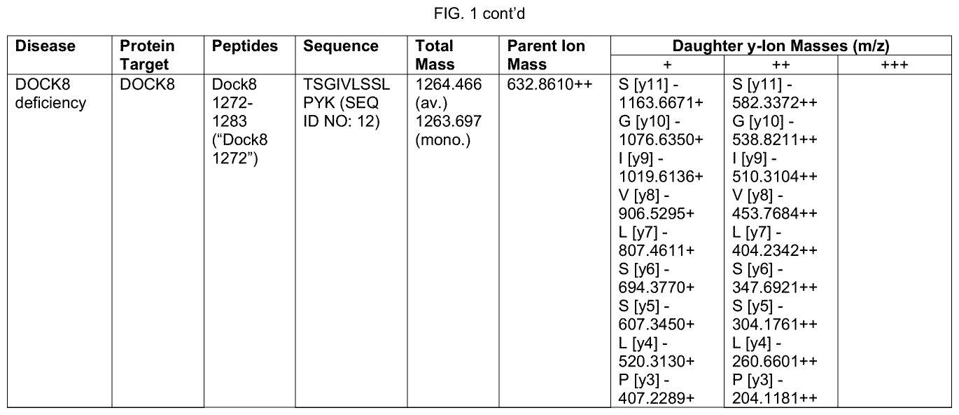

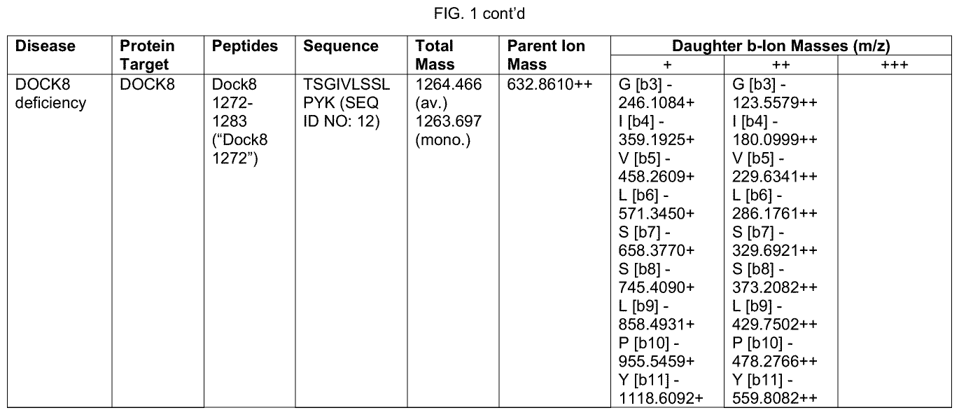

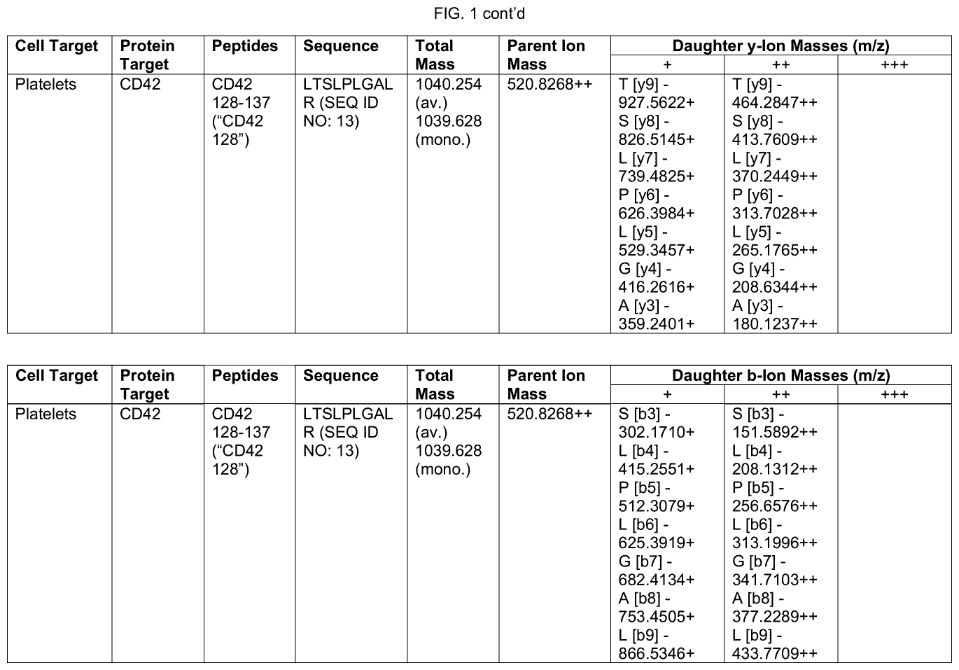

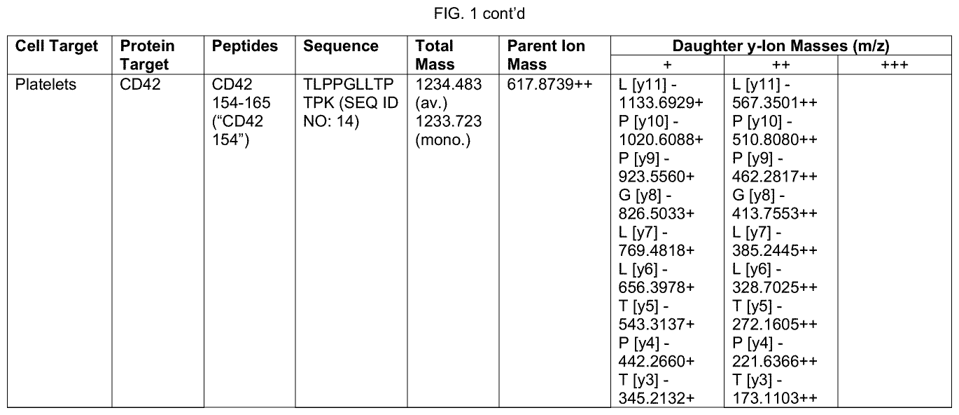

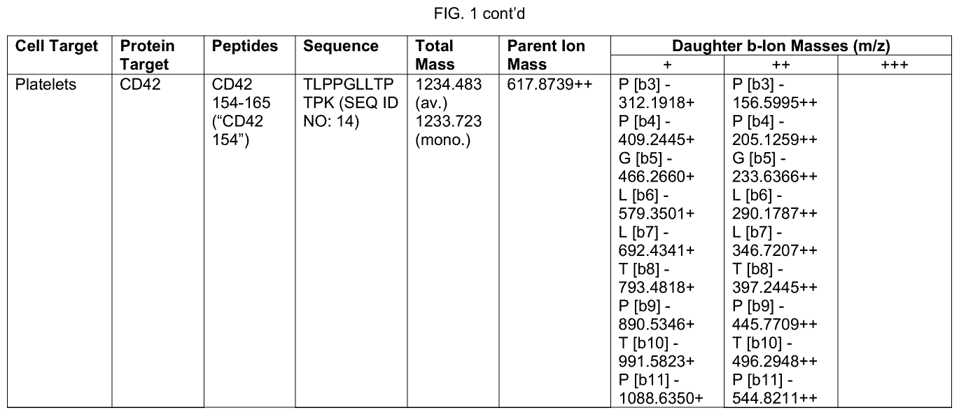

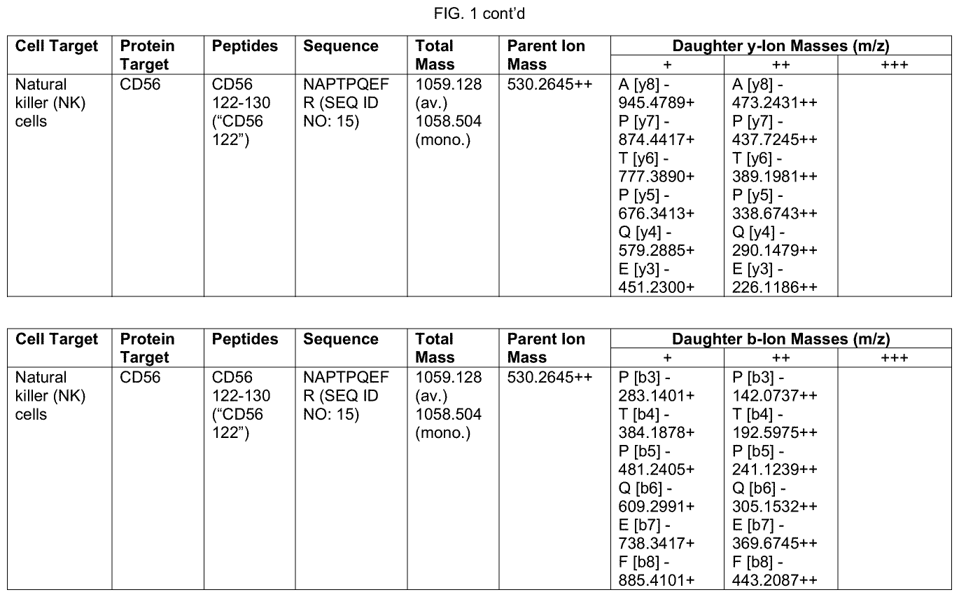

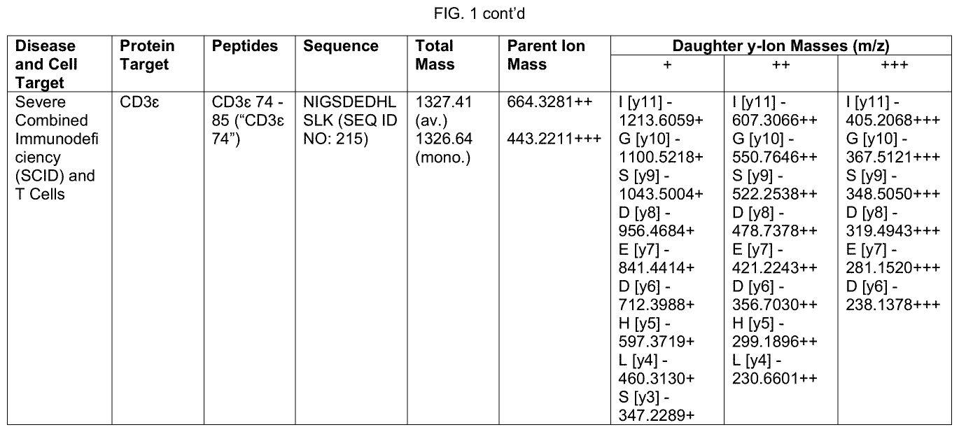

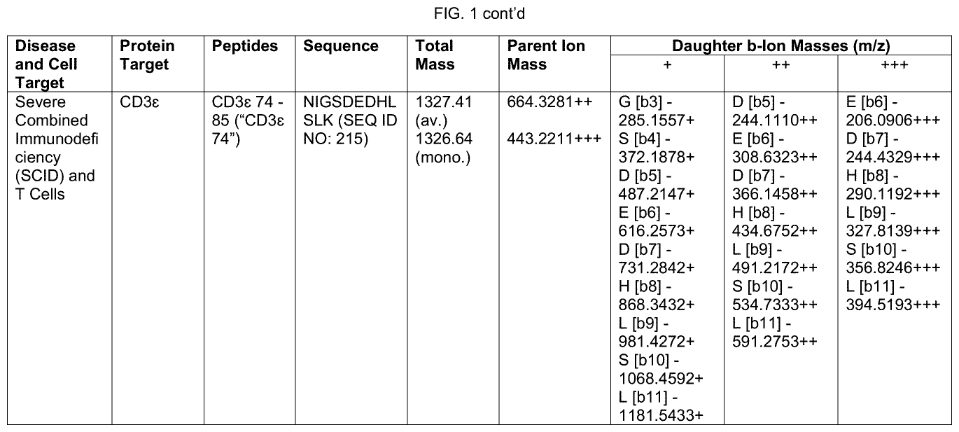

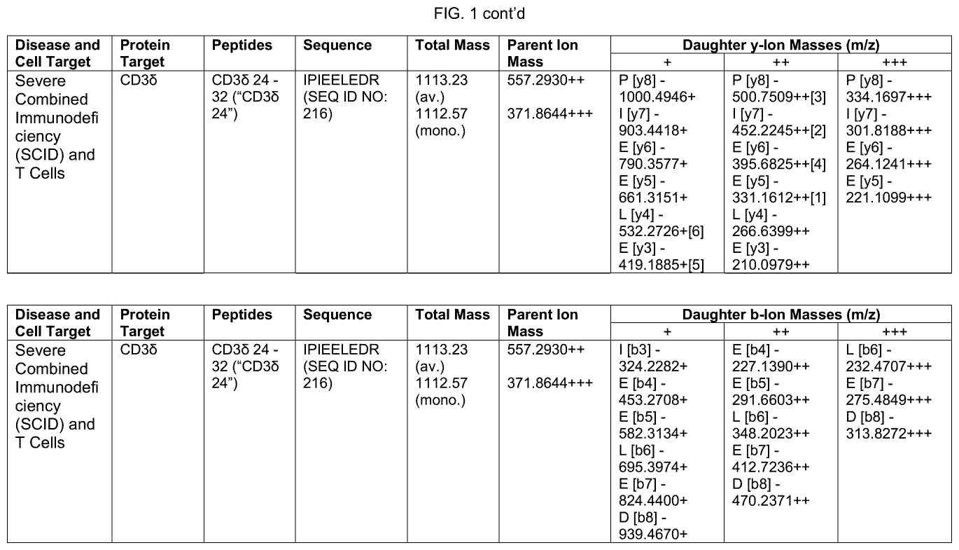

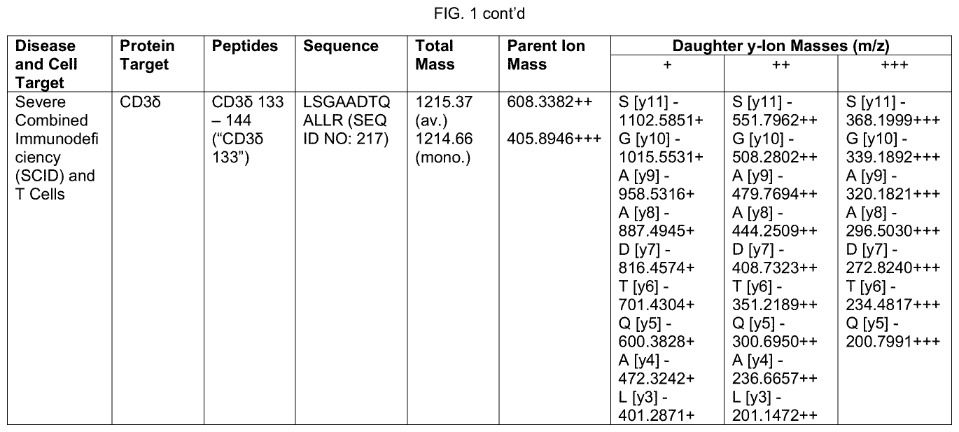

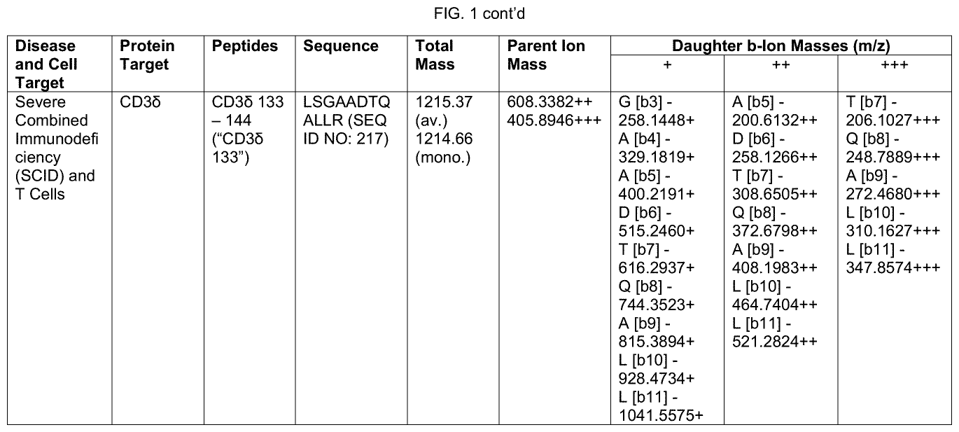

In particular embodiments, the peptides include portions of CYBB, SH2D1A (SAP), PRF-1, ATM, CD19, IL2RG, ADA, DOCKS, CD42, CD56, CD3ε, CD3δ, or a combination thereof. In particular embodiments, the peptides include SEQ ID NOs: 1-15, and 210-217, shown in FIG. 1.

In particular embodiments, exemplary CDR sequences of antibodies of the present disclosure are shown in Table 1A. In particular embodiments, exemplary variable heavy (VH) and variable light (VL) domain sequences of antibodies of the present disclosure are shown in Table 1B. In particular embodiments, SEQ ID NOs of exemplary peptides and antibodies of the present disclosure are shown in Table 1C.

TABLE 1A
Exemplary CDR sequences of antibodies of the present disclosure

| Antibody/ | | |
|---|---|---|
| Disease or | | |
| marker | VH CDRs | VL CDRs |
| Anti-CYBB 509/ | CDR1: RSYWIC | CDR1: QASQSVYNNNALS |
| X-CGD | (SEQ ID NO: 16) | (SEQ ID NO: 19) |
| | CDR2: CIYAGSSGSTYYASWAKG | CDR2: DASDLAP |
| | (SEQ ID NO: 17) | (SEQ ID NO: 20) |
| | CDR3: GNNNIIILDL | CDR3: LGGYSVSSDNA |
| | (SEQ ID NO: 18) | (SEQ ID NO: 21) |
| Anti-SH2D1A 19/ | CDR1: SYDMS | CDR1: QSSQSVYRNKCLA |
| XLP1 | (SEQ ID NO: 160) | (SEQ ID NO: 163) |
| | CDR2: FGNTGGSTYYASWAKG | CDR2: KASTLAS |
| | (SEQ ID NO: 161) | (SEQ ID NO: 164) |
| | CDR3: GAPGWITTNM | CDR3: VGYYNTNTNTTT |
| | (SEQ ID NO: 162) | (SEQ ID NO: 165) |
| Anti-PRF 215/ | CDR1: RHNMG | CDR1: QASQSVYDNNNLA |
| FHL2 | (SEQ ID NO: 34) | (SEQ ID NO: 37) |
| | CDR2: SISSKGKTYYTTWAKG | CDR2: DASKLAS |
| | (SEQ ID NO: 35) | (SEQ ID NO: 38) |
| | CDR3: LYSGSNGYETI | CDR3: LGEFISSSGDCIV |
| | (SEQ ID NO: 36) | (SEQ ID NO: 39) |
| Anti-ATM 1561/ | CDR1: SGGMC | CDR1: QASESLYNNNRLA |
| AT | (SEQ ID NO: 52) | (SEQ ID NO: 55) |
| | CDR2: CIVNGDGSTYFATWAKGR | CDR2: SASTLAS |
| | (SEQ ID NO: 53) | (SEQ ID NO: 56) |
| | CDR3: YWAL | CDR3: VGGRSRVTDGIA |
| | (SEQ ID NO: 54) | (SEQ ID NO: 57) |
| Anti-CD19 41/ | CDR1: | CDR1: |
| CVID | DYSIH | KSSQSLFNSRTRKNYLA |
| | (SEQ ID NO: 70) | (SEQ ID NO: 73) |
| | CDR2: WINTETVEPAYAVDFRG | CDR2: WASTRES |
| | (SEQ ID NO: 71) | (SEQ ID NO: 74) |
| | CDR3: DYSREGVFDY | CDR3: KQSYNLPT |
| | (SEQ ID NO: 72) | (SEQ ID NO: 75) |
| Anti-ADA 93/ | CDR1: NSDWEC | CDR1: QSSQNVVFNNRLA |
| ADA deficiency | (SEQ ID NO: 82) | (SEQ ID NO: 85) |
| | CDR2: CIYGDSRSGYYASWAKGR | CDR2: QASTLAS |
| | (SEQ ID NO: 83) | (SEQ ID NO: 86) |
| | CDR3: NLGSL | CDR3: LGSYDCSSTDCNT |
| | (SEQ ID NO: 84) | (SEQ ID NO: 87) |
| Anti-DOCK8 | CDR1: SYWMH | CDR1: SASSSVSSRYLH |
| 1272/ | (SEQ ID NO: 100) | (SEQ ID NO: 103) |
| Dock8 | CDR2: AIFPGNSNTRYNQKFKG | CDR2: GTSNLSS |
| deficiency | (SEQ ID NO: 101) | (SEQ ID NO: 104) |
| | CDR3: DNAFDY | CDR3: QQYHSDPFT |
| | (SEQ ID NO: 102) | (SEQ ID NO: 105) |
| Anti-CD42 128/ | CDR1: SYNIH | CDR1: TSSQSLLASDGKTYLN |
| marker for | (SEQ ID NO: 112) | (SEQ ID NO: 115) |
| platelets | CDR2: GIYPGNGDTSYNKKFKD | CDR2: LVSKLDS |
| | (SEQ ID NO: 113) | (SEQ ID NO: 116) |
| | CDR3: DGTRY | CDR3: WQGAHSPLT |
| | (SEQ ID NO: 114) | (SEQ ID NO: 117) |
| Anti-CD42 154/ | CDR1: GYNMH | CDR1: RSTKSLLYSDGITYLY |
| marker for | (SEQ ID NO: 124) | (SEQ ID NO: 127) |
| platelets | CDR2: DINPKTGENSYNQKFKG | CDR2: RMSDLAS |
| | (SEQ ID NO: 125) | (SEQ ID NO: 128) |
| | CDR3: WGVYYLDH | CDR3: AQMVEFPPT |
| | (SEQ ID NO: 126) | (SEQ ID NO: 129) |
| Anti-CD56 122/ | CDR1: SNYYMC | CDR1: QASQSIDSWCA |
| marker for NK | (SEQ ID NO: 136) | (SEQ ID NO: 139) |
| cells | CDR2: | CDR2: |
| | CIYSSSLNTWYASWAKGR | KASTLES |
| | (SEQ ID NO: 137) | (SEQ ID NO: 140) |
| | CDR3: GGGVYVGADYGFYFYL | CDR3: QSYYPTTGSDNA |
| | (SEQ ID NO: 138) | (SEQ ID NO: 141) |

TABLE 1B
Exemplary variable heavy (VH) and variable light (VL)
domain sequences of antibodies of the present disclosure

| | SEQ | |
|---|---|---|
| | ID | |
| Variable domain | NO: | Amino acid sequence |
| Anti-CYBB 509 VH | 27 | QSLEESGGGLVKPGASLTLTCTASGFSFSRSYWICWVRQAPGKGLEWLGC |
| | | IYAGSSGSTYYASWAKGRFTISKTSSTTVTLQMTSLTAADTATYFCARGN |
| | | NNIIILDLWGPGILVTVSS |
| Anti-CYBB 509 VL | 33 | AVLTQTPSPVSAAVGGTVTISCQASQSVYNNNALSWYQQKPGQPPKLLIY |
| | | DASDLAPGVPSRFSGSGSGTQFTLTISDVQCDDAATYYCLGGYSVSSDNA |
| | | FGGGTEVVVN |
| Anti-SH2D1A 19 VH | 173 | QSLEESGGRLVTPGGTLTLTCTVSGFSLSSYDMSWVRQAPGKGLEWIGFG |
| | | NTGGSTYYASWAKGRFTISRTSTTVDLRMTSLTAADTATYFCTRGAPGWI |
| | | TTNMWGPGTLVTVSL |
| Anti-SH2D1A 19 VL | 181 | QVLTQTPSPVSAAVGGTVTISCQSSQSVYRNKCLAWYQQKPGQPPKLLIY |
| | | KASTLASGVPSRFSGSGSGTQFTFTISDLECDDSATYYCVGYYNTNTNTT |
| | | TFGGGTEVVVK |
| Anti-PRF 215 VH | 45 | QSVEESGGRLVTPGGTLTLTCTASGFSLSRHNMGWVRQAPGEGLEWIGSI |
| | | SSKGKTYYTTWAKGRFTISKSSTTVDLKITSPTTEDTATYFCARLYSGSN |
| | | GYETIWGPGTLVTVSL |
| Anti-PRF 215 VL | 51 | ADVVMTQTPSPVSAAVGGTVTINCQASQSVYDNNNLAWYQQKPGQPPKLL |
| | | IYDASKLASGVPSRFSGSGSGTQFTLTISDVQCDDAATYYCLGEFISSSG |
| | | DCIVFGGGTEMVVK |
| Anti-ATM 1561 VH | 63 | QEQLVESGGGLVQPGGSLTLTCKASGFDFSSGGMCWVRQAPGKGLEWIGC |
| | | IVNGDGSTYFATWAKGRFTITRTSSTTVTLQMTSLTAADTATYFCWRYWA |
| | | LWGPGTLVTVSS |
| Anti-ATM 1561 VL | 69 | IVMTQTPSSKSVPVGGTVTINCQASESLYNNNRLAWFQQKPGQPPKLLIY |
| | | SASTLASGVPDRFSGSGSGTQFTLTISDVVCDDAATYYCVGGRSRVTDGI |
| | | AFGGGTEAVVK |
| Anti-CD19 41 VH | 78 | QIQLVQSGPELKKPGETVKISCKASDYTFTDYSIHWVKQAPGKGLKWLGW |
| | | INTETVEPAYAVDFRGRFALSLETSANTAYLQINNLKNEDAATYFCTRDY |
| | | SREGVFDYWGQGTTLTVSS |
| Anti-CD19 41 VL | 81 | DNVMSQSPSSLAVSAGEKVTMSCKSSQSLFNSRTRKNYLAWYQQKPGQSP |
| | | KLLVYWASTRESGVPDRFTGSGSGTDFTLTISNVQAEDLAVYYCKQSYNL |
| | | PTFGAGTKLELK |
| Anti-ADA 93 VH | 93 | QEQLEESGGDLVKPGSSLTLTCTASGFSFTNSDWECWVRQAPGKGLEWIA |
| | | CIYGDSRSGYYASWAKGRFTISKTSSTTVTLQMTTLTAADTATYFCARNL |
| | | GSLWGPGTLVTVSS |
| Anti-ADA 93 VL | 99 | QVLTQTPSSVSAAVGGTVTINCQSSQNVVFNNRLAWFQQKPGQPPKLLIY |
| | | QASTLASGVPPRFKGSGSGTQFTLTISDLECDDAATYYCLGSYDCSSTDC |
| | | NTFGGGTEVVVK |
| Anti-DOCK8 | 108 | EVQLQQSGTVLARPGASVKMSCKASGYTFTSYWMHWVKQRPGQGLEWIGA |
| 1272 VH | | IFPGNSNTRYNQKFKGKAKLTAVTSASTAYMELSSLTNEDSAVYYCADDN |
| | | AFDYWGQGTTLTVSS |
| Anti-DOCK8 | 111 | QIVLTQSPAIMSASPGEKVTMTCSASSSVSSRYLHWYQQKSGASPKLWIY |
| 1272 VL | | GTSNLSSGVPDRFSGSGSGTSYSLTISSVETEDAATYYCQQYHSDPFTFG |
| | | TGTKLEIK |
| Anti-CD42 128 VH | 120 | QAYLQQSGAVLVRPGASVKMSCKASGYTFSSYNIHWVKQTPRQGLEWIGG |
| | | IYPGNGDTSYNKKFKDKATLTVDKSSNTAYMHLSSLTSEDSAVFFCASDG |
| | | TRYWGQGTTLTVSS |
| Anti-CD42 128 VL | 123 | EVVMTQTPLTLSVTIGQAVSISCTSSQSLLASDGKTYLNWLLQRPGQSPK |
| | | RLIYLVSKLDSGVPDRFTGSGSGTDFTLKISRVEADDLGVYCCWQGAHSP |
| | | LTFGAGTRLELK |
| Anti-CD42 154 VH | 132 | EVQLQQSGPELVKPGASVKMSCKASGYTFTGYNMHWVKQSHGKSLEWIGD |
| | | INPKTGENSYNQKFKGKATLTISKSSSTAYMELRSLTSEDSAVYYCVRWG |
| | | VYYLDHWGQGTTLTVSS |
| Anti-CD42 154 VL | 135 | DIVMTQAAFSNPVTLGTSASISCRSTKSLLYSDGITYLYWYLQRPGQSPQ |
| | | LLIYRMSDLASGVPDRFSGSGSGTDFTLRISRVEAEDVGVYYCAQMVEFP |
| | | PTFGGGTKLEIK |
| Anti-CD56 122 VH | 147 | QSLEESGGDLVKPGASLTLTCKASGLDFSSNYYMCWVRQAPGKGLEWIAC |
| | | IYSSSLNTWYASWAKGRFTISKSTSLHTVTLQMTSLTAADTATYFCTRGG |
| | | GVYVGADYGFYFYLWGPGTLVTVSS |
| Anti-CD56 122 VL | 153 | ADIVMTQTPSSVEAPVGGTVTIKCQASQSIDSWCAWYQQKPGQPPKLLIY |
| | | KASTLESGVPSRFKGSGSGTEFTLTISDLECADAATYFCQSYYPTTGSDN |
| | | AFGGGTEVVVK |

TABLE 1C
SEQ ID NOs for exemplary peptides and antibodies of the present disclosure

| | | | SEQ ID NOs | | | |
|---|---|---|---|---|---|---|
| | | | | | Heavy | Light |
| | | | | | chain | chain |
| Disease and/or | Target | Peptides | VH | VL | VH | VL |
| Target Cell | Protein | SEQ ID NO | CDRs | CDRs | domain | domain |
| X-linked Chronic | CYBB | CYBB 131-147 | | | | |
| Granulomatous | | SEQ ID NO: 1 | | | | |
| Disease (X-CGD) | | CYBB 509-521 | 16-18 | 19-21 | 22-24, | 28-30, |
| | | SEQ ID NO: 2 | | | 182 | 184 |
| | | | | | 25-27, | 31-33, |
| | | | | | 183 | 185 |
| X-linked | SAP | SH2D1A 19-32 | 160-162 | 163-165 | 166, 167, | 174, 175, |
| lymphoproliferative | | SEQ ID NO: 3 | | | 168, 169 | 176, 177 |
| Syndrome 1 (XLP1) | | | | | 170, 171, | 178, 179, |
| | | | | | 172, 173 | 180, 181 |
| | | SH2D1A 110-118 | | | | |
| | | SEQ ID NO: 4 | | | | |
| Familial | PRF-1 | PRF 215-225 | 34-36 | 37-39 | 40-42, | 46-48, |
| Hemophagocytic | | SEQ ID NO: 5 | | | 186 | 188 |
| Lymphohistiocytosis | | | | | 43-45, | 49-51, |
| (FHL2) | | | | | 187 | 189 |
| | | PRF 441-449 | | | | |
| | | SEQ ID NO: 6 | | | | |
| Ataxia Telangiectasia | ATM | ATM 798-805 | | | | |
| | | SEQ ID NO: 210 | | | | |
| | | ATM 1544-1550 | | | | |
| | | SEQ ID NO: 211 | | | | |
| | | ATM 1561-1572 | 52-54 | 55-57 | 58-60, | 64-66, |
| | | SEQ ID NO: 7 | | | 190 | 192 |
| | | | | | 61-63, | 67-69, |
| | | | | | 191 | 193 |
| Common Variable | CD19 | CD19 41-54 | 70-72 | 73-75 | | |
| Immunodeficiency | | SEQ ID NO: 8 | | | | |
| (CVID) | | | | | 76-78, | 79-81, |
| | | | | | 194 | 195 |
| | | CD19 55-63 | | | | |
| | | SEQ ID NO: 212 | | | | |
| | | CD19 210-220 | | | | |
| | | SEQ ID NO: 9 | | | | |
| | | CD19 505-514 | | | | |
| | | SEQ ID NO: 10 | | | | |
| Severe Combined | IL2RG | IL2RG 295-315 | | | | |
| Immunodeficiency | | SEQ ID NO: 213 | | | | |
| (SCID) | | | | | | |
| SCID | IL2RG | IL2RG 316-328 | | | | |
| | | SEQ ID NO: 214 | | | | |
| SCID and T cells | CD3ε | CD3ε 74-85 | | | | |
| | | SEQ ID NO: 215 | | | | |
| | CD3δ | CD3δ 24-32 | | | | |
| | | SEQ ID NO: 216 | | | | |
| | CD3δ | CD3δ 133-144 | | | | |
| | | SEQ ID NO: 217 | | | | |
| Adenosine deaminase | ADA | ADA 93-101 | 82-84 | 85-87 | 88-90, | 94-96, |
| (ADA) Deficiency | | SEQ ID NO: 11 | | | 196 | 198 |
| | | | | | 91-93, | 97-99, |
| | | | | | 197 | 199 |
| DOCK8 Deficiency | DOCK8 | Dock8 1272-1283 | 100-102 | 103-105 | | |
| | | SEQ ID NO: 12 | | | | |
| | | | | | 106-108, | 109-111, |
| | | | | | 200 | 201 |
| Platelets (secondary | CD42 | CD42 128-137 | 112-114 | 115-117 | | |
| marker) | | SEQ ID NO: 13 | | | | |
| | | | | | 118-120, | 121-123, |
| | | | | | 202 | 203 |
| | | CD42 154-165 | 124-126 | 127-129 | | |
| | | SEQ ID NO: 14 | | | | |
| | | | | | 130-132, | 133-135, |
| | | | | | .204 | 205 |
| Natural killer (NK) cells | CD56 | CD56 122-130 | 136-138 | 139-141 | 142-144, | 148-150, |
| (secondary marker) | | SEQ ID NO: 15 | | | 206 | 208 |
| | | | | | 145-147, | 151-153, |
| | | | | | 207 | 209 |

*Underlined SEQ ID NOs denote nucleotide sequences. Non-underlined SEQ ID NOs denote amino acid sequences.

### (III) ENZYMATIC DIGESTION OF PROTEINS IN A BIOLOGICAL SAMPLE

Proteins in DBS, in cells from a buccal swab sample, in PBMC, or in WBC can be subjected to proteolysis to produce peptides that can be further selected by immunoaffinity purification before analysis by LC-SRM-MS. Proteolysis can be accomplished using site specific endoproteases, such as pepsin, arg-C proteinase, asp-N endopeptidase, BNPS-skatole, caspase 1, caspase 2, caspase 3, caspase 4, caspase 5, caspase 6, caspase 7, caspase 8, caspase 9, caspase 10, chymotrypsin, clostripain (clostridiopeptidase B), enterokinase, factor Xa, glutamyl endopeptidase, granzyme B, lysC, proline-endopeptidase, proteinase K, staphylococcal peptidase I, thermolysin, thrombin, and trypsin. Chemicals which cleave site specifically can also be used. Combinations of enzymes and/or chemicals can be used to obtain desirable analytes.

In particular embodiments, proteins in DBS, in cells from a buccal swab sample, in PBMC, or in WBC can be digested into peptides with trypsin. Trypsin cleaves exclusively C-terminal to arginine and lysine residues and can be a preferred choice to generate peptides because the masses of generated peptides are compatible with the detection ability of most mass spectrometers (up to 2000 m/z) and because there are efficient algorithms available for the generation of databases of theoretical trypsin-generated peptides. High cleavage specificity, availability, and low cost are other advantages of trypsin. Peptides formed by the treatment of a protein with trypsin are known as tryptic peptides.

### (IV) ANTIBODIES TO ENRICH FOR THE PEPTIDE MARKERS

An antibody includes a polypeptide ligand substantially encoded by an immunoglobulin gene or immunoglobulin genes, or fragments thereof, whether natural, or partially or wholly synthetically produced. An antibody specifically (or selectively) binds and recognizes an epitope (e.g., an antigen). An antibody can include any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE, etc. The recognized immunoglobulin genes include the kappa and lambda light chain constant region genes, the alpha, gamma, delta, epsilon and mu heavy chain constant region genes, and the myriad immunoglobulin variable region genes. “Fc” portion of an antibody refers to that portion of an immunoglobulin heavy chain that includes one or more heavy chain constant region domains, CH1, CH2 and CH3, but does not include the heavy chain variable region.

An intact antibody can include at least two heavy (H) chains and two light (L) chains inter-connected by disulfide bonds. Each heavy chain is composed of a heavy chain variable region (abbreviated herein as VH or V_{H}) and a heavy chain constant region. The heavy chain constant region includes three domains, CH1, CH2 and CH3. Each light chain is composed of a light chain variable region (abbreviated herein as VL or V_{L}) and a light chain constant region. The light chain constant region includes one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al. (1997) J Mol Biol 273: 927-948 (Chothia numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); Lefranc M P et al. (2003) Dev Comp Immunol 27(1): 55-77 (IMGT numbering scheme); and Honegger and Pluckthun (2001) J Mol Biol 309(3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering.

An antibody fragment includes any derivative or portion of an antibody that is less than full-length. In particular embodiments, the antibody fragment retains at least a significant portion of the full-length antibody's specific binding ability as a binding partner. Examples of antibody fragments include Fab, Fab′, Fab′-SH, F(ab′)₂, single chain variable fragment (scFv), Fv, dsFv diabody, and Fd fragments, and/or any biologically effective fragments of an immunoglobulin that bind specifically to an epitope described herein. Antibodies or antibody fragments include all or a portion of polyclonal antibodies, monoclonal antibodies, human antibodies, humanized antibodies, synthetic antibodies, chimeric antibodies, bispecific antibodies, mini bodies, and linear antibodies.

A single chain variable fragment (scFv) is a fusion protein of the variable regions of the heavy and light chains of immunoglobulins connected with a short linker peptide. Fv fragments include the VL and VH domains of a single arm of an antibody. Although the two domains of the Fv fragment, VL and VH, are coded by separate genes, they can be joined, using, for example, recombinant methods, by a synthetic linker that enables them to be made as a single protein chain in which the VL and VH regions pair to form monovalent molecules (single chain Fv (scFv)). For additional information regarding Fv and scFv, see e.g., Bird, et al., Science 242 (1988) 423-426; Huston, et al., Proc. Natl. Acad. Sci. USA 85 (1988) 5879-5883; Plueckthun, in The Pharmacology of Monoclonal Antibodies, vol. 113, Rosenburg and Moore (eds.), Springer-Verlag, New York), (1994) 269-315; WO1993/16185; U.S. Pat. Nos. 5,571,894; and 5,587,458.

A Fab fragment is a monovalent antibody fragment including VL, VH, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90 (1993) 6444-6448. Dual affinity retargeting antibodies (DART™; based on the diabody format but featuring a C-terminal disulfide bridge for additional stabilization (Moore et al., Blood 117, 4542-51 (2011)) can also be used. Antibody fragments can also include isolated CDRs. For a review of antibody fragments, see Hudson, et al., Nat. Med. 9 (2003) 129-134.

The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may include a single chain antibody fragment. In another embodiment, the fragment may include multiple chains that are linked together, for example, by disulfide linkages. The fragment may also include a multimolecular complex. A functional antibody fragment may typically include at least 50 amino acids and more typically will include at least 200 amino acids.

In particular embodiments, recombinant immunoglobulins can be produced. See, Cabilly, U.S. Pat. No. 4,816,567, and Queen et al., Proc Natl Acad Sci USA, 86:10029-10033 (1989).

As indicated, in particular embodiments, binding domains of an engineered antibody or antigen binding fragment may be joined through a linker. A linker is an amino acid sequence which can provide flexibility and room for conformational movement between the binding domains of an engineered antibody or antigen binding fragment. Any appropriate linker may be used. Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target. Commonly used flexible linkers include Gly-Ser linkers such as GGSGGGSGGSG (SEQ ID NO: 154), GGSGGGSGSG (SEQ ID NO: 155) and GGSGGGSG (SEQ ID NO: 156). Additional examples include: GGGGSGGGGS (SEQ ID NO: 157); GGGSGGGS (SEQ ID NO: 158); and GGSGGS (SEQ ID NO: 159). Linkers that include one or more antibody hinge regions and/or immunoglobulin heavy chain constant regions, such as CH3 alone or a CH2CH3 sequence can also be used.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

It will also be understood by one of ordinary skill in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation.

A monoclonal antibody includes an antibody obtained from a population of substantially homogeneous antibodies, i.e., the individual antibodies including the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. This type of antibody is produced by the daughter cells of a single antibody-producing hybridoma. A monoclonal antibody typically displays a single binding affinity for any epitope with which it binds.

The modifier “monoclonal” indicates the character of the antibody as being obtained from a homogeneous population of antibodies and is not to be construed as requiring production of the antibody by any particular method. Monoclonal antibodies recognize only one type of antigen. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. Techniques for the production of antibodies are well known in the art and described in, e.g., Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999; Tickle et al. JALA: Journal of the Association for Laboratory Automation. 2009; 14(5): 303-307; Babcook et al. Proc. Natl. Acad. Sci. U.S.A. 1996; 93: 7843-7848; and U.S. Pat. No. 5,627,052.

In particular embodiments “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and peptide). The affinity of a molecule X for its partner Y can generally be represented by the dissociation constant (K_{D}) or the association constant (K_{A}). Affinity can be measured by common methods known in the art.

In particular embodiments, “bind” means that the binding domain of an antibody associates with its target peptide with a dissociation constant (K_{D}) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷ M, in particular embodiments of from 10⁻⁸ M to 10⁻¹³ M, or in particular embodiments of from 10⁻⁹ M to 10⁻¹³ M. The term can be further used to indicate that the binding domain does not bind to other biomolecules present, (e.g., it binds to other biomolecules with a dissociation constant (K_{D}) of 10⁻⁴ M or more, in particular embodiments of from 10⁻⁴ M to 1 M).

In particular embodiments, “bind” means that the binding domain of an antibody associates with its target peptide with an affinity constant (i.e., association constant, K_{A}) of 10⁷ M⁻¹ or more, in particular embodiments of from 10⁵ M⁻¹ to 10¹³ M⁻¹, in particular embodiments of from 10⁵ M⁻¹ to 10¹⁰ M⁻¹, in particular embodiments of from 10⁵ M⁻¹ to 10⁸ M⁻¹, in particular embodiments of from 10⁷ M⁻¹ to 10¹³ M⁻¹, or in particular embodiments of from 10⁷ M⁻¹ to 10⁸ M⁻¹. The term can be further used to indicate that the binding domain does not bind to other biomolecules present, (e.g., it binds to other biomolecules with an association constant (K_{A}) of 10⁴ M⁻¹ or less, in particular embodiments of from 10⁴ M⁻¹ to 1 M⁻¹).

Antibodies of the present disclosure can be used for immunoaffinity enrichment of peptides described herein detected in SRM assays for diagnosis of X-CGD, XLP1, FHL2, ataxia telangiectasia, CVID, SCID, ADA deficiency, and DOCK8 deficiency, and assessment of secondary peptide markers CD42, CD56, CD3ε, and CD3δ. In particular embodiments, CD3ε and CD3δ can be used as primary markers to diagnose SCID. Particular embodiments of the high affinity antibodies include anti-CYBB 131, anti-CYBB 509, anti-SH2D1A 19, anti-SH2D1A 110, anti-PRF 215, anti-PRF 441, anti-ATM 798, anti-ATM 1544, anti-ATM 1561, anti-CD19 41, anti-CD19 55, anti-CD19 210, anti-CD19 505, anti-IL2RG 295, anti-IL2RG 316, anti-ADA 93, anti-Dock8 1272, anti-CD42 128, anti-CD42 154, anti-CD56 122, anti-CD3ε 74, anti-CD3δ 24, and anti-CD3δ 133.

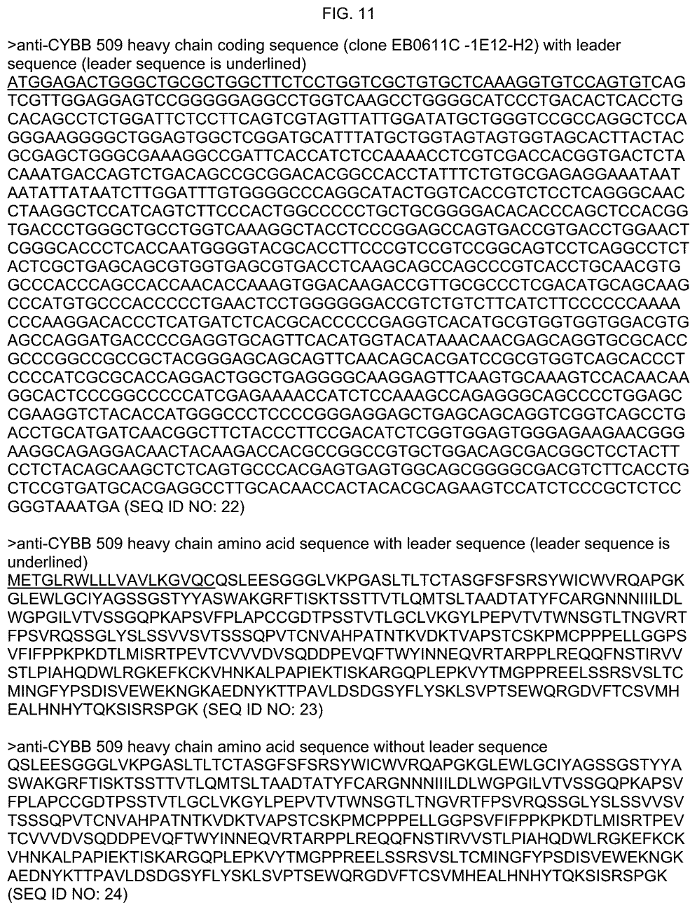

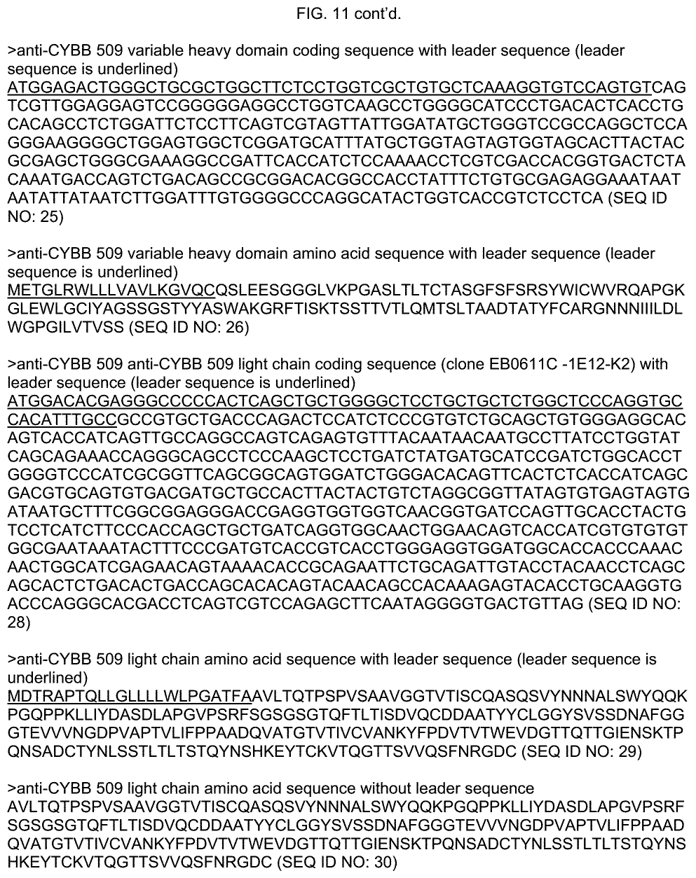

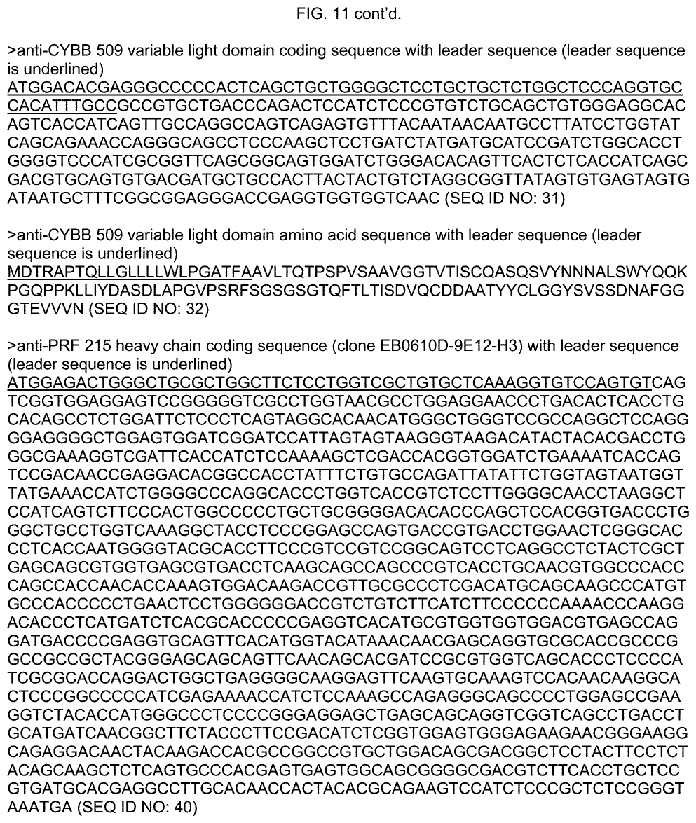

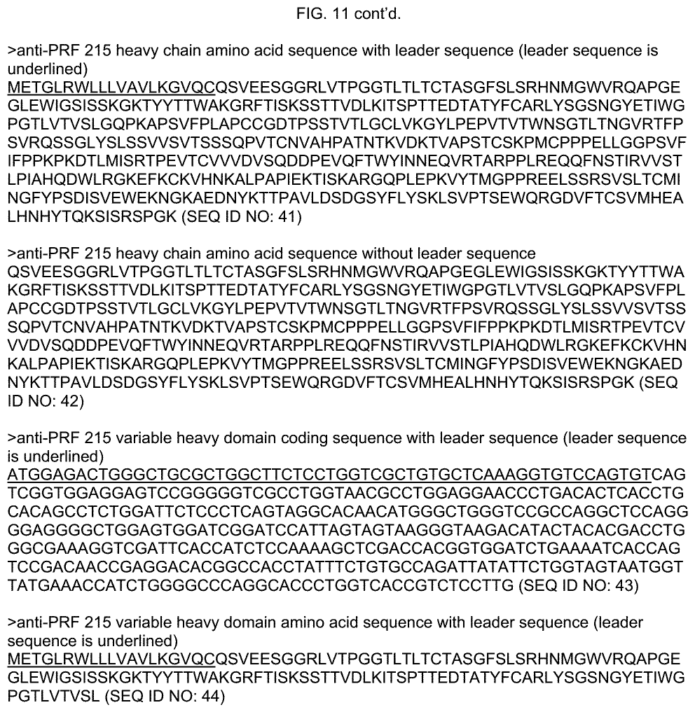

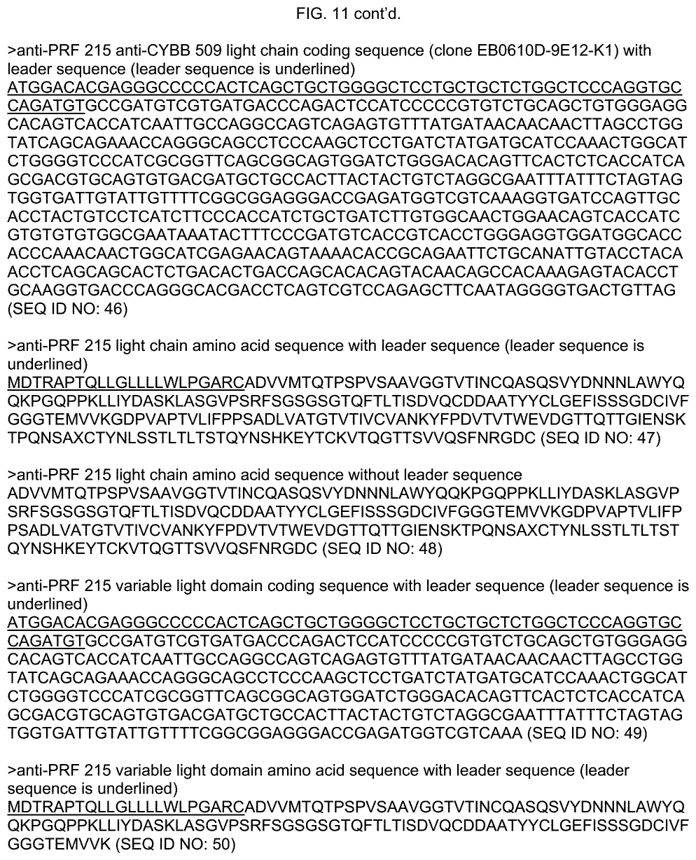

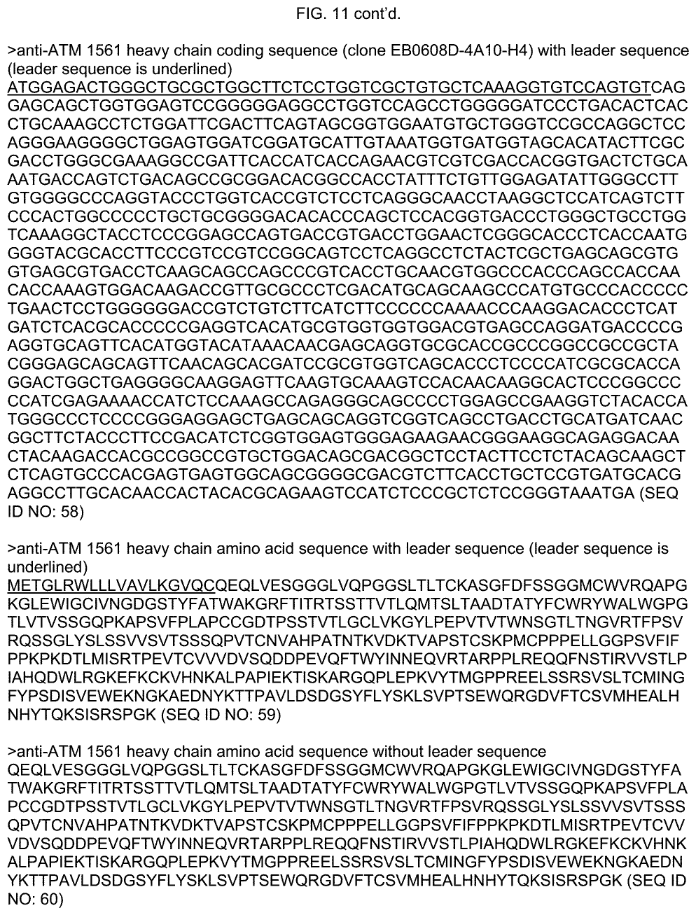

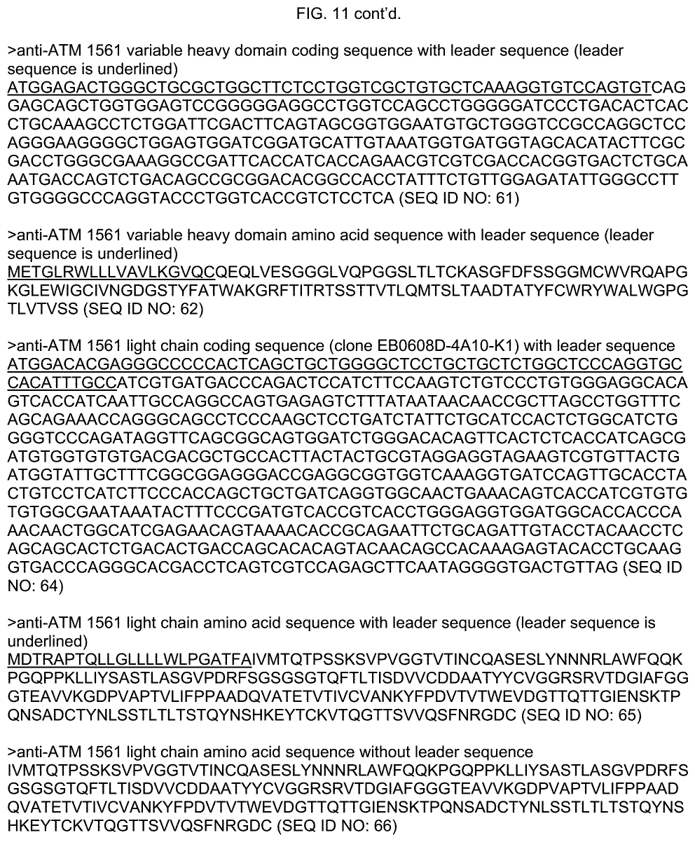

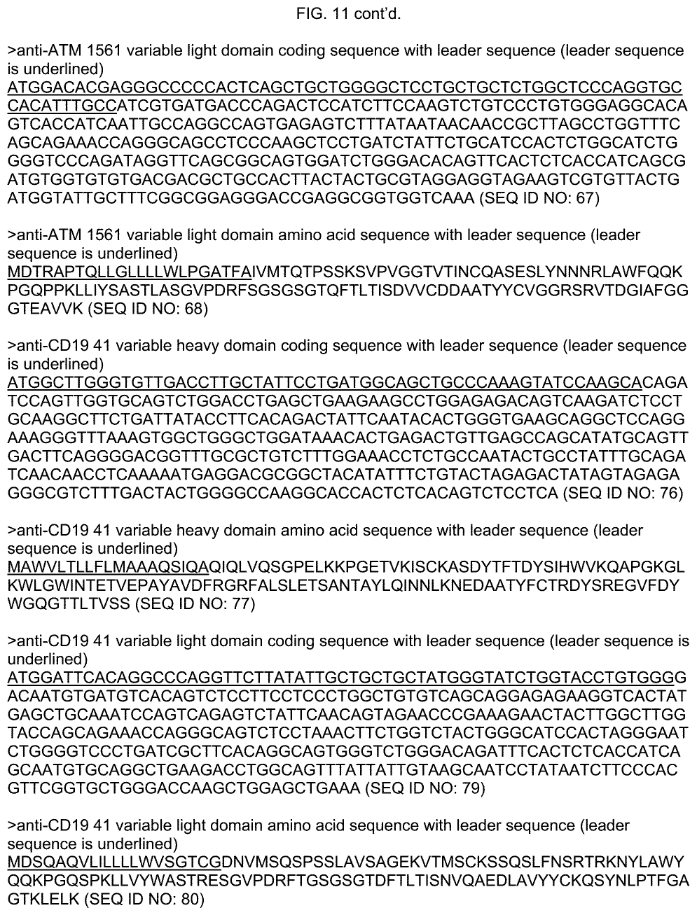

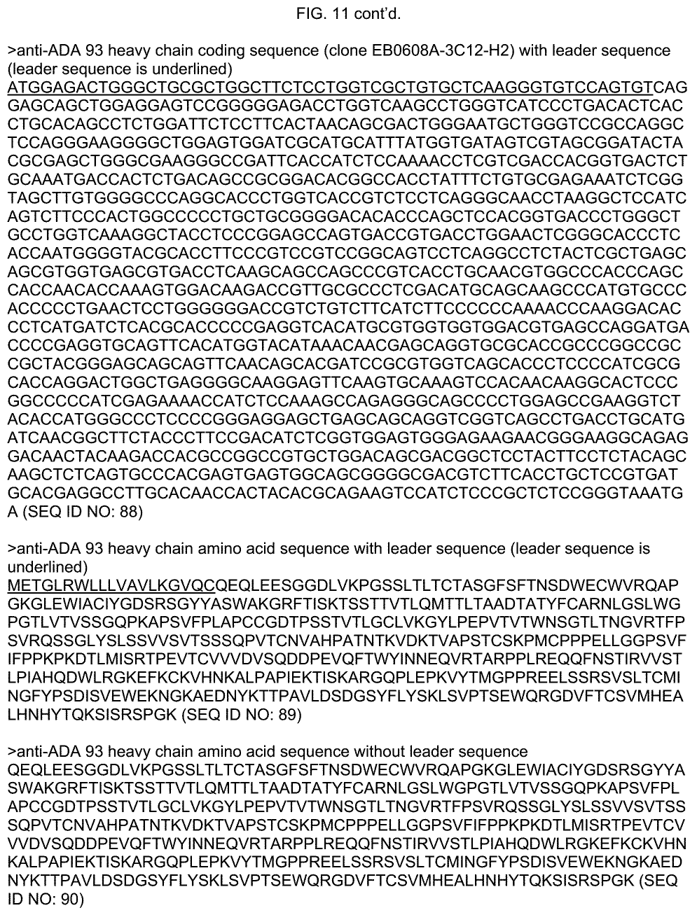

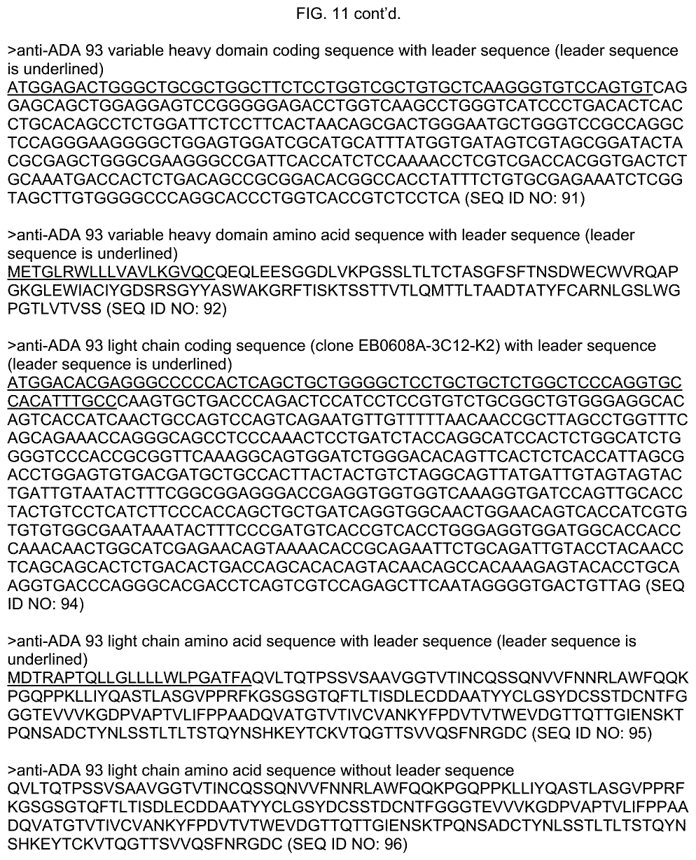

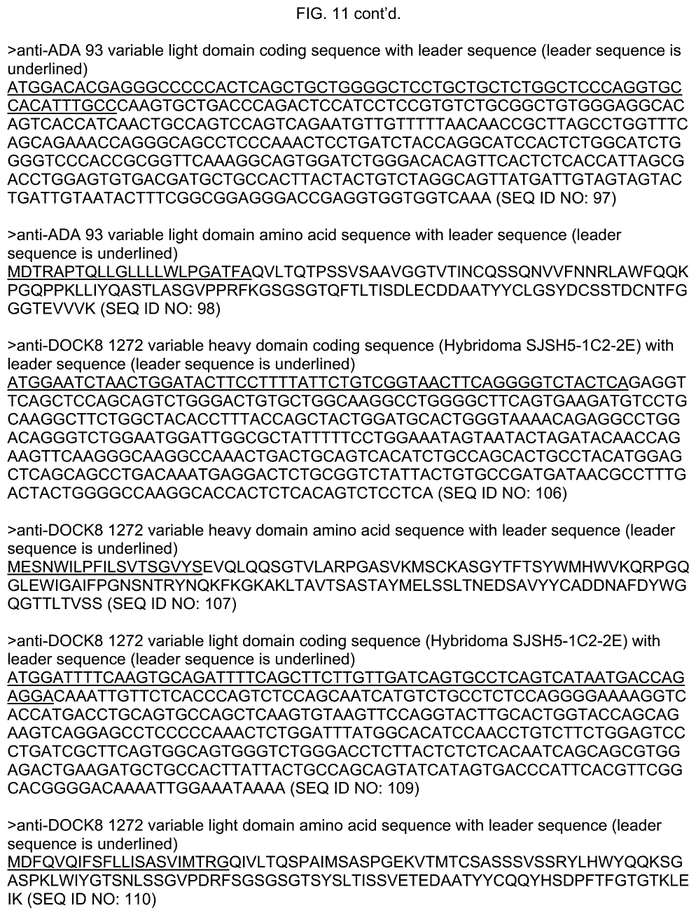

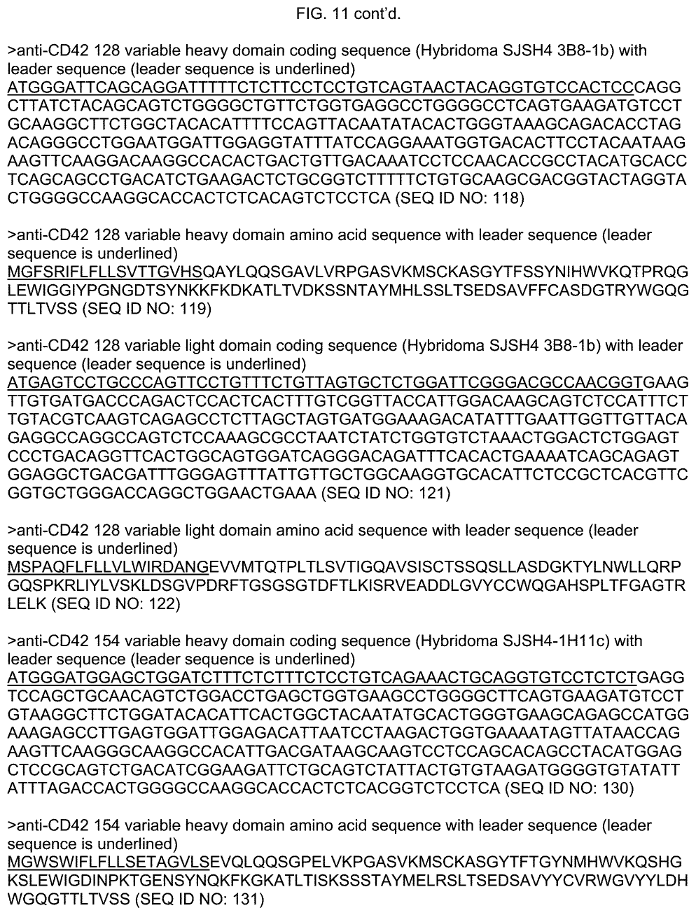

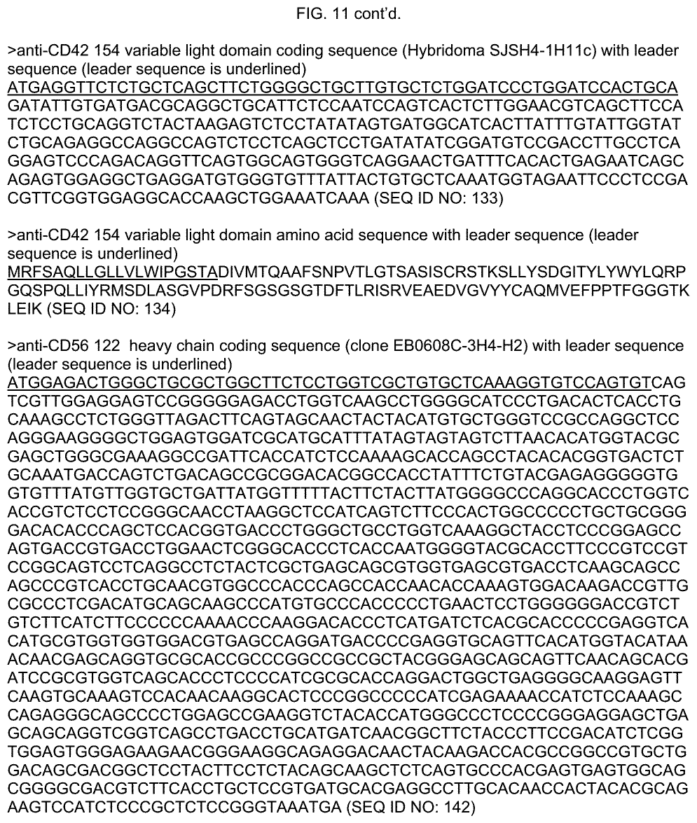

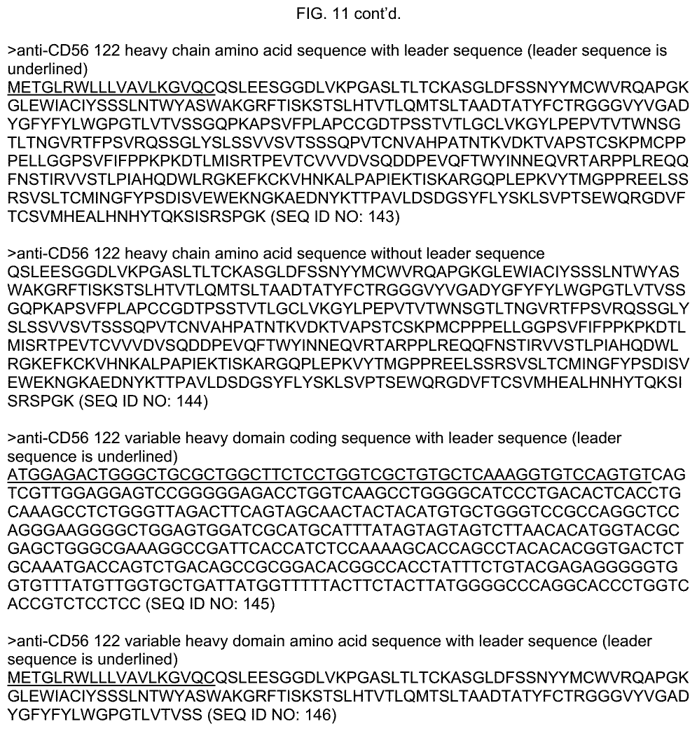

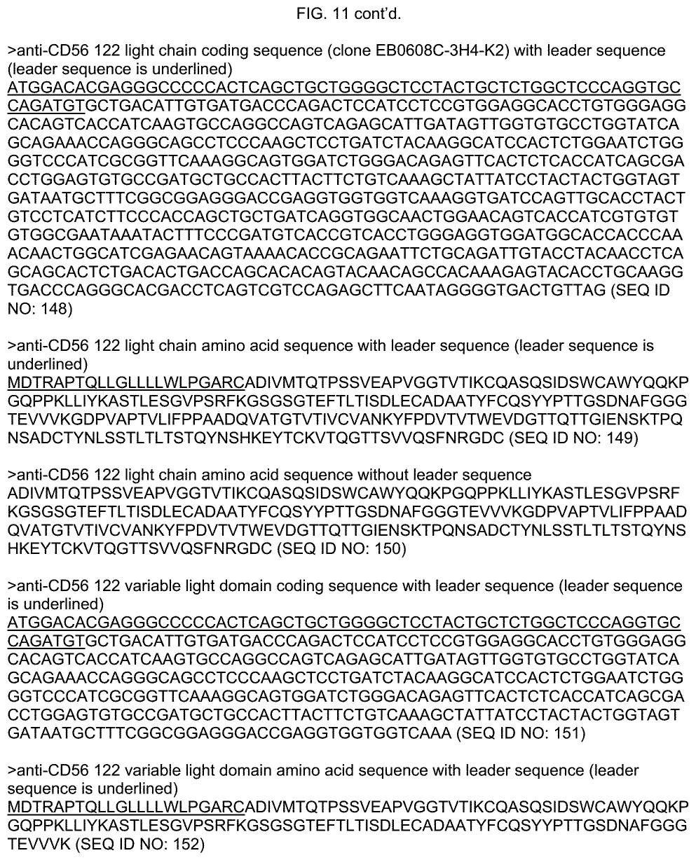

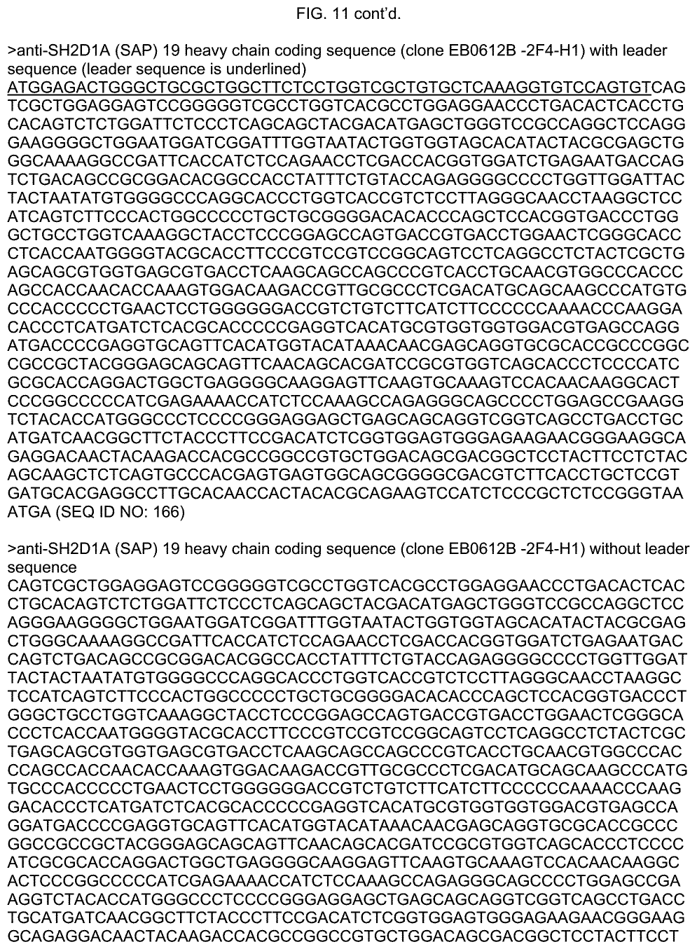

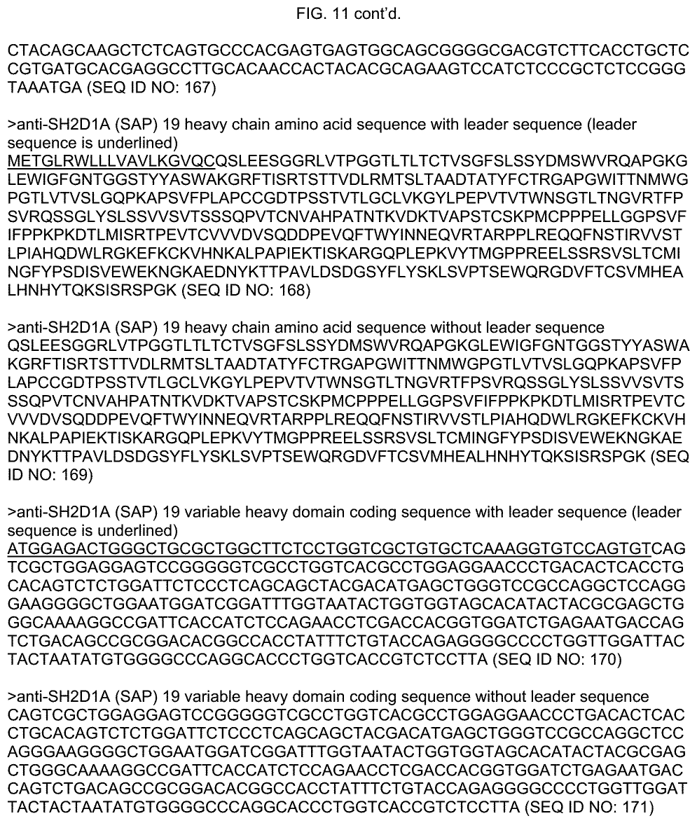

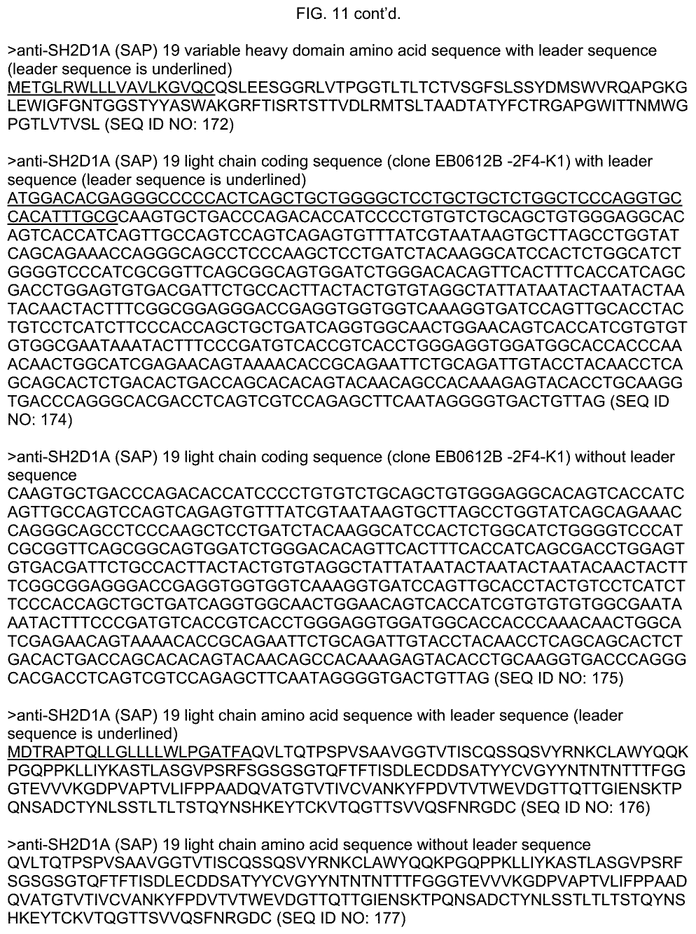

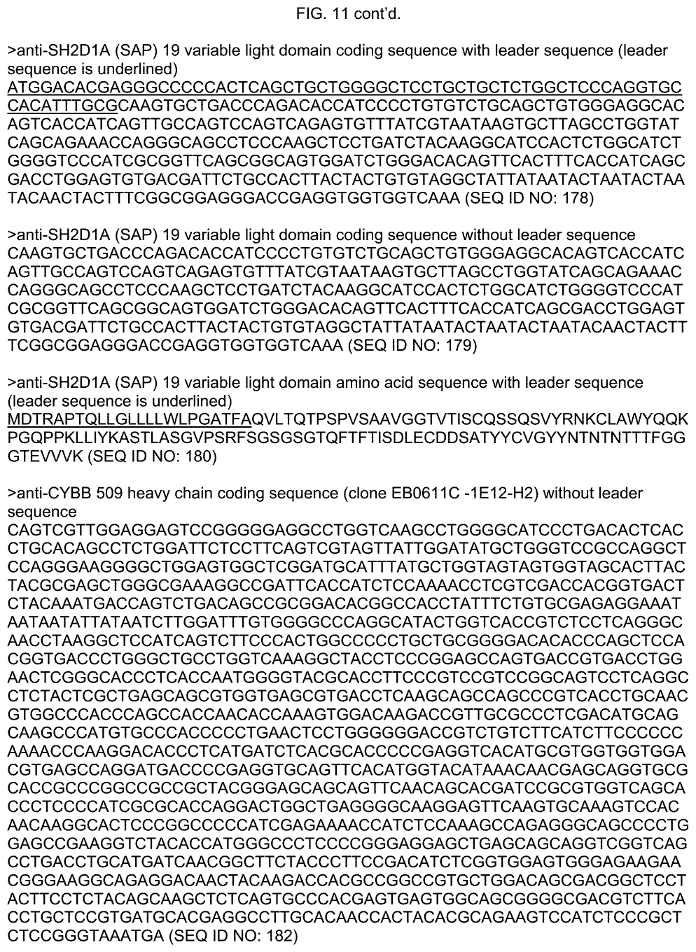

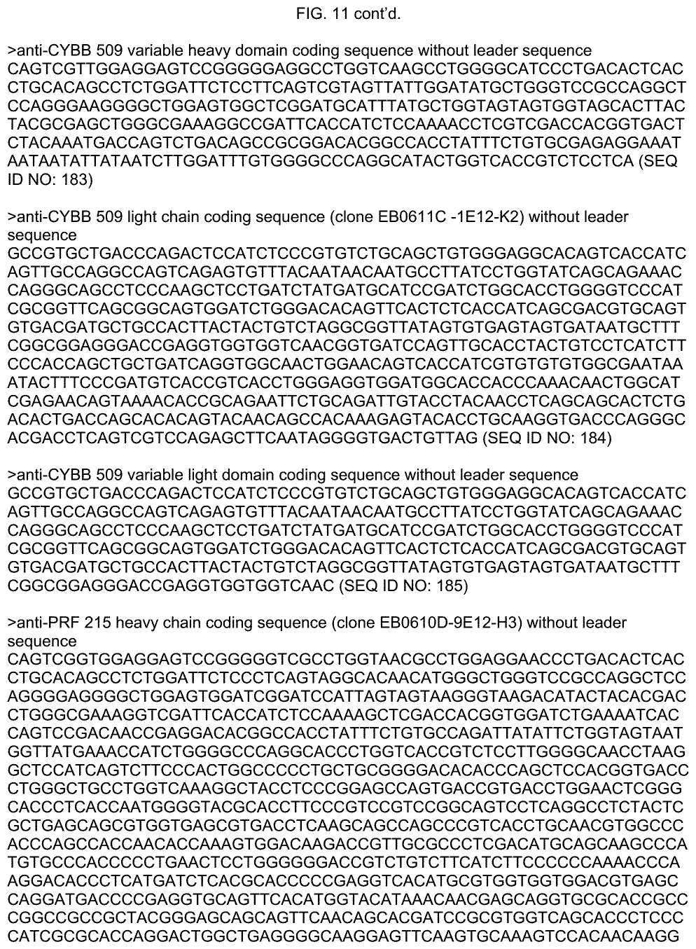

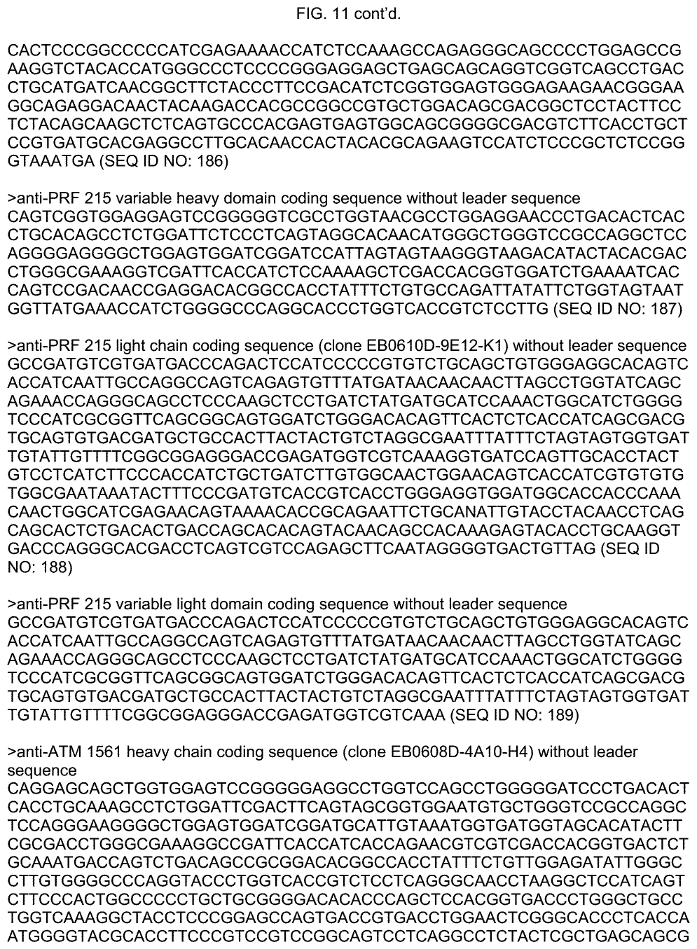

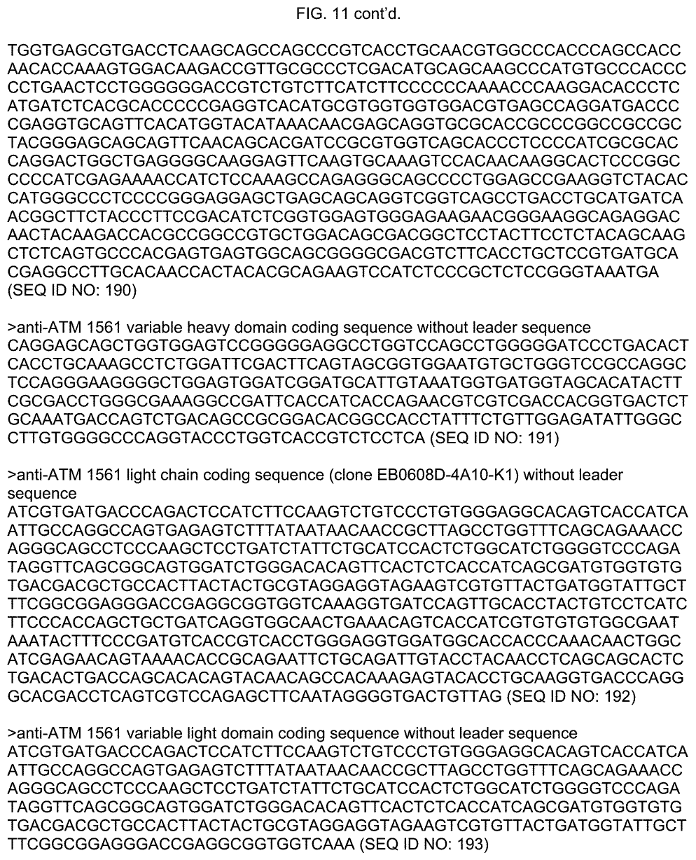

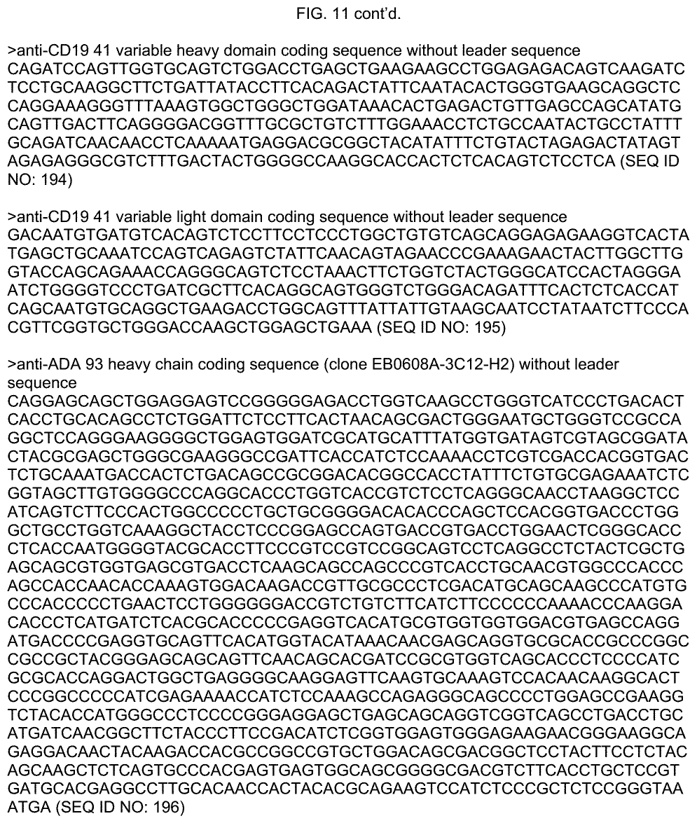

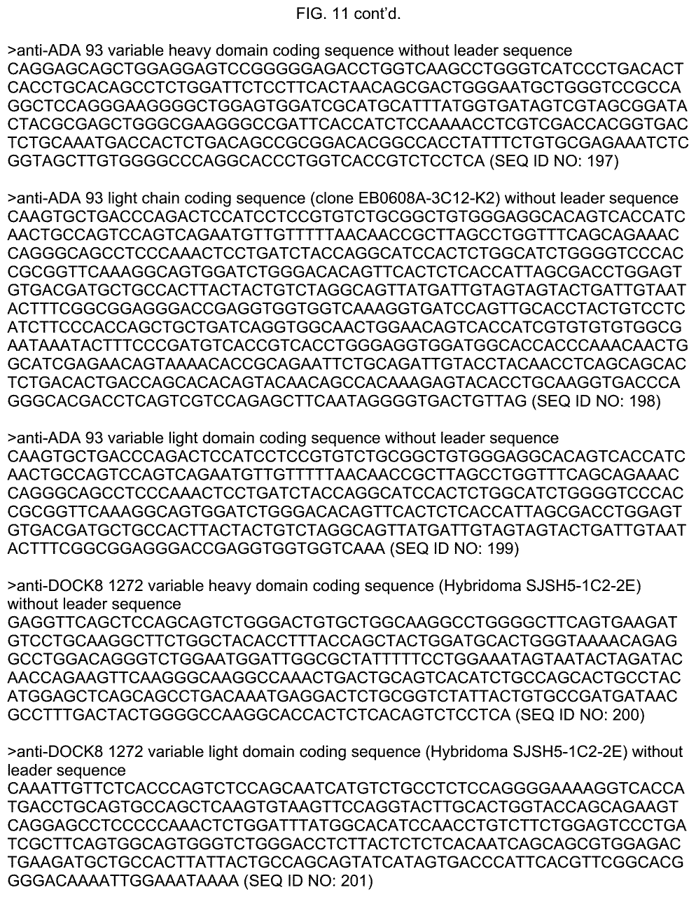

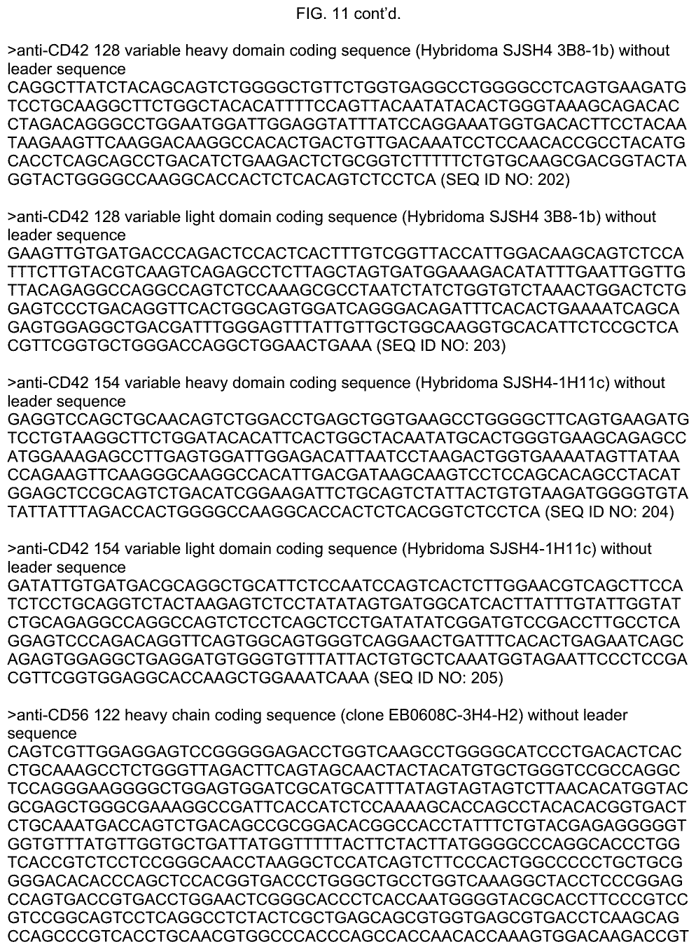

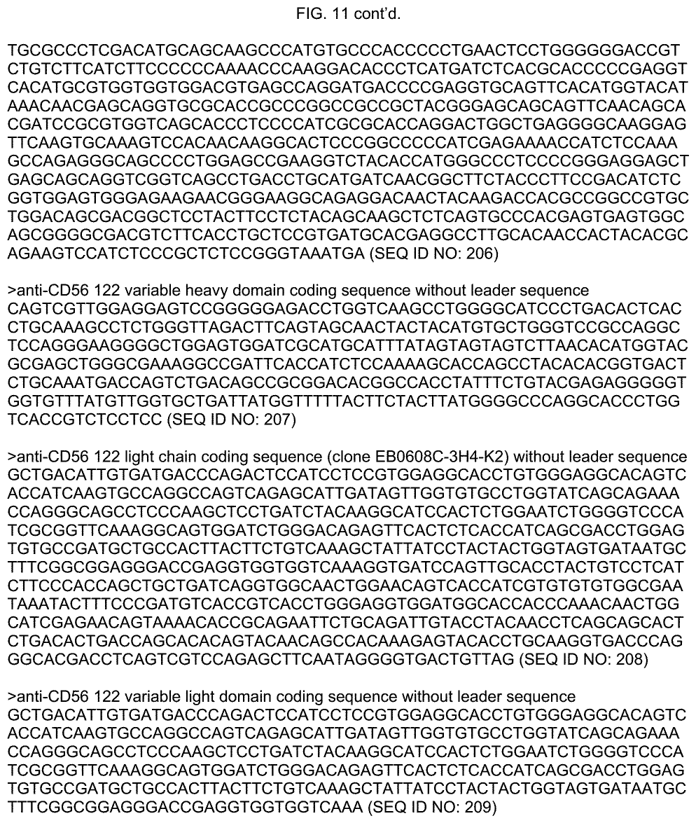

In particular embodiments, the exemplary antibodies include the SEQ ID NOs of VH CDRs, heavy chains, VH domains, LH CDRs, light chains, and VL domains presented in Table 1A-1C and FIG. 11.

In particular embodiments, an exemplary antibody includes a heavy chain or light chain coding sequence with a leader sequence. In particular embodiments, an exemplary antibody includes a variable heavy domain or variable light domain coding sequence with a leader sequence. In particular embodiments, an exemplary antibody includes a heavy chain or light chain amino acid sequence with a leader peptide. In particular embodiments, an exemplary antibody includes a heavy chain or light chain amino acid sequence without a leader peptide. In particular embodiments, an exemplary antibody includes a variable heavy domain or variable light domain amino acid sequence with a leader peptide. In particular embodiments, an exemplary antibody includes a variable heavy domain or variable light domain amino acid sequence without a leader peptide.

### (V) VARIANTS

Variants of the sequences disclosed and referenced herein are also included. Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in a binding study. Functional variants and functional fragments of binding domains bind their cognate antigen or ligand at a level comparable to a wild-type reference.

Guidance in determining which amino acid residues can be substituted, inserted, or deleted without abolishing biological activity can be found using computer programs well known in the art, such as DNASTAR™ (Madison, Wis.) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

In particular embodiments, a binding domain VH region can be derived from or based on a VH of a known antibody or an antibody disclosed herein and can optionally contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the known antibody or antibody disclosed herein. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a VL region in a binding domain is derived from or based on a VL of a known antibody or an antibody disclosed herein and optionally contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known antibody or antibody disclosed herein. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

### (VI) ENRICHMENT STRATEGIES FOR PEPTIDES

Enrichment of a desired peptide target prior to SRM can be accomplished by any means known in the art. A host of enrichment procedures are available, including immuno adsorption-based depletion of abundant protein species from samples, precipitation, chromatography, electrophoresis, solvent partitioning, immunoprecipitation, immunoelectrophoresis, and immunochromatography. In particular embodiments, a SISCAPA method for specific antibody-based capture of individual tryptic peptides from a digest of a sample can be used (Anderson et al., J. Proteome Research 2004; 3: 235-244; U.S. Pat. No. 7,632,686).

In particular embodiments, the antibodies that bind the peptide markers, such as the antibodies disclosed herein, can be attached to a solid support. Particular embodiments use an affinity column, where antibodies are covalently coupled to chromatography media. In particular embodiments, POROS (Applied Biosystems, Foster City, Calif.) nanocolumns can be used in SISCAPA enrichment and features high binding capacity, a relatively high concentration of antibodies allowing for rapid enrichment of target peptides, and the ability to prepare columns with a variety of functionalized groups. Alternatively, antibodies can be attached to beads, magnetic beads, or other solid particles. One means of attachment is conjugation of the antibody to a protein coated on the beads. For example, Protein G coated particles offer the binding of antibodies in a preferred orientation. Other means of attachment can be used, such as direct coating of a bead with the antibody. Magnetic particles are available in a wide array of chemistries allowing for coupling to antibodies. Enrichment with antibodies attached to particles can allow parallel processing of samples. Magnetic particle processing has been automated in 96 well plates for the SISCAPA enrichment step with elution in the plates for analysis by mass spectrometry. Other particular embodiments use a novel bead trap device developed to perform the bead handling steps in line with a nanoflow chromatography system (Anderson et al. Mol Cell Proteomics 2009; 8(5): 995-1005). This minimizes losses of peptides to containers between elution and analysis steps. Peptide enrichment can also be implemented by immobilizing anti-peptide antibodies in pipet tips (Nelson et al. Anal Chem. 1995; 67(7): 1153-1158). After separation of the antibody bound peptide from free peptides, the bound peptide can be eluted. Any elution means can be used. One elution means which has been found to be efficient is 5% acetic acid/3% acetonitrile. Other elution means, including other acids, and other concentrations of acetic acid can be used, as is efficient for a particular peptide.

### (VII) LIQUID CHROMATOGRAPHY (LC)

In particular embodiments, one or more LC purification steps are performed prior to SRM-MS. A mixture of enriched peptides (the mobile phase) can be passed through a column packed with material (stationary phase) to separate the peptides based on their weight and affinity for the mobile and stationary phases of the column. Traditional LC analysis relies on the chemical interactions between sample components and column packing materials, where laminar flow of the sample through the column is the basis for separation of the analyte of interest from the test sample. The skilled artisan will understand that separation in such columns is a diffusional process. A variety of column packing materials are available for chromatographic separation of samples, and selection of an appropriate separation protocol is an empirical process that depends on the sample characteristics, the analyte of interest, the interfering substances present and their characteristics, etc. Various packing chemistries can be used depending on the needs (e.g., structure, polarity, and solubility of compounds being purified). In particular embodiments the columns are polar, ion exchange (both cation and anion), hydrophobic interaction, phenyl, C-2, C-8, C-18 columns, polar coating on porous polymer, or others that are commercially available. During chromatography, the separation of materials is affected by variables such as choice of eluent (also known as a “mobile phase”), choice of gradient elution and the gradient conditions, temperature, etc. In particular embodiments, an analyte may be purified by applying a sample to a column under conditions where the analyte of interest is reversibly retained by the column packing material, while one or more other materials are not retained. In these embodiments, a first mobile phase condition can be employed where the analyte of interest is retained by the column, and a second mobile phase condition can subsequently be employed to remove retained material from the column, once the non-retained materials are washed through. Alternatively, an analyte may be purified by applying a sample to a column under mobile phase conditions where the analyte of interest elutes at a differential rate in comparison to one or more other materials. As discussed above, such procedures may enrich the amount of one or more analytes of interest relative to one or more other components of the sample. In particular embodiments, the LC is microflow LC (microLC). In microflow LC, chromatographic separations are performed using flow rates in the range of low microliter per minute. In particular embodiments, the LC is nanoflow LC (nanoLC). In nanoflow LC (nanoLC) chromatographic separations are performed using a flow rate of 300 nanoliter per minute. The slowed flow rates result in high analytical sensitivity due to the large concentration efficiency afforded by this type of chromatography (Cutillas, Current Nanoscience, 2005; 1: 65-71).

### (VIII) MASS SPECTROMETRY (MS)

A mass spectrometer includes a gas phase ion spectrometer that measures a parameter that can be translated into mass-to-charge (m/z) ratios of gas phase ions. Mass spectrometry refers to the use of a mass spectrometer to detect gas phase ions. Mass spectrometers generally include an ion source and a mass analyzer. Examples of mass spectrometers are time-of-flight (TOF), magnetic sector, quadrupole filter, ion trap, ion cyclotron resonance, electrostatic sector analyzer and hybrids of these. A laser desorption mass spectrometer includes a mass spectrometer that uses laser energy as a means to desorb, volatilize, and ionize an analyte. A tandem mass spectrometer includes any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, triple quadrupole mass spectrometers, and combinations thereof.

Ionization in mass spectrometry includes the process by which analytes in a sample are ionized. Such analytes may become charged molecules used for further analysis. For example, sample ionization may be performed by electrospray ionization (ESI), laserspray ionization (LSI) atmospheric pressure chemical ionization (APCl), photoionization, electron ionization, fast atom bombardment (FAB)/liquid secondary ionization (LSIMS), matrix assisted laser desorption ionization (MALDI), field ionization, field desorption, thermospray/plasmaspray ionization, and particle beam ionization. The skilled artisan will understand that the choice of ionization method can be determined based on the analyte to be measured, type of sample, the type of detector, the choice of positive versus negative mode, etc.

A mass analyzer includes the component of the mass spectrometer that takes ionized masses and separates them based on m/z ratios and outputs them to the detector where they are detected and later converted to a digital output. Suitable mass analyzers for determining m/z ratios include quadrupole mass analyzer, time-of-flight (TOF) mass analyzer, magnetic or electrostatic sector mass analyzer and ion trap (e.g. ion cyclotron resonance) mass analyzer.

A selected reaction monitoring (SRM)-MS assay targets a predetermined set of peptides for a given protein of interest. SRM is a tandem mass spectrometry mode in which an ion of a particular mass, the parent or precursor ion, is selected in the first stage of tandem mass spectrometry, and an ion product of a fragmentation reaction of the precursor ion is selected in the second mass spectrometry stage for detection. The specific pair of m/z values associated with a selected precursor ion and fragment ion is referred to as a transition. For each signature peptide, those fragment ions that provide optimal signal intensity and discriminate the targeted peptide from other species present in the sample are identified. Optimized transitions contribute to an effective SRM assay. Several such transitions (precursor/fragment ion pairs) are monitored over time, yielding a set of chromatographic traces with the retention time and signal intensity for a specific transition as coordinates. SRM-MS analysis of signature peptides is generally performed on a triple quadrupole mass spectrometer (QQQ-MS), an instrument with the capability to selectively isolate precursor ions corresponding to the m/z of the signature peptides and to selectively monitor peptide-specific fragment ions. In SRM analysis, the specificity depends on multiple mass analyzers (mass filters). The first quadrupole is to select the desired parent or precursor ion. The third quadrupole is to monitor the (one or more) fragment ion(s). The fragment ion(s) is generated through collisional induced dissociation in the second quadrupole. The two levels of mass selection allow high selectivity, as co-eluting background ions are filtered out very effectively. Unlike conventional tandem mass spectrometry (MS/MS) experiments that survey all analytes in a sample, SRM analysis selectively targets (filters) particular analytes, which translates into an increased sensitivity by one or two orders of magnitude compared with conventional ‘full scan’ techniques. In addition, SRM provides a linear response over a wide dynamic range up to five orders of magnitude. This enables the detection of low-abundance proteins in highly complex mixtures. Therefore, SRM is a highly specific detection/monitoring method with low background interference. When multiple parent ions are monitored in a single MS run, this type of analysis is known as multiple reaction monitoring (MRM). Using MRM analysis, multiple proteins and multiple regions (signature peptides) of a protein can be monitored in a single mass spectrometry run. Selected reaction monitoring/multiple reaction monitoring mass spectrometry (SRM/MRM-MS) is described in, e.g., U.S. Pat. No. 8,383,417, WO 2013/106603, and US 2013/105684.

In particular embodiments, the following parameters can be used to specify an LC-SRM-MS assay of a protein under a particular LC-SRM-MS system: (1) an enriched tryptic peptide of a given protein; (2) the retention time (RT) of the peptide on an LC column; (3) the m/z value of the peptide precursor ion; (4) the declustering potential used to ionize the precursor ion; (5) the m/z value of a fragment ion generated from the peptide precursor ion; and (6) the collision energy (CE) used to fragment the peptide precursor ion that is optimized for the particular peptide. RT includes the elapsed time between injection and elution of an analyte. Declustering potential (DP) includes a voltage potential to dissolvate and dissociate ion clusters. It is also known as “fragmentor voltage” or “ion transfer capillary offset voltage” depending on the manufacturer. Collision energy (CE) includes the amount of energy precursor ions receive as they are accelerated into the collision cell.

To facilitate accurate quantification of the peptides by the methods disclosed herein, a set of isotopically-labeled synthetic versions of the peptides of interest may be added in known amounts to the sample for use as internal standards. Since the isotopically-labeled peptides have physical and chemical properties identical to the corresponding surrogate peptide, they co-elute from the chromatographic column and are easily identifiable on the resultant mass spectrum (Gerber et al. Proc. Natl. Asso. Sci. 2003; 100: 6940-6945; Kirkpatrick et al. Methods 2005; 35: 265-273). The isotopes with which amino acids in a given peptide can be labeled include ¹³C, ²H, ¹⁵N, ¹⁷O, ¹⁸O and ³⁴S. In particular embodiments, a peptide is labeled with ¹³C and/or ¹⁵N heavy isotopes. The addition of the labeled standards may occur before or after proteolytic digestion. In particular embodiments, the labeled internal standard peptides are added after proteolytic digestion. Methods of synthesizing isotopically-labeled peptides will be known to those of skill in the art. Thus, in particular embodiments, the experimental samples contain internal standard peptides. In particular embodiments, internal standard peptides include reference signature peptides. In particular embodiments, a signature peptide concentration can be determined by combining: (i) a ratio calculated from comparing the peak area of the signature peptide to the peak area of its corresponding reference signature peptide obtained from an LC-MRM-MS assay, and (ii) the known concentration of the reference signature peptide. Peptides selected as reference standards and suitable for quantification are sometimes referred to as quantotypic peptides (Q-peptides). Q-peptides include all of the characteristics of proteotypic peptides but also place restrictions on the residues that can constitute the reference peptide to eradicate artefactual modification and/or incomplete cleavage (Holman et al. Bioanalysis 2012; 4(14): 1763-1786).

Absolute quantitative levels of a given protein, or proteins, can be determined by the SRM/MRM methodology whereby the SRM/MRM signature peak area of an individual peptide from a given protein in one biological sample is compared to the SRM/MRM signature peak area of a known amount of a “spiked” internal standard. In particular embodiments, the internal standard is a synthetic version of the same exact peptide that contains one or more amino acid residues labeled with one or more heavy isotopes. Such isotope labeled internal standards are synthesized so that mass spectrometry analysis generates a predictable and consistent SRM/MRM signature peak that is different and distinct from the native peptide signature peak, and which can be used as a comparator peak. Thus, when the internal standard is spiked in known amounts into a protein preparation from a biological sample and analyzed by mass spectrometry, the signature peak area of the native peptide is compared to the signature peak area of the internal standard peptide, and this numerical comparison indicates either the absolute molarity and/or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in entire cohorts of individual samples.

Another strategy for absolute quantitation of peptides is equimolarity through equalizer peptide. This methodology involves chemically synthesizing the isotopically labeled Q-peptides of interest as dipeptides. A common amino acid sequence is positioned N-terminal to the Q-peptide and is referred to as the equalizer peptide. After solubilization and proteolytic digestion, the amount of Q-peptide can be accurately determined through reference to a single light-labeled peptide. Appropriate amounts of each standard peptide can then be added to a sample of interest (either predigested or prior to proteolysis) to facilitate absolute quantification (Holzmann et al. Anal. Chem. 2009; 81: 10254-10261). Absolute quantification can also employ quantification concatemer (QconCAT) proteins (Beynon et al. Nat. Methods 2005; 2: 587-589; Johnson et al. J. Am. Soc. Mass Spectrom. 2009; 20: 2211-2220; Ding et al. J. Proteome Res. 2011; 10: 3652-3659; Carroll et al. Molecular & Cellular Proteomics 2011; Sep. 19: mcp-M111). In this strategy, a recombinant artificial protein that is an affinity tagged, concatenation of standard peptides from several proteins of interest is heterologously produced in Escherichia coli grown in stable isotopically enriched media. The QconCAT protein is then affinity purified and co-digested with the sample, generating a stoichiometric mixture of all the ‘heavy’ Q-peptides of which it is composed, and the proteolytic peptides from the native proteins and internal standard are subsequently analyzed. A variant of the QconCAT approach, termed peptide-concatenated standards (PCS), uses flanking regions between the Q-peptides in the artificial protein sequence that mirror their endogenous environment (Kito et al. J. Proteome Res. 2007; 6: 792-800). Other particular embodiments use protein standards for absolute quantification (PSAQ) (Brun et al. Mol. Cell. Proteomics 2007; 6: 2139-2149). PSAQ uses recombinant proteins but rather than being a concatenation of peptides from several proteins, the entire protein to be quantified is expressed in stable isotope-labeled form. One or several PSAQs can then be added to the sample pre-digestion to facilitate quantification.

Particular embodiments use label-free strategies for protein quantification such as intensity based measurements (America and Cordewener, Proteomics 2008; 8: 731-749) or spectral counting (Lundgren et al. Expert Rev. Proteomics 2010; 7: 39-53).

To obtain relative quantitative levels of a given peptide, the mass spectrometry-derived signature peak area (or the peak height if the peaks are sufficiently resolved) of an individual peptide, or multiple peptides, from a given protein, in one biological sample can be compared to the signature peak area determined for the same peptide, or peptides, from the same protein, in one or more additional and different biological samples, using the same SRM/MRM methodology. In this way, the amount of a particular peptide, or peptides, from a given protein, is determined relative to the same peptide, or peptides, from the same protein across two or more biological samples under the same experimental conditions. In addition, relative quantitation can be determined for a given peptide, or peptides, from a single protein within a single sample by comparing the signature peak area for that peptide for that given protein by SRM/MRM methodology to the signature peak area for another different peptide, or peptides, from a different protein within the same protein preparation from the biological sample. In this way, the amount of a particular peptide from a given protein, and therefore the amount of the given protein, is determined relative to another protein within the same sample. These approaches generate quantitation of an individual peptide, or peptides, from a given protein to the amount of another peptide, or peptides, from the same protein or from a different protein between samples and within samples wherein the amounts as determined by signature peak area are relative one to another, regardless of the absolute weight to volume or weight to weight amounts of peptides in the protein preparation from the biological sample. Relative quantitative data about individual signature peak areas between different samples can be normalized to the amount of protein analyzed per sample. Relative quantitation can be performed across many peptides simultaneously in a single sample and/or across many samples to gain insight into relative protein amounts.

Signature peptide levels can be expressed in concentration units (e.g., pmol/L). In particular embodiments, the mean concentration of a signature peptide in a test sample derived from a subject being screened for X-CGD, XLP1, FHL2, AT, CVID, SCID, ADA deficiency, DOCK8 deficiency, secondary peptide marker CD42, secondary peptide marker CD56, secondary peptide marker CD3ε, secondary peptide marker CD3δ, or a combination thereof, can be compared to the mean concentration of the corresponding peptide in a normal control sample. In particular embodiments, a normal control sample can be derived from one or more normal control subjects or from a population of normal control subjects. In particular embodiments, a normal control subject includes a subject who does not have or is not known to have X-CGD, XLP1, FHL2, AT, CVID, SCID, ADA deficiency, DOCK8 deficiency, a deficiency of platelets, a deficiency of NK cells, and/or a deficiency of T cells.

In particular embodiments, a normal control subject includes a subject who does not have genetic mutations associated with X-CGD, XLP1, FHL2, AT, CVID, SCID, ADA deficiency, DOCK8 deficiency, a deficiency of CD42, a deficiency of CD56, a deficiency of CD3ε, a deficiency of CD3δ, or a combination thereof.

In particular embodiments, the mean concentration of a CYBB 509 signature peptide in DBS from a population of normal control subjects includes a concentration in a range of 300 pmol/L to 3000 pmol/L, in a range of 500 pmol/L to 2900 pmol/L, and in a range of 700 pmol/L to 2800 pmol/L. In particular embodiments, the mean concentration of a CYBB 509 signature peptide in DBS from a population of normal control subjects includes a concentration of 300 pmol/L, 400 pmol/L, 500 pmol/L, 600 pmol/L, 700 pmol/L, 800 pmol/L, 900 pmol/L, 1000 pmol/L, 1100 pmol/L, 1200 pmol/L, 1300 pmol/L, 1400 pmol/L, 1500 pmol/L, 1600 pmol/L, 1700 pmol/L, 1800 pmol/L, 1900 pmol/L, 2000 pmol/L, 2100 pmol/L, 2200 pmol/L, 2300 pmol/L, 2400 pmol/L, 2500 pmol/L, 2600 pmol/L, 2700 pmol/L, 2800 pmol/L, 2900 pmol/L, 3000 pmol/L, or more.

In particular embodiments, the mean concentration of a ADA 93 signature peptide in DBS from a population of normal control subjects includes a concentration in a range of 900 pmol/L to 5000 pmol/L, in a range of 1000 pmol/L to 4900 pmol/L, and in a range of 1100 pmol/L to 4800 pmol/L. In particular embodiments, the mean concentration of a ADA 93 signature peptide in DBS from a population of normal control subjects includes a concentration of 900 pmol/L, 1000 pmol/L, 1100 pmol/L, 1200 pmol/L, 1300 pmol/L, 1400 pmol/L, 1500 pmol/L, 1600 pmol/L, 1700 pmol/L, 1800 pmol/L, 1900 pmol/L, 2000 pmol/L, 2100 pmol/L, 2200 pmol/L, 2300 pmol/L, 2400 pmol/L, 2500 pmol/L, 2600 pmol/L, 2700 pmol/L, 2800 pmol/L, 2900 pmol/L, 3000 pmol/L, 3100 pmol/L, 3200 pmol/L, 3300 pmol/L, 3400 pmol/L, 3500 pmol/L, 3600 pmol/L, 3700 pmol/L, 3800 pmol/L, 3900 pmol/L, 4000 pmol/L, 4100 pmol/L, 4200 pmol/L, 4300 pmol/L, 4400 pmol/L, 4500 pmol/L, or more.

In particular embodiments, the mean concentration of a DOCK8 1272 signature peptide in DBS from a population of normal control subjects includes a concentration in a range of 70 pmol/L to 550 pmol/L, in a range of 80 pmol/L to 530 pmol/L, and in a range of 90 pmol/L to 500 pmol/L. In particular embodiments, the mean concentration of a DOCK8 1272 signature peptide in DBS from a population of normal control subjects includes a concentration of 70 pmol/L, 75 pmol/L, 80 pmol/L, 85 pmol/L, 90 pmol/L, 95 pmol/L, 100 pmol/L, 125 pmol/L, 150 pmol/L, 175 pmol/L, 200 pmol/L, 225 pmol/L, 250 pmol/L, 275 pmol/L, 300 pmol/L, 325 pmol/L, 350 pmol/L, 375 pmol/L, 400 pmol/L, 425 pmol/L, 450 pmol/L, 475 pmol/L, 500 pmol/L, 525 pmol/L, 550 pmol/L, or more.

In particular embodiments, the mean concentration of a CD42 128 signature peptide in DBS from a population of normal control subjects includes a concentration in a range of 4000 pmol/L to 20000 pmol/L, in a range of 5000 pmol/L to 18000 pmol/L, and in a range of 6000 pmol/L to 17500 pmol/L. In particular embodiments, the mean concentration of a CD42 128 signature peptide in DBS from a population of normal control subjects includes a concentration of 4000 pmol/L, 4500 pmol/L, 5000 pmol/L, 5500 pmol/L, 6000 pmol/L, 6500 pmol/L, 7000 pmol/L, 7500 pmol/L, 8000 pmol/L, 8500 pmol/L, 9000 pmol/L, 9500 pmol/L, 10000 pmol/L, 10500 pmol/L, 11000 pmol/L, 11500 pmol/L, 120000 pmol/L, 12500 pmol/L, 13000 pmol/L, 13500 pmol/L, 14000 pmol/L, 14500 pmol/L, 15000 pmol/L, 15500 pmol/L, 16000 pmol/L, 16500 pmol/L, 17000 pmol/L, 17500 pmol/L, 18000 pmol/L, 18500 pmol/L, 19000 pmol/L, 19500 pmol/L, 20000 pmol/L, or more.

In particular embodiments, the mean concentration of a CD42 154 signature peptide in DBS from a population of normal control subjects includes a concentration in a range of 8000 pmol/L to 30000 pmol/L, in a range of 9000 pmol/L to 29000 pmol/L, and in a range of 10000 pmol/L to 28000 pmol/L. In particular embodiments, the mean concentration of a CD42 154 signature peptide in DBS from a population of normal control subjects includes a concentration of 8000 pmol/L, 9000 pmol/L, 10000 pmol/L, 11000 pmol/L, 12000 pmol/L, 13000 pmol/L, 14000 pmol/L, 15000 pmol/L, 16000 pmol/L, 17000 pmol/L, 18000 pmol/L, 19000 pmol/L, 20000 pmol/L, 21000 pmol/L, 22000 pmol/L, 23000 pmol/L, 24000 pmol/L, 25000 pmol/L, 26000 pmol/L, 27000 pmol/L, 28000 pmol/L, 29000 pmol/L, 30000 pmol/L, or more.

In particular embodiments, the mean concentration of a CD56 122 signature peptide in DBS from a population of normal control subjects includes a concentration in a range of 600 pmol/L to 5000 pmol/L, in a range of 700 pmol/L to 4500 pmol/L, and in a range of 800 pmol/L to 4000 pmol/L. In particular embodiments, the mean concentration of a CD56 122 signature peptide in DBS from a population of normal control subjects includes a concentration of 600 pmol/L, 700 pmol/L, 800 pmol/L, 900 pmol/L, 1000 pmol/L, 1100 pmol/L, 1200 pmol/L, 1300 pmol/L, 1400 pmol/L, 1500 pmol/L, 1600 pmol/L, 1700 pmol/L, 1800 pmol/L, 1900 pmol/L, 2000 pmol/L, 2100 pmol/L, 2200 pmol/L, 2300 pmol/L, 2400 pmol/L, 2500 pmol/L, 2600 pmol/L, 2700 pmol/L, 2800 pmol/L, 2900 pmol/L, 3000 pmol/L, 3100 pmol/L, 3200 pmol/L, 3300 pmol/L, 3400 pmol/L, 3500 pmol/L, 3600 pmol/L, 3700 pmol/L, 3800 pmol/L, 3900 pmol/L, 4000 pmol/L, 4100 pmol/L, 4200 pmol/L, 4300 pmol/L, 4400 pmol/L, 4500 pmol/L, 4600 pmol/L, 4700 pmol/L, 4800 pmol/L, 4900 pmol/L, 5000 pmol/L, or more.

In particular embodiments, the mean concentration of an ATM 1561 signature peptide in DBS from a population of normal control subjects includes a concentration in a range of 20 pmol/L to 100 pmol/L, in a range of 25 pmol/L to 90 pmol/L, and in a range of 30 pmol/L to 80 pmol/L. In particular embodiments, the mean concentration of a ATM 1561 signature peptide in DBS from a population of normal control subjects includes a concentration of 20 pmol/L, 25 pmol/L, 30 pmol/L, 35 pmol/L, 40 pmol/L, 45 pmol/L, 50 pmol/L, 55 pmol/L, 60 pmol/L, 65 pmol/L, 70 pmol/L, 75 pmol/L, 80 pmol/L, 85 pmol/L, 90 pmol/L, 95 pmol/L, 100 pmol/L, or more.

In particular embodiments, the mean concentration of a PRF 215 signature peptide in DBS from a population of normal control subjects includes a concentration in a range of 10 pmol/L to 200 pmol/L, in a range of 11 pmol/L to 100 pmol/L, and in a range of 12 pmol/L to 80 pmol/L. In particular embodiments, the mean concentration of a PRF 215 signature peptide in DBS from a population of normal control subjects includes a concentration of 10 pmol/L, 15 pmol/L, 20 pmol/L, 25 pmol/L, 30 pmol/L, 35 pmol/L, 40 pmol/L, 45 pmol/L, 50 pmol/L, 55 pmol/L, 60 pmol/L, 65 pmol/L, 70 pmol/L, 75 pmol/L, 80 pmol/L, 85 pmol/L, 90 pmol/L, 95 pmol/L, 100 pmol/L, 110 pmol/L, 115 pmol/L, 120 pmol/L, 125 pmol/L, 130 pmol/L, 135 pmol/L, 140 pmol/L, 145 pmol/L, 150 pmol/L, 155 pmol/L, 160 pmol/L, 165 pmol/L, 170 pmol/L, 175 pmol/L, 180 pmol/L, 185 pmol/L, 190 pmol/L, 195 pmol/L, 200 pmol/L, or more.

One or more standard peptides may be synthesized with any method known in the pertinent art. Such synthetic peptides may further include amino acids with one or more natural modifications. Such natural modifications may include deamination of glutamine and asparagine, amination, oxidation, and hydroxylation.

### (IX) METHODS OF USE

The methods of the present disclosure include identifying individuals with one or more of X-CGD, XLP1, FHL2, AT, CVID, SCID, ADA deficiency, and DOCK8 deficiency. In particular embodiments, the methods include identifying individuals with X-linked or IL2RG deficient SCID. In particular embodiments, diagnosing individuals with X-CGD, XLP1, FHL2, AT, CVID, SCID, ADA deficiency, and/or DOCK8 deficiency is performed early, for example, as part of NBS, or before symptoms of a disorder are evident in the individual. In particular embodiments, the identifying or diagnosing includes using secondary peptide markers CD42, CD56, CD3ε, CD3δ, or a combination thereof. In particular embodiments, the secondary marker CD42 can provide information on the level of platelets. In particular embodiments, the secondary marker CD56 can provide information on the level of NK cells. In particular embodiments, CD3ε is used as a secondary marker to provide information on the level of T cells. In particular embodiments, CD3δ is used as a secondary marker to provide information on the level of T cells. In particular embodiments, CD3ε is used as a primary marker to diagnose individuals with SCID. In particular embodiments, CD3δ is used as a primary marker to diagnose individuals with SCID.

The methods of the present disclosure include obtaining DBS, buccal swab, PBMC, or WBC samples. In particular embodiments, DBS, buccal swab, PBMC, or WBC samples are obtained according to methods described herein. In particular embodiments, DBS, buccal swab, PBMC, or WBC samples are obtained from a DBS, a buccal swab, a PBMC, or a WBC repository or lab that stores DBS, buccal swab, PBMC, or WBC samples for future testing.

The methods of the present disclosure include digesting proteins in DBS, cells from buccal swabs, PBMCs, or WBCs with digestion enzymes. In particular embodiments, one or more punches of the DBS, whole DBS, cells from buccal swabs, PBMCs, or WBCs can be solubilized in an appropriate buffer, and an appropriate digestion enzyme described above can be added to digest proteins present in DBS, cells from buccal swabs, PBMCs, or WBCs into peptide fragments. In particular embodiments, DBS, cells from buccal swabs, PBMCs, or WBCs can be solubilized with 0.1% Triton X-100 in 50 mM ammonium bicarbonate and digested with trypsin.

The methods of the present disclosure include enriching for signature peptides that are used in screening for X-CGD, XLP1, FHL2, AT, CVID, ADA deficiency, and/or DOCK8 deficiency. Signature peptides include: CYBB 131 for X-CGD; CYBB 509 for X-CGD; SH2D1A 19 for XLP1; SH2D1A 110 for XLP1; PRF 215 for FHL2; PRF 441 for FHL2; ATM 798 for AT; ATM 1544 for AT; ATM 1561 for AT; CD19 41 for CVID; CD19 55 for CVID; CD19 210 for CVID; CD19 505 for CVID; IL2RG 295 for SCID; IL2RG 316 for SCID; CD3ε 74 for SCID; CD3δ 24 for SCID; CD3δ 133 for SCID; ADA 93 for ADA deficiency; DOCK8 1272 for DOCK8 deficiency; CD42 128 for platelet levels; CD42 154 for platelet levels; CD56 122 for NK cell levels; CD3ε 74 for T cell levels; CD3δ 24 for T cell levels; and CD3δ 133 for T cell levels. In particular embodiments, enriching for signature peptides include contacting mixtures of peptide fragments from a digested biological sample with one or more binding entities that recognize the signature peptides. In particular embodiments, the binding entities are antibodies or antigen binding fragments thereof. In particular embodiments, the antibodies include those disclosed in Table 1A-1C and FIG. 11. In particular embodiments, amino acid sequences of antibodies of the disclosure include SEQ ID NOs: 23, 24, 26, 27, 29, 30, 32, 33, 41, 42, 44, 45, 47, 48, 50, 51, 59, 60, 62, 63, 65, 66, 68, 69, 77, 78, 80, 81, 89, 90, 92, 93, 95, 96, 98, 99, 107, 108, 110, 111, 119, 120, 122, 123, 131, 132, 134, 135, 143, 144, 146, 147, 149, 150, 152, 153, 168, 169, 172, 173, 176, 177, 180, and 181. In particular embodiments, coding sequences of antibodies of the disclosure include SEQ ID NOs: 22, 25, 28, 31, 40, 43, 46, 49, 58, 61, 64, 67, 76, 79, 88, 91, 94, 97, 106, 109, 118, 121, 130, 133, 142, 145, 148, 151, 166, 167, 170, 171, 174, 175, 178, 179, and 182-209. In particular embodiments, the antibodies include antibodies that bind: CYBB 131; CYBB 509; SH2D1A 19; SH2D1A 110; PRF 215; PRF 441; ATM 798; ATM 1544; ATM 1561; CD19 41; CD19 55; CD19 210; CD19 505; IL2RG 295; IL2RG 316; ADA 93; DOCK8 1272; CD42 128; CD42 154; CD56 122; CD3ε 74; CD3δ 24; and CD3δ 133.

In particular embodiments, antibodies are used to enrich for a CYBB peptide including SEQ ID NO: 1. In particular embodiments, antibodies including SEQ ID NOs: 16-21, 23, 24, 26, 27, 29, 30, 32, and 33 are used to enrich for a CYBB peptide including SEQ ID NO: 2.

In particular embodiments, antibodies including SEQ ID NOs: 160-165, 168, 169, 172, 173, 176, 177, 180, and 181 are used to enrich for a SH2D1A peptide including SEQ ID NO: 3. In particular embodiments, antibodies are used to enrich for a SH2D1A peptide including SEQ ID NO: 4.

In particular embodiments, antibodies including SEQ ID NOs: 34-39, 41, 42, 44, 45, 47, 48, 50, and 51 are used to enrich for a PRF-1 peptide including SEQ ID NO: 5. In particular embodiments, antibodies are used to enrich for a PRF-1 peptide including SEQ ID NO: 6.

In particular embodiments, antibodies including SEQ ID NOs: 52-57, 59, 60, 62, 63, 65, 66, 68, and 69 are used to enrich for an ATM peptide including SEQ ID NO: 7. In particular embodiments, antibodies are used to enrich for an ATM peptide including SEQ ID NO: 210. In particular embodiments, antibodies are used to enrich for an ATM peptide including SEQ ID NO: 211.

In particular embodiments, antibodies including SEQ ID NOs: 70-75, 77, 78, 80, and 81 are used to enrich for a CD19 peptide including SEQ ID NO: 8. In particular embodiments, antibodies are used to enrich for a CD19 peptide including SEQ ID NO: 9. In particular embodiments, antibodies are used to enrich for a CD19 peptide including SEQ ID NO: 10. In particular embodiments, antibodies are used to enrich for a CD19 peptide including SEQ ID NO: 212.

In particular embodiments, antibodies are used to enrich for an IL2RG peptide including SEQ ID NO: 213. In particular embodiments, antibodies are used to enrich for an IL2RG peptide including SEQ ID NO: 214.

In particular embodiments, antibodies including SEQ ID NOs: 82-87, 89, 90, 92, 93, 95, 96, 98, and 99 are used to enrich for an ADA peptide including SEQ ID NO: 11.

In particular embodiments, antibodies including SEQ ID NOs: 100-105, 107, 108, 110, and 111 are used to enrich for a DOCK8 peptide including SEQ ID NO: 12.

In particular embodiments, antibodies including SEQ ID NOs: 112-117, 119, 120, 122, and 123 are used to enrich for a CD42 peptide including SEQ ID NO: 13. In particular embodiments, antibodies including SEQ ID NOs: 124-129, 131, 132, 134, and 135 are used to enrich fora CD42 peptide including SEQ ID NO: 14.

In particular embodiments, antibodies including SEQ ID NOs: 136-141, 143, 144, 146, 147, 149, 150, 152, and 153 are used to enrich for a CD56 peptide including SEQ ID NO: 15.

In particular embodiments, antibodies are used to enrich for a CD3ε peptide of SEQ ID NO: 215.

In particular embodiments, antibodies are used to enrich for a CD3δ peptide of SEQ ID NO: 216. In particular embodiments, antibodies are used to enrich for a CD3δ peptide of SEQ ID NO: 217.

In particular embodiments, any combination of one or more antibodies disclosed in Table 1A-1C and FIG. 11 that bind their cognate signature peptides can be used to screen for X-CGD, XLP1, FHL2, AT, CVID, SCID, ADA deficiency, and/or DOCK8 deficiency. In particular embodiments, any combination of one or more antibodies disclosed in Table 1A-1C and FIG. 11 that bind their cognate signature peptides can be used to screen a population for X-CGD, XLP1, FHL2, AT, CVID, SCID, ADA deficiency, and/or DOCK8 deficiency. In particular embodiments, the methods include identifying individuals with X-linked or IL2RG deficient SCID.

The methods of the present disclosure include optionally performing liquid chromatography on the immunoaffinity enriched peptides to separate the peptides prior to MS analysis. Liquid chromatography can separate peptides based on their weight and affinity for the mobile and stationary phases of the column.

The methods of the present disclosure include performing SRM-MS or MRM-MS on the immunoaffinity enriched peptides to quantify the amount of a given signature peptide. In particular embodiments, the SRM-MS or MRM-MS is carried out as described above. In particular embodiments, the quantification of a signature peptide includes using a reference peptide that is introduced into an assay in known amounts. In particular embodiments, a reference peptide can be identical to the signature peptide in every respect except that the reference peptide has been differentially labeled, for example, with one or more heavy isotopes, to distinguish the reference peptide from the signature peptide.

In particular embodiments, SRM-MS or MRM-MS detects a reduction or absence in a CYBB peptide. In particular embodiments, the CYBB peptide includes SEQ ID NO: 1. In particular embodiments, the CYBB peptide includes SEQ ID NO: 2.

In particular embodiments, SRM-MS or MRM-MS detects a reduction or absence in a SH2D1A peptide. In particular embodiments, the SH2D1A peptide includes SEQ ID NO: 3. In particular embodiments, the SH2D1A peptide includes SEQ ID NO: 4.

In particular embodiments, SRM-MS or MRM-MS detects a reduction or absence in a PRF-1 peptide. In particular embodiments, the PRF-1 peptide includes SEQ ID NO: 5. In particular embodiments, the PRF-1 peptide includes SEQ ID NO: 6.

In particular embodiments, SRM-MS or MRM-MS detects a reduction or absence in an ATM peptide. In particular embodiments, the ATM peptide includes SEQ ID NO: 7. In particular embodiments, the ATM peptide includes SEQ ID NO: 210. In particular embodiments, the ATM peptide includes SEQ ID NO: 211.

In particular embodiments, SRM-MS or MRM-MS detects a reduction or absence in a CD19 peptide. In particular embodiments, the CD19 peptide includes SEQ ID NO: 8. In particular embodiments, the CD19 peptide includes SEQ ID NO: 9. In particular embodiments, the CD19 peptide includes SEQ ID NO: 10. In particular embodiments, the CD19 peptide includes SEQ ID NO: 212.

In particular embodiments, SRM-MS or MRM-MS detects a reduction or absence in an IL2RG peptide. In particular embodiments, the IL2RG peptide includes SEQ ID NO: 213. In particular embodiments, the IL2RG peptide includes SEQ ID NO: 214.

In particular embodiments, SRM-MS or MRM-MS detects a reduction or absence in an ADA peptide. In particular embodiments, the ADA peptide includes SEQ ID NO: 11.

In particular embodiments, SRM-MS or MRM-MS detects a reduction or absence in a DOCK8 peptide. In particular embodiments, the DOCK8 peptide includes SEQ ID NO: 12.

In particular embodiments, SRM-MS or MRM-MS detects a reduction or absence in a CD42 peptide. In particular embodiments, the CD42 peptide includes SEQ ID NO: 13. In particular embodiments, the CD42 peptide includes SEQ ID NO: 14.

In particular embodiments, SRM-MS or MRM-MS detects a reduction or absence in a CD56 peptide. In particular embodiments, the CD56 peptide includes SEQ ID NO: 15.

In particular embodiments, SRM-MS or MRM-MS detects a reduction or absence in a CD3ε peptide. In particular embodiments, the CD3ε peptide includes SEQ ID NO: 215.

In particular embodiments, SRM-MS or MRM-MS detects a reduction or absence in a CD3δ peptide. In particular embodiments, the CD3δ peptide SEQ ID NO: 216. In particular embodiments, the CD3δ peptide SEQ ID NO: 217.

Particular embodiments include monitoring subjects for signature peptide levels using immuno-SRM as described herein over a period of time. In particular embodiments, a subject is selected for monitoring according to the systems and methods disclosed herein because they exhibit signs or symptoms of X-CGD, XLP1, FHL2, AT, CVID, SCID, ADA deficiency, and/or DOCK8 deficiency as described herein, or are undergoing treatment for X-CGD, XLP1, FHL2, AT, CVID, SCID, ADA deficiency, and/or DOCK8 deficiency.

Particular embodiments disclosed herein include determining efficacy of a treatment in a subject being treated for X-CGD, XLP1, FHL2, AT, CVID, SCID, ADA deficiency, and/or DOCK8 deficiency including obtaining biological samples derived from the subject prior to one or more treatments and during and/or after one or more treatments; detecting signature peptide levels in the subject prior to the treatment using immuno-SRM described herein; detecting signature peptide levels in the subject during or after the one or more treatments using immuno-SRM described herein; and determining that the treatment is effective if the signature peptide levels during or after the treatment is higher than the signature peptide levels prior to the treatment, or determining that the treatment is not effective if the signature peptide levels during or after the treatment is equal to or lower than the signature peptide levels prior to the treatment. In particular embodiments, the biological sample includes DBS, cells from buccal swabs, PBMC, or WBC. In particular embodiments, the subject is being treated for X-linked or IL2RG deficient SCID.

In particular embodiments, determining efficacy of a treatment in a subject being treated for X-CGD, XLP1, FHL2, AT, CVID, SCID, ADA deficiency, and/or DOCK8 deficiency can guide whether the one or more treatments should be continued or discontinued, or whether a new treatment should be implemented. In particular embodiments, one or more treatments can be continued if the signature peptide levels in the subject during or after the one or more treatments is higher than the signature peptide levels in the subject prior to the one or more treatments. In particular embodiments, the one or more treatments can be discontinued if the signature peptide levels during or after the one or more treatments in the subject is greater than 1%, greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 100%, or more of signature peptide levels measured in a normal control subject or control subject unaffected by X-CGD, XLP1, FHL2, AT, CVID, SCID, ADA deficiency, and/or DOCK8 deficiency. In particular embodiments, a new treatment can be implemented if the signature peptide levels in the subject during or after the treatment is equal to or lower than the signature peptide levels in the subject prior to the treatment or if the signature peptides are absent.

Particular embodiments disclosed herein include measuring pharmacokinetic profiles of protein therapeutics administered to a subject being treated for X-CGD, XLP1, FHL2, AT, CVID, SCID, ADA deficiency, and/or DOCK8 deficiency including obtaining biological samples derived from the subject prior to administration of one or more protein therapeutics and during and/or after administration of one or more protein therapeutics; detecting signature peptide levels in the subject prior to administration of one or more protein therapeutics using immuno-SRM described herein; detecting signature peptide levels in the subject during or after administration of one or more protein therapeutics using immuno-SRM described herein, thereby measuring pharmacokinetic profiles of administered protein therapeutics. In particular embodiments, the biological sample includes DBS, cells from buccal swabs, PBMC, or WBC. In particular embodiments, the subject is being treated for X-linked or IL2RG deficient SCID.

In particular embodiments, “stable” measures of signature peptide levels are measures evaluated in relation to a previous comparison in the same subject and denote a signature peptide level that has not changed significantly (as determined by a statistical measure known in the art such as a t-test or p-value, e.g., p value >0.05) since the last measurement. In particular embodiments, “stable” measures are measures evaluated in relation to a previous comparison in the same patient and denote a signature peptide level that has not changed significantly (as determined by a statistical measure known in the art such as a t-test or p-value, e.g., p value >0.05) since an aggregated or averaged group of previous measurements (e.g., the last 3, 4, or 5 measurements).

“Unchanged” measures of signature peptide levels are measures evaluated in relation to a previous comparison in the same patient and denote a failure to achieve a statistically significant change in a score towards or away from a reference signature peptide level in the particular subject. In particular embodiments, “unchanged” measures are measures evaluated in relation to a previous comparison in the same patient or since an aggregated or averaged group of previous measurements (e.g., the last 3, 4, or 5 measurements).

In particular embodiments, antibodies of the present disclosure can also be used in complimentary clinical tests for the diagnosis of X-CGD, XLP1, FHL2, AT, CVID, SCID, ADA deficiency, and DOCK8 deficiency for those patients with ambiguous biochemical results, and for patients who carry the variants of unknown significance (VUS) from genetic tests. In particular embodiments, subjects having VUS in genes encoding: CYBB of X-CGD; SH2D1A of XLP1; PRF-1 of FHL2; ATM of AT; CD19 of CVID; IL2RG of SCID; ADA of ADA deficiency; and/or DOCK8 of DOCK8 deficiency disclosed herein can be tested with the immuno-SRM assays of the disclosure to determine if the VUS affects the respective signature peptide levels in these subjects.

In particular embodiments, a predetermined cut-off value is used as a threshold fora given signature peptide. A concentration of a given signature peptide above the threshold indicates that the assayed biological sample (e.g., DBS, cells from buccal swabs, PBMC, or WBC) is from an individual not afflicted by X-CGD, XLP1, FHL2, AT, CVID, SCID, ADA deficiency, and/or DOCK8 deficiency. A concentration of a given signature peptide below the threshold or absent indicates that the assayed DBS is from an individual afflicted by X-CGD, XLP1, FHL2, AT, CVID, SCID, ADA deficiency, and/or DOCK8 deficiency. In particular embodiments, the threshold can be determined by analysis of a population of normal controls and calculation of standard deviation (SD) of a concentration of a given signature peptide in this population. The threshold can be set at a certain SD from the mean concentration of the given signature peptide. In particular embodiments, the threshold is −1 SD, −1.1 SD, −1.2 SD, −1.3 SD, −1.4 SD, −1.5 SD, −1.6 SD, −1.7 SD, −1.8 SD, −1.9 SD, −2.0 SD, −2.1 SD, −2.2 SD, −2.3 SD, −2.4 SD, −2.5 SD, −2.6 SD, −2.7 SD, −2.8 SD, −2.9 SD, −3.0 SD, or more SD from the mean concentration of the given signature peptide. In particular embodiments, for diagnosis or screening of X-CGD, XLP1, FHL2, AT, CVID, SCID, ADA deficiency, or DOCK8 deficiency, the threshold can be determined by analysis of a population of normal controls and calculation of standard deviation (SD) of a ratio of a concentration of a given signature peptide to an endogenous concentration of ATP7B (Jung et al., J. Proteome Res. 2017; 16: 862-871) in this population. Peptide concentration cutoffs for each PIDD can be set at a certain SD derived from mean concentration of each signature peptide or ratio of a concentration of a given signature peptide to an endogenous concentration of ATP7B.

In particular embodiments, the threshold concentration for a signature peptide of the disclosure includes −1.0 SD, −1.25 SD, −1.3 SD, −1.35 SD, −1.4 SD, −1.45 SD, −1.5 SD, −1.55 SD, −1.6 SD, −1.65 SD, −1.7 SD, −1.75 SD, −1.8 SD, −1.85 SD, −1.9 SD, −1.95 SD, −2.0 SD, −2.25 SD, −2.3 SD, −2.35 SD, −2.4 SD, −2.45 SD, −2.5 SD, −2.55 SD, −2.6 SD, −2.65 SD, −2.7 SD, −2.75 SD, −2.8 SD, −2.85 SD, −2.9 SD, −2.95 SD, −3.0 SD, or more from the mean concentration of the corresponding signature peptide in a population of normal controls.

In particular embodiments, the threshold concentration for the CYBB 509 peptide includes 250 pmol/L or less, 240 pmol/L or less, 230 pmol/L or less, 220 pmol/L or less, 210 pmol/L or less, 200 pmol/L or less, 190 pmol/L or less, 185 pmol/L or less, 180 pmol/L or less, 175 pmol/L or less, 170 pmol/L or less, 165 pmol/L or less, 160 pmol/L or less, 155 pmol/L or less, 150 pmol/L or less. In particular embodiments, the threshold concentration for the CYBB 509 peptide includes 188 pmol/L or less.

In particular embodiments, the threshold concentration for the ADA 93 peptide includes 500 pmol/L or less, 490 pmol/L or less, 480 pmol/L or less, 470 pmol/L or less, 460 pmol/L or less, 450 pmol/L or less, 440 pmol/L or less, 430 pmol/L or less, 420 pmol/L or less, 410 pmol/L or less, 400 pmol/L or less, 390 pmol/L or less, 380 pmol/L or less, 370 pmol/L or less, 360 pmol/L or less. In particular embodiments, the threshold concentration for the ADA 93 peptide includes 470 pmol/L or less.

In particular embodiments, the threshold concentration for the DOCK8 1272 peptide includes 100 pmol/L or less, 90 pmol/L or less, 80 pmol/L or less, 70 pmol/L or less, 60 pmol/L or less, 50 pmol/L or less, 40 pmol/L or less, 30 pmol/L or less, 20 pmol/L or less, 10 pmol/L or less. In particular embodiments, the threshold concentration for the DOCK8 1272 peptide includes 63 pmol/L or less.

In particular embodiments, the threshold concentration for the CD42 128 peptide includes 4000 pmol/L or less, 3900 pmol/L or less, 3800 pmol/L or less, 3700 pmol/L or less, 3600 pmol/L or less, 3500 pmol/L or less, 3400 pmol/L or less, 3300 pmol/L or less, 3200 pmol/L or less, 3100 pmol/L or less, 3000 pmol/L or less, 2900 pmol/L or less, 2800 pmol/L or less, 2700 pmol/L or less, 2600 pmol/L or less. In particular embodiments, the threshold concentration for the CD42 128 peptide includes 3435 pmol/L or less.

In particular embodiments, the threshold concentration for the CD42 154 peptide includes 8000 pmol/L or less, 7900 pmol/L or less, 7800 pmol/L or less, 7700 pmol/L or less, 7600 pmol/L or less, 7500 pmol/L or less, 7400 pmol/L or less, 7300 pmol/L or less, 7200 pmol/L or less, 7100 pmol/L or less, 7000 pmol/L or less, 6900 pmol/L or less, 6800 pmol/L or less, 6700 pmol/L or less, 6600 pmol/L or less. In particular embodiments, the threshold concentration for the CD42 154 peptide includes 7450 pmol/L or less.

In particular embodiments, the threshold concentration for the CD56 122 peptide includes 600 pmol/L or less, 590 pmol/L or less, 580 pmol/L or less, 570 pmol/L or less, 560 pmol/L or less, 550 pmol/L or less, 540 pmol/L or less, 530 pmol/L or less, 520 pmol/L or less, 510 pmol/L or less, 500 pmol/L or less, 490 pmol/L or less, 480 pmol/L or less, 470 pmol/L or less, 460 pmol/L or less. In particular embodiments, the threshold concentration for the CD56 122 peptide includes 482 pmol/L or less.

In particular embodiments, the threshold concentration for the ATM 1561 peptide includes 18 pmol/L or less, 17 pmol/L or less, 16 pmol/L or less, 15 pmol/L or less, 14 pmol/L or less, 13 pmol/L or less, 12 pmol/L or less, 11 pmol/L or less, 10 pmol/L or less, 9 pmol/L or less, 8 pmol/L or less, 7 pmol/L or less, 6 pmol/L or less, 5 pmol/L or less, 4 pmol/L or less, 3 pmol/L or less, 2 pmol/L or less, 1 pmol/L or less. In particular embodiments, the threshold concentration for the ATM 1561 peptide includes 15 pmol/L or less.

In particular embodiments, the threshold concentration for the PRF 215 peptide includes 11 pmol/L or less, 10 pmol/L or less, 9 pmol/L or less, 8 pmol/L or less, 7 pmol/L or less, 6 pmol/L or less, 5 pmol/L or less, 4 pmol/L or less, 3 pmol/L or less, 2 pmol/L or less, 1 pmol/L or less, 0.9 pmol/L or less, 0.8 pmol/L or less, 0.7 pmol/L or less, 0.6 pmol/L or less, 0.5 pmol/L or less, 0.4 pmol/L or less, 0.3 pmol/L or less, 0.2 pmol/L or less, 0.1 pmol/L or less. In particular embodiments, the threshold concentration for the PRF 215 peptide includes 10 pmol/L or less.

In particular embodiments, a signature peptide can be considered a primary biomarker for diagnosis or screening of a given disease. A primary signature peptide can include peptides that are used first to diagnose or screen for a given disease. In particular embodiments, a primary biomarker can be used to directly diagnose a specific disease. In particular embodiments, a primary biomarker can be reproducibly obtained from a digestion of the corresponding protein, has high affinity antibodies for immunoaffinity enrichment, and/or is reproducible across independent liquid chromatography columns and/or mass spectrometry instruments. In particular embodiments, a CYBB peptide can be used as a primary biomarker to screen for subjects who have X-CGD. In particular embodiments, an SH2D1A peptide can be used as a primary biomarker to screen for subjects who have XLP1. In particular embodiments, a PRF-1 peptide can be used as a primary biomarker to screen for subjects who have FHL2. In particular embodiments, an ATM peptide can be used as a primary biomarker to screen for subjects who have AT. In particular embodiments, a CD19 peptide can be used as a primary biomarker to screen for subjects who have CVID. In particular embodiments, an IL2RG peptide can be used as a primary biomarker to screen for subjects who have SCID. In particular embodiments, a CD3ε peptide can be used as a primary biomarker to screen for subjects who have SCID. In particular embodiments, a CD3δ peptide can be used as a primary biomarker to screen for subjects who have SCID. In particular embodiments, an ADA peptide can be used as a primary biomarker to screen for subjects who have ADA deficiency. In particular embodiments, a DOCK8 peptide can be used as a primary biomarker to screen for subjects who have DOCK8 deficiency. In particular embodiments, a signature peptide can be considered a secondary biomarker for diagnosis or screening of a given disease. A secondary signature peptide can include peptides that are used second to confirm a diagnosis or screening of a given disease with a primary biomarker. In particular embodiments, CD42 can be a secondary biomarker for assessing a level of platelets. In particular embodiments, CD56 can be a secondary biomarker for a level of NK cells. In particular embodiments, CD56 can be a secondary biomarker supportive for various immunodeficiencies. In particular embodiments, CD3ε can be used as a secondary biomarker for assessing a level of T cells. In particular embodiments, CD3δ can be used as a secondary biomarker for assessing a level of T cells.

Methods disclosed herein include treating subjects (e.g., humans) based upon the outcome of screening for X-CGD, XLP1, FHL2, AT, CVID, SCID, ADA deficiency, and/or DOCK8 deficiency with compositions and methods disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an alleviation of symptoms, an elimination of symptoms, or a cure for X-CGD, XLP1, FHL2, AT, CVID, SCID, ADA deficiency, and/or DOCK8 deficiency. Effective amounts are often administered for research purposes. Effective amounts disclosed herein can cause a statistically-significant effect in an animal model or in vitro assay relevant to the assessment of a disease's development, progression, and/or resolution.

Particular embodiments may include administering compositions as a “prophylactic treatment.” Prophylactic treatments include those administered to a subject who does not display signs or symptoms of X-CGD, XLP1, FHL2, AT, CVID, SCID, ADA deficiency, and/or DOCK8 deficiency or displays only early signs or symptoms of X-CGD, XLP1, FHL2, AT, CVID, SCID, ADA deficiency, and/or DOCK8 deficiency, such that treatment is administered for the purpose of diminishing or decreasing the risk of developing the disorder. Thus, a prophylactic treatment functions as a preventative treatment against X-CGD, XLP1, FHL2, AT, CVID, SCID, ADA deficiency, and/or DOCK8 deficiency.

In particular embodiments, a prophylactic treatment can prevent, delay, or reduce the onset of X-CGD, XLP1, FHL2, AT, CVID, SCID, ADA deficiency, and/or DOCK8 deficiency. In particular embodiments, a prophylactic treatment can be given prior, concurrently, or after other preventative measures, such as the use of antibiotics for PIDDs. In particular embodiments, a prophylactic treatment can prevent or reduce the severity of symptoms or complications associated with X-CGD, XLP1, FHL2, AT, CVID, SCID, ADA deficiency, and/or DOCK8 deficiency.

Symptoms and complications for X-CGD include bacterial and fungal infections; granulomas; abscesses of the lungs, liver, spleen, bones, or skin; swollen lymph nodes; persistent diarrhea; and/or chronic runny nose. Symptoms and complications for XLP1 include fever; fatigue, weight loss; loss of appetite; enlargement of lymph nodes; and/or increased risk of developing cancer. Symptoms and complications for FHL2 include fever; edema; hepatosplenomegaly; liver dysfunction; neurologic impairment; seizures; and/or ataxia. Symptoms and complications for AT include decreased coordination of movements; decreasing mental development; delayed walking; and/or discoloration of skin areas exposed to sunlight. Symptoms and complications for CVID include breathing problems; chronic cough; diarrhea that causes weight loss; ear infections; sinus infections; and/or recurring lung infections. Symptoms and complications for SCID include respiratory infection, ear infections, oral thrush, other pathogenic infections, bronchitis, pneumonia, diarrhea, and failure to thrive. Symptoms and complications for ADA deficiency include pneumonia; chronic diarrhea; skin rashes; and/or delayed development. Symptoms and complications for DOCK8 deficiency include eczema; respiratory infections; and/or skin staphylococcus infections.

A “therapeutic treatment” includes a treatment administered to a subject who displays symptoms or signs of X-CGD, XLP1, FHL2, AT, CVID, SCID, ADA deficiency, and/or DOCK8 deficiency and is administered to the subject for the purpose of diminishing or eliminating those signs or symptoms of X-CGD, XLP1, FHL2, AT, CVID, SCID, ADA deficiency, and/or DOCK8 deficiency. In particular embodiments, the therapeutic treatment can provide immune function for subjects diagnosed with X-CGD, XLP1, FHL2, AT, CVID, SCID, ADA deficiency, and/or DOCK8 deficiency. In particular embodiments, the therapeutic treatment can reduce, control, or eliminate symptoms and complications of X-CGD, XLP1, FHL2, AT, CVID, SCID, ADA deficiency, and/or DOCK8 deficiency such as those described herein.

Prophylactic treatments and therapeutic treatments need not be mutually exclusive, and in particular embodiments, administered dosages may accomplish more than one treatment type.

In particular embodiments, therapeutically effective amounts provide immune system function for subjects diagnosed with X-CGD, XLP1, FHL2, AT, CVID, SCID, ADA deficiency, and/or DOCK8 deficiency. Thus, in particular embodiments, methods of treatment disclosed herein include stem cell transplants, immunoglobulin infusions, antibiotic infusions, and/or gene therapy, for disorders such as X-CGD, XLP1, FHL2, AT, CVID, SCID, ADA deficiency, and/or DOCK8 deficiency. In particular embodiments, methods of treatment include enzyme therapy for ADA deficiency. Providing immune function include: decreasing the frequency or number of bacterial, viral, or parasitic infections, increasing life expectancy, and/or increasing growth.

In particular embodiments, administration of a therapeutic composition can be accompanied with administration of a separate adjuvant. Exemplary adjuvants include alum, bentonite, latex, and acrylic particles: incomplete Freund's adjuvant, complete Freund's adjuvant; aluminum-based salts such as aluminum hydroxide; calcium-based salts; silica or any TLR biological ligand(s); Sigma Adjuvant System (SAS); Ribi adjuvants.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, previous or concurrent therapeutic interventions, idiopathy of the subject and route of administration.

Therapeutically effective amounts of cells can range from 10⁴ cells/kg to 10⁹ cells/kg. In particular embodiments, a therapeutically effective amount of cells can include 10⁴ cells/kg, 10⁵ cells/kg, 10⁶ cells/kg, 10⁷ cells/kg, 10⁸ cells/kg, 10⁹ cells/kg, or more.

Useful doses can range from 0.1 to 5 μg/kg or from 0.5 to 1 μg/kg. In particular embodiments, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In particular embodiments, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

### (X) KITS

Kits to test for congenital disorders are also provided. Kits can include lancets to prick for blood, filter cards to collect blood drops, swabs to collect cheek cells, tubes to collect blood, solutions to solubilize DBS or cells, and appropriate buffers and enzymes to digest marker proteins in the DBS or cells. Kits can further include one or more containers including anti-peptide binding agents (e.g., antibodies) and/or reagents or supplies to assess absence or reduction in CYBB, SH2D1A (SAP), PRF-1, ATM, CD19, IL2RG, ADA, DOCKS, CD42, CD56, CD3ε, and/or CD3δ. In particular embodiments, the kits include one or more containers including the following anti-peptide antibodies: anti-CYBB 131, anti-CYBB 509, anti-SH2D1A 19, anti-SH2D1A 110, anti-PRF 215, anti-PRF 441, anti-ATM 798, anti-ATM 1544, anti-ATM 1561, anti-CD19 41, anti-CD19 55, anti-CD19 210, anti-CD19 505, anti-IL2RG 295, anti-IL2RG 316, anti-ADA 93, anti-Dock8 1272, anti-CD42 128, anti-CD42 154, and/or anti-CD56 122, anti-CD3ε 74, anti-CD3δ 24, and/or anti-CD3δ 133. The antibodies may be immobilized on a solid support, such as a column or beads. Kits can further include elution buffers to release peptides from antibodies. In particular embodiments, kits can include one or more labeled reference peptides to perform absolute quantification of the signature peptides. In particular embodiments, kits can also include some or all of the necessary laboratory and/or medical supplies needed to use the kit effectively, such as gauze, sterile adhesive strips, gloves, tubes, and the like. Variations in contents of any of the kits described herein can be made.

Components of the kit can be prepared for storage and later use. Associated with such container(s) can be a notice in the form prescribed by a governmental agency regulating the manufacture, use, or sale of the kit, which notice reflects approval by the agency of manufacture, use, or sale, when required.

Optionally, the kits further include instructions for using the kit in the methods. In various embodiments, the instructions can include appropriate instructions to interpret results associated with using the kit; proper disposal of the related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (XI) Exemplary Embodiments

1. A method of screening for X-linked chronic granulomatous disease (X-CGD), adenosine deaminase (ADA) deficiency, and dedicator of cytokinesis 8 (DOCKS) deficiency in a subject, the method including:
obtaining a biological sample derived from the subject;
digesting proteins from the biological sample with an enzyme to yield one or more peptides; enriching for:
   a CYBB signature peptide of X-CGD of SEQ ID NO: 2 with an antibody or antigen-binding fragment thereof that binds the second CYBB signature peptide and includes: a heavy chain variable (VH) domain including CDR1 of SEQ ID NO: 16, CDR2 of SEQ ID NO: 17, and CDR3 of SEQ ID NO: 18, and a light chain variable (VL) domain including: CDR1 of SEQ ID NO: 19, CDR2 of SEQ ID NO: 20, and CDR3 of SEQ ID NO: 21;
   an ADA signature peptide of ADA deficiency of SEQ ID NO: 11 with an antibody or antigen-binding fragment thereof that binds the ADA signature peptide and includes: a heavy chain variable (VH) domain including CDR1 of SEQ ID NO: 82, CDR2 of SEQ ID NO: 83, and CDR3 of SEQ ID NO: 84, and a light chain variable (VL) domain including: CDR1 of SEQ ID NO: 85, CDR2 of SEQ ID NO: 86, and CDR3 of SEQ ID NO: 87; and
   a DOCK8 signature peptide of DOCK8 deficiency of SEQ ID NO: 12 with an antibody or antigen-binding fragment thereof that binds the DOCK8 signature peptide and includes: a heavy chain variable (VH) domain including CDR1 of SEQ ID NO: 100, CDR2 of SEQ ID NO: 101, and CDR3 of SEQ ID NO: 102, and a light chain variable (VL) domain including: CDR1 of SEQ ID NO: 103, CDR2 of SEQ ID NO: 104, and CDR3 of SEQ ID NO: 105;
performing liquid chromatography-multiple reaction monitoring mass spectrometry (LC-MRM-MS) on the enriched peptides to determine a concentration of each signature peptide; and
diagnosing the subject with:
   X-CGD when the concentration of the CYBB signature peptide is lower than a predetermined threshold concentration or when the CYBB signature peptide is absent;
   ADA deficiency when the concentration of the ADA signature peptide is lower than a predetermined threshold concentration or when the ADA signature peptide is absent; and
   DOCK8 deficiency when the concentration of the DOCK8 signature peptide is lower than a predetermined threshold concentration or when the DOCK8 signature peptide is absent.
      2. The method of embodiment 1, wherein the method further includes enriching for:
a first CD42 signature peptide of SEQ ID NO: 13 with an antibody or antigen-binding fragment thereof that binds the first CD42 signature peptide and includes: a heavy chain variable (VH) domain including CDR1 of SEQ ID NO: 112, CDR2 of SEQ ID NO: 113, and CDR3 of SEQ ID NO: 114, and a light chain variable (VL) domain including: CDR1 of SEQ ID NO: 115, CDR2 of SEQ ID NO: 116, and CDR3 of SEQ ID NO: 117;
a second CD42 signature peptide of SEQ ID NO: 14 with an antibody or antigen-binding fragment thereof that binds the second CD42 signature peptide and includes: a heavy chain variable (VH) domain including CDR1 of SEQ ID NO: 124, CDR2 of SEQ ID NO: 125, and CDR3 of SEQ ID NO: 126, and a light chain variable (VL) domain including: CDR1 of SEQ ID NO: 127, CDR2 of SEQ ID NO: 128, and CDR3 of SEQ ID NO: 129; and
a CD56 signature peptide of SEQ ID NO: 15 with an antibody or antigen-binding fragment thereof that binds the CD56 signature peptide and includes: a heavy chain variable (VH) domain including CDR1 of SEQ ID NO: 136, CDR2 of SEQ ID NO: 137, and CDR3 of SEQ ID NO: 138, and a light chain variable (VL) domain including: CDR1 of SEQ ID NO: 139, CDR2 of SEQ ID NO: 140, and CDR3 of SEQ ID NO: 141;
performing liquid chromatography-multiple reaction monitoring mass spectrometry (LC-MRM-MS) on the enriched first CD42, second CD42, and/or CD56 signature peptides to determine a concentration of each signature peptide; and
comparing the concentrations of the first CD42, second CD42, and CD56 signature peptides to that of corresponding predetermined threshold concentrations.
   3. The method of embodiment 2, wherein the method further includes assessing a platelet level in the subject after the comparing of the first and second CD42 signature peptide concentrations.
   4. The method of embodiment 2 or 3, wherein the method further includes assessing a natural killer cell level in the subject after the comparing of the CD56 signature peptide concentration.
   5. The method of any one of embodiments 1-4, wherein the method is performed as part of a newborn screening (NBS) that additionally screens the subject for one or more of phenylketonuria, primary congenital hypothyroidism, cystic fibrosis, and sickle cell disease.
   6. The method of any one of embodiments 1-5, wherein the biological sample is dried blood spot (DBS), a buccal swab, peripheral blood mononuclear cells (PBMCs), or white blood cells (WBCs).
   7. A method of screening for X-linked chronic granulomatous disease (X-CGD), X-linked lymphoproliferative syndrome 1 (XLP1), familial hemophagocytic lymphohistiocytosis 2 (FHL2), ataxia telangiectasia (AT), common variable immunodeficiency (CVID), severe combined immunodeficiency (SCID), adenosine deaminase (ADA) deficiency, and/or dedicator of cytokinesis 8 (DOCKS) deficiency in a subject, the method including:
obtaining a biological sample derived from the subject;
digesting proteins from the biological sample with an enzyme to yield one or more peptides; enriching for:
   a first CYBB signature peptide of X-CGD of SEQ ID NO: 1 with an antibody or antigen binding fragment thereof that binds the first CYBB signature peptide;
   a second CYBB signature peptide of X-CGD of SEQ ID NO: 2 with an antibody or antigen-binding fragment thereof that binds the second CYBB signature peptide and includes: a heavy chain variable (VH) domain including CDR1 of SEQ ID NO: 16, CDR2 of SEQ ID NO: 17, and CDR3 of SEQ ID NO: 18, and a light chain variable (VL) domain including: CDR1 of SEQ ID NO: 19, CDR2 of SEQ ID NO: 20, and CDR3 of SEQ ID NO: 21;
   a first SH2D1A signature peptide of XLP1 of SEQ ID NO: 3 with an antibody or antigen binding fragment thereof that binds the first SH2D1A signature peptide and includes: a heavy chain variable (VH) domain including CDR1 of SEQ ID NO: 160, CDR2 of SEQ ID NO: 161, and CDR3 of SEQ ID NO: 162, and a light chain variable (VL) domain including: CDR1 of SEQ ID NO: 163, CDR2 of SEQ ID NO: 164, and CDR3 of SEQ ID NO: 165;
   a second SH2D1A signature peptide of XLP1 of SEQ ID NO: 4 with an antibody or antigen binding fragment thereof that binds the second SH2D1A signature peptide;
   a first PRF-1 signature peptide of FHL2 of SEQ ID NO: 5 with an antibody or antigen-binding fragment thereof that binds the first PRF-1 signature peptide and includes: a heavy chain variable (VH) domain including CDR1 of SEQ ID NO: 34, CDR2 of SEQ ID NO: 35, and CDR3 of SEQ ID NO: 36, and a light chain variable (VL) domain including: CDR1 of SEQ ID NO: 37, CDR2 of SEQ ID NO: 38, and CDR3 of SEQ ID NO: 39;
   a second PRF-1 signature peptide of FHL2 of SEQ ID NO: 6 with an antibody or antigen binding fragment thereof that binds the second PRF-1 signature peptide;
   a first ATM signature peptide of ataxia telangiectasia of SEQ ID NO: 210 with an antibody or antigen binding fragment thereof that binds the first ATM signature peptide;
   a second ATM signature peptide of ataxia telangiectasia of SEQ ID NO: 211 with an antibody or antigen binding fragment thereof that binds the second ATM signature peptide;
   a third ATM signature peptide of ataxia telangiectasia of SEQ ID NO: 7 with an antibody or antigen-binding fragment thereof that binds the third ATM signature peptide and includes: a heavy chain variable (VH) domain including CDR1 of SEQ ID NO: 52, CDR2 of SEQ ID NO: 53, and CDR3 of SEQ ID NO: 54, and a light chain variable (VL) domain including: CDR1 of SEQ ID NO: 55, CDR2 of SEQ ID NO: 56, and CDR3 of SEQ ID NO: 57;
   a first CD19 signature peptide of CVID of SEQ ID NO: 8 with an antibody or antigen-binding fragment thereof that binds the first CD19 signature peptide and includes: a heavy chain variable (VH) domain including CDR1 of SEQ ID NO: 70, CDR2 of SEQ ID NO: 71, and CDR3 of SEQ ID NO: 72, and a light chain variable (VL) domain including: CDR1 of SEQ ID NO: 73, CDR2 of SEQ ID NO: 74, and CDR3 of SEQ ID NO: 75;
   a second CD19 signature peptide of CVID of SEQ ID NO: 9 with an antibody or antigen binding fragment thereof that binds the second CD19 signature peptide;
   a third CD19 signature peptide of CVID of SEQ ID NO: 10 with an antibody or antigen binding fragment thereof that binds the third CD19 signature peptide;
   a fourth CD19 signature peptide of CVID of SEQ ID NO: 212 with an antibody or antigen binding fragment thereof that binds the fourth CD19 signature peptide;
   a first IL2RG signature peptide of SCID of SEQ ID NO: 213 with an antibody or antigen binding fragment thereof that binds the first IL2RG signature peptide;
   a second IL2RG signature peptide of SCID of SEQ ID NO: 214 with an antibody or antigen binding fragment thereof that binds the second IL2RG signature peptide;
   a CD3ε signature peptide of SCID of SEQ ID NO: 215 with an antibody or antigen binding fragment thereof that binds the CD3ε signature peptide;
   a first CD3dδ signature peptide of SCID of SEQ ID NO: 216 with an antibody or antigen binding fragment thereof that binds the first CD3dδ signature peptide;
   a second CD3dδ signature peptide of SCID of SEQ ID NO: 217 with an antibody or antigen binding fragment thereof that binds the second CD3dδ signature peptide;
   an ADA signature peptide of ADA deficiency of SEQ ID NO: 11 with an antibody or antigen-binding fragment thereof that binds the ADA signature peptide and includes: a heavy chain variable (VH) domain including CDR1 of SEQ ID NO: 82, CDR2 of SEQ ID NO: 83, and CDR3 of SEQ ID NO: 84, and a light chain variable (VL) domain including: CDR1 of SEQ ID NO: 85, CDR2 of SEQ ID NO: 86, and CDR3 of SEQ ID NO: 87; and/or
   a DOCK8 signature peptide of DOCK8 deficiency of SEQ ID NO: 12 with an antibody or antigen-binding fragment thereof that binds the DOCK8 signature peptide and includes: a heavy chain variable (VH) domain including CDR1 of SEQ ID NO: 100, CDR2 of SEQ ID NO: 101, and CDR3 of SEQ ID NO: 102, and a light chain variable (VL) domain including: CDR1 of SEQ ID NO: 103, CDR2 of SEQ ID NO: 104, and CDR3 of SEQ ID NO: 105;
performing liquid chromatography-multiple reaction monitoring mass spectrometry (LC-MRM-MS) on the enriched peptides to determine a concentration of each signature peptide; and
diagnosing the subject with:
   X-CGD when the concentrations of the first and/or second CYBB signature peptides are lower than corresponding predetermined threshold concentrations or when the first and/or second CYBB signature peptides are absent;
   XLP1 when the concentrations of the first and/or second SH2D1A signature peptides are lower than corresponding predetermined threshold concentrations or when the first and/or second SH2D1A signature peptides are absent;
   FHL2 when the concentrations of the first and/or second PRF-1 signature peptides are lower than corresponding predetermined threshold concentrations or when the first and/or second PRF-1 signature peptides are absent;
   AT when the concentrations of the first, second, and/or third ATM signature peptides are lower than corresponding predetermined threshold concentrations or when the first, second, and/or third ATM signature peptides are absent;
   CVID when the concentrations of the first, second, third, and/or fourth CD19 signature peptides are lower than corresponding predetermined threshold concentrations or when the first, second, third, and/or fourth CD19 signature peptides are absent;
   SCID when the concentrations of the first IL2RG, the second IL2RG, the CD3ε, the first CD3δ, and/or the second CD3δ signature peptides are lower than corresponding predetermined threshold concentrations or when the first IL2RG, the second IL2RG, the CD3ε, the first CD3δ, and/or the second CD3δ signature peptides are absent;
   ADA deficiency when the concentration of the ADA signature peptide is lower than a predetermined threshold concentration or when the ADA signature peptide is absent; and/or
   DOCK8 deficiency when the concentration of the DOCK8 signature peptide is lower than a predetermined threshold concentration or when the DOCK8 signature peptide is absent.
      8. The method of embodiment 7, wherein the method further includes assessing a T cell level in the subject after comparing the CD3ε, the first CD3δ, and/or the second CD3δ signature peptides to that of corresponding predetermined threshold concentrations.
      9. The method of embodiment 7 or 8, wherein the method further includes enriching for:
a first CD42 signature peptide of SEQ ID NO: 13 with an antibody or antigen-binding fragment thereof that binds the first CD42 signature peptide and includes: a heavy chain variable (VH) domain including CDR1 of SEQ ID NO: 112, CDR2 of SEQ ID NO: 113, and CDR3 of SEQ ID NO: 114, and a light chain variable (VL) domain including: CDR1 of SEQ ID NO: 115, CDR2 of SEQ ID NO: 116, and CDR3 of SEQ ID NO: 117;
a second CD42 signature peptide of SEQ ID NO: 14 with an antibody or antigen-binding fragment thereof that binds the second CD42 signature peptide and includes: a heavy chain variable (VH) domain including CDR1 of SEQ ID NO: 124, CDR2 of SEQ ID NO: 125, and CDR3 of SEQ ID NO: 126, and a light chain variable (VL) domain including: CDR1 of SEQ ID NO: 127, CDR2 of SEQ ID NO: 128, and CDR3 of SEQ ID NO: 129; and/or
a CD56 signature peptide of SEQ ID NO: 15 with an antibody or antigen-binding fragment thereof that binds the CD56 signature peptide and includes: a heavy chain variable (VH) domain including CDR1 of SEQ ID NO: 136, CDR2 of SEQ ID NO: 137, and CDR3 of SEQ ID NO: 138, and a light chain variable (VL) domain including: CDR1 of SEQ ID NO: 139, CDR2 of SEQ ID NO: 140, and CDR3 of SEQ ID NO: 141;
performing liquid chromatography-multiple reaction monitoring mass spectrometry (LC-MRM-MS) on the enriched first CD42, second CD42, and/or CD56 signature peptides to determine a concentration of each signature peptide; and
comparing the concentrations of the first CD42, second CD42, and/or CD56 signature peptides to that of corresponding predetermined threshold concentrations.
   10. The method of embodiment 9, wherein the method further includes assessing a platelet level in the subject after the comparing of the first and/or second CD42 signature peptide concentrations.
   11. The method of embodiment 9 or 10, wherein the method further includes assessing a natural killer cell level in the subject after the comparing of the CD56 signature peptide concentration.
   12. The method of any one of embodiments 7-11, wherein the method is performed as part of a newborn screening (NBS) that additionally screens the subject for one or more of phenylketonuria, primary congenital hypothyroidism, cystic fibrosis, and sickle cell disease.
   13. The method of any one of embodiments 7-12, wherein the method is performed in the absence of clinical symptoms of X-CGD, XLP1, FHL2, ataxia telangiectasia, CVID, SCID, ADA deficiency, and/or DOCK8 deficiency in the subject.
   14. The method of any one of embodiments 7-13, wherein the biological sample is dried blood spot (DBS), a buccal swab, peripheral blood mononuclear cells (PBMCs), or white blood cells (WBCs).
   15. The method of any one of embodiments 7-14, wherein the enzyme is trypsin.
   16. The method of any one of embodiments 7-15, wherein the corresponding predetermined threshold concentration for each signature peptide is calculated from a standard deviation (SD) of the mean concentration of each signature peptide in a corresponding biological sample from a population of normal control subjects.
   17. The method of embodiment 16, wherein the mean concentration of the second CYBB signature peptide of X-CGD of SEQ ID NO: 2 in the corresponding biological sample from a population of normal control subjects includes a concentration in a range of 300 pmol/L to 3000 pmol/L.
   18. The method of embodiment 16 or 17, wherein the corresponding predetermined threshold concentration includes −2.5 SD to −2 SD of the mean concentration of the second CYBB signature peptide of X-CGD of SEQ ID NO: 2 in the corresponding biological sample from a population of normal control subjects.
   19. The method of any one of embodiments 16-18, wherein the mean concentration of the first PRF-1 signature peptide of FHL2 of SEQ ID NO: 5 in the corresponding biological sample from a population of normal control subjects includes a concentration in a range of 10 pmol/L to 200 pmol/L.
   20. The method of any one of embodiments 16-19, wherein the corresponding predetermined threshold concentration includes −1.5 SD to −1 SD of the mean concentration of the first PRF-1 signature peptide of FHL2 of SEQ ID NO: 5 in the corresponding biological sample from a population of normal control subjects.
   21. The method of any one of embodiments 16-20, wherein the mean concentration of the third ATM signature peptide of ataxia telangiectasia of SEQ ID NO: 7 in the corresponding biological sample from a population of normal control subjects includes a concentration in a range of 20 pmol/L to 100 pmol/L.
   22. The method of any one of embodiments 16-21, wherein the corresponding predetermined threshold concentration includes −2.25 SD to −1.75 SD of the mean concentration of the third ATM signature peptide of ataxia telangiectasia of SEQ ID NO: 7 in the corresponding biological sample from a population of normal control subjects.
   23. The method of any one of embodiments 16-22, wherein the mean concentration of the ADA signature peptide of ADA deficiency of SEQ ID NO: 11 in the corresponding biological sample from a population of normal control subjects includes a concentration in a range of 900 pmol/L to 5000 pmol/L.
   24. The method of any one of embodiments 16-23, wherein the corresponding predetermined threshold concentration includes −2.1 SD to −1.6 SD of the mean concentration of the ADA signature peptide of ADA deficiency of SEQ ID NO: 11 in the corresponding biological sample from a population of normal control subjects.
   25. The method of any one of embodiments 16-24, wherein the mean concentration of the DOCK8 signature peptide of DOCK8 deficiency of SEQ ID NO: 12 in the corresponding biological sample from a population of normal control subjects includes a concentration in a range of 70 pmol/L to 550 pmol/L.
   26. The method of any one of embodiments 16-25, wherein the corresponding predetermined threshold concentration includes −2.5 SD to −2 SD of the mean concentration of the DOCK8 signature peptide of DOCK8 deficiency of SEQ ID NO: 12 in the corresponding biological sample from a population of normal control subjects.
   27. The method of any one of embodiments 16-26, wherein the mean concentration of the first CD42 signature peptide of SEQ ID NO: 13 in the corresponding biological sample from a population of normal control subjects includes a concentration in a range of 4000 pmol/L to 20000 pmol/L.
   28. The method of any one of embodiments 16-27, wherein the corresponding predetermined threshold concentration includes −2.25 SD to −1.75 SD of the mean concentration of the first CD42 signature peptide of SEQ ID NO: 13 in the corresponding biological sample from a population of normal control subjects.
   29. The method of any one of embodiments 16-28, wherein the mean concentration of the second CD42 signature peptide of SEQ ID NO: 14 in the corresponding biological sample from a population of normal control subjects includes a concentration in a range of 8000 pmol/L to 30000 pmol/L.
   30. The method of any one of embodiments 16-29, wherein the corresponding predetermined threshold concentration includes −1.75 SD to −1.25 SD of the mean concentration of the second CD42 signature peptide of SEQ ID NO: 14 in the corresponding biological sample from a population of normal control subjects.
   31. The method of any one of embodiments 16-30, wherein the mean concentration of the CD56 signature peptide of SEQ ID NO: 15 in the corresponding biological sample from a population of normal control subjects includes a concentration in a range of 600 pmol/L to 5000 pmol/L.
   32. The method of any one of embodiments 16-31, wherein the corresponding predetermined threshold concentration includes −2.75 SD to −2.25 SD of the mean concentration of the CD56 signature peptide of SEQ ID NO: 15 in the corresponding biological sample from a population of normal control subjects.
   33. The method of any one of embodiments 7-32, wherein the antibody or antigen-binding fragment thereof used for enrichment of the second CYBB signature peptide of X-CGD of SEQ ID NO: 2 includes one or more of: a VH domain of SEQ ID NO: 27; a VL domain of SEQ ID NO: 33; a heavy chain of SEQ ID NO: 24; or a light chain of SEQ ID NO: 30.
   34. The method of any one of embodiments 7-33, wherein the antibody or antigen-binding fragment thereof used for enrichment of the first SH2D1A signature peptide of XLP1 of SEQ ID NO: 3 includes one or more of: a VH domain of SEQ ID NO: 173; a VL domain of SEQ ID NO: 181; a heavy chain of SEQ ID NO: 169; or a light chain of SEQ ID NO: 177.
   35. The method of any one of embodiments 7-34, wherein the antibody or antigen-binding fragment thereof used for enrichment of the first PRF-1 signature peptide of FHL2 of SEQ ID NO: 5 includes one or more of: a VH domain of SEQ ID NO: 45; a VL domain of SEQ ID NO: 51; a heavy chain of SEQ ID NO: 42; or a light chain of SEQ ID NO: 48.
   36. The method of any one of embodiments 7-35, wherein the antibody or antigen-binding fragment thereof used for enrichment of the third ATM signature peptide of ataxia telangiectasia of SEQ ID NO: 7 includes one or more of: a VH domain of SEQ ID NO: 63; a VL domain of SEQ ID NO: 69; a heavy chain of SEQ ID NO: 60; or a light chain of SEQ ID NO: 66.
   37. The method of any one of embodiments 7-36, wherein the antibody or antigen-binding fragment thereof used for enrichment of the first CD19 signature peptide of CVID of SEQ ID NO: 8 includes one or more of: a VH domain of SEQ ID NO: 78; or a VL domain of SEQ ID NO: 81.
   38. The method of any one of embodiments 7-37, wherein the antibody or antigen-binding fragment thereof used for enrichment of the ADA signature peptide of ADA deficiency of SEQ ID NO: 11 includes one or more of: a VH domain of SEQ ID NO: 93; a VL domain of SEQ ID NO: 99; a heavy chain of SEQ ID NO: 90; or a light chain of SEQ ID NO: 96.
   39. The method of any one of embodiments 7-38, wherein the antibody or antigen-binding fragment thereof used for enrichment of the DOCK8 signature peptide of DOCK8 deficiency of SEQ ID NO: 12 includes a VH domain of SEQ ID NO: 108 and/or a VL domain of SEQ ID NO: 111.
   40. The method of any one of embodiments 7-39, wherein the antibody or antigen-binding fragment thereof used for enrichment of the first CD42 signature peptide of SEQ ID NO: 13 includes a VH domain of SEQ ID NO: 120 and/or a VL domain of SEQ ID NO: 123.
   41. The method of any one of embodiments 7-40, wherein the antibody or antigen-binding fragment thereof used for enrichment of the second CD42 signature peptide of SEQ ID NO: 14 includes a VH domain of SEQ ID NO: 132 and/or a VL domain of SEQ ID NO: 135.
   42. The method of any one of embodiments 7-41, wherein the antibody or antigen-binding fragment thereof used for enrichment of the CD56 signature peptide of SEQ ID NO: 15 includes one or more of: a VH domain of SEQ ID NO: 147; a VL domain of SEQ ID NO: 153; a heavy chain of SEQ ID NO: 144; or a light chain of SEQ ID NO: 150.
   43. A method of detecting one or more signature peptides of X-linked chronic granulomatous disease (X-CGD), X-linked lymphoproliferative syndrome 1 (XLP1), familial hemophagocytic lymphohistiocytosis 2 (FHL2), ataxia telangiectasia (AT), common variable immunodeficiency (CVID), severe combined immunodeficiency (SCID), adenosine deaminase (ADA) deficiency, and/or dedicator of cytokinesis 8 (DOCK8) deficiency in one or more biological sample, the method including:
obtaining the one or more biological sample;
digesting proteins from blood of each biological sample with an enzyme to yield one or more peptides;
enriching for:
   a first CYBB signature peptide of X-CGD of SEQ ID NO: 1 with an antibody or antigen binding fragment thereof that binds the first CYBB signature peptide;
   a second CYBB signature peptide of X-CGD of SEQ ID NO: 2 with an antibody or antigen-binding fragment thereof that binds the second CYBB signature peptide and includes: a heavy chain variable (VH) domain including CDR1 of SEQ ID NO: 16, CDR2 of SEQ ID NO: 17, and CDR3 of SEQ ID NO: 18, and a light chain variable (VL) domain including: CDR1 of SEQ ID NO: 19, CDR2 of SEQ ID NO: 20, and CDR3 of SEQ ID NO: 21;
   a first SH2D1A signature peptide of XLP1 of SEQ ID NO: 3 with an antibody or antigen binding fragment thereof that binds the first SH2D1A signature peptide and includes: a heavy chain variable (VH) domain including CDR1 of SEQ ID NO: 160, CDR2 of SEQ ID NO: 161, and CDR3 of SEQ ID NO: 162, and a light chain variable (VL) domain including: CDR1 of SEQ ID NO: 163, CDR2 of SEQ ID NO: 164, and CDR3 of SEQ ID NO: 165;
   a second SH2D1A signature peptide of XLP1 of SEQ ID NO: 4 with an antibody or antigen binding fragment thereof that binds the second SH2D1A signature peptide;
   a first PRF-1 signature peptide of FHL2 of SEQ ID NO: 5 with an antibody or antigen-binding fragment thereof that binds the first PRF-1 signature peptide and includes: a heavy chain variable (VH) domain including CDR1 of SEQ ID NO: 34, CDR2 of SEQ ID NO: 35, and CDR3 of SEQ ID NO: 36, and a light chain variable (VL) domain including: CDR1 of SEQ ID NO: 37, CDR2 of SEQ ID NO: 38, and CDR3 of SEQ ID NO: 39;
   a second PRF-1 signature peptide of FHL2 of SEQ ID NO: 6 with an antibody or antigen binding fragment thereof that binds the second PRF-1 signature peptide;
   a first ATM signature peptide of ataxia telangiectasia of SEQ ID NO: 210 with an antibody or antigen binding fragment thereof that binds the first ATM signature peptide;
   a second ATM signature peptide of ataxia telangiectasia of SEQ ID NO: 211 with an antibody or antigen binding fragment thereof that binds the second ATM signature peptide;
   a third ATM signature peptide of ataxia telangiectasia of SEQ ID NO: 7 with an antibody or antigen-binding fragment thereof that binds the third ATM signature peptide and includes: a heavy chain variable (VH) domain including CDR1 of SEQ ID NO: 52, CDR2 of SEQ ID NO: 53, and CDR3 of SEQ ID NO: 54, and a light chain variable (VL) domain including: CDR1 of SEQ ID NO: 55, CDR2 of SEQ ID NO: 56, and CDR3 of SEQ ID NO: 57;
   a first CD19 signature peptide of CVID of SEQ ID NO: 8 with an antibody or antigen-binding fragment thereof that binds the first CD19 signature peptide and includes: a heavy chain variable (VH) domain including CDR1 of SEQ ID NO: 70, CDR2 of SEQ ID NO: 71, and CDR3 of SEQ ID NO: 72, and a light chain variable (VL) domain including: CDR1 of SEQ ID NO: 73, CDR2 of SEQ ID NO: 74, and CDR3 of SEQ ID NO: 75;
   a second CD19 signature peptide of CVID of SEQ ID NO: 9 with an antibody or antigen binding fragment thereof that binds the second CD19 signature peptide;
   a third CD19 signature peptide of CVID of SEQ ID NO: 10 with an antibody or antigen binding fragment thereof that binds the third CD19 signature peptide;
   a fourth CD19 signature peptide of CVID of SEQ ID NO: 212 with an antibody or antigen binding fragment thereof that binds the fourth CD19 signature peptide;
   a first IL2RG signature peptide of SCID of SEQ ID NO: 213 with an antibody or antigen binding fragment thereof that binds the first IL2RG signature peptide;
   a second IL2RG signature peptide of SCID of SEQ ID NO: 214 with an antibody or antigen binding fragment thereof that binds the second IL2RG signature peptide;
   a CD3ε signature peptide of SCID of SEQ ID NO: 215 with an antibody or antigen binding fragment thereof that binds the CD3ε signature peptide;
   a first CD3dδ signature peptide of SCID of SEQ ID NO: 216 with an antibody or antigen binding fragment thereof that binds the first CD3dδ signature peptide;
   a second CD3dδ signature peptide of SCID of SEQ ID NO: 217 with an antibody or antigen binding fragment thereof that binds the second CD3dδ signature peptide;
   an ADA signature peptide of ADA deficiency of SEQ ID NO: 11 with an antibody or antigen-binding fragment thereof that binds the ADA signature peptide and includes: a heavy chain variable (VH) domain including CDR1 of SEQ ID NO: 82, CDR2 of SEQ ID NO: 83, and CDR3 of SEQ ID NO: 84, and a light chain variable (VL) domain including: CDR1 of SEQ ID NO: 85, CDR2 of SEQ ID NO: 86, and CDR3 of SEQ ID NO: 87; and/or
   a DOCK8 signature peptide of DOCK8 deficiency of SEQ ID NO: 12 with an antibody or antigen-binding fragment thereof that binds the DOCK8 signature peptide and includes: a heavy chain variable (VH) domain including CDR1 of SEQ ID NO: 100, CDR2 of SEQ ID NO: 101, and CDR3 of SEQ ID NO: 102, and a light chain variable (VL) domain including: CDR1 of SEQ ID NO: 103, CDR2 of SEQ ID NO: 104, and CDR3 of SEQ ID NO: 105;
and
performing liquid chromatography-multiple reaction monitoring mass spectrometry (LC-MRM-MS) on the enriched peptides to determine a concentration of each signature peptide, thereby detecting one or more signature peptides of X-CGD, XLP1, FHL2, AT, CVID, SCID, ADA deficiency, and/or DOCK8 deficiency in the one or more biological sample.
   44. The method of embodiment 43, wherein the method further includes detecting one or more signature peptides of platelets and/or natural killer (NK) cells by enriching for:
a first CD42 signature peptide of SEQ ID NO: 13 with an antibody or antigen-binding fragment thereof that binds the first CD42 signature peptide and includes: a heavy chain variable (VH) domain including CDR1 of SEQ ID NO: 112, CDR2 of SEQ ID NO: 113, and CDR3 of SEQ ID NO: 114, and a light chain variable (VL) domain including: CDR1 of SEQ ID NO: 115, CDR2 of SEQ ID NO: 116, and CDR3 of SEQ ID NO: 117;
a second CD42 signature peptide of SEQ ID NO: 14 with an antibody or antigen-binding fragment thereof that binds the second CD42 signature peptide and includes: a heavy chain variable (VH) domain including CDR1 of SEQ ID NO: 124, CDR2 of SEQ ID NO: 125, and CDR3 of SEQ ID NO: 126, and a light chain variable (VL) domain including: CDR1 of SEQ ID NO: 127, CDR2 of SEQ ID NO: 128, and CDR3 of SEQ ID NO: 129; and/or
a CD56 signature peptide of SEQ ID NO: 15 with an antibody or antigen-binding fragment thereof that binds the CD56 signature peptide and includes: a heavy chain variable (VH) domain including CDR1 of SEQ ID NO: 136, CDR2 of SEQ ID NO: 137, and CDR3 of SEQ ID NO: 138, and a light chain variable (VL) domain including: CDR1 of SEQ ID NO: 139, CDR2 of SEQ ID NO: 140, and CDR3 of SEQ ID NO: 141;
and
performing liquid chromatography-multiple reaction monitoring mass spectrometry (LC-MRM-MS) on the enriched first CD42, second CD42, and/or CD56 signature peptides to determine a concentration of each signature peptide, thereby detecting one or more signature peptides of platelets and/or natural killer (NK) cells.
   45. The method of embodiment 43 or 44, wherein the one or more biological sample is dried blood spot (DBS), a buccal swab, peripheral blood mononuclear cells (PBMCs), or white blood cells (WBCs).
   46. The method of any one of embodiments 43-45, wherein the enzyme is trypsin.
   47. The method of any one of embodiments 43-46, further including comparing the concentration of each signature peptide to that of a corresponding predetermined threshold concentration.
   48. The method of any one of embodiments 43-47, wherein the corresponding predetermined threshold concentration for each signature peptide is calculated from a standard deviation (SD) of the mean concentration of each signature peptide in the one or more corresponding biological sample from a population of normal control subjects.
   49. The method of embodiment 48, wherein the mean concentration of the second CYBB signature peptide of X-CGD of SEQ ID NO: 2 in the one or more corresponding biological sample from a population of normal control subjects includes a concentration in a range of 300 pmol/L to 3000 pmol/L.
   50. The method of embodiment 48 or 49, wherein the corresponding predetermined threshold concentration includes −2.5 SD to −2 SD of the mean concentration of the second CYBB signature peptide of X-CGD of SEQ ID NO: 2 in the corresponding biological sample from a population of normal control subjects.
   51. The method of any one of embodiments 48-50, wherein the mean concentration of the first PRF-1 signature peptide of FHL2 of SEQ ID NO: 5 in the one or more corresponding biological sample from a population of normal control subjects includes a concentration in a range of 10 pmol/L to 200 pmol/L.
   52. The method of any one of embodiments 48-51, wherein the corresponding predetermined threshold concentration includes −1.5 SD to −1 SD of the mean concentration of the first PRF-1 signature peptide of FHL2 of SEQ ID NO: 5 in the corresponding biological sample from a population of normal control subjects.
   53. The method of any one of embodiments 48-52, wherein the mean concentration of the third ATM signature peptide of ataxia telangiectasia of SEQ ID NO: 7 in the one or more corresponding biological sample from a population of normal control subjects includes a concentration in a range of 20 pmol/L to 100 pmol/L.
   54. The method of any one of embodiments 48-53, wherein the corresponding predetermined threshold concentration includes −2.25 SD to −1.75 SD of the mean concentration of the third ATM signature peptide of ataxia telangiectasia of SEQ ID NO: 7 in the corresponding biological sample from a population of normal control subjects.
   55. The method of any one of embodiments 48-54, wherein the mean concentration of the ADA signature peptide of ADA deficiency of SEQ ID NO: 11 in the one or more corresponding biological sample from a population of normal control subjects includes a concentration in a range of 900 pmol/L to 5000 pmol/L.
   56. The method of any one of embodiments 48-55, wherein the corresponding predetermined threshold concentration includes −2.1 SD to −1.6 SD of the mean concentration of the ADA signature peptide of ADA deficiency of SEQ ID NO: 11 in the corresponding biological sample from a population of normal control subjects.
   57. The method of any one of embodiments 48-56, wherein the mean concentration of the DOCK8 signature peptide of DOCK8 deficiency of SEQ ID NO: 12 in the one or more corresponding biological sample from a population of normal control subjects includes a concentration in a range of 70 pmol/L to 550 pmol/L.
   58. The method of any one of embodiments 48-57, wherein the corresponding predetermined threshold concentration includes −2.5 SD to −2 SD of the mean concentration of the DOCK8 signature peptide of DOCK8 deficiency of SEQ ID NO: 12 in the corresponding biological sample from a population of normal control subjects.
   59. The method of any one of embodiments 48-58, wherein the mean concentration of the first CD42 signature peptide of SEQ ID NO: 13 in the one or more corresponding biological sample from a population of normal control subjects includes a concentration in a range of 4000 pmol/L to 20000 pmol/L.
   60. The method of any one of embodiments 48-59, wherein the corresponding predetermined threshold concentration includes −2.25 SD to −1.75 SD of the mean concentration of the first CD42 signature peptide of SEQ ID NO: 13 in the corresponding biological sample from a population of normal control subjects.
   61. The method of any one of embodiments 48-60, wherein the mean concentration of the second CD42 signature peptide of SEQ ID NO: 14 in the one or more corresponding biological sample from a population of normal control subjects includes a concentration in a range of 8000 pmol/L to 30000 pmol/L.
   62. The method of any one of embodiments 48-61, wherein the corresponding predetermined threshold concentration includes −1.75 SD to −1.25 SD of the mean concentration of the second CD42 signature peptide of SEQ ID NO: 14 in the corresponding biological sample from a population of normal control subjects.
   63. The method of any one of embodiments 48-62, wherein the mean concentration of the CD56 signature peptide of SEQ ID NO: 15 in the one or more corresponding biological sample from a population of normal control subjects includes a concentration in a range of 600 pmol/L to 5000 pmol/L.
   64. The method of any one of embodiments 48-63, wherein the corresponding predetermined threshold concentration includes −2.75 SD to −2.25 SD of the mean concentration of the CD56 signature peptide of SEQ ID NO: 15 in the corresponding biological sample from a population of normal control subjects.
   65. The method of any one of embodiments 43-64, wherein the antibody or antigen-binding fragment thereof used for enrichment of the second CYBB signature peptide of X-CGD of SEQ ID NO: 2 includes one or more of: a VH domain of SEQ ID NO: 27; a VL domain of SEQ ID NO: 33; a heavy chain of SEQ ID NO: 24; or a light chain of SEQ ID NO: 30.
   66. The method of any one of embodiments 43-65, wherein the antibody or antigen-binding fragment thereof used for enrichment of the first SH2D1A signature peptide of XLP1 of SEQ ID NO: 3 includes one or more of: a VH domain of SEQ ID NO: 173; a VL domain of SEQ ID NO: 181; a heavy chain of SEQ ID NO: 169; or a light chain of SEQ ID NO: 177.
   67. The method of any one of embodiments 43-66, wherein the antibody or antigen-binding fragment thereof used for enrichment of the first PRF-1 signature peptide of FHL2 of SEQ ID NO: 5 includes one or more of: a VH domain of SEQ ID NO: 45; a VL domain of SEQ ID NO: 51; a heavy chain of SEQ ID NO: 42; or a light chain of SEQ ID NO: 48.
   68. The method of any one of embodiments 43-67, wherein the antibody or antigen-binding fragment thereof used for enrichment of the third ATM signature peptide of ataxia telangiectasia of SEQ ID NO: 7 includes one or more of: a VH domain of SEQ ID NO: 63; a VL domain of SEQ ID NO: 69; a heavy chain of SEQ ID NO: 60; or a light chain of SEQ ID NO: 66.
   69. The method of any one of embodiments 43-68, wherein the antibody or antigen-binding fragment thereof used for enrichment of the first CD19 signature peptide of CVID of SEQ ID NO: 8 includes one or more of: a VH domain of SEQ ID NO: 78; or a VL domain of SEQ ID NO: 81.
   70. The method of any one of embodiments 43-69, wherein the antibody or antigen-binding fragment thereof used for enrichment of the ADA signature peptide of ADA deficiency of SEQ ID NO: 11 includes one or more of: a VH domain of SEQ ID NO: 93; a VL domain of SEQ ID NO: 99; a heavy chain of SEQ ID NO: 90; or a light chain of SEQ ID NO: 96.
   71. The method of any one of embodiments 43-70, wherein the antibody or antigen-binding fragment thereof used for enrichment of the DOCK8 signature peptide of DOCK8 deficiency of SEQ ID NO: 12 includes a VH domain of SEQ ID NO: 108 and/or a VL domain of SEQ ID NO: 111.
   72. The method of any one of embodiments 43-71, wherein the antibody or antigen-binding fragment thereof used for enrichment of the first CD42 signature peptide of SEQ ID NO: 13 includes a VH domain of SEQ ID NO: 120 and/or a VL domain of SEQ ID NO: 123.
   73. The method of any one of embodiments 43-72, wherein the antibody or antigen-binding fragment thereof used for enrichment of the second CD42 signature peptide of SEQ ID NO: 14 includes a VH domain of SEQ ID NO: 132 and/or a VL domain of SEQ ID NO: 135.
   74. The method of any one of embodiments 43-73, wherein the antibody or antigen-binding fragment thereof used for enrichment of the CD56 signature peptide of SEQ ID NO: 15 includes one or more of: a VH domain of SEQ ID NO: 147; a VL domain of SEQ ID NO: 153; a heavy chain of SEQ ID NO: 144; or a light chain of SEQ ID NO: 150.
   75. A method of determining efficacy of one or more treatments in a subject being treated for X-linked chronic granulomatous disease (X-CGD), X-linked lymphoproliferative syndrome 1 (XLP1), familial hemophagocytic lymphohistiocytosis 2 (FHL2), ataxia telangiectasia (AT), common variable immunodeficiency (CVID), severe combined immunodeficiency (SCID), adenosine deaminase (ADA) deficiency, and/or dedicator of cytokinesis 8 (DOCK8) deficiency, the method including:
obtaining a biological sample derived from the subject prior to the one or more treatments;
digesting proteins from blood of the biological sample with an enzyme to yield one or more peptides;
enriching for:
   a first CYBB signature peptide of X-CGD of SEQ ID NO: 1 with an antibody or antigen binding fragment thereof that binds the first CYBB signature peptide;
   a second CYBB signature peptide of X-CGD of SEQ ID NO: 2 with an antibody or antigen-binding fragment thereof that binds the second CYBB signature peptide and includes: a heavy chain variable (VH) domain including CDR1 of SEQ ID NO: 16, CDR2 of SEQ ID NO: 17, and CDR3 of SEQ ID NO: 18, and a light chain variable (VL) domain including: CDR1 of SEQ ID NO: 19, CDR2 of SEQ ID NO: 20, and CDR3 of SEQ ID NO: 21;
   a first SH2D1A signature peptide of XLP1 of SEQ ID NO: 3 with an antibody or antigen binding fragment thereof that binds the first SH2D1A signature peptide and includes: a heavy chain variable (VH) domain including CDR1 of SEQ ID NO: 160, CDR2 of SEQ ID NO: 161, and CDR3 of SEQ ID NO: 162, and a light chain variable (VL) domain including: CDR1 of SEQ ID NO: 163, CDR2 of SEQ ID NO: 164, and CDR3 of SEQ ID NO: 165;
   a second SH2D1A signature peptide of XLP1 of SEQ ID NO: 4 with an antibody or antigen binding fragment thereof that binds the second SH2D1A signature peptide;
   a first PRF-1 signature peptide of FHL2 of SEQ ID NO: 5 with an antibody or antigen-binding fragment thereof that binds the first PRF-1 signature peptide and includes: a heavy chain variable (VH) domain including CDR1 of SEQ ID NO: 34, CDR2 of SEQ ID NO: 35, and CDR3 of SEQ ID NO: 36, and a light chain variable (VL) domain including: CDR1 of SEQ ID NO: 37, CDR2 of SEQ ID NO: 38, and CDR3 of SEQ ID NO: 39;
   a second PRF-1 signature peptide of FHL2 of SEQ ID NO: 6 with an antibody or antigen binding fragment thereof that binds the second PRF-1 signature peptide;
   a first ATM signature peptide of ataxia telangiectasia of SEQ ID NO: 210 with an antibody or antigen binding fragment thereof that binds the first ATM signature peptide;
   a second ATM signature peptide of ataxia telangiectasia of SEQ ID NO: 211 with an antibody or antigen binding fragment thereof that binds the second ATM signature peptide;
   a third ATM signature peptide of ataxia telangiectasia of SEQ ID NO: 7 with an antibody or antigen-binding fragment thereof that binds the third ATM signature peptide and includes: a heavy chain variable (VH) domain including CDR1 of SEQ ID NO: 52, CDR2 of SEQ ID NO: 53, and CDR3 of SEQ ID NO: 54, and a light chain variable (VL) domain including: CDR1 of SEQ ID NO: 55, CDR2 of SEQ ID NO: 56, and CDR3 of SEQ ID NO: 57;
   a first CD19 signature peptide of CVID of SEQ ID NO: 8 with an antibody or antigen-binding fragment thereof that binds the first CD19 signature peptide and includes: a heavy chain variable (VH) domain including CDR1 of SEQ ID NO: 70, CDR2 of SEQ ID NO: 71, and CDR3 of SEQ ID NO: 72, and a light chain variable (VL) domain including: CDR1 of SEQ ID NO: 73, CDR2 of SEQ ID NO: 74, and CDR3 of SEQ ID NO: 75;
   a second CD19 signature peptide of CVID of SEQ ID NO: 9 with an antibody or antigen binding fragment thereof that binds the second CD19 signature peptide;
   a third CD19 signature peptide of CVID of SEQ ID NO: 10 with an antibody or antigen binding fragment thereof that binds the third CD19 signature peptide;
   a fourth CD19 signature peptide of CVID of SEQ ID NO: 212 with an antibody or antigen binding fragment thereof that binds the fourth CD19 signature peptide;
   a first IL2RG signature peptide of SCID of SEQ ID NO: 213 with an antibody or antigen binding fragment thereof that binds the first IL2RG signature peptide;
   a second IL2RG signature peptide of SCID of SEQ ID NO: 214 with an antibody or antigen binding fragment thereof that binds the second IL2RG signature peptide;
   a CD3ε signature peptide of SCID of SEQ ID NO: 215 with an antibody or antigen binding fragment thereof that binds the CD3ε signature peptide;
   a first CD3dδ signature peptide of SCID of SEQ ID NO: 216 with an antibody or antigen binding fragment thereof that binds the first CD3dδ signature peptide;
   a second CD3dδ signature peptide of SCID of SEQ ID NO: 217 with an antibody or antigen binding fragment thereof that binds the second CD3dδ signature peptide;
   an ADA signature peptide of ADA deficiency of SEQ ID NO: 11 with an antibody or antigen-binding fragment thereof that binds the ADA signature peptide and includes: a heavy chain variable (VH) domain including CDR1 of SEQ ID NO: 82, CDR2 of SEQ ID NO: 83, and CDR3 of SEQ ID NO: 84, and a light chain variable (VL) domain including: CDR1 of SEQ ID NO: 85, CDR2 of SEQ ID NO: 86, and CDR3 of SEQ ID NO: 87; and/or
   a DOCK8 signature peptide of DOCK8 deficiency of SEQ ID NO: 12 with an antibody or antigen-binding fragment thereof that binds the DOCK8 signature peptide and includes: a heavy chain variable (VH) domain including CDR1 of SEQ ID NO: 100, CDR2 of SEQ ID NO: 101, and CDR3 of SEQ ID NO: 102, and a light chain variable (VL) domain including: CDR1 of SEQ ID NO: 103, CDR2 of SEQ ID NO: 104, and CDR3 of SEQ ID NO: 105;
performing liquid chromatography-multiple reaction monitoring mass spectrometry (LC-MRM-MS) on the enriched peptides to determine a concentration of each signature peptide in the subject prior to the one or more treatments;
repeating the obtaining, digesting, enriching, and performing on a biological sample derived from the subject during or after the one or more treatments;
and
determining that the one or more treatments is effective for:
   X-CGD when the concentrations of the first and/or second CYBB signature peptides during or after the one or more treatments are higher than the corresponding concentrations of the first and/or second CYBB signature peptides prior to the one or more treatments;
   XLP1 when the concentrations of the first and/or second SH2D1A signature peptides during or after the one or more treatments are higher than the corresponding concentrations of the first and/or second SH2D1A signature peptides prior to the one or more treatments;
   FHL2 when the concentrations of the first and/or second PRF-1 signature peptides during or after the one or more treatments are higher than the corresponding concentrations of the first and/or second PRF-1 signature peptides prior to the one or more treatments;
   AT when the concentrations of the first, second, and/or third ATM signature peptides during or after the one or more treatments is higher than the concentration of the first, second, and/or third ATM signature peptides prior to the one or more treatments;
   CVID when the concentrations of the first, second, third, and/or fourth CD19 signature peptides during or after the one or more treatments are higher than the corresponding concentrations of the first, second, third, and/or fourth CD19 signature peptides prior to the one or more treatments;
   SCID when the concentrations of the first IL2RG, the second IL2RG, the CD3ε, the first CD3δ, and/or the second CD3δ signature peptides during or after the one or more treatments are higher than the corresponding concentrations of the first IL2RG, the second IL2RG, the CD3ε, the first CD3δ, and/or the second CD3δ signature peptides prior to the one or more treatments;
   ADA deficiency when the concentration of the ADA signature peptide during or after the one or more treatments is higher than the concentration of the ADA signature peptide prior to the one or more treatments; and/or
   DOCK8 deficiency when the concentration of the DOCK8 signature peptide during or after the one or more treatments is equal to or higher than a predetermined threshold concentration prior to the one or more treatments,
or
   determining that the one or more treatments is not effective for:
   X-CGD when the concentrations of the first and/or second CYBB signature peptides during or after the one or more treatments are equal to or lower than the corresponding concentrations of the first and/or second CYBB signature peptides prior to the one or more treatments or when the first and/or second CYBB signature peptides are absent;
   XLP1 when the concentrations of the first and/or second SH2D1A signature peptides during or after the one or more treatments are equal to or lower than the corresponding concentrations of the first and/or second SH2D1A signature peptides prior to the one or more treatments or when the first and/or second SH2D1A signature peptides are absent;
   FHL2 when the concentrations of the first and/or second PRF-1 signature peptides during or after the one or more treatments are equal to or lower than the corresponding concentrations of the first and/or second PRF-1 signature peptides prior to the one or more treatments or when the first and/or second PRF-1 signature peptides are absent;
   AT when the concentrations of the first, second, and/or third ATM signature peptides during or after the one or more treatments is equal to or lower than the corresponding concentrations of the first, second, and/or third ATM signature peptides prior to the one or more treatments or when the first, second, and/or third ATM signature peptides are absent;
   CVID when the concentrations of the first, second, third, and/or fourth CD19 signature peptides during or after the one or more treatments are equal to or lower than the corresponding concentrations of the first, second, third, and/or fourth CD19 signature peptides prior to the one or more treatments or when the first, second, third, and/or fourth CD19 signature peptides are absent;
   SCID when the concentrations of the first IL2RG, the second IL2RG, the CD3ε, the first CD3δ, and/or the second CD3δ signature peptides during or after the one or more treatments are equal to or lower than the corresponding concentrations of the first IL2RG, the second IL2RG, the CD3ε, the first CD3δ, and/or the second CD3δ signature peptides prior to the one or more treatments or when the first IL2RG, the second IL2RG, the CD3ε, the first CD3δ, and/or the second CD3δ signature peptides are absent;
   ADA deficiency when the concentration of the ADA signature peptide during or after the one or more treatments is equal to or lower than the concentration of the ADA signature peptide prior to the one or more treatments or when the ADA signature peptide is absent; and/or
   DOCK8 deficiency when the concentration of the DOCK8 signature peptide during or after the one or more treatments is equal to or lower than the concentration of the DOCK8 signature peptide prior to the one or more treatments or when the DOCK8 signature peptide is absent. 76. The method of embodiment 75, wherein the method further includes determining efficacy of one or more treatments in the subject by enriching for:
   a first CD42 signature peptide of SEQ ID NO: 13 with an antibody or antigen-binding fragment thereof that binds the first CD42 signature peptide and includes: a heavy chain variable (VH) domain including CDR1 of SEQ ID NO: 112, CDR2 of SEQ ID NO: 113, and CDR3 of SEQ ID NO: 114, and a light chain variable (VL) domain including: CDR1 of SEQ ID NO: 115, CDR2 of SEQ ID NO: 116, and CDR3 of SEQ ID NO: 117;
   a second CD42 signature peptide of SEQ ID NO: 14 with an antibody or antigen-binding fragment thereof that binds the second CD42 signature peptide and includes: a heavy chain variable (VH) domain including CDR1 of SEQ ID NO: 124, CDR2 of SEQ ID NO: 125, and CDR3 of SEQ ID NO: 126, and a light chain variable (VL) domain including: CDR1 of SEQ ID NO: 127, CDR2 of SEQ ID NO: 128, and CDR3 of SEQ ID NO: 129; and/or
   a CD56 signature peptide of SEQ ID NO: 15 with an antibody or antigen-binding fragment thereof that binds the CD56 signature peptide and includes: a heavy chain variable (VH) domain including CDR1 of SEQ ID NO: 136, CDR2 of SEQ ID NO: 137, and CDR3 of SEQ ID NO: 138, and a light chain variable (VL) domain including: CDR1 of SEQ ID NO: 139, CDR2 of SEQ ID NO: 140, and CDR3 of SEQ ID NO: 141;
in the biological sample prior to the one or more treatments;
performing liquid chromatography-multiple reaction monitoring mass spectrometry (LC-MRM-MS) on the enriched first CD42, second CD42, and/or CD56 signature peptides to determine a concentration of each signature peptide in the subject prior to the one or more treatments; repeating the obtaining, digesting, enriching, and performing on a biological sample derived from the subject during or after the one or more treatments;
and
determining that the one or more treatments is effective:
   when the concentrations of the first CD42, second CD42, and/or CD56 signature peptides during or after the one or more treatments are higher than the corresponding concentrations of the first CD42, second CD42, and/or CD56 signature peptides prior to the one or more treatments, or
   determining that the one or more treatments is not effective:
   when the concentrations of the first CD42, second CD42, and/or CD56 signature peptides during or after the one or more treatments are equal to or lower than the corresponding concentrations of the first CD42, second CD42, and/or CD56 signature peptides prior to the one or more treatments or when the first CD42, second CD42, and/or CD56 signature peptides are absent.
      77. The method of embodiment 75 or 76, wherein the biological sample is dried blood spot (DBS), a buccal swab, peripheral blood mononuclear cells (PBMCs), or white blood cells (WBCs).
      78. The method of any one of embodiments 75-77, wherein the enzyme is trypsin.
      79. The method of any one of embodiments 75-78, further including comparing the concentration of each signature peptide to that of a corresponding predetermined threshold concentration.
      80. The method of any one of embodiments 75-79, wherein the corresponding predetermined threshold concentration for each signature peptide is calculated from a standard deviation (SD) of the mean concentration of each signature peptide in a corresponding biological sample from a population of normal control subjects.
      81. The method of embodiment 80, wherein the mean concentration of the second CYBB signature peptide of X-CGD of SEQ ID NO: 2 in the corresponding biological sample from a population of normal control subjects includes a concentration in a range of 300 pmol/L to 3000 pmol/L.
      82. The method of embodiment 80 or 81, wherein the corresponding predetermined threshold concentration includes −2.5 SD to −2 SD of the mean concentration of the second CYBB signature peptide of X-CGD of SEQ ID NO: 2 in the corresponding biological sample from a population of normal control subjects.
      83. The method of any one of embodiments 80-82, wherein the mean concentration of the first PRF-1 signature peptide of FHL2 of SEQ ID NO: 5 in the corresponding biological sample from a population of normal control subjects includes a concentration in a range of 10 pmol/L to 200 pmol/L.
      84. The method of any one of embodiments 80-83, wherein the corresponding predetermined threshold concentration includes −1.5 SD to −1 SD of the mean concentration of the first PRF-1 signature peptide of FHL2 of SEQ ID NO: 5 in the corresponding biological sample from a population of normal control subjects.
      85. The method of any one of embodiments 80-84, wherein the mean concentration of the third ATM signature peptide of ataxia telangiectasia of SEQ ID NO: 7 in the corresponding biological sample from a population of normal control subjects includes a concentration in a range of 20 pmol/L to 100 pmol/L.
      86. The method of any one of embodiments 80-85, wherein the corresponding predetermined threshold concentration includes −2.25 SD to −1.75 SD of the mean concentration of the third ATM signature peptide of ataxia telangiectasia of SEQ ID NO: 7 in the corresponding biological sample from a population of normal control subjects.
      87. The method of any one of embodiments 80-86, wherein the mean concentration of the ADA signature peptide of ADA deficiency of SEQ ID NO: 11 in the corresponding biological sample from a population of normal control subjects includes a concentration in a range of 900 pmol/L to 5000 pmol/L.
      88. The method of any one of embodiments 80-87, wherein the corresponding predetermined threshold concentration includes −2.1 SD to −1.6 SD of the mean concentration of the ADA signature peptide of ADA deficiency of SEQ ID NO: 11 in the corresponding biological sample from a population of normal control subjects.
      89. The method of any one of embodiments 80-88, wherein the mean concentration of the DOCK8 signature peptide of DOCK8 deficiency of SEQ ID NO: 12 in the corresponding biological sample from a population of normal control subjects includes a concentration in a range of 70 pmol/L to 550 pmol/L.
      90. The method of any one of embodiments 80-89, wherein the corresponding predetermined threshold concentration includes −2.5 SD to −2 SD of the mean concentration of the DOCK8 signature peptide of DOCK8 deficiency of SEQ ID NO: 12 in the corresponding biological sample from a population of normal control subjects.
      91. The method of any one of embodiments 80-90, wherein the mean concentration of the first CD42 signature peptide of SEQ ID NO: 13 in the corresponding biological sample from a population of normal control subjects includes a concentration in a range of 4000 pmol/L to 20000 pmol/L.
      92. The method of any one of embodiments 80-91, wherein the corresponding predetermined threshold concentration includes −2.25 SD to −1.75 SD of the mean concentration of the first CD42 signature peptide of SEQ ID NO: 13 in the corresponding biological sample from a population of normal control subjects.
      93. The method of any one of embodiments 80-92, wherein the mean concentration of the second CD42 signature peptide of SEQ ID NO: 14 in the corresponding biological sample from a population of normal control subjects includes a concentration in a range of 8000 pmol/L to 30000 pmol/L.
      94. The method of any one of embodiments 80-93, wherein the corresponding predetermined threshold concentration includes −1.75 SD to −1.25 SD of the mean concentration of the second CD42 signature peptide of SEQ ID NO: 14 in the corresponding biological sample from a population of normal control subjects.
      95. The method of any one of embodiments 80-94, wherein the mean concentration of the CD56 signature peptide of SEQ ID NO: 15 in the corresponding biological sample from a population of normal control subjects includes a concentration in a range of 600 pmol/L to 5000 pmol/L.
      96. The method of any one of embodiments 80-95, wherein the corresponding predetermined threshold concentration includes −2.75 SD to −2.25 SD of the mean concentration of the CD56 signature peptide of SEQ ID NO: 15 in the corresponding biological sample from a population of normal control subjects.
      97. The method of any one of embodiments 75-96, wherein the antibody or antigen-binding fragment thereof used for enrichment of the second CYBB signature peptide of X-CGD of SEQ ID NO: 2 includes one or more of: a VH domain of SEQ ID NO: 27; a VL domain of SEQ ID NO: 33; a heavy chain of SEQ ID NO: 24; or a light chain of SEQ ID NO: 30.
      98. The method of any one of embodiments 75-97, wherein the antibody or antigen-binding fragment thereof used for enrichment of the first SH2D1A signature peptide of XLP1 of SEQ ID NO: 3 includes one or more of: a VH domain of SEQ ID NO: 173; a VL domain of SEQ ID NO: 181; a heavy chain of SEQ ID NO: 169; or a light chain of SEQ ID NO: 177.
      99. The method of any one of embodiments 75-98, wherein the antibody or antigen-binding fragment thereof used for enrichment of the first PRF-1 signature peptide of FHL2 of SEQ ID NO: 5 includes one or more of: a VH domain of SEQ ID NO: 45; a VL domain of SEQ ID NO: 51; a heavy chain of SEQ ID NO: 42; or a light chain of SEQ ID NO: 48.
      100. The method of any one of embodiments 75-99, wherein the antibody or antigen-binding fragment thereof used for enrichment of the third ATM signature peptide of ataxia telangiectasia of SEQ ID NO: 7 includes one or more of: a VH domain of SEQ ID NO: 63; a VL domain of SEQ ID NO: 69; a heavy chain of SEQ ID NO: 60; or a light chain of SEQ ID NO: 66.
      101. The method of any one of embodiments 75-100, wherein the antibody or antigen-binding fragment thereof used for enrichment of the first CD19 signature peptide of CVID of SEQ ID NO: 8 includes one or more of: a VH domain of SEQ ID NO: 78; or a VL domain of SEQ ID NO: 81.
      102. The method of any one of embodiments 75-101, wherein the antibody or antigen-binding fragment thereof used for enrichment of the ADA signature peptide of ADA deficiency of SEQ ID NO: 11 includes one or more of: a VH domain of SEQ ID NO: 93; a VL domain of SEQ ID NO: 99; a heavy chain of SEQ ID NO: 90; or a light chain of SEQ ID NO: 96.
      103. The method of any one of embodiments 75-102, wherein the antibody or antigen-binding fragment thereof used for enrichment of the DOCK8 signature peptide of DOCK8 deficiency of SEQ ID NO: 12 includes a VH domain of SEQ ID NO: 108 and/or a VL domain of SEQ ID NO: 111.
      104. The method of any one of embodiments 75-103, wherein the antibody or antigen-binding fragment thereof used for enrichment of the first CD42 signature peptide of SEQ ID NO: 13 includes a VH domain of SEQ ID NO: 120 and/or a VL domain of SEQ ID NO: 123.
      105. The method of any one of embodiments 75-104, wherein the antibody or antigen-binding fragment thereof used for enrichment of the second CD42 signature peptide of SEQ ID NO: 14 includes a VH domain of SEQ ID NO: 132 and/or a VL domain of SEQ ID NO: 135.
      106. The method of any one of embodiments 75-105, wherein the antibody or antigen-binding fragment thereof used for enrichment of the CD56 signature peptide of SEQ ID NO: 15 includes one or more of: a VH domain of SEQ ID NO: 147; a VL domain of SEQ ID NO: 153; a heavy chain of SEQ ID NO: 144; or a light chain of SEQ ID NO: 150.
      107. An assay for the screening of X-linked chronic granulomatous disease (X-CGD), X-linked lymphoproliferative syndrome 1 (XLP1), familial hemophagocytic lymphohistiocytosis 2 (FHL2), ataxia telangiectasia (AT), common variable immunodeficiency (CVID), severe combined immunodeficiency (SCID), adenosine deaminase (ADA) deficiency, and/or dedicator of cytokinesis 8 (DOCK8) deficiency in a subject, the assay including:
(i) an antibody or antigen-binding fragment thereof including:
   a heavy chain variable (VH) domain including CDR1 of SEQ ID NO: 16, CDR2 of SEQ ID NO: 17, and CDR3 of SEQ ID NO: 18, and a light chain variable (VL) domain including: CDR1 of SEQ ID NO: 19, CDR2 of SEQ ID NO: 20, and CDR3 of SEQ ID NO: 21 that binds to a CYBB signature peptide of X-CGD of SEQ ID NO: 2;
   a heavy chain variable (VH) domain including CDR1 of SEQ ID NO: 160, CDR2 of SEQ ID NO: 161, and CDR3 of SEQ ID NO: 162, and a light chain variable (VL) domain including: CDR1 of SEQ ID NO: 163, CDR2 of SEQ ID NO: 164, and CDR3 of SEQ ID NO: 165 that binds to a SH2D1A signature peptide of XLP1 of SEQ ID NO: 3;
   a heavy chain variable (VH) domain including CDR1 of SEQ ID NO: 34, CDR2 of SEQ ID NO: 35, and CDR3 of SEQ ID NO: 36, and a light chain variable (VL) domain including: CDR1 of SEQ ID NO: 37, CDR2 of SEQ ID NO: 38, and CDR3 of SEQ ID NO: 39 that binds to a PRF-1 signature peptide of FHL2 of SEQ ID NO: 5;
   a heavy chain variable (VH) domain including CDR1 of SEQ ID NO: 52, CDR2 of SEQ ID NO: 53, and CDR3 of SEQ ID NO: 54, and a light chain variable (VL) domain including: CDR1 of SEQ ID NO: 55, CDR2 of SEQ ID NO: 56, and CDR3 of SEQ ID NO: 67 that binds to an ATM signature peptide of ataxia telangiectasia of SEQ ID NO: 7;
   a heavy chain variable (VH) domain including CDR1 of SEQ ID NO: 70, CDR2 of SEQ ID NO: 71, and CDR3 of SEQ ID NO: 72, and a light chain variable (VL) domain including: CDR1 of SEQ ID NO: 73, CDR2 of SEQ ID NO: 74, and CDR3 of SEQ ID NO: 75 that binds to a CD19 signature peptide of CVID of SEQ ID NO: 8;
   a heavy chain variable (VH) domain including CDR1 of SEQ ID NO: 82, CDR2 of SEQ ID NO: 83, and CDR3 of SEQ ID NO: 84, and a light chain variable (VL) domain including: CDR1 of SEQ ID NO: 85, CDR2 of SEQ ID NO: 86, and CDR3 of SEQ ID NO: 87 that binds to an ADA signature peptide of ADA deficiency of SEQ ID NO: 11; and/or
   a heavy chain variable (VH) domain including CDR1 of SEQ ID NO: 100, CDR2 of SEQ ID NO: 101, and CDR3 of SEQ ID NO: 102, and a light chain variable (VL) domain including: CDR1 of SEQ ID NO: 103, CDR2 of SEQ ID NO: 104, and CDR3 of SEQ ID NO: 105 that binds to a DOCK8 signature peptide of DOCK8 deficiency of SEQ ID NO: 12;
      and/or
(ii) an antibody or antigen-binding fragment thereof that binds a CYBB signature peptide of X-CGD of SEQ ID NO: 1;
   an antibody or antigen-binding fragment thereof that binds a SH2D1A signature peptide of XLP1 of SEQ ID NO: 4;
   an antibody or antigen-binding fragment thereof that binds a PRF-1 signature peptide of FHL2 of SEQ ID NO: 6;
   an antibody or antigen-binding fragment thereof that binds an ATM signature peptide of AT of SEQ ID NO: 210;
   an antibody or antigen-binding fragment thereof that binds an ATM signature peptide of AT of SEQ ID NO: 211;
   an antibody or antigen-binding fragment thereof that binds a CD19 signature peptide of CVID of SEQ ID NO: 9;
   an antibody or antigen-binding fragment thereof that binds a CD19 signature peptide of CVID of SEQ ID NO: 10;
   an antibody or antigen-binding fragment thereof that binds a CD19 signature peptide of CVID of SEQ ID NO: 212;
   an antibody or antigen-binding fragment thereof that binds an IL2RG signature peptide of SCID of SEQ ID NO: 213;
   an antibody or antigen-binding fragment thereof that binds an IL2RG signature peptide of SCID of SEQ ID NO: 214;
   an antibody or antigen-binding fragment thereof that binds a CD3ε signature peptide of SCID of SEQ ID NO: 215;
   an antibody or antigen-binding fragment thereof that binds a CD3δ signature peptide of SCID of SEQ ID NO: 216; and/or an antibody or antigen-binding fragment thereof that binds a CD3δ signature peptide of SCID of SEQ ID NO: 217;
      and/or
(iii) reference signature peptides including:
   a CYBB signature peptide of X-CGD of SEQ ID NO: 1;
   a CYBB signature peptide of X-CGD of SEQ ID NO: 2;
   a SH2D1A signature peptide of XLP1 of SEQ ID NO: 3;
   a SH2D1A signature peptide of XLP1 of SEQ ID NO: 4;
   a PRF-1 signature peptide of FHL2 of SEQ ID NO: 5;
   a PRF-1 signature peptide of FHL2 of SEQ ID NO: 6;
   an ATM signature peptide of ataxia telangiectasia of SEQ ID NO: 210;
   an ATM signature peptide of ataxia telangiectasia of SEQ ID NO: 211;
   an ATM signature peptide of ataxia telangiectasia of SEQ ID NO: 7;
   a CD19 signature peptide of CVID of SEQ ID NO: 8;
   a CD19 signature peptide of CVID of SEQ ID NO: 9;
   a CD19 signature peptide of CVID of SEQ ID NO: 10;
   a CD19 signature peptide of CVID of SEQ ID NO: 212;
   an IL2RG signature peptide of SCID of SEQ ID NO: 213;
   an IL2RG signature peptide of SCID of SEQ ID NO: 214;
   a CD3ε signature peptide of SCID of SEQ ID NO: 215;
   a CD3δ signature peptide of SCID of SEQ ID NO: 216;
   a CD3δ signature peptide of SCID of SEQ ID NO: 217;
   an ADA signature peptide of ADA deficiency of SEQ ID NO: 11; and/or
   a DOCK8 signature peptide of DOCK8 deficiency of SEQ ID NO: 12.
      108. The assay of embodiment 107 further including:
(iv) an antibody or antigen-binding fragment thereof including:
   a heavy chain variable (VH) domain including CDR1 of SEQ ID NO: 112, CDR2 of SEQ ID NO: 113, and CDR3 of SEQ ID NO: 114, and a light chain variable (VL) domain including: CDR1 of SEQ ID NO: 115, CDR2 of SEQ ID NO: 116, and CDR3 of SEQ ID NO: 117 that binds to a CD42 signature peptide of SEQ ID NO: 13;
   a heavy chain variable (VH) domain including CDR1 of SEQ ID NO: 124, CDR2 of SEQ ID NO: 125, and CDR3 of SEQ ID NO: 126, and a light chain variable (VL) domain including: CDR1 of SEQ ID NO: 127, CDR2 of SEQ ID NO: 128, and CDR3 of SEQ ID NO: 129 that binds to a CD42 signature peptide of SEQ ID NO: 14; and/or
   a heavy chain variable (VH) domain including CDR1 of SEQ ID NO: 136, CDR2 of SEQ ID NO: 137, and CDR3 of SEQ ID NO: 138, and a light chain variable (VL) domain including: CDR1 of SEQ ID NO: 139, CDR2 of SEQ ID NO: 140, and CDR3 of SEQ ID NO: 141 that binds to a CD56 signature peptide of SEQ ID NO: 15;
      and/or
(v) reference signature peptides including:
   a CD42 signature peptide of SEQ ID NO: 13;
   a CD42 signature peptide of SEQ ID NO: 14; and/or
   a CD56 signature peptide of SEQ ID NO: 15.
      109. The assay of embodiment 107 or 108, wherein the reference signature peptides are isotopically labeled.
      110. The assay of any one of embodiments 107-109, wherein the antibodies or antigen-binding fragments thereof are attached to magnetic beads.
      111. A recombinant antibody or antigen binding fragment thereof including: a heavy chain variable (VH) domain including CDR1 of SEQ ID NO: 16, CDR2 of SEQ ID NO: 17, and CDR3 of SEQ ID NO: 18, and a light chain variable (VL) domain including: CDR1 of SEQ ID NO: 19, CDR2 of SEQ ID NO: 20, and CDR3 of SEQ ID NO: 21.
      112. The recombinant antibody or antigen binding fragment thereof of embodiment 111, wherein the VH domain is set forth in SEQ ID NO: 27 and/or the heavy chain is set forth in SEQ ID NO: 24; and
      the VL domain is set forth in SEQ ID NO: 33 and/or the light chain is set forth in SEQ ID NO: 30.
      113. A recombinant antibody or antigen binding fragment thereof including: a heavy chain variable (VH) domain including CDR1 of SEQ ID NO: 160, CDR2 of SEQ ID NO: 161, and CDR3 of SEQ ID NO: 162, and a light chain variable (VL) domain including: CDR1 of SEQ ID NO: 163, CDR2 of SEQ ID NO: 164, and CDR3 of SEQ ID NO: 165.
      114. The recombinant antibody or antigen binding fragment thereof of embodiment 113, wherein the VH domain is set forth in SEQ ID NO: 173 and/or the heavy chain is set forth in SEQ ID NO: 169; and
      the VL domain is set forth in SEQ ID NO: 181 and/or the light chain is set forth in SEQ ID NO: 177.
      115. A recombinant antibody or antigen binding fragment thereof including: a heavy chain variable (VH) domain including CDR1 of SEQ ID NO: 34, CDR2 of SEQ ID NO: 35, and CDR3 of SEQ ID NO: 36, and a light chain variable (VL) domain including: CDR1 of SEQ ID NO: 37, CDR2 of SEQ ID NO: 38, and CDR3 of SEQ ID NO: 39.
      116. The recombinant antibody or antigen binding fragment thereof of embodiment 115, wherein the VH domain is set forth in SEQ ID NO: 45 and/or the heavy chain is set forth in SEQ ID NO: 42; and
      the VL domain is set forth in SEQ ID NO: 51 and/or the light chain is set forth in SEQ ID NO: 48.
      117. A recombinant antibody or antigen binding fragment thereof including: a heavy chain variable (VH) domain including CDR1 of SEQ ID NO: 52, CDR2 of SEQ ID NO: 53, and CDR3 of SEQ ID NO: 54, and a light chain variable (VL) domain including: CDR1 of SEQ ID NO: 55, CDR2 of SEQ ID NO: 56, and CDR3 of SEQ ID NO: 57.
      118. The recombinant antibody or antigen binding fragment thereof of embodiment 117, wherein the VH domain is set forth in SEQ ID NO: 63 and/or the heavy chain is set forth in SEQ ID NO: 60; and
      the VL domain is set forth in SEQ ID NO: 69 and/or the light chain is set forth in SEQ ID NO: 66.
      119. A recombinant antibody or antigen binding fragment thereof including: a heavy chain variable (VH) domain including CDR1 of SEQ ID NO: 70, CDR2 of SEQ ID NO: 71, and CDR3 of SEQ ID NO: 72, and a light chain variable (VL) domain including: CDR1 of SEQ ID NO: 73, CDR2 of SEQ ID NO: 74, and CDR3 of SEQ ID NO: 75.
      120. The recombinant antibody or antigen binding fragment thereof of embodiment 119, wherein the VH domain is set forth in SEQ ID NO: 78 and the VL domain is set forth in SEQ ID NO: 81.
      121. A recombinant antibody or antigen binding fragment thereof including: a heavy chain variable (VH) domain including CDR1 of SEQ ID NO: 82, CDR2 of SEQ ID NO: 83, and CDR3 of SEQ ID NO: 84, and a light chain variable (VL) domain including: CDR1 of SEQ ID NO: 85, CDR2 of SEQ ID NO: 86, and CDR3 of SEQ ID NO: 87.
      122. The recombinant antibody or antigen binding fragment thereof of embodiment 121, wherein the VH domain is set forth in SEQ ID NO: 93 and/or the heavy chain is set forth in SEQ ID NO: 90; and
      the VL domain is set forth in SEQ ID NO: 99 and/or the light chain is set forth in SEQ ID NO: 96.
      123. A recombinant antibody or antigen binding fragment thereof including: a heavy chain variable (VH) domain including CDR1 of SEQ ID NO: 100, CDR2 of SEQ ID NO: 101, and CDR3 of SEQ ID NO: 102, and a light chain variable (VL) domain including: CDR1 of SEQ ID NO: 103, CDR2 of SEQ ID NO: 104, and CDR3 of SEQ ID NO: 105.
      124. The recombinant antibody or antigen binding fragment thereof of embodiment 123, wherein the VH domain is set forth in SEQ ID NO: 108 and the VL domain is set forth in SEQ ID NO: 111.
      125. A recombinant antibody or antigen binding fragment thereof including: a heavy chain variable (VH) domain including CDR1 of SEQ ID NO: 112, CDR2 of SEQ ID NO: 113, and CDR3 of SEQ ID NO: 114, and a light chain variable (VL) domain including: CDR1 of SEQ ID NO: 115, CDR2 of SEQ ID NO: 116, and CDR3 of SEQ ID NO: 117.
      126. The recombinant antibody or antigen binding fragment thereof of embodiment 125, wherein the VH domain is set forth in SEQ ID NO: 120 and the VL domain is set forth in SEQ ID NO: 123.
      127. A recombinant antibody or antigen binding fragment thereof including: a heavy chain variable (VH) domain including CDR1 of SEQ ID NO: 124, CDR2 of SEQ ID NO: 125, and CDR3 of SEQ ID NO: 126, and a light chain variable (VL) domain including: CDR1 of SEQ ID NO: 127, CDR2 of SEQ ID NO: 128, and CDR3 of SEQ ID NO: 129.
      128. The recombinant antibody or antigen binding fragment thereof of embodiment 127, wherein the VH domain is set forth in SEQ ID NO: 132 and the VL domain is set forth in SEQ ID NO: 135.
      129. A recombinant antibody or antigen binding fragment thereof including: a heavy chain variable (VH) domain including CDR1 of SEQ ID NO: 136, CDR2 of SEQ ID NO: 137, and CDR3 of SEQ ID NO: 138, and a light chain variable (VL) domain including: CDR1 of SEQ ID NO: 139, CDR2 of SEQ ID NO: 140, and CDR3 of SEQ ID NO: 141.
      130. The recombinant antibody or antigen binding fragment thereof of embodiment 129, wherein the VH domain is set forth in SEQ ID NO: 147 and/or the heavy chain is set forth in SEQ ID NO: 144; and
      the VL domain is set forth in SEQ ID NO: 153 and/or the light chain is set forth in SEQ ID NO: 150.
      131. A kit including:
(i) an antibody or antigen-binding fragment thereof including:
   a heavy chain variable (VH) domain including CDR1 of SEQ ID NO: 16, CDR2 of SEQ ID NO: 17, and CDR3 of SEQ ID NO: 18, and a light chain variable (VL) domain including: CDR1 of SEQ ID NO: 19, CDR2 of SEQ ID NO: 20, and CDR3 of SEQ ID NO: 21 that binds to a CYBB signature peptide of X-CGD of SEQ ID NO: 2;
   a heavy chain variable (VH) domain including CDR1 of SEQ ID NO: 160, CDR2 of SEQ ID NO: 161, and CDR3 of SEQ ID NO: 162, and a light chain variable (VL) domain including: CDR1 of SEQ ID NO: 163, CDR2 of SEQ ID NO: 164, and CDR3 of SEQ ID NO: 165 that binds to a SH2D1A signature peptide of XLP1 of SEQ ID NO: 3;
   a heavy chain variable (VH) domain including CDR1 of SEQ ID NO: 34, CDR2 of SEQ ID NO: 35, and CDR3 of SEQ ID NO: 36, and a light chain variable (VL) domain including: CDR1 of SEQ ID NO: 37, CDR2 of SEQ ID NO: 38, and CDR3 of SEQ ID NO: 39 that binds to a PRF-1 signature peptide of FHL2 of SEQ ID NO: 5;
   a heavy chain variable (VH) domain including CDR1 of SEQ ID NO: 52, CDR2 of SEQ ID NO: 53, and CDR3 of SEQ ID NO: 54, and a light chain variable (VL) domain including: CDR1 of SEQ ID NO: 55, CDR2 of SEQ ID NO: 56, and CDR3 of SEQ ID NO: 67 that binds to an ATM signature peptide of ataxia telangiectasia of SEQ ID NO: 7;
   a heavy chain variable (VH) domain including CDR1 of SEQ ID NO: 70, CDR2 of SEQ ID NO: 71, and CDR3 of SEQ ID NO: 72, and a light chain variable (VL) domain including: CDR1 of SEQ ID NO: 73, CDR2 of SEQ ID NO: 74, and CDR3 of SEQ ID NO: 75 that binds to a CD19 signature peptide of CVID of SEQ ID NO: 8;
   a heavy chain variable (VH) domain including CDR1 of SEQ ID NO: 82, CDR2 of SEQ ID NO: 83, and CDR3 of SEQ ID NO: 84, and a light chain variable (VL) domain including: CDR1 of SEQ ID NO: 85, CDR2 of SEQ ID NO: 86, and CDR3 of SEQ ID NO: 87 that binds to an ADA signature peptide of ADA deficiency of SEQ ID NO: 11;
   a heavy chain variable (VH) domain including CDR1 of SEQ ID NO: 100, CDR2 of SEQ ID NO: 101, and CDR3 of SEQ ID NO: 102, and a light chain variable (VL) domain including: CDR1 of SEQ ID NO: 103, CDR2 of SEQ ID NO: 104, and CDR3 of SEQ ID NO: 105 that binds to a DOCK8 signature peptide of DOCK8 deficiency of SEQ ID NO: 12;
   a heavy chain variable (VH) domain including CDR1 of SEQ ID NO: 112, CDR2 of SEQ ID NO: 113, and CDR3 of SEQ ID NO: 114, and a light chain variable (VL) domain including: CDR1 of SEQ ID NO: 115, CDR2 of SEQ ID NO: 116, and CDR3 of SEQ ID NO: 117 that binds to a CD42 signature peptide of SEQ ID NO: 13;
   a heavy chain variable (VH) domain including CDR1 of SEQ ID NO: 124, CDR2 of SEQ ID NO: 125, and CDR3 of SEQ ID NO: 126, and a light chain variable (VL) domain including: CDR1 of SEQ ID NO: 127, CDR2 of SEQ ID NO: 128, and CDR3 of SEQ ID NO: 129 that binds to a CD42 signature peptide of SEQ ID NO: 14; and/or
   a heavy chain variable (VH) domain including CDR1 of SEQ ID NO: 136, CDR2 of SEQ ID NO: 137, and CDR3 of SEQ ID NO: 138, and a light chain variable (VL) domain including: CDR1 of SEQ ID NO: 139, CDR2 of SEQ ID NO: 140, and CDR3 of SEQ ID NO: 141 that binds to a CD56 signature peptide of SEQ ID NO: 15;
      and/or
(ii) an antibody or antigen-binding fragment thereof that binds a CYBB signature peptide of X-CGD of SEQ ID NO: 1;
   an antibody or antigen-binding fragment thereof that binds a SH2D1A signature peptide of XLP1 of SEQ ID NO: 4;
   an antibody or antigen-binding fragment thereof that binds a PRF-1 signature peptide of FHL2 of SEQ ID NO: 6;
   an antibody or antigen-binding fragment thereof that binds an ATM signature peptide of AT of SEQ ID NO: 210;
   an antibody or antigen-binding fragment thereof that binds an ATM signature peptide of AT of SEQ ID NO: 211;
   an antibody or antigen-binding fragment thereof that binds a CD19 signature peptide of CVID of SEQ ID NO: 9;
   an antibody or antigen-binding fragment thereof that binds a CD19 signature peptide of CVID of SEQ ID NO: 10;
   an antibody or antigen-binding fragment thereof that binds a CD19 signature peptide of CVID of SEQ ID NO: 212;
   an antibody or antigen-binding fragment thereof that binds an IL2RG signature peptide of SCID of SEQ ID NO: 213;
   an antibody or antigen-binding fragment thereof that binds an IL2RG signature peptide of SCID of SEQ ID NO: 214;
   an antibody or antigen-binding fragment thereof that binds a CD3ε signature peptide of SCID of SEQ ID NO: 215;
   an antibody or antigen-binding fragment thereof that binds a CD3δ signature peptide of SCID of SEQ ID NO: 216; and/or
   an antibody or antigen-binding fragment thereof that binds a CD3δ signature peptide of SCID of SEQ ID NO: 217;
      and/or
(iii) reference signature peptides including:
   a CYBB signature peptide of X-CGD of SEQ ID NO: 1;
   a CYBB signature peptide of X-CGD of SEQ ID NO: 2;
   a SH2D1A signature peptide of XLP1 of SEQ ID NO: 3;
   a SH2D1A signature peptide of XLP1 of SEQ ID NO: 4;
   a PRF-1 signature peptide of FHL2 of SEQ ID NO: 5;
   a PRF-1 signature peptide of FHL2 of SEQ ID NO: 6;
   an ATM signature peptide of ataxia telangiectasia of SEQ ID NO: 210;
   an ATM signature peptide of ataxia telangiectasia of SEQ ID NO: 211;
   an ATM signature peptide of ataxia telangiectasia of SEQ ID NO: 7;
   a CD19 signature peptide of CVID of SEQ ID NO: 8;
   a CD19 signature peptide of CVID of SEQ ID NO: 9;
   a CD19 signature peptide of CVID of SEQ ID NO: 10;
   a CD19 signature peptide of CVID of SEQ ID NO: 212;
   an IL2RG signature peptide of SCID of SEQ ID NO: 213;
   an IL2RG signature peptide of SCID of SEQ ID NO: 214;
   a CD3ε signature peptide of SCID of SEQ ID NO: 215;
   a CD3δ signature peptide of SCID of SEQ ID NO: 216;
   a CD3δ signature peptide of SCID of SEQ ID NO: 217;
   an ADA signature peptide of ADA deficiency of SEQ ID NO: 11;
   a DOCK8 signature peptide of DOCK8 deficiency of SEQ ID NO: 12;
   a CD42 signature peptide of SEQ ID NO: 13;
   a CD42 signature peptide of SEQ ID NO: 14; and/or
   a CD56 signature peptide of SEQ ID NO: 15.
      132. The kit of embodiment 131, further including one or more of: filter paper card; punch tool; buccal swab; cryogenic vial; blood collection tube; digestion enzymes; digestion buffers; solid support for the antibodies or antigen-binding fragments thereof; and elution buffers.
      133. The kit of embodiment 131 or 132, wherein the reference signature peptides are isotopically labeled.
      134. The kit of any one of embodiments 131-133, wherein the antibodies or antigen-binding fragments thereof are attached to magnetic beads.

### (XII) EXPERIMENTAL EXAMPLES

At least some of the data described below in the Experimental Examples has been published in Collins et al., Frontiers in Immunology, Vol. 11, Article 464 (April 2020).

### Example 1. Summary

A multiplexed peptide immunoaffinity enrichment coupled to selected reaction monitoring (immuno-SRM) panel was generated for simultaneous screening of six signature peptides representing three Primary Immunodeficiency Disorders (PIDD)-specific and three cell-type specific proteins from dried blood spots (DBS). In samples from 29 PIDD patient samples including two XLA carriers, representing Wiskott-Aldrich Syndrome (WAS), X-linked agammaglobulinemia (XLA), X-Linked Chronic Granulomatous Disease (X-CGD), Dock8 Deficiency, and adenosine deaminase (ADA) deficiency, peptides representing each disease are significantly reduced relative to normal controls and patient identification had excellent agreement with clinical and molecular diagnosis. Also included in the multiplex panel were cell specific markers for platelets (CD42) and natural killer (NK) cells (CD56). In WAS patients, CD42 levels were found to be significantly reduced consistent with characteristic thrombocytopenia. A WAS patient analyzed before and after bone marrow transplant showed normalized WAS protein and platelet CD42 after treatment highlighting the ability of immuno-SRM to demonstrate and monitor the effects of PIDD treatment. A high-throughput Immuno-SRM method screens PIDD-specific peptides in a 2.5-minute runtime meeting high volume newborn screening (NBS) workflow requirements. This high-throughput method returned identical results to the standard Immuno-SRM PIDD panel. Immuno-SRM peptide analysis represents a robust potential clinical diagnostic for identifying and studying PIDD patients from easily collected and shipped DBS and supports a significant potential for early PIDD diagnosis through NBS.

A multiplexed proteomic diagnostic panel was developed to screen DBS for six molecularly defined PIDDs using six signature peptide biomarkers. The current set of targeted PIDDs includes ADA deficiency (ADA) (Whitmore and Gaspar, Frontiers in immunology, 2016. 7: p. 314-314; Flinn and Gennery, Orphanet Journal of Rare Diseases, 2018. 13(1): p. 65), Dedicator of cytokinesis 8 (DOCKS) deficiency (Aydin et al., J Clin Immunol, 2015. 35(2): p. 189-98; Engelhardt et al., J Allergy Clin Immunol, 2009. 124(6): p. 1289-302 e4), X-Linked Chronic Granulomatous disease (XL-CGD), Ataxia Telangiectasia (AT), X-linked Lymphoproliferative Syndrome (XLP1; SH2D1A deficiency), and Familial Hemophagocytic Lymphohistiocytosis 2 (FHL2). Signature peptides for these conditions serve as primary markers for direct diagnosis of a specific PIDD. Analysis of secondary protein markers such as neural cell adhesion molecule (CD56) and glycoprotein Ib (CD42) provide support for specific diagnoses by generating information as to the counts of Natural Killer (NK) cells and platelets, respectively. Together, six peptide biomarkers associated with the above conditions could be quantified in a multiplex assay simultaneously.

The generation of anti-peptide monoclonal antibodies (mAbs) to signature peptides give greater specificity, reproducibility, and batch-to-batch consistency relative to polyclonal antibodies (pAbs). Normal control ranges have been established and blinded sample sets were used to test the ability to differentiate patients from controls while secondary markers have provided information about the resulting effects on the hematopoietic and immune system. Finally, multiplexed methods have been developed which are highly transferrable and suitable for both diagnostic analysis and high-throughput (HT) NBS.

Materials and Methods.

Dried Blood Spot Samples.

This protocol was approved by the controlling institutional review board. All subjects gave written informed consent in accordance with the Declaration of Helsinki. Normal control blood samples were purchased from BiolVT (Hicksville, N.Y.). Patient samples were provided by the Seattle Children's Immunology Diagnostic Laboratory including samples that originated at the Universidade Federal de Uberlandia (Uberlandia, Brazil). Additional patient samples were collected at the National Institutes of Health (Bethesda, Md.) after written informed consent was obtained. Samples were prepared either by fingerstick or by pipetting 70 μL of blood (per 12 mm spot) onto filter paper cards (Protein Saver 903, Piscayaway, N.J.). The samples were then dried overnight at room temperature, shipped to SCH, and stored at −80° C. until use. DBS samples were analyzed from 175 normal controls. In total, DBS from 29 PIDD patients were analyzed including samples from 8 WAS patient samples, 11 XLA patients and 2 carriers, 1 DOCK8 deficient patient, 3 XL-CGD patients, 1 AR-CGD patient, and 6 ADA deficient patients.

Immuno-SRM Reagents.

Triton X-100 (T9284-100 mL), TPCK-treated Worthington Trypsin (LS003740), and (3-[3-cholamidopropyl=dimethylammonio]-1-propanesulfonate) (CHAPS) (no. 28300) detergent were obtained from Fisher Scientific (Waltham, Mass.). Ammonium bicarbonate (Ambic) (A6141-25G) was purchased from Sigma Aldrich. Acetonitrile (ACN) (no. A955, Optima LC/MS grade), Acetic Acid (AA) (no. A11350, Optima LC/MS grade), water (no. W6, Optima LC/MS grade), formic acid (FA) (no. A117, Optima LC/MS grade), phosphate-buffered saline (1×PBS, no. 10010-023), Dithiothreitol (DTT) and tris(hydroxymethyl) aminomethane (TRIS) buffer were obtained from Fisher Scientific (Waltham, Mass.).

Isotope-labeled internal standard (IS) peptides were purchased from either Atlantic Peptides (Lewisburg, Pa.) or Life Technologies Corporation (Carlsbad, Calif.). IS peptides were purified to >95% and synthesized to incorporate a heavy stable isotope-labeled C-terminal lysine or arginine. The labeling (13C or 15N), results in a mass shift of +8 (Lys) or +10 (Arg) Daltons (Da). Aliquots were stored in 5% ACN/0.1% FA at −80° C. until use.

PIDD internal standard (IS) peptides were stored as 500× mixtures in 1×PBS+15% ACN+0.1% FA+0.03% CHAPS in H₂O and diluted immediately before use. Final peptide concentration in the stock mix and peptide capture experiment are shown in Table 2.

TABLE 2
Stock and final internal peptide concentrations and monoclonal
antibody bead mass per sample for each target peptide.

| | 500x Stock | Optimized Final | |
|---|---|---|---|
| | Concentration | Concentration | mAb Bead Mass |
| | (nM) | (nM) | (μg/sample) |
| CYBB 509 | 5000 | 10 | 0.25 |
| ADA 93 | 2500 | 5 | 0.25 |
| DOCK8 1272 | 312.5 | 0.625 | 2 |
| ATM 1561 | 1250 | 2.5 | 4 |
| PRF 215 | 250 | 0.5 | 4 |
| SH2D1A 19 | 250 | 0.5 | 6 |
| CD42 128 | 20,000 | 20 | 1 |
| CD42 154 | 5000 | 10 | 0.5 |
| CD56 122 | 1250 | 2.5 | 2 |

Selection of Signature Peptides and Antibody Production.

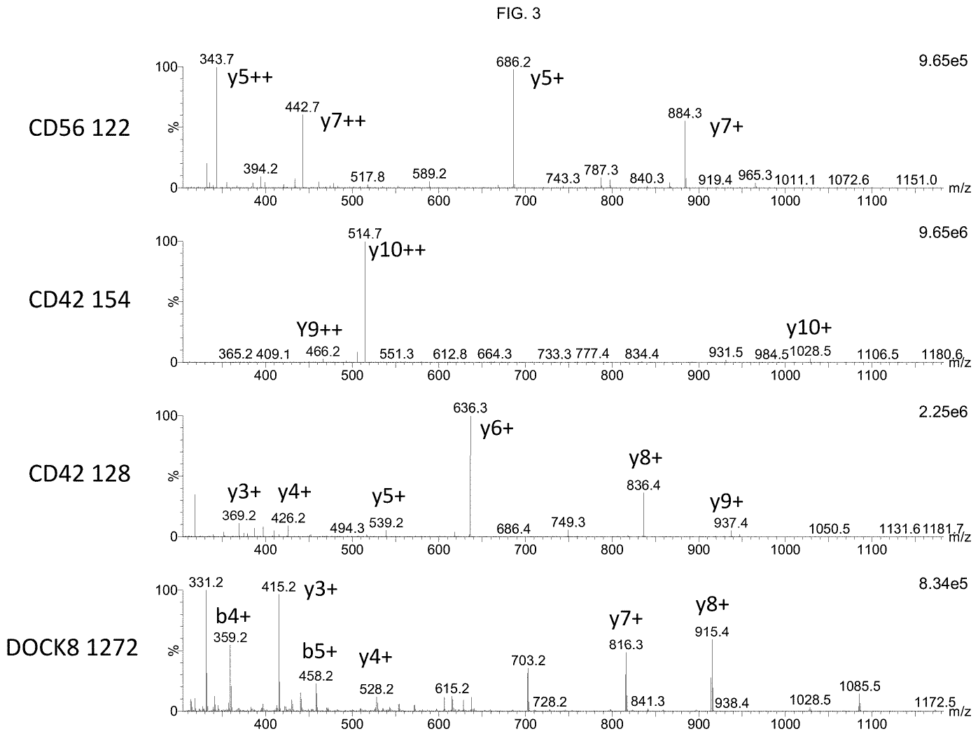

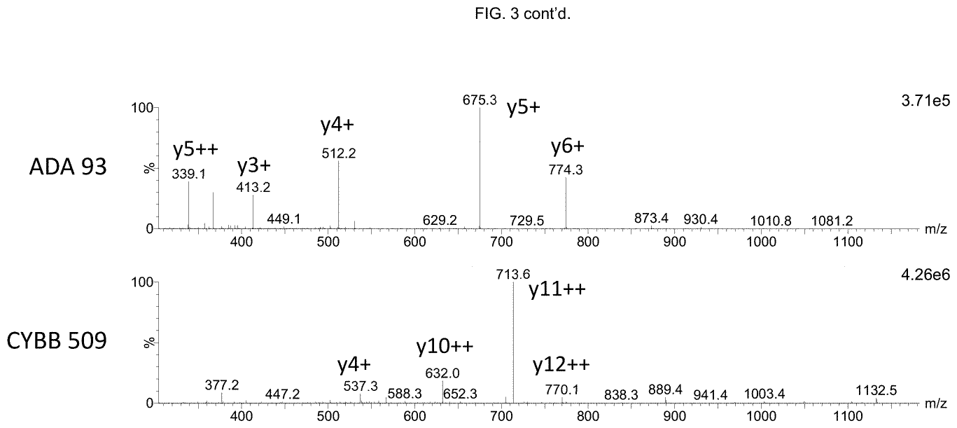

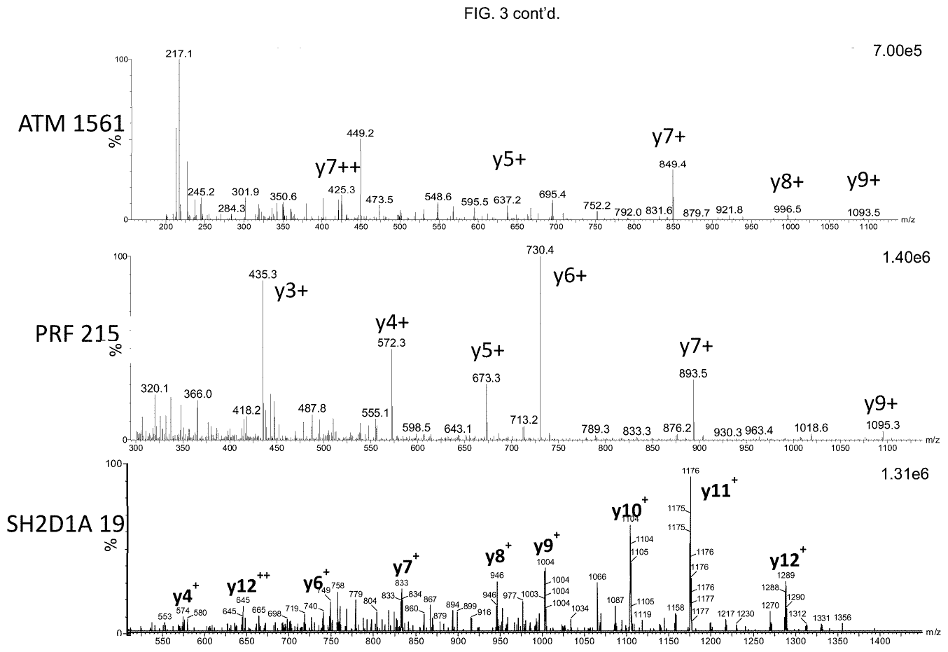

Candidate signature peptide selection was done according to published CPTAC guidelines for SISCAPA assays (Hoofnagle et al., Clin Chem, 2016. 62(1): p. 48-69). In brief, candidates were generated by in silico digestion of proteins to generate tryptic peptides. These sequences were segregated based on length and hydrophobicity. Peptides with potential missed cleavages, methionine, and known post-translational modifications were excluded. Final candidate peptides were screened for uniqueness in the proteome using BLAST tools. If multiple candidate peptides existed, final peptides were selected based on MS response. Final peptide selections for antibody production were made by comparing MS response at equivalent concentrations. Fragmentation patterns for signature peptides are presented in FIG. 3.

Antibody production was performed as follows. Peptide sequences were synthesized with an N-terminal cysteine and conjugated to adjuvant proteins before rabbit immunization. Peripheral blood mononuclear cells (PBMCs) from animals with high titers and activity were isolated and then B-Cells were cultured. Responding isolated B-Cells then had their cDNA cloned and antibodies expressed in a mammalian expression system for further screening. Antibodies were tested after B-Cell culture and antibody clone expression using ELISA and immuno-SRM methods to determine suitability for monoclonal development.

Alternatively, peptide synthesis and adjuvant conjugation were conducted as above before immunization of mice. B-cells from responding animals were isolated and hybridoma cell lines produced. Antibodies were tested at multiple stages in crude bleeds and after hybridoma development using ELISA and immuno-SRM methods to determine suitability for monoclonal development.

Antibody Bead Reagent Production.

Monoclonal Antibody (mAb) beads were produced by incubating with Protein G coated magnetic beads (Dynabeads, no. 10004D) from Invitrogen (Carlsbad, Calif.), which were initially washed with 1×PBS+0.03% CHAPS and magnetic isolation (Millipore, no. 20-4000) (Temecula, Calif.) of the beads to remove the storage buffer. Beads were mixed by pipetting and subsequently isolated by the magnet. This washing procedure was repeated for a total of three times. Finally, appropriate amount of mAb stock solution was added to the isolated beads at a ratio of 1:2.5 μg mAb: μL bead. Then the antibodies and beads were tumbled overnight at 4° C. for coupling. Finally, the immobilized mAb-linked beads were washed twice with 1×PBS+0.03% CHAPS and resuspended at a concentration of 0.4 μg/μL.

DBS Extraction, Trypsin Digestion and Immunoaffinity Enrichment.

Protein extraction and tryptic digestion were done with a modified procedure to those previous reported (Collins et al., Frontiers in Immunology, 2018. 9(2756); Collins et al. Frontiers in Immunology, 2020. 11). Two 6.35 mm diameter punches were taken from the DBS cards and placed into 96-well plates (MASTERBLOCK, Greinier, no. 786201) from Thermo Scientific (Chicago, Ill.). These punches were submerged in 300 μL of 0.1% Triton X-100 in 50 mM ammonium bicarbonate buffer. Dithiothreitol (DTT) was then added to a concentration of 0.2M DTT in H₂O. After vortex, protein extraction proceeded for 30 min at 37° C. with agitation. After protein extraction, 300 μL of extracted supernatant was removed and transferred to a new plate before addition of 60 μg of Trypsin and incubation for 2 hours at 37° C. The resulting peptide mixture was directly used for peptide immunoaffinity enrichment.

Directly to the digested peptide mixture, 15 μL of 1 M TRIS (pH 8, no. 15568-025) and 15 μL of 1× internal standard mix were added. Final IS concentrations for PIDD signature peptide internal standard are shown in Table 2. Next, mAb-beads were directly added at the masses listed in Table 2. The resulting solution was incubated overnight at 4° C. with agitation to allow for peptide capture by anti-peptide antibodies.

After incubation, mAb-beads were pulled down using a 96-well magnetic plate (Alpaqua Magnum EX, no. A000380) (Beverly, Mass.) and washed twice with 1×PBS+0.01% CHAPS to remove off-target peptides. After each wash, mAb-beads were collected by the magnet before resuspension with additional wash buffer. After the final wash, they were collected by the magnet before addition of 30 μL of aqueous elution solution containing 5% AA+3% ACN. Captured peptides were eluted from beads for 5 min at 1000 rpm. Finally, beads were collected by the magnet and the eluted peptide was transferred to an LC/MS vial or a new 96-well plate for analysis. Samples with less than the necessary DBS available were analyzed from either 4 or 5×3.125 mm punches. In these cases, all volumes were adjusted accordingly except for antibody input mass.

Liquid Chromatography Mass Spectrometry.

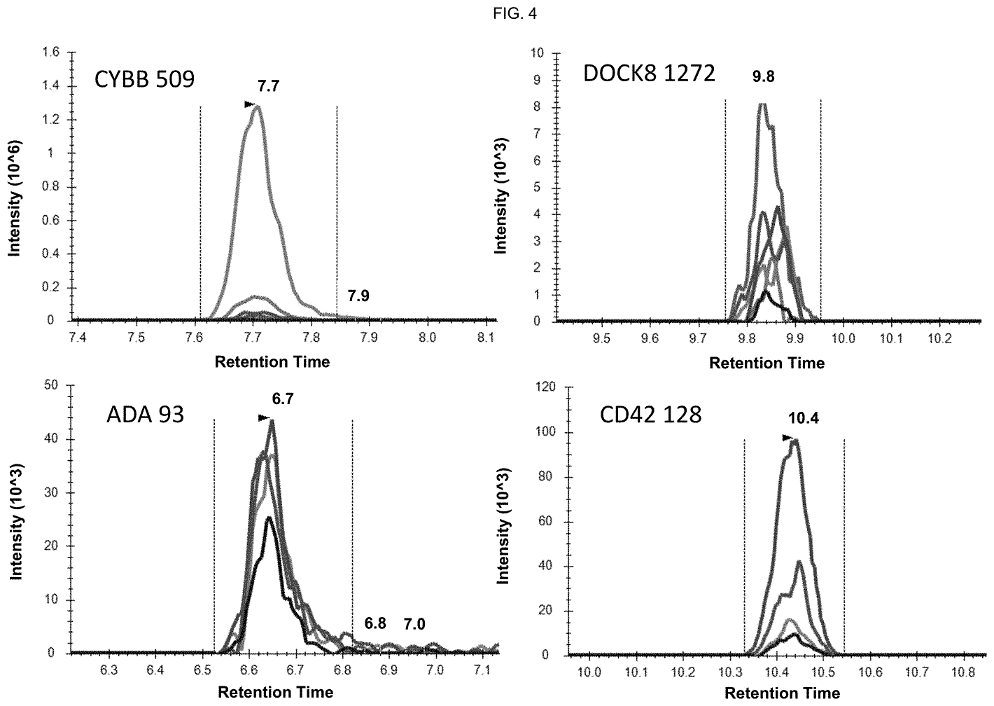

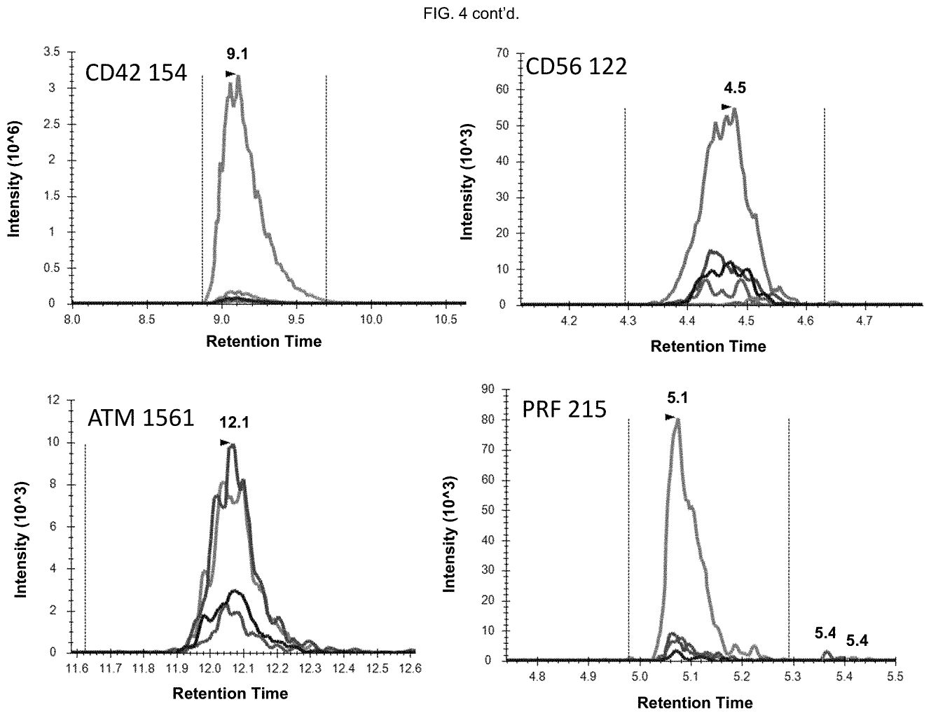

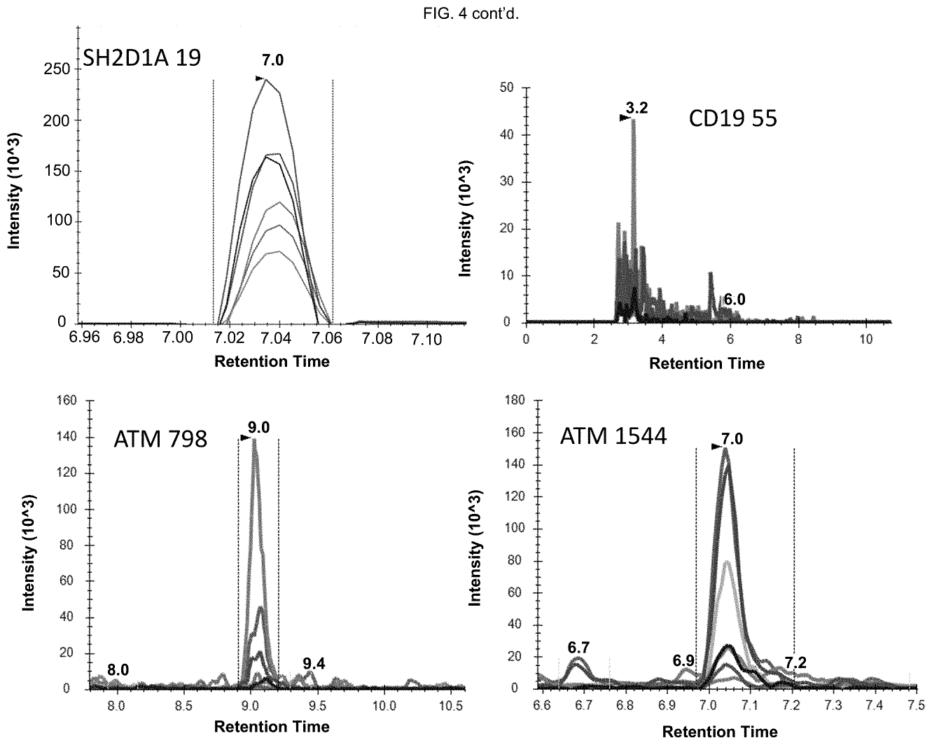

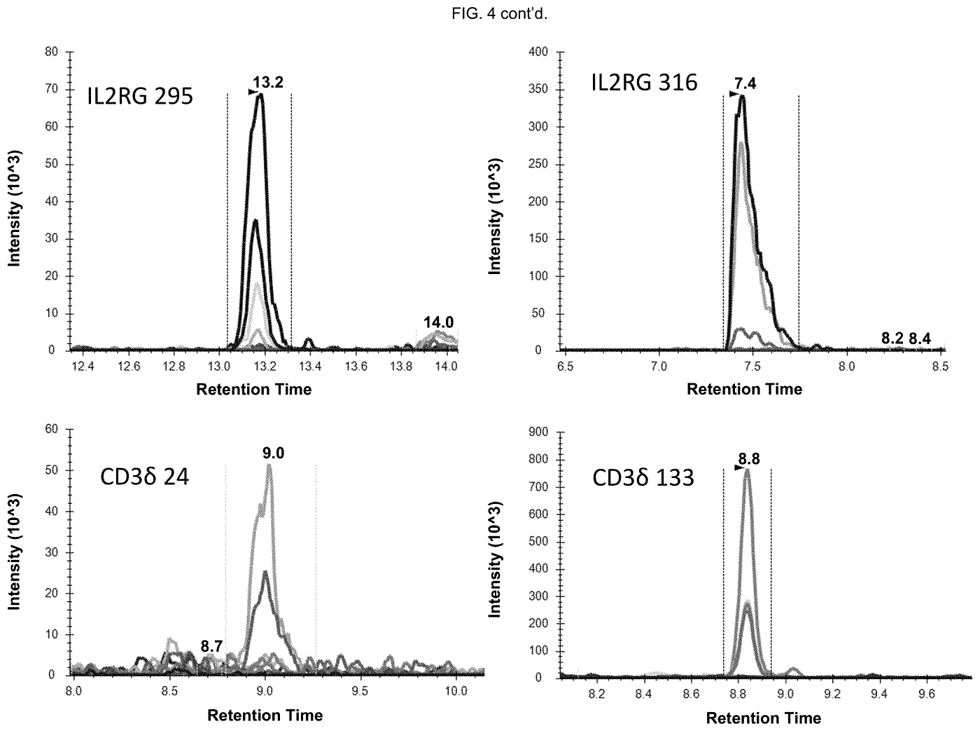

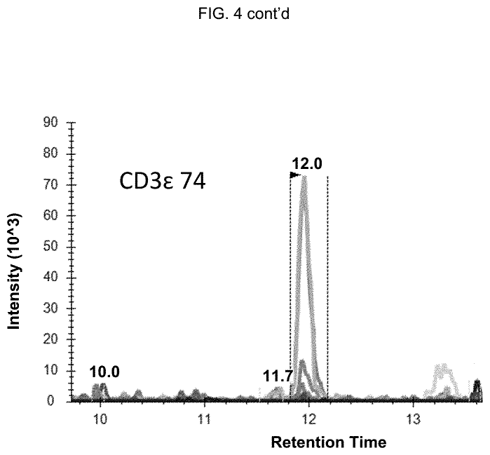

LC-MS/MS of isolated peptide mixtures was performed on a Waters Xevo TQ-XS with lonkey source and dual M-Class gradient and loading chromatography pumps (Milford, Mass.). Chromatographic solvents were A: H₂O+0.1% FA and B: ACN+0.1% FA. As an initial step, peptides are loaded onto an M-Class Trap Symmetry C18 column (300 μM×25 mm, 100A, 5 μM) for three minutes with a constant flow of 98:2 A:B at 20 μL/min. After loading, the flow is reversed. Peptides are eluted from the trapping column and separated using a 150 μM×100 mm BEH C18 ionkey (130A, 1.7 μM). Gradient flow conditions are shown for both a 20 minute and a 2.5-minute gradient (Table 3). SRM transitions were acquired in unit resolution in both Q1 and Q3 quadrupoles. Dwell time was 5 ms with a 3 ms pause between mass ranges and the total cycle time was 1.5 s. Precursor and fragment masses for each peptide were chosen to generate the highest intensity transitions. Precursor mass, fragment mass, and collision energy were tuned to optimize the generated signal. Endogenous SRM traces for each peptide are shown in FIG. 4.

The gradient settings are shown in Table 3. Transitions were acquired at unit/unit resolution with a 10 ms dwell time and 5 ms pause between mass ranges resulting in a cycle time of 0.75 sec.

TABLE 3
Liquid Chromatography conditions for both standard
and high-throughput immuno-SRM analysis.
SCRI

| Standard | | | HT | | |
|---|---|---|---|---|---|
| | Flow | | | Flow | |
| Time (min) | (μL/min) | % B | Time (min) | (μL/min) | % B |
| 0 | 3 | 5 | 0 | 9 | 10 |
| 1 | 3 | 10 | 0.2 | 9 | 10 |
| 11 | 3 | 25 | 0.8 | 9 | 65 |
| 13 | 3 | 85 | 1 | 9 | 95 |
| 15 | 3 | 85 | 1.01 | 4 | 95 |
| 17 | 3 | 5 | 1.5 | 4 | 95 |
| 20 | 3 | 5 | 1.51 | 7 | 10 |
| | | | 1.7 | 7 | 10 |
| | | | 2 | 9 | 10 |
| | | | 2.5 | 9 | 10 |

Data Analysis.

SRM data were analyzed using Skyline (MacCoss Lab, open source software, Seattle, Wash., on World Wide Web at skyline.ms/project/home/begin.view). Concentrations of endogenous signature peptides were generated by comparing endogenous peptide signal to the signal of the isotopic IS added to the peptide extract at a known concentration. Statistical analyses were done using Graphpad Prism (San Diego, Calif.). Peptide reductions were analyzed using one-way ANOVA compared against the normal control for each peptide.

Method Performance Assessment.

A response curve was generated to establish assay linearity, as well as limits of detection and quantification. Punches from a normal control DBS (2×6.35 mm punches) were extracted and digested for each sample. After digestion, samples were pooled and re-aliquoted to generate a consistent endogenous peptide signal. To the aliquoted samples, eight different concentrations (0×, 0.05×, 0.1×, 0.5×, 1×, 2×, 5×, and 50×) of IS were added. Each concentration was added in triplicate. After IS addition, immuno-SRM workflow was completed as described. After peptide elution, samples were split into two vials for LC-MS/MS analysis and run through two separate gradients (20 min and 2.5 min) to compare the respective analytical values. Response curve samples were run through both the standard and high-throughput (HT) gradients.

Intra- and inter-assay precision of the assay was determined by quantifying endogenous peptide concentrations across five separate days. Each day, five replicate assays were conducted through the entire immuno-SRM process. Coefficients of variation (CV) was determined for within day and between days sample sets. CV samples were run through both the standard and HT gradients.

To study endogenous protein and peptide stability, the same DBS card was analyzed over time. Three separate DBS cards were stored at RT and 37° C. and compared to a sample kept at −20° C. to determine the effects of storage temperature. Peptide concentration measurements were made after seven days of incubation. Each sample was analyzed in triplicate.

Patient Sample Analysis. Patient samples were analyzed in a blinded fashion by generated mixed sample sets containing patients and controls. Full sample sets were run through the standard 20-min gradient for initial establishment of diagnosis. After analysis, patient condition was predicted by comparison to diagnostic cutoffs before unblinding. A subset of 17 samples and 20 normal controls were analyzed using the 2.5-min HT-gradient to examine the agreement of the standard and HT methods.

Results

Peptide Selection and Antibody Development.

Signature peptide sequences chosen as representative biomarker peptides are listed in Table 4 along with molecular weights and parent and daughter ions used for quantitative analysis.

Monoclonal antibodies against each sequence have been generated. Final antibodies were chosen for their ability to capture endogenous signature peptides with high affinity and their lack of interfering analytes.

TABLE 4
Signature Peptide Information for Primary and Secondary Markers including
Sequence, Mass, and Parent and Fragment Ions. Full Peptide
Fragment Information is shown in FIG. 1.

| | | | | | | Parent | Fragment |
|---|---|---|---|---|---|---|---|
| Disease Or | Marker | | | | Mass | Ion | Ion |
| Cell Target | Type | Protein | Peptide | Sequence | (Da) | (m/z) | (m/z) |
| X-Linked | Primary | CYBB | CYBB | TLYGRPNWDNEFK | 1639.767 | 547.26 | [y8]- |
| Chronic | | | 509-521 | (SEQ ID NO: 2) | | 70 | 1049.4687+, |
| Granulomatous | | | | | | +++ | [y4]- |
| Disease | | | | | | | 537.2667+, |
| | | | | | | | [y12]- |
| | | | | | | | 769.8730++, |
| | | | | | | | [y11]- |
| | | | | | | | 713.3309++, |
| | | | | | | | [y10]- |
| | | | | | | | 631.7993++ |
| Adenosine | Primary | ADA | ADA | EGVVYVEVR | 1049.173 | 525.28 | [y7]- |
| Deaminase | | | 93-101 | (SEQ ID NO: 11) | | 49 | 863.4985+, |
| Deficiency | | | | | | ++ | [y6]- |
| | | | | | | | 764.4301+, |
| | | | | | | | [y5]- |
| | | | | | | | 665.3617+, |
| | | | | | | | [y3]- |
| | | | | | | | 403.2300+, |
| | | | | | | | [b3]- |
| | | | | | | | 286.1397+ |
| DOCK8 | Primary | DOCK8 | DOCK8 | TSGIVLSSLPYK | 1264.466 | 632.86 | [y10]- |
| Deficiency | | | 1272-1283 | (SEQ ID NO: 12) | | 10 | 1076.6350+, |
| | | | | | | ++ | [y8]- |
| | | | | | | | 906.5295+, |
| | | | | | | | [y7]- |
| | | | | | | | 807.4611+, |
| | | | | | | | [y6]- |
| | | | | | | | 694.3770+, |
| | | | | | | | [y5]- |
| | | | | | | | 607.3450+, |
| | | | | | | | [b4]- |
| | | | | | | | 359.1925+ |
| Ataxia | Primary | ATM | ATM | LLDPFPDHVVFK | 1426.658 | 476.26 | [y7]- |
| Telangiect- | | | 1561-1572 | (SEQ ID NO: 7) | | 25 | 841.4567+, |
| asia | | | | | | +++ | [y11]- |
| | | | | | | | 657.3481++ |
| | | | | | | | [y10]- |
| | | | | | | | 600.8060++, |
| | | | | | | | [y9]- |
| | | | | | | | 543.2926++ |
| Familial | Primary | PRF-1 | PRF | LISNYGTHFIR | 1320.495 | 440.90 | [y7]- |
| Hemophagocytic | | | 215-225 | (SEQ ID NO: 5) | | 68 | 893.4628+, |
| Lymphohist- | | | | | | +++ | [y6]- |
| iocytosis 2 | | | | | | | 730.3995+ |
| | | | | | | | [y10]- |
| | | | | | | | 604.3146++, |
| | | | | | | | [y9]- |
| | | | | | | | 547.7725++ |
| X-Lymphopro- | Primary | SAP | SH2D1A | LLLATGLDGSYLLR | 1504.771 | 752.94 | [y11]- |
| liferative | | | 19-32 | (SEQ ID NO: 3) | | 03 | 1165.6212+, |
| Syndrome | | | | | | ++ | [y10]- |
| 1 | | | | | | | 1094.5840+ |
| | | | | | | | [y9]- |
| | | | | | | | 993.5364+, |
| | | | | | | | [y7]- |
| | | | | | | | 823.4308+ |
| | | | | | | | [y6]- |
| | | | | | | | 708.4039+, |
| | | | | | | | [b3]- |
| | | | | | | | 340.2595+ |
| | | | | | | | [b11]- |
| | | | | | | | 1104.5936+ |
| Platelets | Secondary | CD42 | CD42 | LTSLPLGALR | 1040.254 | 520.82 | [y9]- |
| | | | 128-137 | (SEQ ID NO: 13) | | 68 | 927.5622+, |
| | | | | | | ++ | [y8]- |
| | | | | | | | 826.5145+, |
| | | | | | | | [y6]- |
| | | | | | | | 626.3984+, |
| | | | | | | | [y4]- |
| | | | | | | | 416.2616+ |
| | Secondary | CD42 | CD42 | TLPPGLLTPTPK | 1234.483 | 617.87 | [y10]- |
| | | | 154-165 | (SEQ ID NO: 14) | | 39 | 1020.6088+, |
| | | | | | | ++ | [y9]- |
| | | | | | | | 923.5560+, |
| | | | | | | | [y8]- |
| | | | | | | | 826.5033+, |
| | | | | | | | [y10]- |
| | | | | | | | 510.8080++, |
| | | | | | | | [y9]- |
| | | | | | | | 462.2817++ |
| NK Cells | Secondary | CD56 | CD56 | NAPTPQEFR | 1059.128 | 530.26 | [y7]- |
| | | | 122-130 | (SEQ ID NO: 15) | | 45 | 874.4417+, |
| | | | | | | ++ | [y6]- |
| | | | | | | | 777.3890+, |
| | | | | | | | [y5]- |
| | | | | | | | 676.3413+, |
| | | | | | | | [y4]- |
| | | | | | | | 579.2885+, |
| | | | | | | | [y7]- |
| | | | | | | | 437.7245++, |
| | | | | | | | [y6]- |
| | | | | | | | 389.1981++, |
| | | | | | | | [y5]- |
| | | | | | | | 338.6743++ |

Primary markers are used for direct diagnosis of the specific associated PIDD. Secondary markers provide information related to markers of hematopoiesis associated targets.

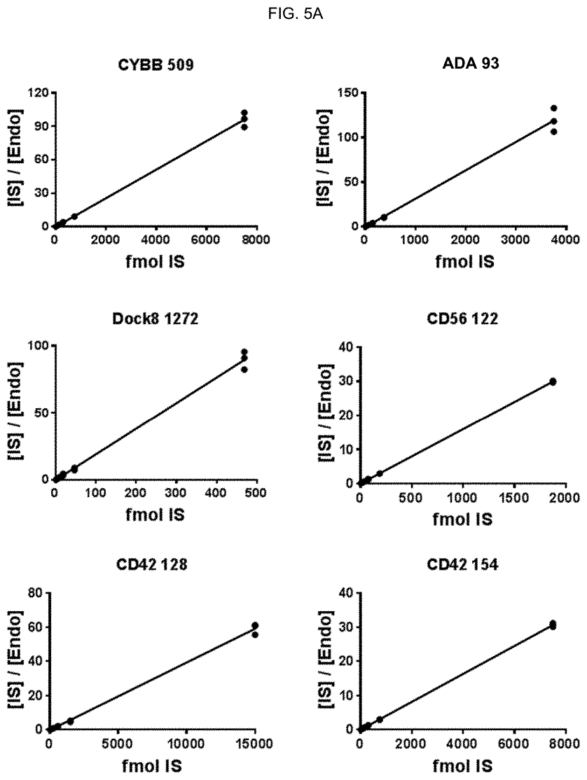

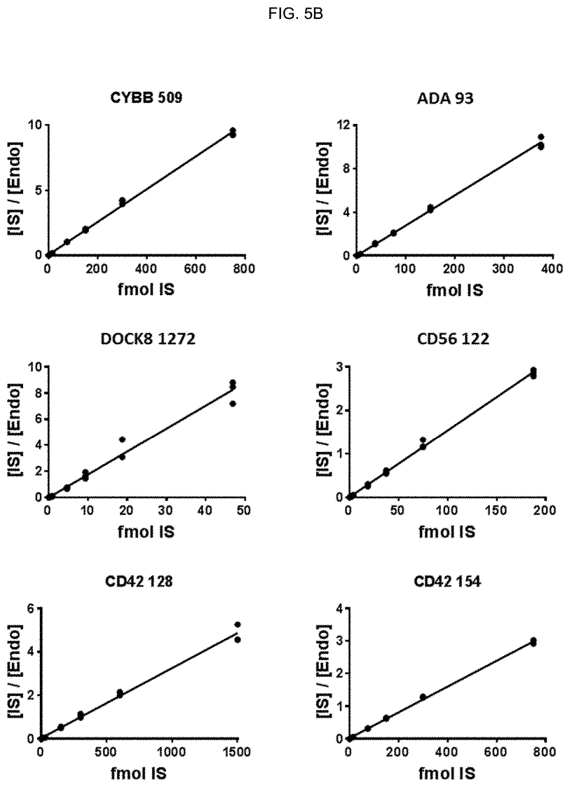

Method Performance Assessment. Analytical figures including linearity, LOD, LOQ, intra-assay and inter-assay CV for standard gradient conditions, and stability are shown in Table 5. All peptides had LOD and LOQ values of less than 10 fmol except for CD42 128 which had an LOD of 17.6 fmol and an LOQ of 30 fmol. Linear range plots are shown in FIGS. 5A, 5B. All peptide concentrations were reproducible with CVs<20%. Typically, this is the accepted limit of variation between sample runs. Analytical figures for HT gradient analysis are shown in Table 6. The LOD values were equivalent in between the 2.5- and 20-minute runs while the LOQ values increased in each case except for CD56 122, ADA 93 and CYBB 509 when moving to the HT method. Intra-assay CV tended to increase with a faster method, while remaining less than 20% for all peptides except for DOCK8 1272.

Additionally, there is a less than 20% change in peptide concentration over a seven-day period of storage at RT or 37° C. relative to a sample stored at −20° C. in all cases except for CD42 128. This platelet marker showed increases of 25.5% and 27.9% when stored at RT and 37° C., respectively.

TABLE 5
Analytical Figures of Merit for Standard PIDD Gradient.

| | | | | | | Intra- | | | |
|---|---|---|---|---|---|---|---|---|---|
| | | | | | | assay | Inter- | | |
| | | | | | | CV | assay | Relative | Relative |
| Marker | | | LLOD | LLOQ | ULOD | (Average, | CV | Difference | Difference |
| Type | Protein | Peptide | (fmol) | (fmol) | (fmol) | %) | (%) | (RT, %) | (37° C., %) |
| Primary | CYBB | CYBB | 4.2 | 7.5 | 7500.0 | 5.3 | 4.4 | 9.9 | −19.0 |
| | | 509-521 | | | | | | | |
| Primary | ADA | ADA | 0.8 | 3.8 | 3750.0 | 7.9 | 13.5 | 15.8 | 18.4 |
| | | 93-101 | | | | | | | |
| Primary | DOCK8 | DOCK8 | 1.1 | 4.7 | 468.8 | 15.3 | 18.4 | 7.9 | −9.6 |
| | | 1272-1283 | | | | | | | |
| Secondary | CD42 | CD42 | 17.6 | 30.0 | 15000.0 | 5.4 | 10.9 | 25.5 | 27.9 |
| | | 128-137 | | | | | | | |
| Secondary | CD42 | CD42 | 1.1 | 7.5 | 7500.0 | 6.1 | 5.8 | 14.0 | −10.7 |
| | | 154-165 | | | | | | | |
| Secondary | CD56 | CD56 | 2.7 | 3.8 | 1875.0 | 10.3 | 9.7 | 17.5 | 2.9 |
| | | 122-130 | | | | | | | |

Primary markers are used for direct diagnosis of the associated PIDDs. Secondary markers provide information related to markers of hematopoiesis associated targets.

TABLE 6
Comparison of LLOD, LLOQ, and CV of the Standard
20-minute and HT 2.5-minute Gradients.

| | | | | | | Intra-assay | | | |
|---|---|---|---|---|---|---|---|---|---|
| | | | | | | CV | | Inter-assay | |
| | | LLOD (fmol) | | LLOQ (fmol) | | (Average, %) | | CV (%) | |
| Marker | | 20 | 2.5 | 20 | 2.5 | 20 | 2.5 | 20 | 2.5 |
| Type | Peptide | min | min | min | min | min | min | min | min |
| Primary | CYBB 509-521 | 4.2 | 0.6 | 7.5 | 7.5 | 5.3 | 7.6 | 4.4 | 3.2 |
| Primary | ADA 93-101 | 0.8 | 6.9 | 3.8 | 3.8 | 7.9 | 11.6 | 13.5 | 13.2 |
| Primary | DOCK8 1272-1283 | 1.1 | 1.6 | 4.7 | 18.8 | 15.3 | 29.5 | 18.4 | 11.0 |
| Secondary | CD42 128-137 | 17.6 | 9.9 | 30.0 | 150.0 | 5.4 | 12.7 | 10.9 | 5.0 |
| Secondary | CD42 154-165 | 1.1 | 2.0 | 7.5 | 15.0 | 6.1 | 7.2 | 5.8 | 4.5 |
| Secondary | CD56 122-130 | 2.7 | 5.4 | 3.8 | 3.8 | 10.3 | 12.5 | 9.7 | 11.0 |

Primary markers are used for direct diagnosis of the associated PIDDs. Secondary markers provide information related to markers of hematopoiesis associated targets.

Normal Control Ranges.

Immuno-SRM analysis of 125 normal control samples was performed to set normal ranges. These ranges were used to establish diagnostic cutoff values by which patients could be identified. Average values, standard deviations, and diagnostic cutoff values for each peptide in each condition are shown in Table 7.

TABLE 7
Average normal concentration values for signature
peptides (n = 125) and current diagnostic cutoffs.

| | | | Diagnostic | Diagnostic |
|---|---|---|---|---|
| | Average ± SD | Number of | Cutoff | Cutoff |
| | (pmol/L) | Individuals | (pmol/L) | (SD) |
| CYBB 509 | 2056.2 ± 830.6 | 125 | 187.4 | −2.25 SD |
| ADA 93 | 2914.0 ± 1321.6 | 176 | 469.1 | −1.85 SD |
| DOCK8 1272 | 365.3 ± 134.7 | 175 | 62.2 | −2.25 SD |
| CD42 128 | 11544.3 ± 4055.7 | 176 | 3433.0 | −2.00 SD |
| CD42 154 | 18523.0 ± 7534.3 | 175 | 7447.6 | −1.47 SD |
| CD56 122 | 2494.0 ± 804.8 | 175 | 482.0 | −2.50 SD |
| ATM 1561 | 53.3 ± 19.54 | 50 | 14.2 | −2.00 SD |
| PRF 215 | 38.8 ± 23.4 | 35.00 | 9.60 | −1.25 SD |

Patient Samples.

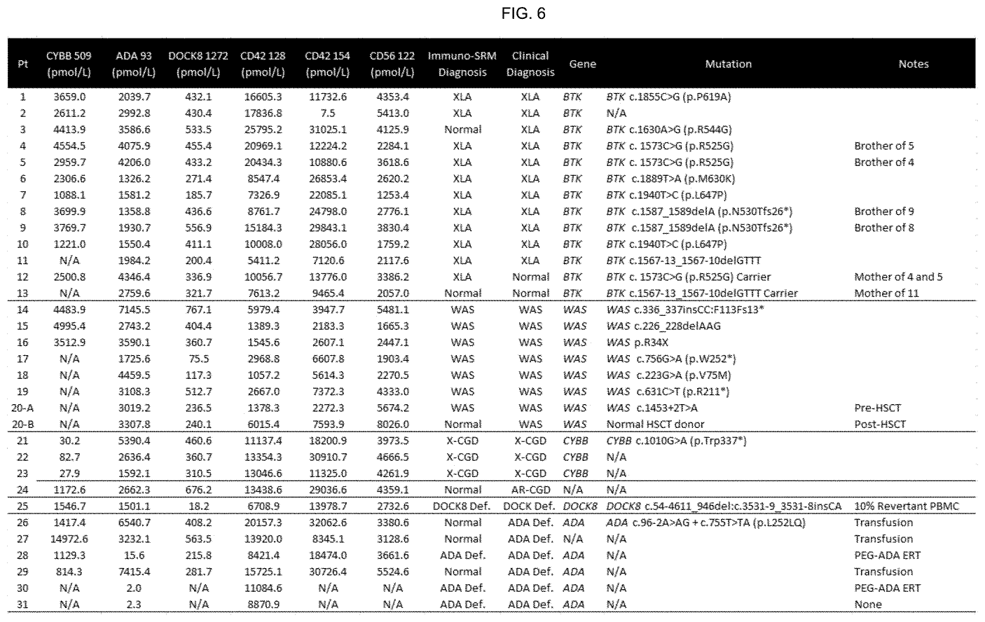

Signature peptide concentrations, immuno-SRM and clinical diagnoses, genetic information and treatments are shown in FIG. 6.

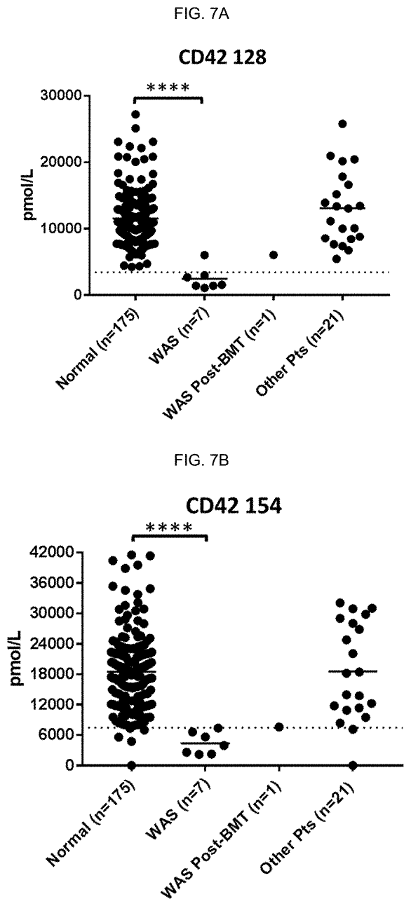

For CD42 128, six of seven untreated WAS patients were below diagnostic cutoffs while all other patients in the study had normal level of CD42 128 (FIG. 7A). For CD42 154, all untreated WAS patients were below the defined diagnostic cutoff (FIG. 7B). CD42 154 however, had a greater number of false positives and samples with no detectable peptide. This is potentially due to a known polymorphism causing mass changes and interfering with peptide detection. Finally, samples 20-A and 20-B were collected from the same patient before and after curative BMT. After BMT, this patient had CD42 128 and CD42 154 levels above the diagnostic cutoff for each peptide (FIGS. 7A, 7B). Finally, patient 14 had CD42 128 levels above the diagnostic cutoff. All non-target peptides were within the normal range for these patients and no other patient had levels of CD42 128 below diagnostic cutoffs.

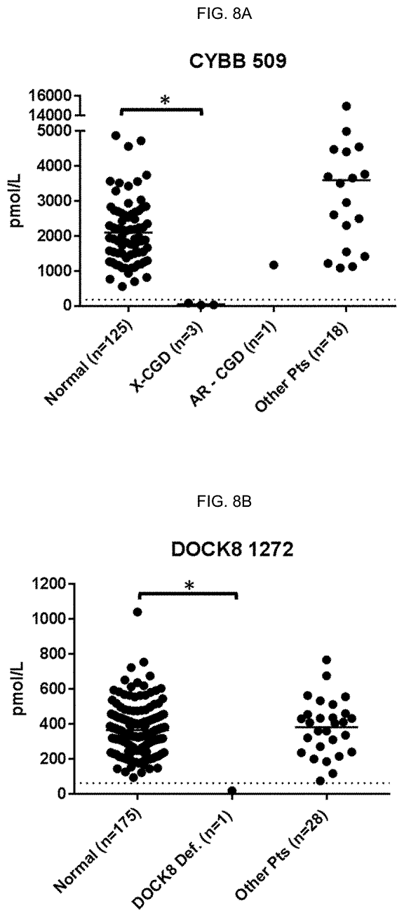

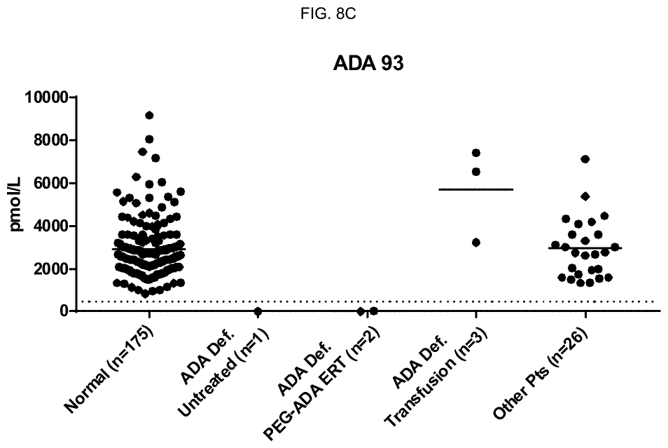

DBS of confirmed X-CGD patients were found to have CYBB concentrations reduced from normal. The average concentration of CYBB in X-CGD patients was 46.9 pmol/L (FIG. 8A). Autosomal recessive (AR)-CGD patient 24 had a CYBB concentration of 1,172.6 pmol/L well above the diagnostic cutoff of 187.4 pmol/L CYBB 509. All non-target peptides were within the normal range for these patients.

The DOCK8 deficiency patient (DBS 25) (FIG. 8B) had a DOCK8 1272 peptide concentration of 18.2 pmol/L. This is significantly reduced when compared to a normal control average of 365.3 pmol/L (p<0.05) and a diagnostic cutoff of 62.2 pmol/L. All non-target peptides were within the normal range for this patient and no other patients had below cutoff levels of DOCK8 1272.

DBS from ADA deficiency patients 26-31 had variable ADA 93 values (FIG. 8C). Patient 28, 30, and 31 had ADA 93 concentration of 15.6 pmol/L, 2.0 pmol/L, and 2.3 pmol/L, well below the diagnostic cutoff set at 469.1 pmol/L. Patients 26, 27, and 29 had peptide concentrations above cutoff levels. Patient 27 had a near average ADA 93 concentration of 3232.1 pmol/L while patient 26 and 29 had elevated peptide concentrations at 6540.7 pmol/L and 7415.1 pmol/L, respectively. Five patients were undergoing some form of treatment. Patient 28 and 30 are currently on PEG-ADA ERT and patients 26, 27, and 29 had undergone regular RBC transfusions to manage their ADA deficiency. Patient 31 was untreated and ADA deficient. No other PIDD patient or normal control samples had ADA 93 levels below cutoff concentrations.

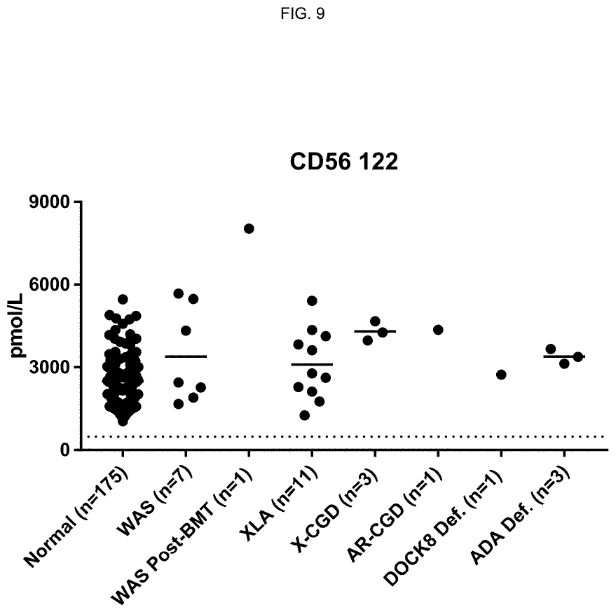

All patient samples had NK cell marker CD56 122 levels above the diagnostic cutoff determined by normal controls (FIG. 9).

High-throughput (HT) Analysis of Patient Samples.

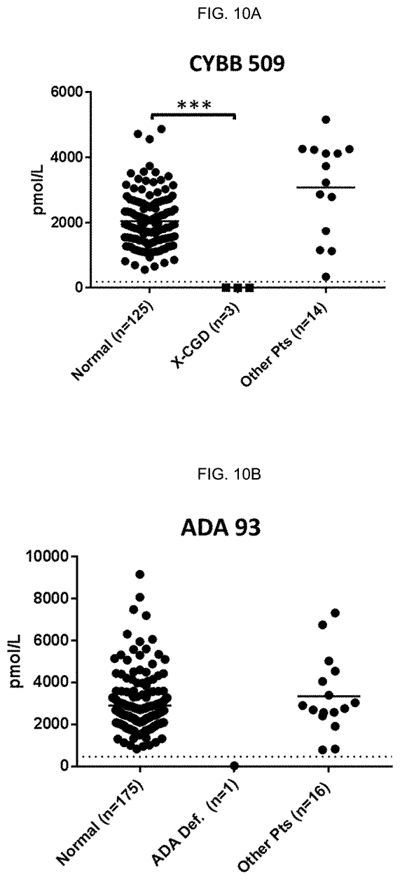

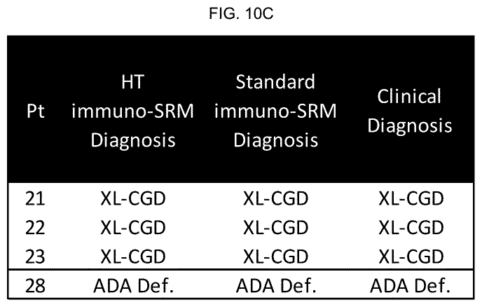

A subset of 17 patient samples were analyzed using an optimized 2.5-minute HT gradient amenable to population screening. No additional false positives were created with a HT analysis and diagnoses matched that of the standard LC-MS/MS method (FIGS. 10A-10C). High CVs were the reason for excluding DOCK8 1272 from HT method analysis.

The present study demonstrated that immuno-SRM is a highly sensitive assay that correctly identifies patients with three genetically defined PIDDs by directly quantifying low abundance proteins present in DBS. Patients with ADA deficiency, DOCK8 deficiency, and X-CGD can be screened simultaneously. New and supportive information is gained through analysis of secondary (i.e. not directly diagnostic) protein markers CD42 for platelets and CD56 for NK cells. The data also indicate that this method is highly sensitive, is reproducible over time, has a wide linear range, and is easily transferrable.

All WAS patients were genetically and biochemically confirmed cases. Secondary platelet markers CD42 128 and CD42 154 were reduced in WAS patients, consistent with a reduction in circulating platelets. Patient sample 14 was found to be the only WAS patient with levels of CD42 128 that, while low, were above the diagnostic cutoff. It is not known if patient 14 had undergone splenectomy or platelet transfusion at the time of analysis. CD42 signature peptides are therefore representative of platelet levels in DBS. Interestingly, when the same patient was analyzed before (patient 20-A) and after curative HSCT (patient 20-B), secondary platelet markers CD42 128 and CD42 154 were elevated above the diagnostic cutoff following HSCT.

X-linked CGD patients were also readily identified using immuno-SRM analysis of a CYBB signature peptide (FIG. 8A). CYBB 509 concentrations for patients 21-23 were well below the diagnostic cutoff of 187.4 pmol/L. This analysis will be specific to X-CGD patients with mutations in the CYBB gene but not for autosomal recessive forms of the disease, which are brought on by loss of other components in the NADPH complex. This is evident in the analysis of AR-CGD patient 24 who showed normal levels of CYBB 509 (FIG. 6). Other signature peptides representing CYBA or NCF1-3 could be added for individual NADPH components to identify AR forms of CGD. Because NADPH oxidase measurement by flow cytometry requires venous blood drawn by venipuncture and neutrophils have a limited viability, immuno-SRM from DBS represents a powerful alternative technology for clinical screening.

Analysis of primary markers for DOCK8 deficiency show that immuno-SRM can readily differentiate a patient from controls (FIG. 8B). For analysis of DOCK8 1272, healthy controls showed an average peptide concentration of 365.3±134.7 pmol/L. A diagnostic cutoff was set at 62.2 pmol/L or 2.25 SD below average concentrations. The DOCK8 sample analyzed had a very low concentration of DOCK8 1272 by these methods when measuring from DBS. Interestingly, the patient studied here was found to have revertant phenotype with 10% DOCK8 positive PBMCs by flow cytometry. Diagnosis of DOCK8 deficiency can often be difficult due to reversion which needs specialized flow cytometry workflows so a robust assay capable of clearly differentiating most or all patient phenotypes, regardless of reversion, would greatly simplify identification (Jing et al., The Journal of allergy and clinical immunology, 2014. 133(6): p. 1667-1675). In addition, sequencing can be challenging due to the frequent presence of large deletions in one allele. It is possible that immuno-SRM diagnostics will be more tolerant of reversion because this assay is a global analysis of all cells present in the DBS. Thus, while certain PBMC subsets may exhibit revertant expression of DOCK8, overall the total protein concentration is dramatically reduced. Because previous studies have suggested that nearly all DOCK8 patients have very low DOCK8 protein expression (Engelhardt et al., J Allergy Clin Immunol, 2009. 124(6): p. 1289-302 e4), it is expected that most patients will be screened positive by immuno-SRM. All other patients showed normal levels of DOCK8 1272, suggesting that this biomarker is characteristic for DOCK8 deficiency.

One ADA patient was untreated and had extremely reduced ADA 93 concentrations of 2.3 pmol/L. Five ADA deficiency patient screened was on some form of enzyme replacement treatment (ERT) for their disorder and the form of the treatment impacted the ability of immuno-SRM to identify patients (FIG. 8C). Patients 28 and 30 were correctly diagnosed using signature peptide ADA 93 despite being on pegylated-ADA ERT (Adagen®; pegademase bovine, Leadiant Biosciences, Inc., Gaithersburg, Md.). In these patients, ADA levels were extremely low at 15.6 pmol/L and 2.0 pmol/L (for patients 28 and 30, respectively) and well below cutoff values of 469.1 pmol/L. The recombinant ADA enzyme used for treatment is bovine in origin and does not interfere with a quantification of endogenous human ADA by immuno-SRM (Booth and Gaspar, Biologics: targets & therapy, 2009. 3: p. 349-358). Patients 26, 27, and 29 from Brazil, however, were being treated with erythrocyte transfusion to provide exogeneous enzyme since RBCs have significant levels of ADA (Fargo et al., British Journal of Haematology, 2013. 160(4): p. 547-554). With a half-life of 90 days, ADA-deficient patients who receive RBC transfusions at weekly intervals will have stable levels of RBC-associated human ADA in their blood and as a consequence appear normal in the immuno-SRM assay. This is consistent with the ADA 93 levels measured by immuno-SRM of 6540.7, 3232.1, and 7415.4 pmol/L in patients 26, 27, and 29, respectively. The protein levels in patient 26 are in fact significantly elevated from the average normal values of 2914.0 pmol/L ADA 93 which may be due to an increased RBC number upon transfusion. These results suggest that immuno-SRM may also function as a tool for determining the efficacy of treatments such as gene therapy or BMT or measuring pharmacokinetic profiles of exogenously delivered protein therapeutics.

Additional evidence of ADA treatment here may come in the form of normal NK cell marker CD56 122 concentrations (FIG. 9). ADA deficiency is a T-B-NK- type of SCID that would normally result in a reduction of NK cell concentrations in patients. Patient 28 had been receiving PEG-ADA (Adagen®) treatment at the time of sample collection which has been shown to increase lymphocyte concentrations in patients with treatment (Booth and Gaspar, Biologics: targets & therapy, 2009. 3: p. 349-358). Likewise, patients 26 and 27 transfusion treatments can increase absolute lymphocyte numbers (Schmalstieg et al., Pediatric Research, 1977. 11(4): p. 493-493). Together, these data provide information about the success of therapeutic intervention and the potential consequences on the hematopoietic and immune system.

While a 20-minute runtime will be sufficient for immuno-SRM analysis to be used as a clinical diagnostic tool, this analysis time will need to be significantly reduced in order to achieve the sample throughput necessary for NBS. To address this problem, an HT gradient that utilizes a 2.5-minute sample runtime was designed. Peptide transitions and retention time windows were optimized to maintain sufficient peptide signal and separation within a short analytical run. Overall, because of MS scan rates, it was necessary to reduce the number of monitored transitions, therefore reducing overall signal. Despite this reduction, the limits of detection for HT methods were similar in all cases except for ADA 93, CD42 154 and CD56 122 in which they were significantly higher (Table 6). In contrast, LLOQs for all peptides except for CYBB 509, ADA 93, and CD56 122 increased several-fold. These results are likely due to a reduced overall signal with HT analysis because of decreased transitions monitored, a decrease in data points measured over each peak, and increased peptide co-elution. Intra-assay CV increased in most cases but remained below 20% variation in every case except for DOCK8. DOCK8 1272 concentration analysis was poorly reproduced in HT analysis due to shifting peptide retention times and loss of resolution. Under the current gradient conditions, DOCK8 1272 would be a poor signature peptide candidate for NBS and needs to be analyzed under standard conditions. Additional candidate peptides for DOCK8 will be investigated for HT analysis.

A blinded set of samples from previous runs were analyzed by 2.5-minute HT-gradient methods (FIGS. 10A-10C). Of the patients analyzed in this sample set, the indicated conditions matched that of the standard gradient. Results for these patients and normal controls are shown in FIGS. 10A-10C. For the primary markers CYBB 509 and ADA 93, no false positives were created by HT analysis. These results suggest that immuno-SRM and the currently studied peptide biomarkers are amenable to an HT analysis required for population-based NBS studies.

These target PIDD disorders were chosen as representing strong potential candidates for newborn screening. They are relatively frequent disorders for which effective treatments, including prophylactic antibiotics and IV immunoglobulin, or curative options, including HSCT or gene therapy, exist. Without a robust newborn screening method, it often takes too much time before these relatively rare congenital PIDDs are suspected and steps taken to establish the correct diagnosis and initiate effective treatments or curative procedures. During this time, patients are prone to life-threatening infections and other negative consequences of their untreated conditions within the first year of life. Genetic counseling for families identified by NBS can also be of significant value. These facts suggest that the PIDDs included in this study are excellent candidates for newborn screening and would bring significant benefit to the patient population and healthcare system. Immuno-SRM is an attractive potential platform for NBS screening of PIDDs because it is operationally simple, rapid, low cost, and multiplexed to include multiple conditions per patient in a single run from DBS. A 2.5-minute HT run equates to a runtime of 2 conditions per minute or 3.2 biomarkers per minute if secondary cell markers are included to provide clinicians with increased context upon referral.

Immuno-SRM can effectively identify 3 congenital PIDDs simultaneously from DBS, while providing additional context about the immune system. Straightforward testing for a number of relatively rare immune disorders would create significant diagnostic value when immunodeficiencies are suspected. This immuno-SRM PIDD panel is able to produce clear results for multiple conditions in a highly-multiplexed overnight assay and can be further extended to other PIDDs where the gene product is frequently absent. Immuno-SRM extraction, digestion and enrichment are operationally straightforward and, in most cases, amenable to current clinical workflows. The ability to perform this assay from small blood volumes extracted from DBS is important and convenient as DBS cards can be collected non-invasively and mailed easily at room temperature. An immuno-SRM result suggestive of PIDDs would be invaluable in determining the need for further testing and genetic sequencing. In addition, the assay can provide contextual secondary information about the immune system analogous to flow cytometry or lymphocyte subset information. Immuno-SRM provides clear results for diseases that can often be difficult or time-consuming to diagnose and is a robust addition to current clinical workflows.

### Prophetic Example 2

Immuno-SRM assay of ATM signature peptide for diagnosis of ataxia telangiectasia (AT). DBS (as described above), buccal swab samples, peripheral blood mononuclear cells (PBMCs), or white blood cells (WBCs) from patients with AT or from patients suspected of having AT, along with the corresponding samples from normal controls, will be analyzed by immuno-SRM using signature peptides ATM 798, ATM 1544, ATM 1561, or a combination thereof, as described in Example 1 and elsewhere herein.

Buccal Swab Samples.

The controlling institutional review board approves the protocol for buccal swab samples and all subjects give written informed consent. Normal control buccal swab samples are obtained from commercial vendors. All buccal swab samples are stored in the lab at −20° C. or −80° C. Blind samples are labeled with an ID provided by the sender and identified and consented patient samples are given a lab ID upon receipt. Nylon Flocked Dry Swabs in Peel Pouches, Copan Diagnostics 502CS01 are obtained from Fisher Scientific (Chicago, Ill.; Cat no. 23-600-951). 2-mL Cryogenic Storage Vials Internal Thread are obtained from Fisher Scientific (Chicago, Ill.; Cat no. 12-567-501). Buccal swab sample collection can follow protocols described in: CHLA. (2016, Apr. 4). Buccal Swab Collection Procedure. CHLA-Clinical Pathology; (2016, Jul. 27). Buccal DNA Collection Instructions. Pathway Genomics; (2017, Dec. 14). Instruction for Buccal Swab Sample Collection. Otogenetics; PDXL PDXL. (2017, Nov. 28). Buccal Swab collection procedure—PersonalizedDx Labs [Video]. YouTube. On World Wide Web at youtube/3ftvHkfM71o?t=146; and Centers of Disease Control and Prevention (CDC). (2020, Jul. 8). Interim Guidelines for collecting, handling, and testing clinical specimens for Covid-19. On World Wide Web at cdc.gov/coronavirus/2019-ncov/lab/guidelines-clinical-specimens.html. The tip of a buccal swab containing cells can be clipped into a tube for solubilization and digestion as described above for DBS.

Peripheral Blood Mononuclear Cells (PBMCs) and White Blood Cells (WBCs).

PBMCs and WBCs can be collected by protocols known in the art, such as ones described in Kerfoot et al., Proteomics Clin Appl, 2012. 6(7-8):394-402; Grievink et al. (2016) Biopresery Biobank 14(5):410-415; Corkum et al. (2015) BMC Immunol. 16:48; and Jia et al. (2018) Biopresery Biobank 16(2):82-91; and Zhou et al. (2012) Clinical and Vaccine Immunology 19(7):1065-1074. The isolated PBMCs or WBCs can be solubilized and proteins from the cells digested as described above for DBS.

The immuno-SRM diagnoses will be compared to clinical diagnoses. If available, genetic information for the ATM gene and treatment information will be obtained for each patient. The immuno-SRM assay can be multiplexed with other signature peptides, including signature peptides to test for: X-CGD (CYBB 131, CYBB 509, or a combination thereof); SCID (IL2RG 295, IL2RG 316, CD3ε 74, CD3δ 24, CD3δ 133, or a combination thereof); ADA deficiency (ADA 93); DOCK8 deficiency (Dock8 1272); XLP1 (SH2D1A 19, SH2D1A 110, or a combination thereof); FHL2 (PRF 215, PRF 441, or a combination thereof); CVID (CD19 41, CD19 55, CD19 210, CD19 505, or a combination thereof); platelet levels (CD42 128, CD42 154, or a combination thereof); NK cell levels (CD56 122); T cell levels (CD3ε 74, CD3δ 24, CD3δ 133, or a combination thereof); or a combination thereof.

### Prophetic Example 3

Immuno-SRM assay of PRF signature peptide(s) for diagnosis of familial hemophagocytic lymphohistiocytosis 2 (FHL2). DBS, buccal swab samples, PBMCs, or WBCs (as described herein) from patients with FHL2 or from patients suspected of having FHL2, along with the corresponding samples from normal controls, will be analyzed by immuno-SRM using PRF 215 and/or PRF 441 signature peptides as described in Example 1 and elsewhere herein. The immuno-SRM diagnoses will be compared to clinical diagnoses. If available, genetic information for the PRF-1 gene and treatment information will be obtained for each patient. The immuno-SRM assay can be multiplexed with other signature peptides, including signature peptides to test for: X-CGD (CYBB 131, CYBB 509, or a combination thereof); SCID (IL2RG 295, IL2RG 316, CD3ε 74, CD3δ 24, CD3δ 133, or a combination thereof); ADA deficiency (ADA 93); DOCK8 deficiency (Dock8 1272); XLP1 (SH2D1A 19, SH2D1A 110, or a combination thereof); AT (ATM 798, ATM 1544, ATM 1561, or a combination thereof); CVID (CD19 41, CD19 55, CD19 210, CD19 505, or a combination thereof); platelet levels (CD42 128, CD42 154, or a combination thereof); NK cell levels (CD56 122); T cell levels (CD3ε 74, CD3δ 24, CD3δ 133, or a combination thereof); or a combination thereof.

### Prophetic Example 4

Immuno-SRM assay of SH2D1A (SAP) signature peptides for diagnosis of X-linked lymphoproliferative syndrome 1 (XLP1). DBS, buccal swab samples, PBMCs, or WBCs (as described herein) from patients with XLP1 or from patients suspected of having XLP1, along with the corresponding samples from normal controls, will be analyzed by immuno-SRM using SH2D1A 19 and/or SH2D1A 110 signature peptides as described in Example 1 and elsewhere herein. The immuno-SRM diagnoses will be compared to clinical diagnoses. If available, genetic information for the SAP gene and treatment information will be obtained for each patient. The immuno-SRM assay can be multiplexed with other signature peptides, including signature peptides to test for: X-CGD (CYBB 131, CYBB 509, or a combination thereof); SCID (IL2RG 295, IL2RG 316, CD3ε 74, CD3δ 24, CD3δ 133, or a combination thereof); ADA deficiency (ADA 93); DOCK8 deficiency (Dock8 1272); FHL2 (PRF 215, PRF 441, or a combination thereof); AT (ATM 798, ATM 1544, ATM 1561, or a combination thereof); CVID (CD19 41, CD19 55, CD19 210, CD19 505, or a combination thereof); platelet levels (CD42 128, CD42 154, or a combination thereof); NK cell levels (CD56 122); T cell levels (CD3ε 74, CD3δ 24, CD3δ 133, or a combination thereof); or a combination thereof.

### Prophetic Example 5

Immuno-SRM assay of CD19 signature peptides for diagnosis of common variable immunodeficiency (CVID). DBS, buccal swab samples, PBMCs, or WBCs (as described herein) from patients with CVID or from patients suspected of having CVID, along with the corresponding samples from normal controls, will be analyzed by immuno-SRM using CD19 41, CD19 55, CD19 210, and/or CD19 505 signature peptides as described herein. The immuno-SRM diagnoses will be compared to clinical diagnoses. If available, genetic information for the CD19 gene and treatment information will be obtained for each patient. The immuno-SRM assay can be multiplexed with other signature peptides, including signature peptides to test for: X-CGD (CYBB 131, CYBB 509, or a combination thereof); SCID (IL2RG 295, IL2RG 316, CD3ε 74, CD3δ 24, CD3δ 133, or a combination thereof); ADA deficiency (ADA 93); DOCK8 deficiency (Dock8 1272); FHL2 (PRF 215, PRF 441, or a combination thereof); AT (ATM 798, ATM 1544, ATM 1561, or a combination thereof); XLP1 (SH2D1A 19, SH2D1A 110, or a combination thereof); platelet levels (CD42 128, CD42 154, or a combination thereof); NK cell levels (CD56 122); T cell levels (CD3ε 74, CD3δ 24, CD3δ 133, or a combination thereof); or a combination thereof.

### Prophetic Example 6

Immuno-SRM assay of IL2RG and/or CD3 signature peptides for diagnosis of severe combined immune deficiency (SCID). DBS, buccal swab samples, PBMCs, or WBCs (as described herein) from patients with SCID or from patients suspected of having SCID, along with the corresponding samples from normal controls, will be analyzed by immuno-SRM using IL2RG 295, IL2RG 316, CD3ε 74, CD3δ 24, and/or CD3δ 133 signature peptides as described herein. The immuno-SRM diagnoses will be compared to clinical diagnoses. If available, genetic information for the IL2RG, CD3ε, CD3δ, and/or other genes mutated in SCID and treatment information will be obtained for each patient. The immuno-SRM assay can be multiplexed with other signature peptides, including signature peptides to test for: X-CGD (CYBB 131, CYBB 509, or a combination thereof); ADA deficiency (ADA 93); DOCK8 deficiency (Dock8 1272); FHL2 (PRF 215, PRF 441, or a combination thereof); AT (ATM 798, ATM 1544, ATM 1561, or a combination thereof); XLP1 (SH2D1A 19, SH2D1A 110, or a combination thereof); CVID (CD19 41, CD19 55, CD19 210, CD19 505, or a combination thereof); platelet levels (CD42 128, CD42 154, or a combination thereof); NK cell levels (CD56 122); T cell levels (CD3ε 74, CD3δ 24, CD3δ 133, or a combination thereof); or a combination thereof.

### Prophetic Example 7

Immuno-SRM assay of CD3 signature peptides for measuring levels of T cells. DBS, buccal swab samples, PBMCs, or WBCs (as described herein) from subjects and normal controls will be analyzed by immuno-SRM using CD3ε 74, CD3δ 24, and/or CD3δ 133 signature peptide to measure the level of T cells in any subject. In particular embodiments, the immuno-SRM assays use the CD3ε 74, CD3δ 24, and/or CD3δ 133 signature peptide as a secondary biomarker to support diagnosis of SCID. In the case where the CD3ε 74, CD3δ 24, and/or CD3δ 133 signature peptide is used as a secondary biomarker to support diagnosis of SCID, primary signature peptides to diagnose SCID can include: IL2RG 295, IL2RG 316, CD3ε 74, CD3δ 24, and/or CD3δ 133. The immuno-SRM assay can be multiplexed with other signature peptides, including signature peptides to test for: X-CGD (CYBB 131, CYBB 509, or a combination thereof); ADA deficiency (ADA 93); DOCK8 deficiency (Dock8 1272); FHL2 (PRF 215, PRF 441, or a combination thereof); AT (ATM 798, ATM 1544, ATM 1561, or a combination thereof); XLP1 (SH2D1A 19, SH2D1A 110, or a combination thereof); CVID (CD19 41, CD19 55, CD19 210, CD19 505, or a combination thereof); platelet levels (CD42 128, CD42 154, or a combination thereof); NK cell levels (CD56 122); or a combination thereof.

### (XIII) CLOSING PARAGRAPHS

Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to reliably diagnose X-CGD, XLP1, FHL2, AT, CVID, ADA deficiency, and/or DOCK8 deficiency utilizing DBS obtained from a newborn, the antibodies disclosed herein, and immuno-SRM.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1.-90. (canceled)

91. A method of detecting a signature peptide of X-linked chronic granulomatous disease (X-CGD), X-linked lymphoproliferative syndrome 1 (XLP1), familial hemophagocytic lymphohistiocytosis 2 (FHL2), ataxia telangiectasia (AT), common variable immunodeficiency (CVID), severe combined immunodeficiency (SCID), adenosine deaminase (ADA) deficiency, and/or dedicator of cytokinesis 8 (DOCK8) deficiency in a biological sample, the method comprising:
obtaining the biological sample;
digesting proteins of the biological sample with an enzyme to yield a digested biological sample comprising peptides;
enriching for:
(A) a CYBB signature peptide of X-CGD of SEQ ID NO: 2 with an antibody or antigen-binding fragment thereof that binds the CYBB signature peptide and comprises: a heavy chain variable (VH) domain comprising a complementarity determining region (CDR)1 as set forth in SEQ ID NO: 16, a CDR2 as set forth in SEQ ID NO: 17, and a CDR3 as set forth in SEQ ID NO: 18, and a light chain variable (VL) domain comprising: a CDR1 as set forth in SEQ ID NO: 19, a CDR2 as set forth in SEQ ID NO: 20, and a CDR3 as set forth in SEQ ID NO: 21;
an ADA signature peptide of ADA deficiency of SEQ ID NO: 11 with an antibody or antigen-binding fragment thereof that binds the ADA signature peptide and comprises: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
a DOCK8 signature peptide of DOCK8 deficiency of SEQ ID NO: 12 with an antibody or antigen-binding fragment thereof that binds the DOCK8 signature peptide and comprises: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(B) a CYBB signature peptide of X-CGD of SEQ ID NO: 2 with an antibody or antigen-binding fragment thereof that binds the CYBB signature peptide and comprises: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
a SH2D1A signature peptide of XLP1 of SEQ ID NO: 3 with an antibody or antigen binding fragment thereof that binds the SH2D1A signature peptide and comprises: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
a PRF-1 signature peptide of FHL2 of SEQ ID NO: 5 with an antibody or antigen-binding fragment thereof that binds the PRF-1 signature peptide and comprises: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an ATM signature peptide of ataxia telangiectasia of SEQ ID NO: 7 with an antibody or antigen-binding fragment thereof that binds the ATM signature peptide and comprises: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
an ADA signature peptide of ADA deficiency of SEQ ID NO: 11 with an antibody or antigen-binding fragment thereof that binds the ADA signature peptide and comprises: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
a DOCK8 signature peptide of DOCK8 deficiency of SEQ ID NO: 12 with an antibody or antigen-binding fragment thereof that binds the DOCK8 signature peptide and comprises: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(C) a CYBB signature peptide of X-CGD of SEQ ID NO: 2 with an antibody or antigen-binding fragment thereof that binds the CYBB signature peptide and comprises: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
a SH2D1A signature peptide of XLP1 of SEQ ID NO: 3 with an antibody or antigen binding fragment thereof that binds the SH2D1A signature peptide and comprises: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
a PRF-1 signature peptide of FHL2 of SEQ ID NO: 5 with an antibody or antigen-binding fragment thereof that binds the PRF-1 signature peptide and comprises: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an ATM signature peptide of ataxia telangiectasia of SEQ ID NO: 7 with an antibody or antigen-binding fragment thereof that binds the ATM signature peptide and comprises: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
a CD19 signature peptide of CVID of SEQ ID NO: 212 with an antibody or antigen binding fragment thereof that binds the CD19 signature peptide;
a first IL2RG signature peptide of SCID of SEQ ID NO: 213 with an antibody or antigen binding fragment thereof that binds the first IL2RG signature peptide;
an ADA signature peptide of ADA deficiency of SEQ ID NO: 11 with an antibody or antigen-binding fragment thereof that binds the ADA signature peptide and comprises: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
a DOCK8 signature peptide of DOCK8 deficiency of SEQ ID NO: 12 with an antibody or antigen-binding fragment thereof that binds the DOCK8 signature peptide and comprises: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(D) a CYBB signature peptide of X-CGD of SEQ ID NO: 2 with an antibody or antigen-binding fragment thereof that binds the CYBB signature peptide and comprises: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
a SH2D1A signature peptide of XLP1 of SEQ ID NO: 3 with an antibody or antigen binding fragment thereof that binds the SH2D1A signature peptide and comprises: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
a PRF-1 signature peptide of FHL2 of SEQ ID NO: 5 with an antibody or antigen-binding fragment thereof that binds the PRF-1 signature peptide and comprises: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an ATM signature peptide of ataxia telangiectasia of SEQ ID NO: 7 with an antibody or antigen-binding fragment thereof that binds the ATM signature peptide and comprises: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
a CD19 signature peptide of CVID of SEQ ID NO: 212 with an antibody or antigen binding fragment thereof that binds the CD19 signature peptide;
a second IL2RG signature peptide of SCID of SEQ ID NO: 214 with an antibody or antigen binding fragment thereof that binds the second IL2RG signature peptide;
an ADA signature peptide of ADA deficiency of SEQ ID NO: 11 with an antibody or antigen-binding fragment thereof that binds the ADA signature peptide and comprises: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
a DOCK8 signature peptide of DOCK8 deficiency of SEQ ID NO: 12 with an antibody or antigen-binding fragment thereof that binds the DOCK8 signature peptide and comprises: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(E) a CYBB signature peptide of X-CGD of SEQ ID NO: 2 with an antibody or antigen-binding fragment thereof that binds the CYBB signature peptide and comprises: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
a SH2D1A signature peptide of XLP1 of SEQ ID NO: 3 with an antibody or antigen binding fragment thereof that binds the SH2D1A signature peptide and comprises: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
a PRF-1 signature peptide of FHL2 of SEQ ID NO: 5 with an antibody or antigen-binding fragment thereof that binds the PRF-1 signature peptide and comprises: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an ATM signature peptide of ataxia telangiectasia of SEQ ID NO: 7 with an antibody or antigen-binding fragment thereof that binds the ATM signature peptide and comprises: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
a CD19 signature peptide of CVID of SEQ ID NO: 212 with an antibody or antigen binding fragment thereof that binds the CD19 signature peptide;
a CD3ε signature peptide of SCID of SEQ ID NO: 215 with an antibody or antigen binding fragment thereof that binds the CD3ε signature peptide;
an ADA signature peptide of ADA deficiency of SEQ ID NO: 11 with an antibody or antigen-binding fragment thereof that binds the ADA signature peptide and comprises: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
a DOCK8 signature peptide of DOCK8 deficiency of SEQ ID NO: 12 with an antibody or antigen-binding fragment thereof that binds the DOCK8 signature peptide and comprises: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(F) a CYBB signature peptide of X-CGD of SEQ ID NO: 2 with an antibody or antigen-binding fragment thereof that binds the CYBB signature peptide and comprises: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
a SH2D1A signature peptide of XLP1 of SEQ ID NO: 3 with an antibody or antigen binding fragment thereof that binds the SH2D1A signature peptide and comprises: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
a PRF-1 signature peptide of FHL2 of SEQ ID NO: 5 with an antibody or antigen-binding fragment thereof that binds the PRF-1 signature peptide and comprises: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an ATM signature peptide of ataxia telangiectasia of SEQ ID NO: 7 with an antibody or antigen-binding fragment thereof that binds the ATM signature peptide and comprises: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
a CD19 signature peptide of CVID of SEQ ID NO: 212 with an antibody or antigen binding fragment thereof that binds the CD19 signature peptide;
a first CD3dδ signature peptide of SCID of SEQ ID NO: 216 with an antibody or antigen binding fragment thereof that binds the first CD3dδ signature peptide;
an ADA signature peptide of ADA deficiency of SEQ ID NO: 11 with an antibody or antigen-binding fragment thereof that binds the ADA signature peptide and comprises: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
a DOCK8 signature peptide of DOCK8 deficiency of SEQ ID NO: 12 with an antibody or antigen-binding fragment thereof that binds the DOCK8 signature peptide and comprises: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(G) a CYBB signature peptide of X-CGD of SEQ ID NO: 2 with an antibody or antigen-binding fragment thereof that binds the CYBB signature peptide and comprises: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
a SH2D1A signature peptide of XLP1 of SEQ ID NO: 3 with an antibody or antigen binding fragment thereof that binds the SH2D1A signature peptide and comprises: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
a PRF-1 signature peptide of FHL2 of SEQ ID NO: 5 with an antibody or antigen-binding fragment thereof that binds the PRF-1 signature peptide and comprises: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an ATM signature peptide of ataxia telangiectasia of SEQ ID NO: 7 with an antibody or antigen-binding fragment thereof that binds the ATM signature peptide and comprises: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
a CD19 signature peptide of CVID of SEQ ID NO: 212 with an antibody or antigen binding fragment thereof that binds the CD19 signature peptide;
a second CD3dδ signature peptide of SCID of SEQ ID NO: 217 with an antibody or antigen binding fragment thereof that binds the second CD3dδ signature peptide;
an ADA signature peptide of ADA deficiency of SEQ ID NO: 11 with an antibody or antigen-binding fragment thereof that binds the ADA signature peptide and comprises: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
a DOCK8 signature peptide of DOCK8 deficiency of SEQ ID NO: 12 with an antibody or antigen-binding fragment thereof that binds the DOCK8 signature peptide and comprises: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
and
performing liquid chromatography-multiple reaction monitoring mass spectrometry (LC-MRM-MS) on the enriched peptides to determine a concentration of each signature peptide, thereby detecting one or more signature peptides of X-CGD, XLP1, FHL2, AT, CVID, SCID, ADA deficiency, and/or DOCK8 deficiency in the biological sample.

92. The method of claim 91, wherein the method further comprises detecting one or more signature peptides of platelets and/or natural killer (NK) cells by enriching for:
a first CD42 signature peptide of SEQ ID NO: 13 with an antibody or antigen-binding fragment thereof that binds the first CD42 signature peptide and comprises: a VH domain comprising a CDR1 of SEQ ID NO: 112, a CDR2 of SEQ ID NO: 113, and a CDR3 of SEQ ID NO: 114, and a VL domain comprising: a CDR1 of SEQ ID NO: 115, a CDR2 of SEQ ID NO: 116, and a CDR3 of SEQ ID NO: 117;
a second CD42 signature peptide of SEQ ID NO: 14 with an antibody or antigen-binding fragment thereof that binds the second CD42 signature peptide and comprises: a VH domain comprising a CDR1 of SEQ ID NO: 124, a CDR2 of SEQ ID NO: 125, and a CDR3 of SEQ ID NO: 126, and a VL domain comprising: a CDR1 of SEQ ID NO: 127, a CDR2 of SEQ ID NO: 128, and a CDR3 of SEQ ID NO: 129; and/or
a CD56 signature peptide of SEQ ID NO: 15 with an antibody or antigen-binding fragment thereof that binds the CD56 signature peptide and comprises: a VH domain comprising a CDR1 of SEQ ID NO: 136, a CDR2 of SEQ ID NO: 137, and a CDR3 of SEQ ID NO: 138, and a VL domain comprising: a CDR1 of SEQ ID NO: 139, a CDR2 of SEQ ID NO: 140, and a CDR3 of SEQ ID NO: 141;
and
performing liquid chromatography-multiple reaction monitoring mass spectrometry (LC-MRM-MS) on the enriched first CD42, second CD42, and/or CD56 signature peptides to determine a concentration of each signature peptide, thereby detecting one or more signature peptides of platelets and/or NK cells.

93. The method of claim 91, wherein the biological sample is dried blood spot (DBS), a buccal swab, peripheral blood mononuclear cells (PBMCs), or white blood cells (WBCs).

94. The method of claim 92, further comprising comparing the concentration of each signature peptide to that of a corresponding predetermined threshold concentration.

95. The method of claim 94, further comprising assessing a T cell level in the subject after comparing the CD3ε, the first CD3δ, and/or the second CD3δ signature peptides to that of corresponding predetermined threshold concentrations.

96. The method of claim 94, further comprising diagnosing the subject with:
X-CGD when the concentration of the CYBB signature peptide is lower than the predetermined threshold concentration or when the CYBB signature peptide is absent;
XLP1 when the concentration of the SH2D1A signature peptide is lower than the predetermined threshold concentration or when the SH2D1A signature peptide is absent;
FHL2 when the concentration of the PRF-1 signature peptide is lower than the predetermined threshold concentration or when the PRF-1 signature peptide is absent;
AT when the concentration of the ATM signature peptide is lower than the predetermined threshold concentration or when the ATM signature peptide is absent;
CVID when the concentration of the CD19 signature peptide is lower than the predetermined threshold concentration or when the CD19 signature peptide is absent;
SCID when the concentrations of the first IL2RG, the second IL2RG, the CD3ε, the first CD3δ, and/or the second CD3δ signature peptides are lower than corresponding predetermined threshold concentrations or when the first IL2RG, the second IL2RG, the CD3ε, the first CD3δ, and/or the second CD3δ signature peptides are absent;
ADA deficiency when the concentration of the ADA signature peptide is lower than the predetermined threshold concentration or when the ADA signature peptide is absent; and/or
DOCK8 deficiency when the concentration of the DOCK8 signature peptide is lower than the predetermined threshold concentration or when the DOCK8 signature peptide is absent.

97. The method of claim 96, wherein the method is performed as part of a newborn screening (NBS) that additionally screens the subject for one or more of phenylketonuria, primary congenital hypothyroidism, cystic fibrosis, and sickle cell disease.

98. The method of claim 96, wherein the method is performed in the absence of clinical symptoms of X-CGD, XLP1, FHL2, ataxia telangiectasia, CVID, SCID, ADA deficiency, and/or DOCK8 deficiency in the subject.

99. The method of claim 94, wherein the corresponding predetermined threshold concentration for each signature peptide is calculated from a standard deviation of the mean concentration of each signature peptide from a population of normal control subjects.

100. The method of claim 99, wherein the mean concentration of the CYBB signature peptide of X-CGD of SEQ ID NO: 2 from a population of normal control subjects comprises a concentration in a range of 300 pmol/L to 3000 pmol/L.

101. The method of claim 99, wherein the mean concentration of the PRF-1 signature peptide of FHL2 of SEQ ID NO: 5 from a population of normal control subjects comprises a concentration in a range of 10 pmol/L to 200 pmol/L.

102. The method of claim 99, wherein the mean concentration of the ATM signature peptide of ataxia telangiectasia of SEQ ID NO: 7 from a population of normal control subjects comprises a concentration in a range of 20 pmol/L to 100 pmol/L.

103. The method of claim 99, wherein the mean concentration of the ADA signature peptide of ADA deficiency of SEQ ID NO: 11 from a population of normal control subjects comprises a concentration in a range of 900 pmol/L to 5000 pmol/L.

104. The method of claim 99, wherein the mean concentration of the DOCK8 signature peptide of DOCK8 deficiency of SEQ ID NO: 12 from a population of normal control subjects comprises a concentration in a range of 70 pmol/L to 550 pmol/L.

105. The method of claim 99, wherein the mean concentration of the first CD42 signature peptide of SEQ ID NO: 13 from a population of normal control subjects comprises a concentration in a range of 4000 pmol/L to 20000 pmol/L.

106. The method of claim 100, wherein the mean concentration of the second CD42 signature peptide of SEQ ID NO: 14 from a population of normal control subjects comprises a concentration in a range of 8000 pmol/L to 30000 pmol/L.

107. The method of claim 100, wherein the mean concentration of the CD56 signature peptide of SEQ ID NO: 15 from a population of normal control subjects comprises a concentration in a range of 600 pmol/L to 5000 pmol/L.

108. An assay for the screening of X-linked chronic granulomatous disease (X-CGD), X-linked lymphoproliferative syndrome 1 (XLP1), familial hemophagocytic lymphohistiocytosis 2 (FHL2), ataxia telangiectasia (AT), common variable immunodeficiency (CVID), severe combined immunodeficiency (SCID), adenosine deaminase (ADA) deficiency, and/or dedicator of cytokinesis 8 (DOCKS) deficiency in a subject, the assay comprising:
(i) (A) an antibody or antigen-binding fragment thereof comprising: a heavy chain variable (VH) domain comprising a complementarity determining region (CDR)1 as set forth in SEQ ID NO: 16, a CDR2 as set forth in SEQ ID NO: 17, and a CDR3 as set forth in SEQ ID NO: 18, and a light chain variable (VL) domain comprising: a CDR1 as set forth in SEQ ID NO: 19, a CDR2 as set forth in SEQ ID NO: 20, and a CDR3 as set forth in SEQ ID NO: 21;
an antibody or antigen-binding fragment thereof comprising: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
an antibody or antigen-binding fragment thereof comprising: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(B) an antibody or antigen-binding fragment thereof comprising: a VH domain comprising a CDR1 as set forth in SEQ ID NO: 16, a CDR2 as set forth in SEQ ID NO:
17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
an antibody or antigen binding fragment thereof comprising: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
an antibody or antigen-binding fragment thereof comprising: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an antibody or antigen-binding fragment thereof comprising: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
an antibody or antigen-binding fragment thereof comprising: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
an antibody or antigen-binding fragment thereof comprising: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(C) an antibody or antigen-binding fragment thereof comprising: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
an antibody or antigen binding fragment thereof comprising: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
an antibody or antigen-binding fragment thereof comprising: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an antibody or antigen-binding fragment thereof comprising: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
an antibody or antigen binding fragment thereof that binds a CD19 signature peptide of CVID of SEQ ID NO: 212;
an antibody or antigen binding fragment thereof that binds a first IL2RG signature peptide of SCID of SEQ ID NO: 213;
an antibody or antigen-binding fragment thereof comprising: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
an antibody or antigen-binding fragment thereof comprising: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(D) an antibody or antigen-binding fragment thereof comprising: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
an antibody or antigen binding fragment thereof comprising: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
an antibody or antigen-binding fragment thereof comprising: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an antibody or antigen-binding fragment thereof comprising: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
an antibody or antigen binding fragment thereof that binds a CD19 signature peptide of CVID of SEQ ID NO: 212;
an antibody or antigen binding fragment thereof that binds a second IL2RG signature peptide of SCID of SEQ ID NO: 214;
an antibody or antigen-binding fragment thereof comprising: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
an antibody or antigen-binding fragment thereof comprising: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(E) an antibody or antigen-binding fragment thereof comprising: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
an antibody or antigen binding fragment thereof comprising: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
an antibody or antigen-binding fragment thereof comprising: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an antibody or antigen-binding fragment thereof comprising: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
an antibody or antigen binding fragment thereof that binds a CD19 signature peptide of CVID of SEQ ID NO: 212;
an antibody or antigen binding fragment thereof that binds a CD3ε signature peptide of SCID of SEQ ID NO: 215;
an antibody or antigen-binding fragment thereof comprising: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
an antibody or antigen-binding fragment thereof comprising: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(F) an antibody or antigen-binding fragment thereof comprising: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
an antibody or antigen binding fragment thereof comprising: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
an antibody or antigen-binding fragment thereof comprising: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an antibody or antigen-binding fragment thereof comprising: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
an antibody or antigen binding fragment thereof that binds a CD19 signature peptide of CVID of SEQ ID NO: 212;
an antibody or antigen binding fragment thereof that binds a first CD3dδ signature peptide of SCID of SEQ ID NO: 216;
an antibody or antigen-binding fragment thereof comprising: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
an antibody or antigen-binding fragment thereof comprising: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
or
(G) an antibody or antigen-binding fragment thereof comprising: a VH domain comprising a CDR1 as set forth in SEQ ID NO: 16, a CDR2 as set forth in SEQ ID NO: 17, and a CDR3 as set forth in SEQ ID NO: 18, and a VL domain comprising: a CDR1 as set forth in SEQ ID NO: 19, a CDR2 as set forth in SEQ ID NO: 20, and a CDR3 as set forth in SEQ ID NO: 21;
an antibody or antigen binding fragment thereof comprising: a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
an antibody or antigen-binding fragment thereof comprising: a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
an antibody or antigen-binding fragment thereof comprising: a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
an antibody or antigen binding fragment thereof that binds a CD19 signature peptide of CVID of SEQ ID NO: 212;
an antibody or antigen binding fragment thereof that binds a second CD3dδ signature peptide of SCID of SEQ ID NO: 217;
an antibody or antigen-binding fragment thereof comprising: a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87; and
an antibody or antigen-binding fragment thereof comprising: a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
and/or
(ii) reference signature peptides comprising:
a CYBB signature peptide of X-CGD of SEQ ID NO: 2;
a SH2D1A signature peptide of XLP1 of SEQ ID NO: 3;
a PRF-1 signature peptide of FHL2 of SEQ ID NO: 5;
an ATM signature peptide of ataxia telangiectasia of SEQ ID NO: 7;
a CD19 signature peptide of CVID of SEQ ID NO: 212;
an IL2RG signature peptide of SCID of SEQ ID NO: 213;
an IL2RG signature peptide of SCID of SEQ ID NO: 214;
a CD3ε signature peptide of SCID of SEQ ID NO: 215;
a CD3δ signature peptide of SCID of SEQ ID NO: 216;
a CD3δ signature peptide of SCID of SEQ ID NO: 217;
an ADA signature peptide of ADA deficiency of SEQ ID NO: 11; and/or
a DOCK8 signature peptide of DOCK8 deficiency of SEQ ID NO: 12.

109. A recombinant antibody or antigen binding fragment thereof comprising:
(A) a heavy chain variable (VH) domain comprising a complementarity determining region (CDR)1 as set forth in SEQ ID NO: 16, a CDR2 as set forth in SEQ ID NO: 17, and a CDR3 as set forth in SEQ ID NO: 18, and a light chain variable (VL) domain comprising: a CDR1 as set forth in SEQ ID NO: 19, a CDR2 as set forth in SEQ ID NO: 20, and a CDR3 as set forth in SEQ ID NO: 21;
(B) a VH domain comprising a CDR1 as set forth in SEQ ID NO: 160, a CDR2 as set forth in SEQ ID NO: 161, and a CDR3 as set forth in SEQ ID NO: 162, and a VL domain comprising: a CDR1 as set forth in SEQ ID NO: 163, a CDR2 as set forth in SEQ ID NO: 164, and a CDR3 as set forth in SEQ ID NO: 165;
(C) a VH domain comprising a CDR1 as set forth in SEQ ID NO: 34, a CDR2 as set forth in SEQ ID NO: 35, and a CDR3 as set forth in SEQ ID NO: 36, and a VL domain comprising: a CDR1 as set forth in SEQ ID NO: 37, a CDR2 as set forth in SEQ ID NO: 38, and a CDR3 as set forth in SEQ ID NO: 39;
(D) a VH domain comprising a CDR1 as set forth in SEQ ID NO: 52, a CDR2 as set forth in SEQ ID NO: 53, and a CDR3 as set forth in SEQ ID NO: 54, and a VL domain comprising: a CDR1 as set forth in SEQ ID NO: 55, a CDR2 as set forth in SEQ ID NO: 56, and a CDR3 as set forth in SEQ ID NO: 57;
(E) a VH domain comprising a CDR1 as set forth in SEQ ID NO: 82, a CDR2 as set forth in SEQ ID NO: 83, and a CDR3 as set forth in SEQ ID NO: 84, and a VL domain comprising: a CDR1 as set forth in SEQ ID NO: 85, a CDR2 as set forth in SEQ ID NO: 86, and a CDR3 as set forth in SEQ ID NO: 87;
(F) a VH domain comprising a CDR1 as set forth in SEQ ID NO: 100, a CDR2 as set forth in SEQ ID NO: 101, and a CDR3 as set forth in SEQ ID NO: 102, and a VL domain comprising: a CDR1 as set forth in SEQ ID NO: 103, a CDR2 as set forth in SEQ ID NO: 104, and a CDR3 as set forth in SEQ ID NO: 105;
(G) a VH domain comprising a CDR1 as set forth in SEQ ID NO: 112, a CDR2 as set forth in SEQ ID NO: 113, and a CDR3 as set forth in SEQ ID NO: 114, and a VL domain comprising: a CDR1 as set forth in SEQ ID NO: 115, a CDR2 as set forth in SEQ ID NO: 116, and a CDR3 as set forth in SEQ ID NO: 117;
(H) a VH domain comprising a CDR1 as set forth in SEQ ID NO: 124, a CDR2 as set forth in SEQ ID NO: 125, and a CDR3 as set forth in SEQ ID NO: 126, and a VL domain comprising: a CDR1 as set forth in SEQ ID NO: 127, a CDR2 as set forth in SEQ ID NO: 128, and a CDR3 as set forth in SEQ ID NO: 129; or
(I) a VH domain comprising a CDR1 as set forth in SEQ ID NO: 136, a CDR2 as set forth in SEQ ID NO: 137, and a CDR3 as set forth in SEQ ID NO: 138, and a VL domain comprising: a CDR1 as set forth in SEQ ID NO: 139, a CDR2 as set forth in SEQ ID NO: 140, and a CDR3 as set forth in SEQ ID NO: 141,
each according to Kabat numbering.

110. The recombinant antibody or antigen binding fragment thereof of claim 109, wherein
(A) the VH domain is set forth in SEQ ID NO: 27 and/or the heavy chain is set forth in SEQ ID NO: 24; and the VL domain is set forth in SEQ ID NO: 33 and/or the light chain is set forth in SEQ ID NO: 30;
(B) the VH domain is set forth in SEQ ID NO: 173 and/or the heavy chain is set forth in SEQ ID NO: 169; and the VL domain is set forth in SEQ ID NO: 181 and/or the light chain is set forth in SEQ ID NO: 177;
(C) the VH domain is set forth in SEQ ID NO: 45 and/or the heavy chain is set forth in SEQ ID NO: 42; and the VL domain is set forth in SEQ ID NO: 51 and/or the light chain is set forth in SEQ ID NO: 48;
(D) the VH domain is set forth in SEQ ID NO: 63 and/or the heavy chain is set forth in SEQ ID NO: 60; and the VL domain is set forth in SEQ ID NO: 69 and/or the light chain is set forth in SEQ ID NO: 66;
(E) the VH domain is set forth in SEQ ID NO: 93 and/or the heavy chain is set forth in SEQ ID NO: 90; and the VL domain is set forth in SEQ ID NO: 99 and/or the light chain is set forth in SEQ ID NO: 96;
(F) the VH domain is set forth in SEQ ID NO: 108 and the VL domain is set forth in SEQ ID NO: 111;
(G) the VH domain is set forth in SEQ ID NO: 120 and the VL domain is set forth in SEQ ID NO: 123;
(H) the VH domain is set forth in SEQ ID NO: 132 and the VL domain is set forth in SEQ ID NO: 135;
(I) the VH domain is set forth in SEQ ID NO: 147 and/or the heavy chain is set forth in SEQ ID NO: 144; and the VL domain is set forth in SEQ ID NO: 153 and/or the light chain is set forth in SEQ ID NO: 150.


SEQUENCE LISTING (217 sequences)

SEQ ID NO: 1 - protein, 17 aa, Homo sapiens
VNNSDPYSVALSELGDR

SEQ ID NO: 2 - protein, 13 aa, Homo sapiens
TLYGRPNWDNEFK

SEQ ID NO: 3 - protein, 14 aa, Homo sapiens
LLLATGLDGSYLLR

SEQ ID NO: 4 - protein, 9 aa, Homo sapiens
STQGTTGIR

SEQ ID NO: 5 - protein, 11 aa, Homo sapiens
LISNYGTHFIR

SEQ ID NO: 6 - protein, 9 aa, Homo sapiens
LFFGGQELR

SEQ ID NO: 7 - protein, 12 aa, Homo sapiens
LLDPFPDHVVFK

SEQ ID NO: 8 - protein, 14 aa, Homo sapiens
GTSDGPTQQLTWSR

SEQ ID NO: 9 - protein, 11 aa, Homo sapiens
GPLSWTHVHPK

SEQ ID NO: 10 - protein, 10 aa, Homo sapiens
GILYAAPQLR

SEQ ID NO: 11 - protein, 9 aa, Homo sapiens
EGVVYVEVR

SEQ ID NO: 12 - protein, 12 aa, Homo sapiens
TSGIVLSSLPYK

SEQ ID NO: 13 - protein, 10 aa, Homo sapiens
LTSLPLGALR

SEQ ID NO: 14 - protein, 12 aa, Homo sapiens
TLPPGLLTPTPK

SEQ ID NO: 15 - protein, 9 aa, Homo sapiens
NAPTPQEFR

SEQ ID NO: 16 - protein, 6 aa, Artificial Sequence
  anti-CYBB 509 VH CDR1
RSYWIC

SEQ ID NO: 17 - protein, 18 aa, Artificial Sequence
  anti-CYBB 509 VH CDR2
CIYAGSSGSTYYASWAKG

SEQ ID NO: 18 - protein, 10 aa, Artificial Sequence
  anti-CYBB 509 VH CDR3
GNNNIIILDL

SEQ ID NO: 19 - protein, 13 aa, Artificial Sequence
  anti-CYBB 509 VL CDR1
QASQSVYNNNALS

SEQ ID NO: 20 - protein, 7 aa, Artificial Sequence
  anti-CYBB 509 VL CDR2
DASDLAP

SEQ ID NO: 21 - protein, 11 aa, Artificial Sequence
  anti-CYBB 509 VL CDR3
LGGYSVSSDNA

SEQ ID NO: 22 - DNA, 1386 bp, Artificial Sequence
  anti-CYBB 509 heavy chain coding sequence (clone EB0611C -1E12-H2) with leader sequence
atggagactgggctgcgctggcttctcctggtcgctgtgctcaaaggtgtccagtgtcagtcgttggaggagtccgggggaggcctggtcaagcctggggcatccctgacactcacctgcacagcctctggattctccttcagtcgtagttattggatatgctgggtccgccaggctccagggaaggggctggagtggctcggatgcatttatgctggtagtagtggtagcacttactacgcgagctgggcgaaaggccgattcaccatctccaaaacctcgtcgaccacggtgactctacaaatgaccagtctgacagccgcggacacggccacctatttctgtgcgagaggaaataataatattataatcttggatttgtggggcccaggcatactggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 23 - protein, 461 aa, Artificial Sequence
  anti-CYBB 509 heavy chain amino acid sequence with leader sequence
METGLRWLLLVAVLKGVQCQSLEESGGGLVKPGASLTLTCTASGFSFSRSYWICWVRQAPGKGLEWLGCIYAGSSGSTYYASWAKGRFTISKTSSTTVTLQMTSLTAADTATYFCARGNNNIIILDLWGPGILVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 24 - protein, 442 aa, Artificial Sequence
  anti-CYBB 509 heavy chain amino acid sequence without leader sequence
QSLEESGGGLVKPGASLTLTCTASGFSFSRSYWICWVRQAPGKGLEWLGCIYAGSSGSTYYASWAKGRFTISKTSSTTVTLQMTSLTAADTATYFCARGNNNIIILDLWGPGILVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 25 - DNA, 414 bp, Artificial Sequence
  anti-CYBB 509 variable heavy domain coding sequence with leader sequence
atggagactgggctgcgctggcttctcctggtcgctgtgctcaaaggtgtccagtgtcagtcgttggaggagtccgggggaggcctggtcaagcctggggcatccctgacactcacctgcacagcctctggattctccttcagtcgtagttattggatatgctgggtccgccaggctccagggaaggggctggagtggctcggatgcatttatgctggtagtagtggtagcacttactacgcgagctgggcgaaaggccgattcaccatctccaaaacctcgtcgaccacggtgactctacaaatgaccagtctgacagccgcggacacggccacctatttctgtgcgagaggaaataataatattataatcttggatttgtggggcccaggcatactggtcaccgtctcctca

SEQ ID NO: 26 - protein, 138 aa, Artificial Sequence
  anti-CYBB 509 variable heavy domain amino acid sequence with leader sequence
METGLRWLLLVAVLKGVQCQSLEESGGGLVKPGASLTLTCTASGFSFSRSYWICWVRQAPGKGLEWLGCIYAGSSGSTYYASWAKGRFTISKTSSTTVTLQMTSLTAADTATYFCARGNNNIIILDLWGPGILVTVSS

SEQ ID NO: 27 - protein, 119 aa, Artificial Sequence
  anti-CYBB 509 variable heavy domain amino acid sequence without leader sequence
QSLEESGGGLVKPGASLTLTCTASGFSFSRSYWICWVRQAPGKGLEWLGCIYAGSSGSTYYASWAKGRFTISKTSSTTVTLQMTSLTAADTATYFCARGNNNIIILDLWGPGILVTVSS

SEQ ID NO: 28 - DNA, 714 bp, Artificial Sequence
  anti-CYBB 509 light chain coding sequence (clone EB0611C -1E12-K2) with leader sequence
atggacacgagggcccccactcagctgctggggctcctgctgctctggctcccaggtgccacatttgccgccgtgctgacccagactccatctcccgtgtctgcagctgtgggaggcacagtcaccatcagttgccaggccagtcagagtgtttacaataacaatgccttatcctggtatcagcagaaaccagggcagcctcccaagctcctgatctatgatgcatccgatctggcacctggggtcccatcgcggttcagcggcagtggatctgggacacagttcactctcaccatcagcgacgtgcagtgtgacgatgctgccacttactactgtctaggcggttatagtgtgagtagtgataatgctttcggcggagggaccgaggtggtggtcaacggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 29 - protein, 237 aa, Artificial Sequence
  anti-CYBB 509 light chain amino acid sequence with leader sequence
MDTRAPTQLLGLLLLWLPGATFAAVLTQTPSPVSAAVGGTVTISCQASQSVYNNNALSWYQQKPGQPPKLLIYDASDLAPGVPSRFSGSGSGTQFTLTISDVQCDDAATYYCLGGYSVSSDNAFGGGTEVVVNGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 30 - protein, 214 aa, Artificial Sequence
  anti-CYBB 509 light chain amino acid sequence without leader sequence
AVLTQTPSPVSAAVGGTVTISCQASQSVYNNNALSWYQQKPGQPPKLLIYDASDLAPGVPSRFSGSGSGTQFTLTISDVQCDDAATYYCLGGYSVSSDNAFGGGTEVVVNGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 31 - DNA, 399 bp, Artificial Sequence
  anti-CYBB 509 variable light domain coding sequence with leader sequence
atggacacgagggcccccactcagctgctggggctcctgctgctctggctcccaggtgccacatttgccgccgtgctgacccagactccatctcccgtgtctgcagctgtgggaggcacagtcaccatcagttgccaggccagtcagagtgtttacaataacaatgccttatcctggtatcagcagaaaccagggcagcctcccaagctcctgatctatgatgcatccgatctggcacctggggtcccatcgcggttcagcggcagtggatctgggacacagttcactctcaccatcagcgacgtgcagtgtgacgatgctgccacttactactgtctaggcggttatagtgtgagtagtgataatgctttcggcggagggaccgaggtggtggtcaac

SEQ ID NO: 32 - protein, 133 aa, Artificial Sequence
  anti-CYBB 509 variable light domain amino acid sequence with leader sequence
MDTRAPTQLLGLLLLWLPGATFAAVLTQTPSPVSAAVGGTVTISCQASQSVYNNNALSWYQQKPGQPPKLLIYDASDLAPGVPSRFSGSGSGTQFTLTISDVQCDDAATYYCLGGYSVSSDNAFGGGTEVVVN

SEQ ID NO: 33 - protein, 110 aa, Artificial Sequence
  anti-CYBB 509 variable light domain amino acid sequence without leader sequence
AVLTQTPSPVSAAVGGTVTISCQASQSVYNNNALSWYQQKPGQPPKLLIYDASDLAPGVPSRFSGSGSGTQFTLTISDVQCDDAATYYCLGGYSVSSDNAFGGGTEVVVN

SEQ ID NO: 34 - protein, 5 aa, Artificial Sequence
  anti-PRF 215 VH CDR1
RHNMG

SEQ ID NO: 35 - protein, 16 aa, Artificial Sequence
  anti-PRF 215 VH CDR2
SISSKGKTYYTTWAKG

SEQ ID NO: 36 - protein, 11 aa, Artificial Sequence
  anti-PRF 215 VH CDR3
LYSGSNGYETI

SEQ ID NO: 37 - protein, 13 aa, Artificial Sequence
  anti-PRF 215 VL CDR1
QASQSVYDNNNLA

SEQ ID NO: 38 - protein, 7 aa, Artificial Sequence
  anti-PRF 215 VL CDR2
DASKLAS

SEQ ID NO: 39 - protein, 13 aa, Artificial Sequence
  anti-PRF 215 VL CDR3
LGEFISSSGDCIV

SEQ ID NO: 40 - DNA, 1377 bp, Artificial Sequence
  anti-PRF 215 heavy chain coding sequence (clone EB0610D-9E12-H3) with leader sequence
atggagactgggctgcgctggcttctcctggtcgctgtgctcaaaggtgtccagtgtcagtcggtggaggagtccgggggtcgcctggtaacgcctggaggaaccctgacactcacctgcacagcctctggattctccctcagtaggcacaacatgggctgggtccgccaggctccaggggaggggctggagtggatcggatccattagtagtaagggtaagacatactacacgacctgggcgaaaggtcgattcaccatctccaaaagctcgaccacggtggatctgaaaatcaccagtccgacaaccgaggacacggccacctatttctgtgccagattatattctggtagtaatggttatgaaaccatctggggcccaggcaccctggtcaccgtctccttggggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 41 - protein, 458 aa, Artificial Sequence
  anti-PRF 215 heavy chain amino acid sequence with leader sequence
METGLRWLLLVAVLKGVQCQSVEESGGRLVTPGGTLTLTCTASGFSLSRHNMGWVRQAPGEGLEWIGSISSKGKTYYTTWAKGRFTISKSSTTVDLKITSPTTEDTATYFCARLYSGSNGYETIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 42 - protein, 439 aa, Artificial Sequence
  anti-PRF 215 heavy chain amino acid sequence without leader sequence
QSVEESGGRLVTPGGTLTLTCTASGFSLSRHNMGWVRQAPGEGLEWIGSISSKGKTYYTTWAKGRFTISKSSTTVDLKITSPTTEDTATYFCARLYSGSNGYETIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 43 - DNA, 405 bp, Artificial Sequence
  anti-PRF 215 variable heavy domain coding sequence with leader sequence
atggagactgggctgcgctggcttctcctggtcgctgtgctcaaaggtgtccagtgtcagtcggtggaggagtccgggggtcgcctggtaacgcctggaggaaccctgacactcacctgcacagcctctggattctccctcagtaggcacaacatgggctgggtccgccaggctccaggggaggggctggagtggatcggatccattagtagtaagggtaagacatactacacgacctgggcgaaaggtcgattcaccatctccaaaagctcgaccacggtggatctgaaaatcaccagtccgacaaccgaggacacggccacctatttctgtgccagattatattctggtagtaatggttatgaaaccatctggggcccaggcaccctggtcaccgtctccttg

SEQ ID NO: 44 - protein, 135 aa, Artificial Sequence
  anti-PRF 215 variable heavy domain amino acid sequence with leader sequence
METGLRWLLLVAVLKGVQCQSVEESGGRLVTPGGTLTLTCTASGFSLSRHNMGWVRQAPGEGLEWIGSISSKGKTYYTTWAKGRFTISKSSTTVDLKITSPTTEDTATYFCARLYSGSNGYETIWGPGTLVTVSL

SEQ ID NO: 45 - protein, 116 aa, Artificial Sequence
  anti-PRF 215 variable heavy domain amino acid sequence without leader sequence
QSVEESGGRLVTPGGTLTLTCTASGFSLSRHNMGWVRQAPGEGLEWIGSISSKGKTYYTTWAKGRFTISKSSTTVDLKITSPTTEDTATYFCARLYSGSNGYETIWGPGTLVTVSL

SEQ ID NO: 46 - DNA, 723 bp, Artificial Sequence
  anti-PRF 215 light chain coding sequence (clone EB0610D-9E12-K1) with leader sequence
  misc_feature (592)..(592): n is a, c, g, or t
atggacacgagggcccccactcagctgctggggctcctgctgctctggctcccaggtgccagatgtgccgatgtcgtgatgacccagactccatcccccgtgtctgcagctgtgggaggcacagtcaccatcaattgccaggccagtcagagtgtttatgataacaacaacttagcctggtatcagcagaaaccagggcagcctcccaagctcctgatctatgatgcatccaaactggcatctggggtcccatcgcggttcagcggcagtggatctgggacacagttcactctcaccatcagcgacgtgcagtgtgacgatgctgccacttactactgtctaggcgaatttatttctagtagtggtgattgtattgttttcggcggagggaccgagatggtcgtcaaaggtgatccagttgcacctactgtcctcatcttcccaccatctgctgatcttgtggcaactggaacagtcaccatcgtgtgtgtggcgaataaatactttcccgatgtcaccgtcacctgggaggtggatggcaccacccaaacaactggcatcgagaacagtaaaacaccgcagaattctgcanattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 47 - protein, 240 aa, Artificial Sequence
  anti-PRF 215 light chain amino acid sequence with leader sequence
  misc_feature (198)..(198): Xaa can be any naturally occurring amino acid
MDTRAPTQLLGLLLLWLPGARCADVVMTQTPSPVSAAVGGTVTINCQASQSVYDNNNLAWYQQKPGQPPKLLIYDASKLASGVPSRFSGSGSGTQFTLTISDVQCDDAATYYCLGEFISSSGDCIVFGGGTEMVVKGDPVAPTVLIFPPSADLVATGTVTIVCVANKYFPDVTVTWEVDGTTQTTGIENSKTPQNSAXCTYNLSSTLTLTSTQYNSHKEYTCKVTQGTTSVVQSFNRGDC

SEQ ID NO: 48 - protein, 218 aa, Artificial Sequence
  anti-PRF 215 light chain amino acid sequence without leader sequence
  misc_feature (176)..(176): Xaa can be any naturally occurring amino acid
ADVVMTQTPSPVSAAVGGTVTINCQASQSVYDNNNLAWYQQKPGQPPKLLIYDASKLASGVPSRFSGSGSGTQFTLTISDVQCDDAATYYCLGEFISSSGDCIVFGGGTEMVVKGDPVAPTVLIFPPSADLVATGTVTIVCVANKYFPDVTVTWEVDGTTQTTGIENSKTPQNSAXCTYNLSSTLTLTSTQYNSHKEYTCKVTQGTTSVVQSFNRGDC

SEQ ID NO: 49 - DNA, 408 bp, Artificial Sequence
  anti-PRF 215 variable light domain coding sequence with leader sequence
atggacacgagggcccccactcagctgctggggctcctgctgctctggctcccaggtgccagatgtgccgatgtcgtgatgacccagactccatcccccgtgtctgcagctgtgggaggcacagtcaccatcaattgccaggccagtcagagtgtttatgataacaacaacttagcctggtatcagcagaaaccagggcagcctcccaagctcctgatctatgatgcatccaaactggcatctggggtcccatcgcggttcagcggcagtggatctgggacacagttcactctcaccatcagcgacgtgcagtgtgacgatgctgccacttactactgtctaggcgaatttatttctagtagtggtgattgtattgttttcggcggagggaccgagatggtcgtcaaa

SEQ ID NO: 50 - protein, 136 aa, Artificial Sequence
  anti-PRF 215 variable light domain amino acid sequence with leader sequence
MDTRAPTQLLGLLLLWLPGARCADVVMTQTPSPVSAAVGGTVTINCQASQSVYDNNNLAWYQQKPGQPPKLLIYDASKLASGVPSRFSGSGSGTQFTLTISDVQCDDAATYYCLGEFISSSGDCIVFGGGTEMVVK

SEQ ID NO: 51 - protein, 114 aa, Artificial Sequence
  anti-PRF 215 variable light domain amino acid sequence without leader sequence
ADVVMTQTPSPVSAAVGGTVTINCQASQSVYDNNNLAWYQQKPGQPPKLLIYDASKLASGVPSRFSGSGSGTQFTLTISDVQCDDAATYYCLGEFISSSGDCIVFGGGTEMVVK

SEQ ID NO: 52 - protein, 5 aa, Artificial Sequence
  anti-ATM 1561 VH CDR1
SGGMC

SEQ ID NO: 53 - protein, 18 aa, Artificial Sequence
  anti-ATM 1561 VH CDR2
CIVNGDGSTYFATWAKGR

SEQ ID NO: 54 - protein, 4 aa, Artificial Sequence
  anti-ATM 1561 VH CDR3
YWAL

SEQ ID NO: 55 - protein, 13 aa, Artificial Sequence
  anti-ATM 1561 VL CDR1
QASESLYNNNRLA

SEQ ID NO: 56 - protein, 7 aa, Artificial Sequence
  anti-ATM 1561 VL CDR2
SASTLAS

SEQ ID NO: 57 - protein, 12 aa, Artificial Sequence
  anti-ATM 1561 VL CDR3
VGGRSRVTDGIA

SEQ ID NO: 58 - DNA, 1365 bp, Artificial Sequence
  anti-ATM 1561 heavy chain coding sequence (clone EB0608D-4A10-H4) with leader sequence
atggagactgggctgcgctggcttctcctggtcgctgtgctcaaaggtgtccagtgtcaggagcagctggtggagtccgggggaggcctggtccagcctgggggatccctgacactcacctgcaaagcctctggattcgacttcagtagcggtggaatgtgctgggtccgccaggctccagggaaggggctggagtggatcggatgcattgtaaatggtgatggtagcacatacttcgcgacctgggcgaaaggccgattcaccatcaccagaacgtcgtcgaccacggtgactctgcaaatgaccagtctgacagccgcggacacggccacctatttctgttggagatattgggccttgtggggcccaggtaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 59 - protein, 454 aa, Artificial Sequence
  anti-ATM 1561 heavy chain amino acid sequence with leader sequence
METGLRWLLLVAVLKGVQCQEQLVESGGGLVQPGGSLTLTCKASGFDFSSGGMCWVRQAPGKGLEWIGCIVNGDGSTYFATWAKGRFTITRTSSTTVTLQMTSLTAADTATYFCWRYWALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 60 - protein, 435 aa, Artificial Sequence
  anti-ATM 1561 heavy chain amino acid sequence without leader sequence
QEQLVESGGGLVQPGGSLTLTCKASGFDFSSGGMCWVRQAPGKGLEWIGCIVNGDGSTYFATWAKGRFTITRTSSTTVTLQMTSLTAADTATYFCWRYWALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 61 - DNA, 393 bp, Artificial Sequence
  anti-ATM 1561 variable heavy domain coding sequence with leader sequence
atggagactgggctgcgctggcttctcctggtcgctgtgctcaaaggtgtccagtgtcaggagcagctggtggagtccgggggaggcctggtccagcctgggggatccctgacactcacctgcaaagcctctggattcgacttcagtagcggtggaatgtgctgggtccgccaggctccagggaaggggctggagtggatcggatgcattgtaaatggtgatggtagcacatacttcgcgacctgggcgaaaggccgattcaccatcaccagaacgtcgtcgaccacggtgactctgcaaatgaccagtctgacagccgcggacacggccacctatttctgttggagatattgggccttgtggggcccaggtaccctggtcaccgtctcctca

SEQ ID NO: 62 - protein, 131 aa, Artificial Sequence
  anti-ATM 1561 variable heavy domain amino acid sequence with leader sequence
METGLRWLLLVAVLKGVQCQEQLVESGGGLVQPGGSLTLTCKASGFDFSSGGMCWVRQAPGKGLEWIGCIVNGDGSTYFATWAKGRFTITRTSSTTVTLQMTSLTAADTATYFCWRYWALWGPGTLVTVSS

SEQ ID NO: 63 - protein, 112 aa, Artificial Sequence
  anti-ATM 1561 variable heavy domain amino acid sequence without leader sequence
QEQLVESGGGLVQPGGSLTLTCKASGFDFSSGGMCWVRQAPGKGLEWIGCIVNGDGSTYFATWAKGRFTITRTSSTTVTLQMTSLTAADTATYFCWRYWALWGPGTLVTVSS

SEQ ID NO: 64 - DNA, 717 bp, Artificial Sequence
  anti-ATM 1561 light chain coding sequence (clone EB0608D-4A10-K1) with leader sequence
atggacacgagggcccccactcagctgctggggctcctgctgctctggctcccaggtgccacatttgccatcgtgatgacccagactccatcttccaagtctgtccctgtgggaggcacagtcaccatcaattgccaggccagtgagagtctttataataacaaccgcttagcctggtttcagcagaaaccagggcagcctcccaagctcctgatctattctgcatccactctggcatctggggtcccagataggttcagcggcagtggatctgggacacagttcactctcaccatcagcgatgtggtgtgtgacgacgctgccacttactactgcgtaggaggtagaagtcgtgttactgatggtattgctttcggcggagggaccgaggcggtggtcaaaggtgatccagttgcacctactgtcctcatcttcccaccagctgctgatcaggtggcaactgaaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 65 - protein, 238 aa, Artificial Sequence
  anti-ATM 1561 light chain amino acid sequence with leader sequence
MDTRAPTQLLGLLLLWLPGATFAIVMTQTPSSKSVPVGGTVTINCQASESLYNNNRLAWFQQKPGQPPKLLIYSASTLASGVPDRFSGSGSGTQFTLTISDVVCDDAATYYCVGGRSRVTDGIAFGGGTEAVVKGDPVAPTVLIFPPAADQVATETVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 66 - protein, 215 aa, Artificial Sequence
  anti-ATM 1561 light chain amino acid sequence without leader sequence
IVMTQTPSSKSVPVGGTVTINCQASESLYNNNRLAWFQQKPGQPPKLLIYSASTLASGVPDRFSGSGSGTQFTLTISDVVCDDAATYYCVGGRSRVTDGIAFGGGTEAVVKGDPVAPTVLIFPPAADQVATETVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 67 - DNA, 402 bp, Artificial Sequence
  anti-ATM 1561 variable light domain coding sequence with leader sequence
atggacacgagggcccccactcagctgctggggctcctgctgctctggctcccaggtgccacatttgccatcgtgatgacccagactccatcttccaagtctgtccctgtgggaggcacagtcaccatcaattgccaggccagtgagagtctttataataacaaccgcttagcctggtttcagcagaaaccagggcagcctcccaagctcctgatctattctgcatccactctggcatctggggtcccagataggttcagcggcagtggatctgggacacagttcactctcaccatcagcgatgtggtgtgtgacgacgctgccacttactactgcgtaggaggtagaagtcgtgttactgatggtattgctttcggcggagggaccgaggcggtggtcaaa

SEQ ID NO: 68 - protein, 134 aa, Artificial Sequence
  anti-ATM 1561 variable light domain amino acid sequence with leader sequence
MDTRAPTQLLGLLLLWLPGATFAIVMTQTPSSKSVPVGGTVTINCQASESLYNNNRLAWFQQKPGQPPKLLIYSASTLASGVPDRFSGSGSGTQFTLTISDVVCDDAATYYCVGGRSRVTDGIAFGGGTEAVVK

SEQ ID NO: 69 - protein, 111 aa, Artificial Sequence
  anti-ATM 1561 variable light domain amino acid sequence without leader sequence
IVMTQTPSSKSVPVGGTVTINCQASESLYNNNRLAWFQQKPGQPPKLLIYSASTLASGVPDRFSGSGSGTQFTLTISDVVCDDAATYYCVGGRSRVTDGIAFGGGTEAVVK

SEQ ID NO: 70 - protein, 5 aa, Artificial Sequence
  anti-CD19 41 VH CDR1
DYSIH

SEQ ID NO: 71 - protein, 17 aa, Artificial Sequence
  anti-CD19 41 VH CDR2
WINTETVEPAYAVDFRG

SEQ ID NO: 72 - protein, 10 aa, Artificial Sequence
  anti-CD19 41 VH CDR3
DYSREGVFDY

SEQ ID NO: 73 - protein, 17 aa, Artificial Sequence
  anti-CD19 41 VL CDR1
KSSQSLFNSRTRKNYLA

SEQ ID NO: 74 - protein, 7 aa, Artificial Sequence
  anti-CD19 41 VL CDR2
WASTRES

SEQ ID NO: 75 - protein, 8 aa, Artificial Sequence
  anti-CD19 41 VL CDR3
KQSYNLPT

SEQ ID NO: 76 - DNA, 414 bp, Artificial Sequence
  anti-CD19 41 variable heavy domain coding sequence with leader sequence
atggcttgggtgttgaccttgctattcctgatggcagctgcccaaagtatccaagcacagatccagttggtgcagtctggacctgagctgaagaagcctggagagacagtcaagatctcctgcaaggcttctgattataccttcacagactattcaatacactgggtgaagcaggctccaggaaagggtttaaagtggctgggctggataaacactgagactgttgagccagcatatgcagttgacttcaggggacggtttgcgctgtctttggaaacctctgccaatactgcctatttgcagatcaacaacctcaaaaatgaggacgcggctacatatttctgtactagagactatagtagagagggcgtctttgactactggggccaaggcaccactctcacagtctcctca

SEQ ID NO: 77 - protein, 138 aa, Artificial Sequence
  anti-CD19 41 variable heavy domain amino acid sequence with leader sequence
MAWVLTLLFLMAAAQSIQAQIQLVQSGPELKKPGETVKISCKASDYTFTDYSIHWVKQAPGKGLKWLGWINTETVEPAYAVDFRGRFALSLETSANTAYLQINNLKNEDAATYFCTRDYSREGVFDYWGQGTTLTVSS

SEQ ID NO: 78 - protein, 119 aa, Artificial Sequence
  anti-CD19 41 variable heavy domain amino acid sequence without leader sequence
QIQLVQSGPELKKPGETVKISCKASDYTFTDYSIHWVKQAPGKGLKWLGWINTETVEPAYAVDFRGRFALSLETSANTAYLQINNLKNEDAATYFCTRDYSREGVFDYWGQGTTLTVSS

SEQ ID NO: 79 - DNA, 396 bp, Artificial Sequence
  anti-CD19 41 variable light domain coding sequence with leader sequence
atggattcacaggcccaggttcttatattgctgctgctatgggtatctggtacctgtggggacaatgtgatgtcacagtctccttcctccctggctgtgtcagcaggagagaaggtcactatgagctgcaaatccagtcagagtctattcaacagtagaacccgaaagaactacttggcttggtaccagcagaaaccagggcagtctcctaaacttctggtctactgggcatccactagggaatctggggtccctgatcgcttcacaggcagtgggtctgggacagatttcactctcaccatcagcaatgtgcaggctgaagacctggcagtttattattgtaagcaatcctataatcttcccacgttcggtgctgggaccaagctggagctgaaa

SEQ ID NO: 80 - protein, 132 aa, Artificial Sequence
  anti-CD19 41 variable light domain amino acid sequence with leader sequence
MDSQAQVLILLLLWVSGTCGDNVMSQSPSSLAVSAGEKVTMSCKSSQSLFNSRTRKNYLAWYQQKPGQSPKLLVYWASTRESGVPDRFTGSGSGTDFTLTISNVQAEDLAVYYCKQSYNLPTFGAGTKLELK

SEQ ID NO: 81 - protein, 112 aa, Artificial Sequence
  anti-CD19 41 variable light domain amino acid sequence without leader sequence
DNVMSQSPSSLAVSAGEKVTMSCKSSQSLFNSRTRKNYLAWYQQKPGQSPKLLVYWASTRESGVPDRFTGSGSGTDFTLTISNVQAEDLAVYYCKQSYNLPTFGAGTKLELK

SEQ ID NO: 82 - protein, 6 aa, Artificial Sequence
  anti-ADA 93 VH CDR1
NSDWEC

SEQ ID NO: 83 - protein, 18 aa, Artificial Sequence
  anti-ADA 93 VH CDR2
CIYGDSRSGYYASWAKGR

SEQ ID NO: 84 - protein, 5 aa, Artificial Sequence
  anti-ADA 93 VH CDR3
NLGSL

SEQ ID NO: 85 - protein, 13 aa, Artificial Sequence
  anti-ADA 93 VL CDR1
QSSQNVVFNNRLA

SEQ ID NO: 86 - protein, 7 aa, Artificial Sequence
  anti-ADA 93 VL CDR2
QASTLAS

SEQ ID NO: 87 - protein, 13 aa, Artificial Sequence
  anti-ADA 93 VL CDR3
LGSYDCSSTDCNT

SEQ ID NO: 88 - DNA, 1371 bp, Artificial Sequence
  anti-ADA 93 heavy chain coding sequence (clone EB0608A-3C12-H2) with leader sequence
atggagactgggctgcgctggcttctcctggtcgctgtgctcaagggtgtccagtgtcaggagcagctggaggagtccgggggagacctggtcaagcctgggtcatccctgacactcacctgcacagcctctggattctccttcactaacagcgactgggaatgctgggtccgccaggctccagggaaggggctggagtggatcgcatgcatttatggtgatagtcgtagcggatactacgcgagctgggcgaagggccgattcaccatctccaaaacctcgtcgaccacggtgactctgcaaatgaccactctgacagccgcggacacggccacctatttctgtgcgagaaatctcggtagcttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 89 - protein, 456 aa, Artificial Sequence
  anti-ADA 93 heavy chain amino acid sequence with leader sequence
METGLRWLLLVAVLKGVQCQEQLEESGGDLVKPGSSLTLTCTASGFSFTNSDWECWVRQAPGKGLEWIACIYGDSRSGYYASWAKGRFTISKTSSTTVTLQMTTLTAADTATYFCARNLGSLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 90 - protein, 437 aa, Artificial Sequence
  anti-ADA 93 heavy chain amino acid sequence without leader sequence
QEQLEESGGDLVKPGSSLTLTCTASGFSFTNSDWECWVRQAPGKGLEWIACIYGDSRSGYYASWAKGRFTISKTSSTTVTLQMTTLTAADTATYFCARNLGSLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 91 - DNA, 399 bp, Artificial Sequence
  anti-ADA 93 variable heavy domain coding sequence with leader sequence
atggagactgggctgcgctggcttctcctggtcgctgtgctcaagggtgtccagtgtcaggagcagctggaggagtccgggggagacctggtcaagcctgggtcatccctgacactcacctgcacagcctctggattctccttcactaacagcgactgggaatgctgggtccgccaggctccagggaaggggctggagtggatcgcatgcatttatggtgatagtcgtagcggatactacgcgagctgggcgaagggccgattcaccatctccaaaacctcgtcgaccacggtgactctgcaaatgaccactctgacagccgcggacacggccacctatttctgtgcgagaaatctcggtagcttgtggggcccaggcaccctggtcaccgtctcctca

SEQ ID NO: 92 - protein, 133 aa, Artificial Sequence
  anti-ADA 93 variable heavy domain amino acid sequence with leader sequence
METGLRWLLLVAVLKGVQCQEQLEESGGDLVKPGSSLTLTCTASGFSFTNSDWECWVRQAPGKGLEWIACIYGDSRSGYYASWAKGRFTISKTSSTTVTLQMTTLTAADTATYFCARNLGSLWGPGTLVTVSS

SEQ ID NO: 93 - protein, 114 aa, Artificial Sequence
  anti-ADA 93 variable heavy domain amino acid sequence without leader sequence
QEQLEESGGDLVKPGSSLTLTCTASGFSFTNSDWECWVRQAPGKGLEWIACIYGDSRSGYYASWAKGRFTISKTSSTTVTLQMTTLTAADTATYFCARNLGSLWGPGTLVTVSS

SEQ ID NO: 94 - DNA, 720 bp, Artificial Sequence
  anti-ADA 93 light chain coding sequence (clone EB0608A-3C12-K2) with leader sequence
atggacacgagggcccccactcagctgctggggctcctgctgctctggctcccaggtgccacatttgcccaagtgctgacccagactccatcctccgtgtctgcggctgtgggaggcacagtcaccatcaactgccagtccagtcagaatgttgtttttaacaaccgcttagcctggtttcagcagaaaccagggcagcctcccaaactcctgatctaccaggcatccactctggcatctggggtcccaccgcggttcaaaggcagtggatctgggacacagttcactctcaccattagcgacctggagtgtgacgatgctgccacttactactgtctaggcagttatgattgtagtagtactgattgtaatactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 95 - protein, 239 aa, Artificial Sequence
  anti-ADA 93 light chain amino acid sequence with leader sequence
MDTRAPTQLLGLLLLWLPGATFAQVLTQTPSSVSAAVGGTVTINCQSSQNVVFNNRLAWFQQKPGQPPKLLIYQASTLASGVPPRFKGSGSGTQFTLTISDLECDDAATYYCLGSYDCSSTDCNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 96 - protein, 216 aa, Artificial Sequence
  anti-ADA 93 light chain amino acid sequence without leader sequence
QVLTQTPSSVSAAVGGTVTINCQSSQNVVFNNRLAWFQQKPGQPPKLLIYQASTLASGVPPRFKGSGSGTQFTLTISDLECDDAATYYCLGSYDCSSTDCNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 97 - DNA, 405 bp, Artificial Sequence
  anti-ADA 93 variable light domain coding sequence with leader sequence
atggacacgagggcccccactcagctgctggggctcctgctgctctggctcccaggtgccacatttgcccaagtgctgacccagactccatcctccgtgtctgcggctgtgggaggcacagtcaccatcaactgccagtccagtcagaatgttgtttttaacaaccgcttagcctggtttcagcagaaaccagggcagcctcccaaactcctgatctaccaggcatccactctggcatctggggtcccaccgcggttcaaaggcagtggatctgggacacagttcactctcaccattagcgacctggagtgtgacgatgctgccacttactactgtctaggcagttatgattgtagtagtactgattgtaatactttcggcggagggaccgaggtggtggtcaaa

SEQ ID NO: 98 - protein, 135 aa, Artificial Sequence
  anti-ADA 93 variable light domain amino acid sequence with leader sequence
MDTRAPTQLLGLLLLWLPGATFAQVLTQTPSSVSAAVGGTVTINCQSSQNVVFNNRLAWFQQKPGQPPKLLIYQASTLASGVPPRFKGSGSGTQFTLTISDLECDDAATYYCLGSYDCSSTDCNTFGGGTEVVVK

SEQ ID NO: 99 - protein, 112 aa, Artificial Sequence
  anti-ADA 93 variable light domain amino acid sequence without leader sequence
QVLTQTPSSVSAAVGGTVTINCQSSQNVVFNNRLAWFQQKPGQPPKLLIYQASTLASGVPPRFKGSGSGTQFTLTISDLECDDAATYYCLGSYDCSSTDCNTFGGGTEVVVK

SEQ ID NO: 100 - protein, 5 aa, Artificial Sequence
  anti-DOCK8 1272 VH CDR1
SYWMH

SEQ ID NO: 101 - protein, 17 aa, Artificial Sequence
  anti-DOCK8 1272 VH CDR2
AIFPGNSNTRYNQKFKG

SEQ ID NO: 102 - protein, 6 aa, Artificial Sequence
  anti-DOCK8 1272 VH CDR3
DNAFDY

SEQ ID NO: 103 - protein, 12 aa, Artificial Sequence
  anti-DOCK8 1272 VL CDR1
SASSSVSSRYLH

SEQ ID NO: 104 - protein, 7 aa, Artificial Sequence
  anti-DOCK8 1272 VL CDR2
GTSNLSS

SEQ ID NO: 105 - protein, 9 aa, Artificial Sequence
  anti-DOCK8 1272 VL CDR3
QQYHSDPFT

SEQ ID NO: 106 - DNA, 402 bp, Artificial Sequence
  anti-DOCK8 1272 variable heavy domain coding sequence (Hybridoma SJSH5-1C2-2E) with leader sequence
atggaatctaactggatacttccttttattctgtcggtaacttcaggggtctactcagaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttttcctggaaatagtaatactagatacaaccagaagttcaagggcaaggccaaactgactgcagtcacatctgccagcactgcctacatggagctcagcagcctgacaaatgaggactctgcggtctattactgtgccgatgataacgcctttgactactggggccaaggcaccactctcacagtctcctca

SEQ ID NO: 107 - protein, 134 aa, Artificial Sequence
  anti-DOCK8 1272 variable heavy domain amino acid sequence with leader sequence
MESNWILPFILSVTSGVYSEVQLQQSGTVLARPGASVKMSCKASGYTFTSYWMHWVKQRPGQGLEWIGAIFPGNSNTRYNQKFKGKAKLTAVTSASTAYMELSSLTNEDSAVYYCADDNAFDYWGQGTTLTVSS

SEQ ID NO: 108 - protein, 115 aa, Artificial Sequence
  anti-DOCK8 1272 variable heavy domain amino acid sequence without leader sequence
EVQLQQSGTVLARPGASVKMSCKASGYTFTSYWMHWVKQRPGQGLEWIGAIFPGNSNTRYNQKFKGKAKLTAVTSASTAYMELSSLTNEDSAVYYCADDNAFDYWGQGTTLTVSS

SEQ ID NO: 109 - DNA, 390 bp, Artificial Sequence
  anti-DOCK8 1272 variable light domain coding sequence (Hybridoma SJSH5-1C2-2E) with leader sequence
atggattttcaagtgcagattttcagcttcttgttgatcagtgcctcagtcataatgaccagaggacaaattgttctcacccagtctccagcaatcatgtctgcctctccaggggaaaaggtcaccatgacctgcagtgccagctcaagtgtaagttccaggtacttgcactggtaccagcagaagtcaggagcctcccccaaactctggatttatggcacatccaacctgtcttctggagtccctgatcgcttcagtggcagtgggtctgggacctcttactctctcacaatcagcagcgtggagactgaagatgctgccacttattactgccagcagtatcatagtgacccattcacgttcggcacggggacaaaattggaaataaaa

SEQ ID NO: 110 - protein, 130 aa, Artificial Sequence
  anti-DOCK8 1272 variable light domain amino acid sequence with leader sequence
MDFQVQIFSFLLISASVIMTRGQIVLTQSPAIMSASPGEKVTMTCSASSSVSSRYLHWYQQKSGASPKLWIYGTSNLSSGVPDRFSGSGSGTSYSLTISSVETEDAATYYCQQYHSDPFTFGTGTKLEIK

SEQ ID NO: 111 - protein, 108 aa, Artificial Sequence
  anti-DOCK8 1272 variable light domain amino acid sequence without leader sequence
QIVLTQSPAIMSASPGEKVTMTCSASSSVSSRYLHWYQQKSGASPKLWIYGTSNLSSGVPDRFSGSGSGTSYSLTISSVETEDAATYYCQQYHSDPFTFGTGTKLEIK

SEQ ID NO: 112 - protein, 5 aa, Artificial Sequence
  anti-CD42 128 VH CDR1
SYNIH

SEQ ID NO: 113 - protein, 17 aa, Artificial Sequence
  anti-CD42 128 VH CDR2
GIYPGNGDTSYNKKFKD

SEQ ID NO: 114 - protein, 5 aa, Artificial Sequence
  anti-CD42 128 VH CDR3
DGTRY

SEQ ID NO: 115 - protein, 16 aa, Artificial Sequence
  anti-CD42 128 VL CDR1
TSSQSLLASDGKTYLN

SEQ ID NO: 116 - protein, 7 aa, Artificial Sequence
  anti-CD42 128 VL CDR2
LVSKLDS

SEQ ID NO: 117 - protein, 9 aa, Artificial Sequence
  anti-CD42 128 VL CDR3
WQGAHSPLT

SEQ ID NO: 118 - DNA, 399 bp, Artificial Sequence
  anti-CD42 128 variable heavy domain coding sequence (Hybridoma SJSH4 3B8-1b) with leader sequence
atgggattcagcaggatttttctcttcctcctgtcagtaactacaggtgtccactcccaggcttatctacagcagtctggggctgttctggtgaggcctggggcctcagtgaagatgtcctgcaaggcttctggctacacattttccagttacaatatacactgggtaaagcagacacctagacagggcctggaatggattggaggtatttatccaggaaatggtgacacttcctacaataagaagttcaaggacaaggccacactgactgttgacaaatcctccaacaccgcctacatgcacctcagcagcctgacatctgaagactctgcggtctttttctgtgcaagcgacggtactaggtactggggccaaggcaccactctcacagtctcctca

SEQ ID NO: 119 - protein, 133 aa, Artificial Sequence
  anti-CD42 128 variable heavy domain amino acid sequence with leader sequence
MGFSRIFLFLLSVTTGVHSQAYLQQSGAVLVRPGASVKMSCKASGYTFSSYNIHWVKQTPRQGLEWIGGIYPGNGDTSYNKKFKDKATLTVDKSSNTAYMHLSSLTSEDSAVFFCASDGTRYWGQGTTLTVSS

SEQ ID NO: 120 - protein, 114 aa, Artificial Sequence
  anti-CD42 128 variable heavy domain amino acid sequence without leader sequence
QAYLQQSGAVLVRPGASVKMSCKASGYTFSSYNIHWVKQTPRQGLEWIGGIYPGNGDTSYNKKFKDKATLTVDKSSNTAYMHLSSLTSEDSAVFFCASDGTRYWGQGTTLTVSS

SEQ ID NO: 121 - DNA, 393 bp, Artificial Sequence
  anti-CD42 128 variable light domain coding sequence (Hybridoma SJSH4 3B8-1b) with leader sequence
atgagtcctgcccagttcctgtttctgttagtgctctggattcgggacgccaacggtgaagttgtgatgacccagactccactcactttgtcggttaccattggacaagcagtctccatttcttgtacgtcaagtcagagcctcttagctagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgacgatttgggagtttattgttgctggcaaggtgcacattctccgctcacgttcggtgctgggaccaggctggaactgaaa

SEQ ID NO: 122 - protein, 131 aa, Artificial Sequence
  anti-CD42 128 variable light domain amino acid sequence with leader sequence
MSPAQFLFLLVLWIRDANGEVVMTQTPLTLSVTIGQAVSISCTSSQSLLASDGKTYLNWLLQRPGQSPKRLIYLVSKLDSGVPDRFTGSGSGTDFTLKISRVEADDLGVYCCWQGAHSPLTFGAGTRLELK

SEQ ID NO: 123 - protein, 112 aa, Artificial Sequence
  anti-CD42 128 variable light domain amino acid sequence without leader sequence
EVVMTQTPLTLSVTIGQAVSISCTSSQSLLASDGKTYLNWLLQRPGQSPKRLIYLVSKLDSGVPDRFTGSGSGTDFTLKISRVEADDLGVYCCWQGAHSPLTFGAGTRLELK

SEQ ID NO: 124 - protein, 5 aa, Artificial Sequence
  anti-CD42 154 VH CDR1
GYNMH

SEQ ID NO: 125 - protein, 17 aa, Artificial Sequence
  anti-CD42 154 VH CDR2
DINPKTGENSYNQKFKG

SEQ ID NO: 126 - protein, 8 aa, Artificial Sequence
  anti-CD42 154 VH CDR3
WGVYYLDH

SEQ ID NO: 127 - protein, 16 aa, Artificial Sequence
  anti-CD42 154 VL CDR1
RSTKSLLYSDGITYLY

SEQ ID NO: 128 - protein, 7 aa, Artificial Sequence
  anti-CD42 154 VL CDR2
RMSDLAS

SEQ ID NO: 129 - protein, 9 aa, Artificial Sequence
  anti-CD42 154 VL CDR3
AQMVEFPPT

SEQ ID NO: 130 - DNA, 408 bp, Artificial Sequence
  anti-CD42 154 variable heavy domain coding sequence (Hybridoma SJSH4-1H11c) with leader sequence
atgggatggagctggatctttctctttctcctgtcagaaactgcaggtgtcctctctgaggtccagctgcaacagtctggacctgagctggtgaagcctggggcttcagtgaagatgtcctgtaaggcttctggatacacattcactggctacaatatgcactgggtgaagcagagccatggaaagagccttgagtggattggagacattaatcctaagactggtgaaaatagttataaccagaagttcaagggcaaggccacattgacgataagcaagtcctccagcacagcctacatggagctccgcagtctgacatcggaagattctgcagtctattactgtgtaagatggggtgtatattatttagaccactggggccaaggcaccactctcacggtctcctca

SEQ ID NO: 131 - protein, 136 aa, Artificial Sequence
  anti-CD42 154 variable heavy domain amino acid sequence with leader sequence
MGWSWIFLFLLSETAGVLSEVQLQQSGPELVKPGASVKMSCKASGYTFTGYNMHWVKQSHGKSLEWIGDINPKTGENSYNQKFKGKATLTISKSSSTAYMELRSLTSEDSAVYYCVRWGVYYLDHWGQGTTLTVSS

SEQ ID NO: 132 - protein, 117 aa, Artificial Sequence
  anti-CD42 154 variable heavy domain amino acid sequence without leader sequence
EVQLQQSGPELVKPGASVKMSCKASGYTFTGYNMHWVKQSHGKSLEWIGDINPKTGENSYNQKFKGKATLTISKSSSTAYMELRSLTSEDSAVYYCVRWGVYYLDHWGQGTTLTVSS

SEQ ID NO: 133 - DNA, 396 bp, Artificial Sequence
  anti-CD42 154 variable light domain coding sequence (Hybridoma SJSH4-1H11c) with leader sequence
atgaggttctctgctcagcttctggggctgcttgtgctctggatccctggatccactgcagatattgtgatgacgcaggctgcattctccaatccagtcactcttggaacgtcagcttccatctcctgcaggtctactaagagtctcctatatagtgatggcatcacttatttgtattggtatctgcagaggccaggccagtctcctcagctcctgatatatcggatgtccgaccttgcctcaggagtcccagacaggttcagtggcagtgggtcaggaactgatttcacactgagaatcagcagagtggaggctgaggatgtgggtgtttattactgtgctcaaatggtagaattccctccgacgttcggtggaggcaccaagctggaaatcaaa

SEQ ID NO: 134 - protein, 132 aa, Artificial Sequence
  anti-CD42 154 variable light domain amino acid sequence with leader sequence
MRFSAQLLGLLVLWIPGSTADIVMTQAAFSNPVTLGTSASISCRSTKSLLYSDGITYLYWYLQRPGQSPQLLIYRMSDLASGVPDRFSGSGSGTDFTLRISRVEAEDVGVYYCAQMVEFPPTFGGGTKLEIK

SEQ ID NO: 135 - protein, 112 aa, Artificial Sequence
  anti-CD42 154 variable light domain amino acid sequence without leader sequence
DIVMTQAAFSNPVTLGTSASISCRSTKSLLYSDGITYLYWYLQRPGQSPQLLIYRMSDLASGVPDRFSGSGSGTDFTLRISRVEAEDVGVYYCAQMVEFPPTFGGGTKLEIK

SEQ ID NO: 136 - protein, 6 aa, Artificial Sequence
  anti-CD56 122 VH CDR1
SNYYMC

SEQ ID NO: 137 - protein, 18 aa, Artificial Sequence
  anti-CD56 122 VH CDR2
CIYSSSLNTWYASWAKGR

SEQ ID NO: 138 - protein, 16 aa, Artificial Sequence
  anti-CD56 122 VH CDR3
GGGVYVGADYGFYFYL

SEQ ID NO: 139 - protein, 11 aa, Artificial Sequence
  anti-CD56 122 VL CDR1
QASQSIDSWCA

SEQ ID NO: 140 - protein, 7 aa, Artificial Sequence
  anti-CD56 122 VL CDR2
KASTLES

SEQ ID NO: 141 - protein, 12 aa, Artificial Sequence
  anti-CD56 122 VL CDR3
QSYYPTTGSDNA

SEQ ID NO: 142 - DNA, 1404 bp, Artificial Sequence
  anti-CD56 122  heavy chain coding sequence (clone EB0608C-3H4-H2) with leader sequence
atggagactgggctgcgctggcttctcctggtcgctgtgctcaaaggtgtccagtgtcagtcgttggaggagtccgggggagacctggtcaagcctggggcatccctgacactcacctgcaaagcctctgggttagacttcagtagcaactactacatgtgctgggtccgccaggctccagggaaggggctggagtggatcgcatgcatttatagtagtagtcttaacacatggtacgcgagctgggcgaaaggccgattcaccatctccaaaagcaccagcctacacacggtgactctgcaaatgaccagtctgacagccgcggacacggccacctatttctgtacgagagggggtggtgtttatgttggtgctgattatggtttttacttctacttatggggcccaggcaccctggtcaccgtctcctccgggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 143 - protein, 467 aa, Artificial Sequence
  anti-CD56 122 heavy chain amino acid sequence with leader sequence
METGLRWLLLVAVLKGVQCQSLEESGGDLVKPGASLTLTCKASGLDFSSNYYMCWVRQAPGKGLEWIACIYSSSLNTWYASWAKGRFTISKSTSLHTVTLQMTSLTAADTATYFCTRGGGVYVGADYGFYFYLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 144 - protein, 448 aa, Artificial Sequence
  anti-CD56 122 heavy chain amino acid sequence without leader sequence
QSLEESGGDLVKPGASLTLTCKASGLDFSSNYYMCWVRQAPGKGLEWIACIYSSSLNTWYASWAKGRFTISKSTSLHTVTLQMTSLTAADTATYFCTRGGGVYVGADYGFYFYLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 145 - DNA, 432 bp, Artificial Sequence
  anti-CD56 122 variable heavy domain coding sequence with leader sequence
atggagactgggctgcgctggcttctcctggtcgctgtgctcaaaggtgtccagtgtcagtcgttggaggagtccgggggagacctggtcaagcctggggcatccctgacactcacctgcaaagcctctgggttagacttcagtagcaactactacatgtgctgggtccgccaggctccagggaaggggctggagtggatcgcatgcatttatagtagtagtcttaacacatggtacgcgagctgggcgaaaggccgattcaccatctccaaaagcaccagcctacacacggtgactctgcaaatgaccagtctgacagccgcggacacggccacctatttctgtacgagagggggtggtgtttatgttggtgctgattatggtttttacttctacttatggggcccaggcaccctggtcaccgtctcctcc

SEQ ID NO: 146 - protein, 144 aa, Artificial Sequence
  anti-CD56 122 variable heavy domain amino acid sequence with leader sequence
METGLRWLLLVAVLKGVQCQSLEESGGDLVKPGASLTLTCKASGLDFSSNYYMCWVRQAPGKGLEWIACIYSSSLNTWYASWAKGRFTISKSTSLHTVTLQMTSLTAADTATYFCTRGGGVYVGADYGFYFYLWGPGTLVTVSS

SEQ ID NO: 147 - protein, 125 aa, Artificial Sequence
  anti-CD56 122 variable heavy domain amino acid sequence without leader sequence
QSLEESGGDLVKPGASLTLTCKASGLDFSSNYYMCWVRQAPGKGLEWIACIYSSSLNTWYASWAKGRFTISKSTSLHTVTLQMTSLTAADTATYFCTRGGGVYVGADYGFYFYLWGPGTLVTVSS

SEQ ID NO: 148 - DNA, 714 bp, Artificial Sequence
  anti-CD56 122 light chain coding sequence (clone EB0608C-3H4-K2) with leader sequence
atggacacgagggcccccactcagctgctggggctcctactgctctggctcccaggtgccagatgtgctgacattgtgatgacccagactccatcctccgtggaggcacctgtgggaggcacagtcaccatcaagtgccaggccagtcagagcattgatagttggtgtgcctggtatcagcagaaaccagggcagcctcccaagctcctgatctacaaggcatccactctggaatctggggtcccatcgcggttcaaaggcagtggatctgggacagagttcactctcaccatcagcgacctggagtgtgccgatgctgccacttacttctgtcaaagctattatcctactactggtagtgataatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 149 - protein, 237 aa, Artificial Sequence
  anti-CD56 122 light chain amino acid sequence with leader sequence
MDTRAPTQLLGLLLLWLPGARCADIVMTQTPSSVEAPVGGTVTIKCQASQSIDSWCAWYQQKPGQPPKLLIYKASTLESGVPSRFKGSGSGTEFTLTISDLECADAATYFCQSYYPTTGSDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 150 - protein, 215 aa, Artificial Sequence
  anti-CD56 122 light chain amino acid sequence without leader sequence
ADIVMTQTPSSVEAPVGGTVTIKCQASQSIDSWCAWYQQKPGQPPKLLIYKASTLESGVPSRFKGSGSGTEFTLTISDLECADAATYFCQSYYPTTGSDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 151 - DNA, 399 bp, Artificial Sequence
  anti-CD56 122 variable light domain coding sequence with leader sequence
atggacacgagggcccccactcagctgctggggctcctactgctctggctcccaggtgccagatgtgctgacattgtgatgacccagactccatcctccgtggaggcacctgtgggaggcacagtcaccatcaagtgccaggccagtcagagcattgatagttggtgtgcctggtatcagcagaaaccagggcagcctcccaagctcctgatctacaaggcatccactctggaatctggggtcccatcgcggttcaaaggcagtggatctgggacagagttcactctcaccatcagcgacctggagtgtgccgatgctgccacttacttctgtcaaagctattatcctactactggtagtgataatgctttcggcggagggaccgaggtggtggtcaaa

SEQ ID NO: 152 - protein, 133 aa, Artificial Sequence
  anti-CD56 122 variable light domain amino acid sequence with leader sequence
MDTRAPTQLLGLLLLWLPGARCADIVMTQTPSSVEAPVGGTVTIKCQASQSIDSWCAWYQQKPGQPPKLLIYKASTLESGVPSRFKGSGSGTEFTLTISDLECADAATYFCQSYYPTTGSDNAFGGGTEVVVK

SEQ ID NO: 153 - protein, 111 aa, Artificial Sequence
  anti-CD56 122 variable light domain amino acid sequence without leader sequence
ADIVMTQTPSSVEAPVGGTVTIKCQASQSIDSWCAWYQQKPGQPPKLLIYKASTLESGVPSRFKGSGSGTEFTLTISDLECADAATYFCQSYYPTTGSDNAFGGGTEVVVK

SEQ ID NO: 154 - protein, 11 aa, Artificial Sequence
  linker
GGSGGGSGGSG

SEQ ID NO: 155 - protein, 10 aa, Artificial Sequence
  linker
GGSGGGSGSG

SEQ ID NO: 156 - protein, 8 aa, Artificial Sequence
  linker
GGSGGGSG

SEQ ID NO: 157 - protein, 10 aa, Artificial Sequence
  linker
GGGGSGGGGS

SEQ ID NO: 158 - protein, 8 aa, Artificial Sequence
  linker
GGGSGGGS

SEQ ID NO: 159 - protein, 6 aa, Artificial Sequence
  linker
GGSGGS

SEQ ID NO: 160 - protein, 5 aa, Artificial Sequence
  anti-SH2D1A (SAP) 19  VH CDR1
SYDMS

SEQ ID NO: 161 - protein, 16 aa, Artificial Sequence
  anti-SH2D1A (SAP) 19  VH CDR2
FGNTGGSTYYASWAKG

SEQ ID NO: 162 - protein, 10 aa, Artificial Sequence
  anti-SH2D1A (SAP) 19  VH CDR3
GAPGWITTNM

SEQ ID NO: 163 - protein, 13 aa, Artificial Sequence
  anti-SH2D1A (SAP) 19  VL CDR1
QSSQSVYRNKCLA

SEQ ID NO: 164 - protein, 7 aa, Artificial Sequence
  anti-SH2D1A (SAP) 19  VL CDR2
KASTLAS

SEQ ID NO: 165 - protein, 12 aa, Artificial Sequence
  anti-SH2D1A (SAP) 19  VL CDR3
VGYYNTNTNTTT

SEQ ID NO: 166 - DNA, 1374 bp, Artificial Sequence
  anti-SH2D1A (SAP) 19 heavy chain coding sequence (clone EB0612B -2F4-H1) with leader sequence
atggagactgggctgcgctggcttctcctggtcgctgtgctcaaaggtgtccagtgtcagtcgctggaggagtccgggggtcgcctggtcacgcctggaggaaccctgacactcacctgcacagtctctggattctccctcagcagctacgacatgagctgggtccgccaggctccagggaaggggctggaatggatcggatttggtaatactggtggtagcacatactacgcgagctgggcaaaaggccgattcaccatctccagaacctcgaccacggtggatctgagaatgaccagtctgacagccgcggacacggccacctatttctgtaccagaggggcccctggttggattactactaatatgtggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 167 - DNA, 1317 bp, Artificial Sequence
  anti-SH2D1A (SAP) 19 heavy chain coding sequence (clone EB0612B -2F4-H1) without leader sequence
cagtcgctggaggagtccgggggtcgcctggtcacgcctggaggaaccctgacactcacctgcacagtctctggattctccctcagcagctacgacatgagctgggtccgccaggctccagggaaggggctggaatggatcggatttggtaatactggtggtagcacatactacgcgagctgggcaaaaggccgattcaccatctccagaacctcgaccacggtggatctgagaatgaccagtctgacagccgcggacacggccacctatttctgtaccagaggggcccctggttggattactactaatatgtggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 168 - protein, 457 aa, Artificial Sequence
  anti-SH2D1A (SAP) 19 heavy chain amino acid sequence with leader sequence
METGLRWLLLVAVLKGVQCQSLEESGGRLVTPGGTLTLTCTVSGFSLSSYDMSWVRQAPGKGLEWIGFGNTGGSTYYASWAKGRFTISRTSTTVDLRMTSLTAADTATYFCTRGAPGWITTNMWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 169 - protein, 438 aa, Artificial Sequence
  anti-SH2D1A (SAP) 19 heavy chain amino acid sequence without leader sequence
QSLEESGGRLVTPGGTLTLTCTVSGFSLSSYDMSWVRQAPGKGLEWIGFGNTGGSTYYASWAKGRFTISRTSTTVDLRMTSLTAADTATYFCTRGAPGWITTNMWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 170 - DNA, 402 bp, Artificial Sequence
  anti-SH2D1A (SAP) 19 variable heavy domain coding sequence with leader sequence
atggagactgggctgcgctggcttctcctggtcgctgtgctcaaaggtgtccagtgtcagtcgctggaggagtccgggggtcgcctggtcacgcctggaggaaccctgacactcacctgcacagtctctggattctccctcagcagctacgacatgagctgggtccgccaggctccagggaaggggctggaatggatcggatttggtaatactggtggtagcacatactacgcgagctgggcaaaaggccgattcaccatctccagaacctcgaccacggtggatctgagaatgaccagtctgacagccgcggacacggccacctatttctgtaccagaggggcccctggttggattactactaatatgtggggcccaggcaccctggtcaccgtctcctta

SEQ ID NO: 171 - DNA, 345 bp, Artificial Sequence
  anti-SH2D1A (SAP) 19 variable heavy domain coding sequence without leader sequence
cagtcgctggaggagtccgggggtcgcctggtcacgcctggaggaaccctgacactcacctgcacagtctctggattctccctcagcagctacgacatgagctgggtccgccaggctccagggaaggggctggaatggatcggatttggtaatactggtggtagcacatactacgcgagctgggcaaaaggccgattcaccatctccagaacctcgaccacggtggatctgagaatgaccagtctgacagccgcggacacggccacctatttctgtaccagaggggcccctggttggattactactaatatgtggggcccaggcaccctggtcaccgtctcctta

SEQ ID NO: 172 - protein, 134 aa, Artificial Sequence
  anti-SH2D1A (SAP) 19 variable heavy domain amino acid sequence with leader sequence
METGLRWLLLVAVLKGVQCQSLEESGGRLVTPGGTLTLTCTVSGFSLSSYDMSWVRQAPGKGLEWIGFGNTGGSTYYASWAKGRFTISRTSTTVDLRMTSLTAADTATYFCTRGAPGWITTNMWGPGTLVTVSL

SEQ ID NO: 173 - protein, 115 aa, Artificial Sequence
  anti-SH2D1A (SAP) 19 variable heavy domain amino acid sequence without leader sequence
QSLEESGGRLVTPGGTLTLTCTVSGFSLSSYDMSWVRQAPGKGLEWIGFGNTGGSTYYASWAKGRFTISRTSTTVDLRMTSLTAADTATYFCTRGAPGWITTNMWGPGTLVTVSL

SEQ ID NO: 174 - DNA, 717 bp, Artificial Sequence
  anti-SH2D1A (SAP) 19 light chain coding sequence (clone EB0612B -2F4-K1) with leader sequence
atggacacgagggcccccactcagctgctggggctcctgctgctctggctcccaggtgccacatttgcgcaagtgctgacccagacaccatcccctgtgtctgcagctgtgggaggcacagtcaccatcagttgccagtccagtcagagtgtttatcgtaataagtgcttagcctggtatcagcagaaaccagggcagcctcccaagctcctgatctacaaggcatccactctggcatctggggtcccatcgcggttcagcggcagtggatctgggacacagttcactttcaccatcagcgacctggagtgtgacgattctgccacttactactgtgtaggctattataatactaatactaatacaactactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 175 - DNA, 648 bp, Artificial Sequence
  anti-SH2D1A (SAP) 19 light chain coding sequence (clone EB0612B -2F4-K1) without leader sequence
caagtgctgacccagacaccatcccctgtgtctgcagctgtgggaggcacagtcaccatcagttgccagtccagtcagagtgtttatcgtaataagtgcttagcctggtatcagcagaaaccagggcagcctcccaagctcctgatctacaaggcatccactctggcatctggggtcccatcgcggttcagcggcagtggatctgggacacagttcactttcaccatcagcgacctggagtgtgacgattctgccacttactactgtgtaggctattataatactaatactaatacaactactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 176 - protein, 238 aa, Artificial Sequence
  anti-SH2D1A (SAP) 19 light chain amino acid sequence with leader sequence
MDTRAPTQLLGLLLLWLPGATFAQVLTQTPSPVSAAVGGTVTISCQSSQSVYRNKCLAWYQQKPGQPPKLLIYKASTLASGVPSRFSGSGSGTQFTFTISDLECDDSATYYCVGYYNTNTNTTTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 177 - protein, 215 aa, Artificial Sequence
  anti-SH2D1A (SAP) 19 light chain amino acid sequence without leader sequence
QVLTQTPSPVSAAVGGTVTISCQSSQSVYRNKCLAWYQQKPGQPPKLLIYKASTLASGVPSRFSGSGSGTQFTFTISDLECDDSATYYCVGYYNTNTNTTTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 178 - DNA, 402 bp, Artificial Sequence
  anti-SH2D1A (SAP) 19 variable light domain coding sequence with leader sequence
atggacacgagggcccccactcagctgctggggctcctgctgctctggctcccaggtgccacatttgcgcaagtgctgacccagacaccatcccctgtgtctgcagctgtgggaggcacagtcaccatcagttgccagtccagtcagagtgtttatcgtaataagtgcttagcctggtatcagcagaaaccagggcagcctcccaagctcctgatctacaaggcatccactctggcatctggggtcccatcgcggttcagcggcagtggatctgggacacagttcactttcaccatcagcgacctggagtgtgacgattctgccacttactactgtgtaggctattataatactaatactaatacaactactttcggcggagggaccgaggtggtggtcaaa

SEQ ID NO: 179 - DNA, 333 bp, Artificial Sequence
  anti-SH2D1A (SAP) 19 variable light domain coding sequence without leader sequence
caagtgctgacccagacaccatcccctgtgtctgcagctgtgggaggcacagtcaccatcagttgccagtccagtcagagtgtttatcgtaataagtgcttagcctggtatcagcagaaaccagggcagcctcccaagctcctgatctacaaggcatccactctggcatctggggtcccatcgcggttcagcggcagtggatctgggacacagttcactttcaccatcagcgacctggagtgtgacgattctgccacttactactgtgtaggctattataatactaatactaatacaactactttcggcggagggaccgaggtggtggtcaaa

SEQ ID NO: 180 - protein, 134 aa, Artificial Sequence
  anti-SH2D1A (SAP) 19 variable light domain amino acid sequence with leader sequence
MDTRAPTQLLGLLLLWLPGATFAQVLTQTPSPVSAAVGGTVTISCQSSQSVYRNKCLAWYQQKPGQPPKLLIYKASTLASGVPSRFSGSGSGTQFTFTISDLECDDSATYYCVGYYNTNTNTTTFGGGTEVVVK

SEQ ID NO: 181 - protein, 111 aa, Artificial Sequence
  anti-SH2D1A (SAP) 19 variable light domain amino acid sequence without  leader sequence
QVLTQTPSPVSAAVGGTVTISCQSSQSVYRNKCLAWYQQKPGQPPKLLIYKASTLASGVPSRFSGSGSGTQFTFTISDLECDDSATYYCVGYYNTNTNTTTFGGGTEVVVK

SEQ ID NO: 182 - DNA, 1329 bp, Artificial Sequence
  anti-CYBB 509 heavy chain coding sequence (clone EB0611C -1E12-H2) without leader sequence
cagtcgttggaggagtccgggggaggcctggtcaagcctggggcatccctgacactcacctgcacagcctctggattctccttcagtcgtagttattggatatgctgggtccgccaggctccagggaaggggctggagtggctcggatgcatttatgctggtagtagtggtagcacttactacgcgagctgggcgaaaggccgattcaccatctccaaaacctcgtcgaccacggtgactctacaaatgaccagtctgacagccgcggacacggccacctatttctgtgcgagaggaaataataatattataatcttggatttgtggggcccaggcatactggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 183 - DNA, 357 bp, Artificial Sequence
  anti-CYBB 509 variable heavy domain coding sequence without leader sequence
cagtcgttggaggagtccgggggaggcctggtcaagcctggggcatccctgacactcacctgcacagcctctggattctccttcagtcgtagttattggatatgctgggtccgccaggctccagggaaggggctggagtggctcggatgcatttatgctggtagtagtggtagcacttactacgcgagctgggcgaaaggccgattcaccatctccaaaacctcgtcgaccacggtgactctacaaatgaccagtctgacagccgcggacacggccacctatttctgtgcgagaggaaataataatattataatcttggatttgtggggcccaggcatactggtcaccgtctcctca

SEQ ID NO: 184 - DNA, 645 bp, Artificial Sequence
  anti-CYBB 509 light chain coding sequence (clone EB0611C -1E12-K2) without leader sequence
gccgtgctgacccagactccatctcccgtgtctgcagctgtgggaggcacagtcaccatcagttgccaggccagtcagagtgtttacaataacaatgccttatcctggtatcagcagaaaccagggcagcctcccaagctcctgatctatgatgcatccgatctggcacctggggtcccatcgcggttcagcggcagtggatctgggacacagttcactctcaccatcagcgacgtgcagtgtgacgatgctgccacttactactgtctaggcggttatagtgtgagtagtgataatgctttcggcggagggaccgaggtggtggtcaacggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 185 - DNA, 330 bp, Artificial Sequence
  anti-CYBB 509 variable light domain coding sequence without leader sequence
gccgtgctgacccagactccatctcccgtgtctgcagctgtgggaggcacagtcaccatcagttgccaggccagtcagagtgtttacaataacaatgccttatcctggtatcagcagaaaccagggcagcctcccaagctcctgatctatgatgcatccgatctggcacctggggtcccatcgcggttcagcggcagtggatctgggacacagttcactctcaccatcagcgacgtgcagtgtgacgatgctgccacttactactgtctaggcggttatagtgtgagtagtgataatgctttcggcggagggaccgaggtggtggtcaac

SEQ ID NO: 186 - DNA, 1320 bp, Artificial Sequence
  anti-PRF 215 heavy chain coding sequence (clone EB0610D-9E12-H3) without leader sequence
cagtcggtggaggagtccgggggtcgcctggtaacgcctggaggaaccctgacactcacctgcacagcctctggattctccctcagtaggcacaacatgggctgggtccgccaggctccaggggaggggctggagtggatcggatccattagtagtaagggtaagacatactacacgacctgggcgaaaggtcgattcaccatctccaaaagctcgaccacggtggatctgaaaatcaccagtccgacaaccgaggacacggccacctatttctgtgccagattatattctggtagtaatggttatgaaaccatctggggcccaggcaccctggtcaccgtctccttggggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 187 - DNA, 348 bp, Artificial Sequence
  anti-PRF 215 variable heavy domain coding sequence without leader sequence
cagtcggtggaggagtccgggggtcgcctggtaacgcctggaggaaccctgacactcacctgcacagcctctggattctccctcagtaggcacaacatgggctgggtccgccaggctccaggggaggggctggagtggatcggatccattagtagtaagggtaagacatactacacgacctgggcgaaaggtcgattcaccatctccaaaagctcgaccacggtggatctgaaaatcaccagtccgacaaccgaggacacggccacctatttctgtgccagattatattctggtagtaatggttatgaaaccatctggggcccaggcaccctggtcaccgtctccttg

SEQ ID NO: 188 - DNA, 657 bp, Artificial Sequence
  anti-PRF 215 light chain coding sequence (clone EB0610D-9E12-K1) without leader sequence
  misc_feature (526)..(526): n is a, c, g, or t
gccgatgtcgtgatgacccagactccatcccccgtgtctgcagctgtgggaggcacagtcaccatcaattgccaggccagtcagagtgtttatgataacaacaacttagcctggtatcagcagaaaccagggcagcctcccaagctcctgatctatgatgcatccaaactggcatctggggtcccatcgcggttcagcggcagtggatctgggacacagttcactctcaccatcagcgacgtgcagtgtgacgatgctgccacttactactgtctaggcgaatttatttctagtagtggtgattgtattgttttcggcggagggaccgagatggtcgtcaaaggtgatccagttgcacctactgtcctcatcttcccaccatctgctgatcttgtggcaactggaacagtcaccatcgtgtgtgtggcgaataaatactttcccgatgtcaccgtcacctgggaggtggatggcaccacccaaacaactggcatcgagaacagtaaaacaccgcagaattctgcanattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 189 - DNA, 342 bp, Artificial Sequence
  anti-PRF 215 variable light domain coding sequence without leader sequence
gccgatgtcgtgatgacccagactccatcccccgtgtctgcagctgtgggaggcacagtcaccatcaattgccaggccagtcagagtgtttatgataacaacaacttagcctggtatcagcagaaaccagggcagcctcccaagctcctgatctatgatgcatccaaactggcatctggggtcccatcgcggttcagcggcagtggatctgggacacagttcactctcaccatcagcgacgtgcagtgtgacgatgctgccacttactactgtctaggcgaatttatttctagtagtggtgattgtattgttttcggcggagggaccgagatggtcgtcaaa

SEQ ID NO: 190 - DNA, 1308 bp, Artificial Sequence
  anti-ATM 1561 heavy chain coding sequence (clone EB0608D-4A10-H4) without leader sequence
caggagcagctggtggagtccgggggaggcctggtccagcctgggggatccctgacactcacctgcaaagcctctggattcgacttcagtagcggtggaatgtgctgggtccgccaggctccagggaaggggctggagtggatcggatgcattgtaaatggtgatggtagcacatacttcgcgacctgggcgaaaggccgattcaccatcaccagaacgtcgtcgaccacggtgactctgcaaatgaccagtctgacagccgcggacacggccacctatttctgttggagatattgggccttgtggggcccaggtaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 191 - DNA, 336 bp, Artificial Sequence
  anti-ATM 1561 variable heavy domain coding sequence without leader sequence
caggagcagctggtggagtccgggggaggcctggtccagcctgggggatccctgacactcacctgcaaagcctctggattcgacttcagtagcggtggaatgtgctgggtccgccaggctccagggaaggggctggagtggatcggatgcattgtaaatggtgatggtagcacatacttcgcgacctgggcgaaaggccgattcaccatcaccagaacgtcgtcgaccacggtgactctgcaaatgaccagtctgacagccgcggacacggccacctatttctgttggagatattgggccttgtggggcccaggtaccctggtcaccgtctcctca

SEQ ID NO: 192 - DNA, 648 bp, Artificial Sequence
  anti-ATM 1561 light chain coding sequence (clone EB0608D-4A10-K1) without leader sequence
atcgtgatgacccagactccatcttccaagtctgtccctgtgggaggcacagtcaccatcaattgccaggccagtgagagtctttataataacaaccgcttagcctggtttcagcagaaaccagggcagcctcccaagctcctgatctattctgcatccactctggcatctggggtcccagataggttcagcggcagtggatctgggacacagttcactctcaccatcagcgatgtggtgtgtgacgacgctgccacttactactgcgtaggaggtagaagtcgtgttactgatggtattgctttcggcggagggaccgaggcggtggtcaaaggtgatccagttgcacctactgtcctcatcttcccaccagctgctgatcaggtggcaactgaaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 193 - DNA, 333 bp, Artificial Sequence
  anti-ATM 1561 variable light domain coding sequence without leader sequence
atcgtgatgacccagactccatcttccaagtctgtccctgtgggaggcacagtcaccatcaattgccaggccagtgagagtctttataataacaaccgcttagcctggtttcagcagaaaccagggcagcctcccaagctcctgatctattctgcatccactctggcatctggggtcccagataggttcagcggcagtggatctgggacacagttcactctcaccatcagcgatgtggtgtgtgacgacgctgccacttactactgcgtaggaggtagaagtcgtgttactgatggtattgctttcggcggagggaccgaggcggtggtcaaa

SEQ ID NO: 194 - DNA, 357 bp, Artificial Sequence
  anti-CD19 41 variable heavy domain coding sequence without leader sequence
cagatccagttggtgcagtctggacctgagctgaagaagcctggagagacagtcaagatctcctgcaaggcttctgattataccttcacagactattcaatacactgggtgaagcaggctccaggaaagggtttaaagtggctgggctggataaacactgagactgttgagccagcatatgcagttgacttcaggggacggtttgcgctgtctttggaaacctctgccaatactgcctatttgcagatcaacaacctcaaaaatgaggacgcggctacatatttctgtactagagactatagtagagagggcgtctttgactactggggccaaggcaccactctcacagtctcctca

SEQ ID NO: 195 - DNA, 336 bp, Artificial Sequence
  anti-CD19 41 variable light domain coding sequence without leader sequence
gacaatgtgatgtcacagtctccttcctccctggctgtgtcagcaggagagaaggtcactatgagctgcaaatccagtcagagtctattcaacagtagaacccgaaagaactacttggcttggtaccagcagaaaccagggcagtctcctaaacttctggtctactgggcatccactagggaatctggggtccctgatcgcttcacaggcagtgggtctgggacagatttcactctcaccatcagcaatgtgcaggctgaagacctggcagtttattattgtaagcaatcctataatcttcccacgttcggtgctgggaccaagctggagctgaaa

SEQ ID NO: 196 - DNA, 1314 bp, Artificial Sequence
  anti-ADA 93 heavy chain coding sequence (clone EB0608A-3C12-H2) without leader sequence
caggagcagctggaggagtccgggggagacctggtcaagcctgggtcatccctgacactcacctgcacagcctctggattctccttcactaacagcgactgggaatgctgggtccgccaggctccagggaaggggctggagtggatcgcatgcatttatggtgatagtcgtagcggatactacgcgagctgggcgaagggccgattcaccatctccaaaacctcgtcgaccacggtgactctgcaaatgaccactctgacagccgcggacacggccacctatttctgtgcgagaaatctcggtagcttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 197 - DNA, 342 bp, Artificial Sequence
  anti-ADA 93 variable heavy domain coding sequence without leader sequence
caggagcagctggaggagtccgggggagacctggtcaagcctgggtcatccctgacactcacctgcacagcctctggattctccttcactaacagcgactgggaatgctgggtccgccaggctccagggaaggggctggagtggatcgcatgcatttatggtgatagtcgtagcggatactacgcgagctgggcgaagggccgattcaccatctccaaaacctcgtcgaccacggtgactctgcaaatgaccactctgacagccgcggacacggccacctatttctgtgcgagaaatctcggtagcttgtggggcccaggcaccctggtcaccgtctcctca

SEQ ID NO: 198 - DNA, 651 bp, Artificial Sequence
  anti-ADA 93 light chain coding sequence (clone EB0608A-3C12-K2) without leader sequence
caagtgctgacccagactccatcctccgtgtctgcggctgtgggaggcacagtcaccatcaactgccagtccagtcagaatgttgtttttaacaaccgcttagcctggtttcagcagaaaccagggcagcctcccaaactcctgatctaccaggcatccactctggcatctggggtcccaccgcggttcaaaggcagtggatctgggacacagttcactctcaccattagcgacctggagtgtgacgatgctgccacttactactgtctaggcagttatgattgtagtagtactgattgtaatactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 199 - DNA, 336 bp, Artificial Sequence
  anti-ADA 93 variable light domain coding sequence without leader sequence
caagtgctgacccagactccatcctccgtgtctgcggctgtgggaggcacagtcaccatcaactgccagtccagtcagaatgttgtttttaacaaccgcttagcctggtttcagcagaaaccagggcagcctcccaaactcctgatctaccaggcatccactctggcatctggggtcccaccgcggttcaaaggcagtggatctgggacacagttcactctcaccattagcgacctggagtgtgacgatgctgccacttactactgtctaggcagttatgattgtagtagtactgattgtaatactttcggcggagggaccgaggtggtggtcaaa

SEQ ID NO: 200 - DNA, 345 bp, Artificial Sequence
  anti-DOCK8 1272 variable heavy domain coding sequence (Hybridoma SJSH5-1C2-2E) without leader sequence
gaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttttcctggaaatagtaatactagatacaaccagaagttcaagggcaaggccaaactgactgcagtcacatctgccagcactgcctacatggagctcagcagcctgacaaatgaggactctgcggtctattactgtgccgatgataacgcctttgactactggggccaaggcaccactctcacagtctcctca

SEQ ID NO: 201 - DNA, 324 bp, Artificial Sequence
  anti-DOCK8 1272 variable light domain coding sequence (Hybridoma SJSH5-1C2-2E) without leader sequence
caaattgttctcacccagtctccagcaatcatgtctgcctctccaggggaaaaggtcaccatgacctgcagtgccagctcaagtgtaagttccaggtacttgcactggtaccagcagaagtcaggagcctcccccaaactctggatttatggcacatccaacctgtcttctggagtccctgatcgcttcagtggcagtgggtctgggacctcttactctctcacaatcagcagcgtggagactgaagatgctgccacttattactgccagcagtatcatagtgacccattcacgttcggcacggggacaaaattggaaataaaa

SEQ ID NO: 202 - DNA, 342 bp, Artificial Sequence
  anti-CD42 128 variable heavy domain coding sequence (Hybridoma SJSH4 3B8-1b) without leader sequence
caggcttatctacagcagtctggggctgttctggtgaggcctggggcctcagtgaagatgtcctgcaaggcttctggctacacattttccagttacaatatacactgggtaaagcagacacctagacagggcctggaatggattggaggtatttatccaggaaatggtgacacttcctacaataagaagttcaaggacaaggccacactgactgttgacaaatcctccaacaccgcctacatgcacctcagcagcctgacatctgaagactctgcggtctttttctgtgcaagcgacggtactaggtactggggccaaggcaccactctcacagtctcctca

SEQ ID NO: 203 - DNA, 336 bp, Artificial Sequence
  anti-CD42 128 variable light domain coding sequence (Hybridoma SJSH4 3B8-1b) without leader sequence
gaagttgtgatgacccagactccactcactttgtcggttaccattggacaagcagtctccatttcttgtacgtcaagtcagagcctcttagctagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgacgatttgggagtttattgttgctggcaaggtgcacattctccgctcacgttcggtgctgggaccaggctggaactgaaa

SEQ ID NO: 204 - DNA, 351 bp, Artificial Sequence
  anti-CD42 154 variable heavy domain coding sequence (Hybridoma SJSH4-1H11c) without leader sequence
gaggtccagctgcaacagtctggacctgagctggtgaagcctggggcttcagtgaagatgtcctgtaaggcttctggatacacattcactggctacaatatgcactgggtgaagcagagccatggaaagagccttgagtggattggagacattaatcctaagactggtgaaaatagttataaccagaagttcaagggcaaggccacattgacgataagcaagtcctccagcacagcctacatggagctccgcagtctgacatcggaagattctgcagtctattactgtgtaagatggggtgtatattatttagaccactggggccaaggcaccactctcacggtctcctca

SEQ ID NO: 205 - DNA, 336 bp, Artificial Sequence
  anti-CD42 154 variable light domain coding sequence (Hybridoma SJSH4-1H11c) without leader sequence
gatattgtgatgacgcaggctgcattctccaatccagtcactcttggaacgtcagcttccatctcctgcaggtctactaagagtctcctatatagtgatggcatcacttatttgtattggtatctgcagaggccaggccagtctcctcagctcctgatatatcggatgtccgaccttgcctcaggagtcccagacaggttcagtggcagtgggtcaggaactgatttcacactgagaatcagcagagtggaggctgaggatgtgggtgtttattactgtgctcaaatggtagaattccctccgacgttcggtggaggcaccaagctggaaatcaaa

SEQ ID NO: 206 - DNA, 1347 bp, Artificial Sequence
  anti-CD56 122 heavy chain coding sequence (clone EB0608C-3H4-H2) without leader sequence
cagtcgttggaggagtccgggggagacctggtcaagcctggggcatccctgacactcacctgcaaagcctctgggttagacttcagtagcaactactacatgtgctgggtccgccaggctccagggaaggggctggagtggatcgcatgcatttatagtagtagtcttaacacatggtacgcgagctgggcgaaaggccgattcaccatctccaaaagcaccagcctacacacggtgactctgcaaatgaccagtctgacagccgcggacacggccacctatttctgtacgagagggggtggtgtttatgttggtgctgattatggtttttacttctacttatggggcccaggcaccctggtcaccgtctcctccgggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 207 - DNA, 375 bp, Artificial Sequence
  anti-CD56 122 variable heavy domain coding sequence without leader sequence
cagtcgttggaggagtccgggggagacctggtcaagcctggggcatccctgacactcacctgcaaagcctctgggttagacttcagtagcaactactacatgtgctgggtccgccaggctccagggaaggggctggagtggatcgcatgcatttatagtagtagtcttaacacatggtacgcgagctgggcgaaaggccgattcaccatctccaaaagcaccagcctacacacggtgactctgcaaatgaccagtctgacagccgcggacacggccacctatttctgtacgagagggggtggtgtttatgttggtgctgattatggtttttacttctacttatggggcccaggcaccctggtcaccgtctcctcc

SEQ ID NO: 208 - DNA, 648 bp, Artificial Sequence
  anti-CD56 122 light chain coding sequence (clone EB0608C-3H4-K2) without leader sequence
gctgacattgtgatgacccagactccatcctccgtggaggcacctgtgggaggcacagtcaccatcaagtgccaggccagtcagagcattgatagttggtgtgcctggtatcagcagaaaccagggcagcctcccaagctcctgatctacaaggcatccactctggaatctggggtcccatcgcggttcaaaggcagtggatctgggacagagttcactctcaccatcagcgacctggagtgtgccgatgctgccacttacttctgtcaaagctattatcctactactggtagtgataatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 209 - DNA, 333 bp, Artificial Sequence
  anti-CD56 122 variable light domain coding sequence without leader sequence
gctgacattgtgatgacccagactccatcctccgtggaggcacctgtgggaggcacagtcaccatcaagtgccaggccagtcagagcattgatagttggtgtgcctggtatcagcagaaaccagggcagcctcccaagctcctgatctacaaggcatccactctggaatctggggtcccatcgcggttcaaaggcagtggatctgggacagagttcactctcaccatcagcgacctggagtgtgccgatgctgccacttacttctgtcaaagctattatcctactactggtagtgataatgctttcggcggagggaccgaggtggtggtcaaa

SEQ ID NO: 210 - protein, 8 aa, Homo sapiens
IASGFFLR

SEQ ID NO: 211 - protein, 7 aa, Homo sapiens
YLVIDNK

SEQ ID NO: 212 - protein, 9 aa, Homo sapiens
ESPLKPFLK

SEQ ID NO: 213 - protein, 21 aa, Homo sapiens
NLEDLVTEYHGNFSAWSGVSK

SEQ ID NO: 214 - protein, 13 aa, Homo sapiens
GLAESLQPDYSER

SEQ ID NO: 215 - protein, 12 aa, Homo sapiens
NIGSDEDHLSLK

SEQ ID NO: 216 - protein, 9 aa, Homo sapiens
IPIEELEDR

SEQ ID NO: 217 - protein, 12 aa, Homo sapiens
LSGAADTQALLR